(19) United States
(12) Patent Application Publication
(10) Pub. No.: US 2025/0011381 A1
(43) Pub. Date: Jan. 9, 2025

(54) POLYPEPTIDES CAPABLE OF PRODUCING GLUCANS HAVING ALPHA (1-->2) LINKAGES AND USE OF THE SAME

(71) Applicant: NUTRITION & BIOSCIENCES USA 4, INC., Rochester, NY (US)

(72) Inventors: QIONG CHENG, WILMINGTON, DE (US); ROBERT DICOSIMO, CHADDS FORD, PA (US); JAHNAVI CHANDRA PRASAD, WILMINGTON, DE (US); ZHENGHONG ZHANG, SHANGHAI (CN)

(21) Appl. No.: 18/661,777

(22) Filed: May 13, 2024

Related U.S. Application Data
(63) Continuation of application No. 17/084,945, filed on Oct. 30, 2020, now Pat. No. 11,981,712.
Application No. 17/084,945 is a continuation of application No. 15/767,689, filed on Apr. 12, 2018, now Pat. No. 10,822,383.

(30) Foreign Application Priority Data
Nov. 26, 2015 (WO) PCT/CN2015/095687
Jun. 13, 2016 (WO) PCT/CN2016/085547

(51) Int. Cl. C07K 14/47; A23L 33/10; A23L 33/135; A23L 33/21; A61K 8/60; A61K 8/64; A61K 8/73; A61K 31/716; A61Q 19/00; C08B 37/00; C08L 5/00; C08L 5/02; C11D 3/22; C12N 9/10; C12P 19/04; C12P 19/18

(52) U.S. Cl. CPC C07K 14/4725; A23L 33/10; A23L 33/135; A23L 33/21; A61K 8/60; A61K 8/64; A61K 8/73; A61K 31/716; A61Q 19/00; C08B 37/0009; C08L 5/00; C08L 5/02; C12N 9/1051; C12P 19/04; C12P 19/18; C12Y 204/00; C12Y 204/01; C12Y 204/01005; C12Y 302/01; A23V 2002/00; A23V 2200/00; A61K 2800/10; C11D 3/22

(57) ABSTRACT

Disclosed herein are proteins capable of forming glucans having alpha-1,2 linkages/branches, reactions and methods for producing such glucan, compositions comprising such glucan, and various applications thereof.

This application claims the benefit of International Application Nos. PCT/CN2015/095687 (filed Nov. 26, 2015) and PCT/CN2016/085547 (filed Jun. 13, 2016), both of which are incorporated herein by reference in their entireties.

### FIELD OF INVENTION

The disclosure relates to, for example, proteins that are capable of forming glucan having alpha-1,2 linkages, reactions and methods for producing such glucan, compositions comprising this glucan, and various applications of using this glucan.

### REFERENCE TO SEQUENCE LISTING SUBMITTED ELECTRONICALLY

The official copy of the sequence listing is submitted electronically via EFS-Web as an ASCII formatted sequence listing with a file named 20161122_CL6550WOPCT3_SequenceListing.txt created on Nov. 21, 2016 and having a size of 413 kilobytes and is filed concurrently with the specification. The sequence listing contained in this ASCII-formatted document is part of the specification and is herein incorporated by reference in its entirety.

### BACKGROUND

The enzymatic addition of alpha-1,2 branching to dextrans has been reported. A glucosyltransferase (DsrE) from Leuconostoc mesenteroides NRRL B-1299 has a second catalytic domain (“CD2”) capable of adding alpha-1,2 branching to dextrans (U.S. Pat. Nos. 7,439,049 and 5,141,858; U.S. Patent Appl. Publ. No. 2009-0123448; Bozonnet et al., J. Bacteriol. 184:5753-5761, 2002). U.S. Patent Appl. Pub. No. 2010-0284972 describes methods and compositions for improving the health of a subject by administering compositions comprising alpha-1,2-branched alpha-1,6 dextrans. Sarbini et al. (Appl. Environ. Microbiol. 77:5307-5315, 2011) describes in vitro fermentation of dextran and alpha-1,2-branched dextrans by human fecal microbiota. Brison et al. (J. Biol. Chem. 287:7915-7924, 2012) describes a truncated form of the DsrE glucosyltransferase from Leuconostoc mesenteroides NRRL B-1299 (a glucan binding domain [GBD] coupled to the second catalytic domain, CD2 [i.e., GBD-CD2]) that is capable of adding alpha-1,2 branching to dextrans. Despite these reports, there remains a need to identify further enzymes that are capable of producing glucans having alpha-1,2 linkages.

### SUMMARY

In one aspect, the present disclosure regards a reaction composition comprising at least water, sucrose, an alpha-glucan substrate, and a polypeptide that is capable of forming at least one alpha-1,2 branch from the alpha-glucan substrate, wherein the polypeptide comprises an amino acid sequence that is at least 90% identical to:
(i) the mature form of a sequence selected from the group consisting of SEQ ID NOs:4, 1, 2, 3, 5, 6, 7, 8, 9, 10, 11, 12, and 13;
(ii) SEQ ID NO:27 or a sub-sequence within any one of SEQ ID NOs:4, 2, 3, 5, 6, 7, 8, 9, 10, 11, 12, or 13 that aligns with SEQ ID NO:27; and/or
(iii) a sequence selected from the group consisting of SEQ ID NOs:4, 1, 2, 3, 5, 6, 7, 8, 9, 10, 11, 12, and 13.

The present disclosure also concerns a method of producing a glucan composition that comprises alpha-1,2 linkages, the method comprising:
(a) providing at least the following reaction components: water, sucrose, an alpha-glucan substrate, and a polypeptide that is capable of forming at least one alpha-1,2 branch from the alpha-glucan substrate, wherein the polypeptide comprises an amino acid sequence that is at least 90% identical to:
   (i) the mature form of a sequence selected from the group consisting of SEQ ID NOs:4, 1, 2, 3, 5, 6, 7, 8, 9, 10, 11, 12, and 13;
   (ii) SEQ ID NO:27 or a sub-sequence within any one of SEQ ID NOs:4, 2, 3, 5, 6, 7, 8, 9, 10, 11, 12, or 13 that aligns with SEQ ID NO:27; and/or
   (iii) a sequence selected from the group consisting of SEQ ID NOs:4, 1, 2, 3, 5, 6, 7, 8, 9, 10, 11, 12, and 13;
(b) combining the reaction components under suitable conditions whereby the polypeptide catalyzes the synthesis of at least one alpha-1,2 branch from the alpha-glucan substrate, thereby forming a glucan composition comprising alpha-1,2 linkages; and
(c) optionally isolating the glucan composition comprising alpha-1,2 linkages.

The present disclosure also concerns a composition that comprises a glucan composition comprising one or more alpha-1,2 linkages produced by a method or reaction as described herein, preferably wherein the composition is in the form of a food product, pharmaceutical product, personal care product, household care product, or industrial product, optionally wherein the composition comprises about 0.01 to 99 wt % (dry solids basis) of the glucan composition.

The present disclosure also concerns a method comprising enterally administering a substance to a mammal, wherein the substance comprises a glucan composition comprising alpha-1,2 linkages, wherein the administering results in less or slower blood glucose elevation in the mammal as compared to a mammal that is enterally administered a substance that lacks the glucan composition but instead contains a same amount of a readily digestible glucose-containing carbohydrate, wherein the glucan composition is produced by a method or reaction as described herein, optionally wherein the mammal is a human, and optionally wherein the readily digestible glucose-containing carbohydrate is sucrose, free glucose, or starch.

The present disclosure also concerns a method of producing a food or beverage, the method comprising incorporating a glucan composition comprising alpha-1,2 linkages into the food or beverage, wherein the glycemic index of the resulting food or beverage is not increased, or only marginally increased, compared to a food or beverage that lacks the glucan composition, and wherein the glucan composition is produced by a method or reaction as described herein.

### BRIEF DESCRIPTION OF THE BIOLOGICAL SEQUENCES

The identification (gi) and accession numbers provided below are from GENEBANK (available at National Center for Biotechnology Information [NCBI] website).

SEQ ID NO:1 is the amino acid sequence of full length GTFJ18 (old gi: 356644413, new gi: 504090610, Acc. No. WP_014324604.1, Leuconostoc mesenteroides). The predicted mature secreted form of GTFJ18 is believed to correspond to positions 21-2771 of SEQ ID NO:1. SEQ ID NOs:1 and 13 are identical.

SEQ ID NO:2 is the amino acid sequence from gi: 116096814 (GENBANK Acc. No. ABJ61965.1, Leuconostoc mesenteroides; also referred to herein as GTF6814). The predicted mature secreted form of GTF6814 is believed to correspond to positions 21-2821 of SEQ ID NO:2.

SEQ ID NO:3 is the amino acid sequence from gi: 916260333 (GENBANK Acc. No. WP_050995379.1, Leuconostoc carnosum; also referred to herein as GTF0333). The predicted secreted mature form of GTF0333 is believed to correspond to positions 41-2844 of SEQ ID NO:3.

SEQ ID NO:4 is the amino acid sequence from gi: 902949905 (GENBANK Acc. No. GAP05007.1, Fructobacillus tropaeoli; also referred to herein as GTF9905 or FtrGtf1). The predicted expressed form (contemplated to be a secreted, mature form) of GTF9905 is believed to correspond to positions 36-1672 of SEQ ID NO:4.

SEQ ID NO:5 is the amino acid sequence from gi: 938153845 (GENBANK Acc. No. WP_054608463.1, Lactobacillus kunkeei; also referred to herein as GTF3845). The predicted mature secreted form of GTF3845 is believed to correspond to positions 51-1632 of SEQ ID NO:5.

SEQ ID NO:6 is the amino acid sequence from gi: 938153846 (GENBANK Acc. No. WP_054608464.1, Lactobacillus kunkeei; also referred to herein as GTF3846). The predicted mature secreted form of GTF3846 is believed to correspond to positions 51-1318 of SEQ ID NO:6.

SEQ ID NO:7 is the amino acid sequence from gi: 927068954 (GENBANK Acc. No. KOY70706.1, Lactobacillus kunkeei; also referred to herein as GTF8954). The predicted mature secreted form of GTF8954 is believed to correspond to positions 51-1139 of SEQ ID NO:7.

SEQ ID NO:8 is the amino acid sequence from gi: 927268464 (GENBANK Acc. No. WP_053795842.1, Lactobacillus kunkeei; also referred to herein as GTF8464). The predicted mature secreted form of GTF8464 is believed to correspond to positions 51-1463 of SEQ ID NO:8.

SEQ ID NO:9 is the amino acid sequence from gi: 908395133 (GENBANK Acc. No. WP_049752804.1, Leuconostoc mesenteroides; also referred to herein as GTF5133). The predicted mature secreted form of GTF5133 is believed to correspond to positions 41-2841 of SEQ ID NO:9.

SEQ ID NO:10 is the amino acid sequence from gi: 935566432 (GENBANK Acc. No. WP_054450649.1, Lactobacillus kunkeei; also referred to herein as GTF6432). The predicted mature secreted form of GTF6432 is believed to correspond to positions 46-2580 of SEQ ID NO:10.

SEQ ID NO:11 is the amino acid sequence from gi: 916985575 (GENBANK Acc. No. WP_051592287.1, Lactobacillus kunkeei; also referred to herein as GTF5575). The predicted mature secreted form of GTF5575 is believed to correspond to positions 51-1463 of SEQ ID NO:11.

SEQ ID NO:12 is the amino acid sequence from gi: 407242790 (GENBANK Acc. No. AFT82440.1, Leuconostoc carnosum JB16; also referred to herein as GTF2790). The predicted mature secreted form of GTF2790 is believed to correspond to positions 21-2824 of SEQ ID NO: 12.

SEQ ID NO:13 is the amino acid sequence from gi: 504090610 (GENBANK Acc. No. WP_014324604.1, Leuconostoc mesenteroides; also referred to herein as GTF0610). The predicted mature secreted form of GTF0610 is believed to correspond to positions 21-2771 of SEQ ID NO:13.

SEQ ID NO:14 is the DNA sequence encoding full length GTFJ18 (SEQ ID NO:1).

SEQ ID NO:15 is the DNA sequence of nucleotides 846926-855391 from gb: CP000414.1, and encodes GTF6814 (SEQ ID NO:2).

SEQ ID NO:16 is the DNA sequence of the complementary sequence to nucleotides 1620046-1611512 from gb: CP003851.1, and encodes GTF0333 (SEQ ID NO:3).

SEQ ID NO:17 is the DNA sequence of nucleotides 237-5252 from gi: 850934366, and encodes GTF9905 (SEQ ID NO:4). SEQ ID NO:34 is a codon-optimized sequence encoding the expressed form of GTF9905 (SEQ ID NO:4).

SEQ ID NO:18 is the DNA sequence of the complementary sequence to nucleotides 13658-8760 from gb: JXDF01000026.1, and encodes GTF3845 (SEQ ID NO:5).

SEQ ID NO:19 is the DNA sequence of the complementary sequence of nucleotides 17742-13786 from gb: JXDF01000026.1, and encodes GTF3846 (SEQ ID NO:6).

SEQ ID NO:20 is the DNA sequence of the complementary sequence to nucleotides 99099-95680 from gb: JXCW01000006.1, and encodes GTF8954 (SEQ ID NO:7).

SEQ ID NO:21 is the DNA sequence of the complementary sequence to nucleotides 23080-18689 from gb: JXCU01000040.1, and encodes GTF8464 (SEQ ID NO:8).

SEQ ID NO:22 is the DNA sequence of nucleotides 846866-855391 from gb: CP000414.1, and encodes GTF5133 (SEQ ID NO:9).

SEQ ID NO:23 is the DNA sequence of the complementary sequence to nucleotides 7742-3 from gb: JXDB01000011.1, and encodes GTF6432 (SEQ ID NO:10).

SEQ ID NO:24 is the DNA sequence of the complementary sequence to nucleotides 4456-65 from gb: AZBY01000038.1, and encodes GTF5575 (SEQ ID NO:11).

SEQ ID NO:25 is the DNA sequence of the complementary sequence to nucleotides 1619986-1611512 from gb: CP003851.1, and encodes GTF2790 (SEQ ID NO:12).

SEQ ID NO:26 is the DNA sequence of nucleotides 845078-853513 from gb: CP003101.3, and encodes GTF0610 (SEQ ID NO: 13).

SEQ ID NO:27 is the amino acid sequence of GTFJ18T1, which is represents an N-terminal-truncated (first 1664 residues removed) form of GTFJ18 (SEQ ID NO: 1).

SEQ ID NO:28 is the amino acid sequence of the GTFJ18 CD2.

SEQ ID NO:29 is a codon-optimized nucleotide sequence encoding mature GTF8117 (SEQ ID NO:30) (with an added start-methionine) of the Lactobacillus animalis KCTC 3501 protein of GENBANK Acc. No. KRM57462.1.

SEQ ID NO:31 is a nucleotide sequence encoding mature GTF6831 (SEQ ID NO:32) of the Streptococcus salivarius M18 protein of GENBANK Acc. No. WP_004182667.1.

SEQ ID NO:33 is the amino acid sequence of GTF5604, which is derived from Streptococcus criceti HS-6 (GENBANK® Acc. No. WP_004226213.1, old gi: 357235604; also referred to as SG1018 glucosyltransferase or GtfHS6). The mature form of GTF5604 is predicted to begin at amino acid position 37.

SEQ ID NO:34 is a codon-optimized sequence encoding the expressed form of GTF9905 (SEQ ID NO:4).

### DETAILED DESCRIPTION

The disclosures of all cited patent and non-patent literature are incorporated herein by reference in their entirety.

The articles “a”, “an”, and “the” preceding an element or component are intended to be nonrestrictive regarding the number of instances (i.e., occurrences) of the element or component. Therefore “a”, “an”, and “the” should be read to include one or at least one, and the singular word form of the element or component also includes the plural unless the number is obviously meant to be singular.

Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like.

The terms “alpha-glucan”, “alpha-glucan polymer” and the like are used interchangeably herein. An alpha-glucan is a polymer comprising glucose monomeric units linked together by alpha-glycosidic linkages (can also be referred to as alpha-glucosidic linkages).

The terms “linkage”, “glycosidic linkages”, “glycosidic bonds” and the like refer to the covalent bonds connecting the sugar monomers within a saccharide compound (oligosaccharides and/or polysaccharides). Examples of glycosidic linkages include alpha-linked glucose oligomers with 1,6-alpha-D-glycosidic linkages (herein also referred to as “alpha-1,6” linkages); 1,3-alpha-D-glycosidic linkages (herein also referred to as “alpha-1,3” linkages); 1,4-alpha-D-glycosidic linkages (herein also referred to as “alpha-1,4” linkages); 1,2-alpha-D-glycosidic linkages (herein also referred to as “alpha-1,2” linkages); and combinations of such linkages typically associated with branched saccharide oligomers.

The terms “glucosyltransferase”, “glucosyltransferase enzyme”, “GTF”, “glucansucrase” and the like are used interchangeably herein. The activity of a glucosyltransferase herein catalyzes the reaction of the substrate sucrose to make the products alpha-glucan and fructose. Some byproducts of a glucosyltransferase reaction may include glucose and/or leucrose, for example. Wild type forms of glucosyltransferase enzymes generally contain (in the N-terminal to C-terminal direction) a signal peptide, a variable domain, a catalytic domain, and a glucan-binding domain. An example of a glucosyltransferase herein is a 1,2-branching enzyme.

The terms “reaction composition”, “enzymatic reaction”, “glucosyltransferase reaction”, “glucan synthesis reaction”, and the like are used interchangeably herein and generally refer to a reaction that initially comprises water, sucrose, at least one active glucosyltransferase enzyme, and optionally other components. For a 1,2-branching reaction composition herein, another component that is included is an alpha-glucan substrate. Components that can be further present in a glucosyltransferase reaction typically after it has commenced include the products fructose and alpha-glucan, and optionally byproducts such as glucose and leucrose. It would be understood that in embodiments in which a glucosyltransferase has 1,2-branching activity, the alpha-glucan product can represent the 1,2-branch material synthesized by the enzyme, and/or the entire alpha-glucan product itself (i.e., alpha-glucan substrate plus added 1,2-branches). The term “under suitable conditions” as used herein refers to reaction conditions that support conversion of sucrose to products fructose and alpha-glucan via glucosyltransferase enzyme activity.

The term “polypeptide that is capable of forming at least one alpha-1,2 branch from an alpha-glucan substrate” refers to a catalytically active glucosyltransferase (or active fragment thereof) capable of introducing one or more alpha-1,2 glycosidic linkages (using sucrose as an additional substrate) to an alpha-glucan substrate (or “alpha-glucan backbone”) via one or more branches (can also be referred to herein as a “1,2-branching enzyme” or with other like terms). The polypeptide is believed to add one glucose group per branch. In certain embodiments, such a polypeptide is a truncated glucosyltransferase that includes a catalytic domain capable of performing alpha-1,2 branching from an alpha-glucan substrate. It would be recognized by one of skill in the art that truncations can encompass the deletion of amino acids in either or both N and C-terminal directions relative to the catalytic domain capable of adding alpha-1,2 branching that are present in a wild-type sequence. For example, N-terminal truncations may be produced from genes which encode a glucosyltransferase starting from a delayed start codon and C-terminal truncations may be produced from genes which encode a glucosyltransferase ending at a premature stop codon. In certain embodiments, a polypeptide includes at least one glucan binding domain in addition to the catalytic domain. In certain embodiments, a polypeptide is a truncated glucosyltransferase that includes a catalytic domain capable of adding alpha-1,2 branching to an alpha-glucan substrate backbone alone or in combination with a glucan binding domain and that does not include a domain capable of synthesizing linkages other than alpha-1,2 glycosidic linkages.

An “alpha-glucan substrate” or “alpha-glucan backbone” (and like terms) as referred to herein can comprise (i) alpha-1,6 or (ii) alpha-1,6 and alpha-1,3 glycosidic linkages, for example, and typically has a degree of polymerization (DP) of at least 3 and is typically water-soluble. In typical embodiments, an alpha-glucan substrate is capable of being modified (i.e., the addition of at least one alpha-1,2 glycosidic linkage) under aqueous reaction conditions by a polypeptide having alpha-1,2 branching activity in the presence of sucrose. “Beta-glucan” is typically excluded as being part of an alpha-glucan substrate herein.

An “alpha-1,2 branch” (and like terms) as referred to herein can be from an alpha-glucan substrate backbone herein, for example. Where an alpha-1,2 branch stems from a 1,6-linked glucose of a backbone, such 1,2-linkage can also be referred to as alpha-1,2,6. A branch that is alpha-1,2-inked to an alpha-glucan backbone herein typically has one glucose group (can optionally be referred to as a pendant glucose). The percent of 1,2-branching in a glucan herein refers to that percentage of all the linkages in the glucan that represent 1,2-branch points (e.g., 1,2,6).

Herein, “alpha-1,3,6” refers to a branch point in which the branch glucose is alpha-1,3-linked to a 1,6-linked glucose monomer of a backbone.

A glucan product of a 1,2-branching reaction herein (e.g., “glucan composition comprising alpha-1,2 linkages”, “alpha-1,2-branched glucan composition”, “a glucan herein” and like terms) can be characterized, for example, in terms of (i) any alpha-glucan substrate herein plus (ii) any added 1,2-branching herein.

The terms “mature”, “secreted”, “mature secreted” and the like are used interchangeably herein. A mature protein is one that can pass through the cellular membrane of a cell, particularly a bacterial cell. A mature protein in some aspects results from post-translational removal (cleavage away) of a “signal sequence” (or “signal peptide”) from the N-terminus of the protein's immature (preprocessed) form. A signal sequence typically directs an immature protein to the cell membrane, and is removed from the protein during transit thereof through the membrane (i.e., during the protein secretion process). Heterologous expression herein of a mature protein can employ a signal sequence, in which case the likely goal is secretion of the protein to the surrounding media. Alternatively, heterologous expression can employ expressing a protein designed to already lack its signal sequence (a start methionine is typically added to the N-terminus in such embodiments); such mature protein expression typically entails lysing cells to release the protein, since it is not secreted. A signal sequence herein can either be native or heterologous with respect to the protein with which it is optionally employed.

The term “dextran” herein refers to a water-soluble alpha-glucan comprising at least 50% alpha-1,6 glycosidic linkages (typically with up to 49% alpha-1,3 glycosidic linkages, some of which may occur at branching points). Dextrans often have an average molecular weight above 1000 kDa. Enzymes capable of synthesizing dextran from sucrose may be described as “dextransucrases” (EC 2.4.1.5). A dextran is an example of a suitable alpha-glucan substrate herein.

The terms “alpha-glucanohydrolase”, “glucanohydrolase” and the like as used herein refer to an enzyme capable of endo- or exo-hydrolyzing an alpha-glucan oligomer. A glucanohydrolase may be defined by its hydrolysis activity towards certain alpha-glycosidic linkages. Examples may include, but are not limited to, dextranases (EC 3.2.1.1; capable of endohydrolyzing alpha-1,6-linked glycosidic bonds), mutanases (EC 3.2.1.59; capable of endohydrolyzing alpha-1,3-linked glycosidic bonds), and alternanases (EC 3.2.1.-; capable of endohydrolytically cleaving alternan). Various factors including, but not limited to, the level of branching, type of branching, and relative branch length within certain alpha-glucans may adversely impact the ability of an alpha-glucanohydrolase to hydrolyze some glycosidic linkages.

The molecular weight of a glucan herein (e.g., alpha-glucan substrate or glucan composition comprising alpha-1,2 linkages) can be represented as degree of polymerization (DP), Daltons, or as grams/mole. DP refers to the number of glucoses comprised within a glucan (e.g., a glucan of DP 10 means that the glucan contains 10 glucoses). Various means are known in the art for calculating molecular weight measurements such as with high-pressure liquid chromatography (HPLC), size exclusion chromatography (SEC), or gel permeation chromatography (GPC).

The term “water-soluble” as used herein characterizes a glucan that has the capability of dissolving in water and/or an aqueous solution herein, where the entire glucan molecule is dissolvable. Typically, the conditions for such solubility include a water/solution temperature range of about 1 to 85° C., which includes temperatures suitable for various uses, such as in beverages and/or household care applications.

The terms “percent by volume”, “volume percent”, “vol %”, “v/v %” and the like are used interchangeably herein. The percent by volume of a solute in a solution can be determined using the formula: [(volume of solute)/(volume of solution)] x 100%.

The terms “percent by weight”, “weight percentage (wt %)”, “weight-weight percentage (% w/w)” and the like are used interchangeably herein. Percent by weight refers to the percentage of a material on a mass basis as it is comprised in a composition, mixture, or solution.

A “mammal” herein can be a human, pet (e.g., feline, canine), domesticated/raised mammal (e.g., bovine, porcine, equine, ovine), or rodent or other small mammal (e.g., mouse, rat, rabbit) for example.

The term “enteral administration” and like terms refer to feeding or drug administration via the gastrointestinal (GI) tract. This contrasts with parenteral administration, which occurs from routes outside the GI tract (e.g., intravenous). While most instances herein of enteral administration are performed by ingestion (i.e., by mouth; orally), some instances can be via direct delivery to the esophagus or stomach (e.g., using a feeding tube).

The term “glycemic index” as used herein refers to a number associated with a particular type of food that indicates the food's effect on a mammal's blood glucose (blood sugar) level. A value of 100 represents the standard, which is an equivalent amount of pure glucose. A low glycemic index is typically about 55 or less, a medium glycemic index is typically about 56 to 69, and a high glycemic index is typically about 70 or above. The term “glycemic response” herein refers to the change in blood glucose levels after consuming a particular food or combination of foods. A “marginal increase” in glycemic index of a food product herein refers to an increase in glycemic index of less than about 1%, 2%, 3%, 4%, 5%, 6%, 7%, 8%, 9%, or 10%, for example.

A “readily digestible glucose-containing carbohydrate” herein refers to a carbohydrate that, upon ingestion by a mammal, quickly raises blood glucose levels in the mammal as compared to ingestion of a “slow glucose release glucan composition” herein. Examples of a readily digestible glucose-containing carbohydrate include sucrose, free glucose, and starch. “Free glucose” herein refers to glucose in free form that is not in glycosidic linkage with another sugar.

The terms “dietary fiber”, “glucan fiber” and like terms herein refer to a glucan herein that is indigestible and/or that does not increase blood-glucose levels when enterally administered to a mammal. In general, a dietary fiber herein is not significantly hydrolyzed by endogenous enzymes in the upper gastrointestinal tract of mammals such as humans.

The terms “aqueous conditions”, “aqueous reaction conditions”, “aqueous setting”, “aqueous system” and the like are used interchangeably herein. Aqueous conditions herein refer to a solution or mixture in which the solvent is at least about 60 wt % water, for example. A branching reaction herein typically is performed under aqueous conditions.

An “aqueous composition” herein has a liquid component that comprises at least about 10 wt % water, for example. Examples of aqueous compositions include mixtures, solutions, dispersions (e.g., colloidal dispersions), suspensions and emulsions, for example. An “aqueous solution” herein refers to a solution in which the solvent comprises water. An aqueous solution can serve as a dispersant in certain aspects herein. An alpha-1,2-branched glucan in certain embodiments can be dissolved within an aqueous solution in certain aspects.

The term “household care product” and like terms refer to products, goods and services relating to the treatment, cleaning, caring and/or conditioning of the home and its contents. The foregoing include, for example, chemicals, compositions, products, or combinations thereof having application in such care.

The terms “fabric”, “textile”, “cloth” and the like are used interchangeably herein to refer to a woven material having a network of natural and/or artificial fibers. Such fibers can be in the form of thread or yarn, for example.

A “fabric care composition” and like terms refer to any composition suitable for treating fabric in some manner. Examples of such a composition include laundry detergents and fabric softeners, which are examples of laundry care compositions.

The terms “heavy duty detergent”, “all-purpose detergent” and the like are used interchangeably herein to refer to a detergent useful for regular washing of white and colored textiles at any temperature. The terms “low duty detergent”, “fine fabric detergent” and the like are used interchangeably herein to refer to a detergent useful for the care of delicate fabrics such as viscose, wool, silk, microfiber or other fabric requiring special care. “Special care” can include conditions of using excess water, low agitation, and/or no bleach, for example.

A “detergent composition” herein typically comprises at least a surfactant (detergent compound) and/or a builder. A “surfactant” herein refers to a substance that tends to reduce the surface tension of a liquid in which the substance is dissolved. A surfactant may act as a detergent, wetting agent, emulsifier, foaming agent, and/or dispersant, for example.

The term “personal care product” and like terms refer to products, goods and services relating to the treatment, cleaning, cleansing, caring or conditioning of the person. The foregoing include, for example, chemicals, compositions, products, or combinations thereof having application in such care.

An “oral care composition” herein is any composition suitable for treating a soft or hard surface in the oral cavity such as dental (teeth) and/or gum surfaces.

The terms “polynucleotide”, “polynucleotide sequence”, “nucleic acid molecule” and the like are used interchangeably herein. These terms encompass nucleotide sequences and the like. A polynucleotide may be a polymer of DNA or RNA that is single- or double-stranded, that optionally contains synthetic, non-natural or altered nucleotide bases. A polynucleotide may be comprised of one or more segments of cDNA, genomic DNA, synthetic DNA, or mixtures thereof.

The term “gene” as used herein refers to a DNA polynucleotide sequence that expresses an RNA (RNA is transcribed from the DNA polynucleotide sequence) from a coding region, which RNA can be a messenger RNA (encoding a protein) or a non-protein-coding RNA. A gene may refer to the coding region alone, or may include regulatory sequences upstream and/or downstream to the coding region (e.g., promoters, 5′-untranslated regions, 3′-transcription terminator regions). A coding region encoding a protein can alternatively be referred to herein as an “open reading frame” (ORF). A gene that is “native” or “endogenous” refers to a gene as found in nature with its own regulatory sequences; such a gene is located in its natural location in the genome of a host cell. A “chimeric” gene refers to any gene that is not a native gene, comprising regulatory and coding sequences that are not found together in nature (i.e., the regulatory and coding regions are heterologous with each other). Accordingly, a chimeric gene may comprise regulatory sequences and coding sequences that are derived from different sources, or regulatory sequences and coding sequences derived from the same source, but arranged in a manner different than that found in nature. A “foreign” or “heterologous” gene can refer to a gene that is introduced into the host organism by gene transfer. Foreign/heterologous genes can comprise native genes inserted into a non-native organism, native genes introduced into a new location within the native host, or chimeric genes. The polynucleotide sequences in certain embodiments disclosed herein are heterologous. A “transgene” is a gene that has been introduced into the genome by a gene delivery procedure (e.g., transformation). A “codon-optimized” open reading frame has its frequency of codon usage designed to mimic the frequency of preferred codon usage of the host cell.

The term “heterologous” means not naturally found in the location of interest. For example, a heterologous gene can be one that is not naturally found in a host organism, but that is introduced into the host organism by gene transfer. As another example, a nucleic acid molecule that is present in a chimeric gene can be characterized as being heterologous, as such a nucleic acid molecule is not naturally associated with the other segments of the chimeric gene (e.g., a promoter can be heterologous to a coding sequence).

A “non-native” amino acid sequence or polynucleotide sequence comprised in a cell or organism herein does not occur in a native (natural) counterpart of such cell or organism. Such an amino acid sequence or polynucleotide sequence can also be referred to as being heterologous to the cell or organism.

“Regulatory sequences” as used herein refer to nucleotide sequences located upstream of a gene's transcription start site (e.g., promoter), 5′ untranslated regions, introns, and 3′ non-coding regions, and which may influence the transcription, processing or stability, and/or translation of an RNA transcribed from the gene. Regulatory sequences herein may include promoters, enhancers, silencers, 5′ untranslated leader sequences, introns, polyadenylation recognition sequences, RNA processing sites, effector binding sites, stem-loop structures, and other elements involved in regulation of gene expression. One or more regulatory elements herein may be heterologous to a coding region herein.

A “promoter” as used herein refers to a DNA sequence capable of controlling the transcription of RNA from a gene. In general, a promoter sequence is upstream of the transcription start site of a gene. Promoters may be derived in their entirety from a native gene, or be composed of different elements derived from different promoters found in nature, or even comprise synthetic DNA segments. Promoters that cause a gene to be expressed in a cell at most times under all circumstances are commonly referred to as “constitutive promoters”. One or more promoters herein may be heterologous to a coding region herein.

A “strong promoter” as used herein refers to a promoter that can direct a relatively large number of productive initiations per unit time, and/or is a promoter driving a higher level of gene transcription than the average transcription level of the genes in a cell.

The terms “3′ non-coding sequence”, “transcription terminator” and “terminator” as used herein refer to DNA sequences located downstream of a coding sequence. This includes polyadenylation recognition sequences and other sequences encoding regulatory signals capable of affecting mRNA processing or gene expression.

The terms “upstream” and “downstream” as used herein with respect to polynucleotides refer to “5′ of” and “3′ of”, respectively.

The term “expression” as used herein refers to (i) transcription of RNA (e.g., mRNA or a non-protein-coding RNA) from a coding region, and/or (ii) translation of a polypeptide from mRNA. Expression of a coding region of a polynucleotide sequence can be up-regulated or down-regulated in certain embodiments.

The term “operably linked” as used herein refers to the association of two or more nucleic acid sequences such that the function of one is affected by the other. For example, a promoter is operably linked with a coding sequence when it is capable of affecting the expression of that coding sequence. That is, the coding sequence is under the transcriptional control of the promoter. A coding sequence can be operably linked to one (e.g., promoter) or more (e.g., promoter and terminator) regulatory sequences, for example.

The term “recombinant” when used herein to characterize a DNA sequence such as a plasmid, vector, or construct refers to an artificial combination of two otherwise separated segments of sequence, e.g., by chemical synthesis and/or by manipulation of isolated segments of nucleic acids by genetic engineering techniques.

The term “transformation” as used herein refers to the transfer of a nucleic acid molecule into a host organism or host cell by any method. A nucleic acid molecule that has been transformed into an organism/cell may be one that replicates autonomously in the organism/cell, or that integrates into the genome of the organism/cell, or that exists transiently in the cell without replicating or integrating. Non-limiting examples of nucleic acid molecules suitable for transformation are disclosed herein, such as plasmids and linear DNA molecules. Host organisms/cells herein containing a transforming nucleic acid sequence can be referred to as “transgenic”, “recombinant”, “transformed”, “engineered”, as a “transformant”, and/or as being “modified for exogenous gene expression”, for example.

The terms “sequence identity”, “identity” and the like as used herein with respect to polynucleotide or polypeptide sequences refer to the nucleic acid residues or amino acid residues in two sequences that are the same when aligned for maximum correspondence over a specified comparison window. Thus, “percentage of sequence identity”, “percent identity” and the like refer to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. It would be understood that, when calculating sequence identity between a DNA sequence and an RNA sequence, T residues of the DNA sequence align with, and can be considered “identical” with, U residues of the RNA sequence. For purposes of determining “percent complementarity” of first and second polynucleotides, one can obtain this by determining (i) the percent identity between the first polynucleotide and the complement sequence of the second polynucleotide (or vice versa), for example, and/or (ii) the percentage of bases between the first and second polynucleotides that would create canonical Watson and Crick base pairs.

Percent identity can be readily determined by any known method, including but not limited to those described in: 1) Computational Molecular Biology (Lesk, A. M., Ed.) Oxford University: NY (1988); 2) Biocomputing: Informatics and Genome Projects (Smith, D. W., Ed.) Academic: NY (1993); 3) Computer Analysis of Sequence Data, Part I (Griffin, A. M., and Griffin, H. G., Eds.) Humana: NJ (1994); 4) Sequence Analysis in Molecular Biology (von Heinje, G., Ed.) Academic (1987); and 5) Sequence Analysis Primer (Gribskov, M. and Devereux, J., Eds.) Stockton: NY (1991), all of which are incorporated herein by reference.

Preferred methods for determining percent identity are designed to give the best match between the sequences tested. Methods of determining identity and similarity are codified in publicly available computer programs, for example. Sequence alignments and percent identity calculations can be performed using the MEGALIGN program of the LASERGENE bioinformatics computing suite (DNASTAR Inc., Madison, WI), for example. Multiple alignment of sequences can be performed, for example, using the Clustal method of alignment which encompasses several varieties of the algorithm including the Clustal V method of alignment (described by Higgins and Sharp, CABIOS. 5:151-153 (1989); Higgins, D. G. et al., Comput. Appl. Biosci., 8:189-191 (1992)) and found in the MEGALIGN v8.0 program of the LASERGENE bioinformatics computing suite (DNASTAR Inc.). For multiple alignments, the default values can correspond to GAP PENALTY=10 and GAP LENGTH PENALTY=10. Default parameters for pairwise alignments and calculation of percent identity of protein sequences using the Clustal method can be KTUPLE=1, GAP PENALTY=3, WINDOW=5 and DIAGONALS SAVED=5. For nucleic acids, these parameters can be KTUPLE=2, GAP PENALTY=5, WINDOW=4 and DIAGONALS SAVED=4. Additionally the Clustal W method of alignment can be used (described by Higgins and Sharp, CABIOS. 5:151-153 (1989); Higgins, D. G. et al., Comput. Appl. Biosci. 8:189-191(1992); Thompson, J. D. et al, Nucleic Acids Research, 22 (22): 4673-4680, 1994) and found in the MEGALIGN v8.0 program of the LASERGENE bioinformatics computing suite (DNASTAR Inc.). Default parameters for multiple alignment (protein/nucleic acid) can be: GAP PENALTY=10/15, GAP LENGTH PENALTY=0.2/6.66, Delay Divergen Seqs (%)=30/30, DNA Transition Weight=0.5, Protein Weight Matrix=Gonnet Series, DNA Weight Matrix=IUB.

Various polypeptide amino acid sequences and polynucleotide sequences are disclosed herein as features of certain embodiments. Variants of these sequences that are at least about 70-85%, 85-90%, or 90%-95% identical to the sequences disclosed herein can be used or referenced. Alternatively, a variant amino acid sequence or polynucleotide sequence can have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity with a sequence disclosed herein. The variant amino acid sequence or polynucleotide sequence has the same function/activity of the disclosed sequence, or at least about 80%, 81%, 82%, 83%, 84%, 85%, 86%, 87%, 88%, 89%, 90%, 91%, 92%, 93%, 94%, 95%, 96%, 97%, 98%, or 99% of the function/activity of the disclosed sequence. Any polypeptide amino acid sequence disclosed herein not beginning with a methionine can typically further comprise at least a start-methionine at the N-terminus of the amino acid sequence.

All the amino acid residues at each amino acid position of the proteins disclosed herein are examples. Given that certain amino acids share similar structural and/or charge features with each other (i.e., conserved), the amino acid at each position of a protein herein can be as provided in the disclosed sequences or substituted with a conserved amino acid residue (“conservative amino acid substitution”) as follows:
1. The following small aliphatic, nonpolar or slightly polar residues can substitute for each other: Ala (A), Ser (S), Thr (T), Pro (P), Gly (G);
2. The following polar, negatively charged residues and their amides can substitute for each other: Asp (D), Asn (N), Glu (E), Gln (Q);
3. The following polar, positively charged residues can substitute for each other: His (H), Arg (R), Lys (K);
4. The following aliphatic, nonpolar residues can substitute for each other: Ala (A), Leu (L), Ile (I), Val (V), Cys (C), Met (M); and
5. The following large aromatic residues can substitute for each other: Phe (F), Tyr (Y), Trp (W).

The terms “aligns with”, “corresponds with”, and the like can be used interchangeably herein. Some embodiments herein relate to a sub-sequence within any one of SEQ ID NOs:4, 2, 3 and 5-13 that aligns with SEQ ID NO:27. A “sub-sequence” herein simply is a portion of any one of SEQ ID NOs:4, 2, 3 and 5-13. A sub-sequence can be characterized to align with SEQ ID NO:27 if it is at least about 50% identical with SEQ ID NO:27, or at least about 65% similar (percent total of both identical sites and conserved sites) with SEQ ID NO:27. In general, one can align the amino acid sequence of a sub-sequence with SEQ ID NO:27 using an alignment algorithm and/or software described herein (e.g., BLASTP, ClustalW, ClustalV, EMBOSS) to determine percent identity and/or similarity.

The term “isolated” means a substance in a form or environment that does not occur in nature. Non-limiting examples of isolated substances include (1) any non-naturally occurring substance, (2) any substance including, but not limited to, any host cell, enzyme, variant, nucleic acid, protein, peptide, cofactor, or carbohydrate/saccharide that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated. It is believed that the embodiments (e.g., reaction compositions and products thereof) disclosed herein are synthetic/man-made, and/or have properties that are not naturally occurring.

The term “increased” as used herein can refer to a quantity or activity that is at least about 1%, 2%, 3%, 4%, 5%, 6%, 7%, 8%, 9%, 10%, 11%, 12%, 13%, 14%, 15%, 16%, 17%, 18%, 19%, 20%, 50%, 100%, or 200% more than the quantity or activity for which the increased quantity or activity is being compared. The terms “increased”, “elevated”, “enhanced”, “greater than”, “improved” and the like are used interchangeably herein. These terms can be used to characterize the “over-expression” or “up-regulation” of a polynucleotide encoding a protein, for example.

Some aspects of the present disclosure regard a reaction composition comprising at least water, sucrose, an alpha-glucan substrate, and a polypeptide that is capable of forming at least one alpha-1,2 branch from the alpha-glucan substrate, wherein the polypeptide comprises an amino acid sequence that is at least 90% identical to:
(i) the mature form of a sequence selected from the group consisting of SEQ ID NOs:4, 1, 2, 3, 5, 6, 7, 8, 9, 10, 11, 12, and 13;
(ii) SEQ ID NO:27 or a sub-sequence within any one of SEQ ID NOs:4, 2, 3, 5, 6, 7, 8, 9, 10, 11, 12, or 13 that aligns with SEQ ID NO:27; and/or
(iii) a sequence selected from the group consisting of SEQ ID NOs:4, 1, 2, 3, 5, 6, 7, 8, 9, 10, 11, 12, and 13.
   A reaction as described immediately above can optionally be characterized herein as an alpha-1,2 branching reaction. A product of such a reaction can be referred to as a glucan composition (or glucan product) comprising alpha-1,2 linkages, for example.

A polypeptide capable of forming at least one alpha-1,2 branch from an alpha-glucan substrate in some aspects of the present disclosure can comprise, or consist of, an amino acid sequence that is 100% identical to, or at least about 90%, 91%, 92%, 93%, 94%, 95%, 96%, 97%, 98%, or 99% identical to, the predicted mature form of a sequence selected from the group consisting of SEQ ID NOs:4, 1, 2, 3 and 5-13. The following sequences are believed to be examples of mature forms (expressed forms) of these polypeptides, respectively: positions 36 to 1672 of SEQ ID NO:4, positions 21 to 2771 of SEQ ID NO: 1, positions 21 to 2821 of SEQ ID NO:2, positions 41 to 2844 of SEQ ID NO:3, positions 51 to 1632 of SEQ ID NO:5, positions 51 to 1318 of SEQ ID NO:6, positions 51 to 1139 of SEQ ID NO:7, positions 51 to 1463 of SEQ ID NO:8, positions 41 to 2841 of SEQ ID NO:9, positions 46 to 2580 of SEQ ID NO:10, positions 51 to 1463 of SEQ ID NO: 11, positions 21 to 2824 of SEQ ID NO:12, positions 21 to 2771 of SEQ ID NO:13. Any of these sequences may optionally further comprise an added N-terminal methionine residue.

In some aspects, a polypeptide capable of forming at least one alpha-1,2 branch from an alpha-glucan substrate comprises, or consists of, an amino acid sequence that is 100% identical to, or at least about 90%, 91%, 92%, 93%, 94%, 95%, 96%, 97%, 98%, or 99% identical to, a sequence selected from the group consisting of SEQ ID NOs:4, 1, 2, 3 and 5-13.

SEQ ID NO:27 (GTFJ18T1), which is an N-terminally shortened version of SEQ ID NO: 1, is shown in the Examples below to be capable of forming at least one alpha-1,2 branch from an alpha-glucan substrate. Further, SEQ ID NO:4 (GTF9905), which contains a sub-sequence that is relatively similar to SEQ ID NO:27, also is shown herein to be capable of forming at least one alpha-1,2 branch from an alpha-glucan substrate. Based on this information, it is believed that a suitable polypeptide in certain embodiments can comprise an amino acid sequence that is 100% identical to, or at least about 90%, 91%, 92%, 93%, 94%, 95%, 96%, 97%, 98%, or 99% identical to, SEQ ID NO:27 or a sub-sequence within any one of SEQ ID NOs:4, 2, 3 and 5-13 that aligns with SEQ ID NO:27. In some aspects, a suitable sub-sequence that aligns with SEQ ID NO:27 is as listed in the following table:

| | % Identity^{a} w/ | % Similarity^{a} w/ |
|---|---|---|
| Sub-sequence | SEQ ID NO: 27 | SEQ ID NO: 27 |
| positions 36 to 1115 | 60.6 | 74.8 |
| of SEQ ID NO: 4 | | |
| positions 1715 to 2821 | 98.1 | 98.9 |
| of SEQ ID NO: 2 | | |
| positions 1735 to 2834 | 73.7 | 85.3 |
| of SEQ ID NO: 3 | | |
| positions 51 to 1167 | 56.8 | 71.0 |
| of SEQ ID NO: 5 | | |
| positions 93 to 1178 | 56.4 | 71.3 |
| of SEQ ID NO: 6 | | |
| positions 51 to 1130 | 56.2 | 69.9 |
| of SEQ ID NO: 7 | | |
| positions 51 to 1158 | 57.7 | 72.8 |
| of SEQ ID NO: 8 | | |
| positions 1735 to 2841 | 98.1 | 98.9 |
| of SEQ ID NO: 9 | | |
| positions 1274 to 2413 | 54.6 | 67.6 |
| of SEQ ID NO: 10 | | |
| positions 51 to 1158 | 57.6 | 72.7 |
| of SEQ ID NO: 11 | | |
| positions 1715 to 2821 | 73.7 | 85.4 |
| of SEQ ID NO: 12 | | |
| positions 1665 to 2771 | 100.0 | 100.0 |
| of SEQ ID NO: 13 | | |

^{a}Percent identity and similarity values per EMBOSS alignment.

It should be apparent from some of the above embodiments that a polypeptide capable of forming at least one alpha-1,2 branch from an alpha-glucan substrate can include an amino acid sequence that is truncated relative to any of SEQ ID NOs:1-13. In certain embodiments, a truncated amino acid sequence includes a domain from any of SEQ ID NOs:1-13 that catalyzes the synthesis of glucan having alpha-1,2 linkages. Such a truncated amino acid sequence can optionally include one or more glucan binding domain. In certain embodiments, a truncated amino acid sequence does not include a domain that catalyzes synthesis of a glucan having linkages other than alpha-1,2 linkages.

Some embodiments disclosed herein concern a polynucleotide comprising a nucleotide sequence that encodes any 1,2-branching enzyme (e.g., parts i, ii, or iii above as related to any of SEQ ID NOs:4, 1, 2, 3, 5-13 and 27) or other glucosyltransferase as presently disclosed. Optionally, one or more regulatory sequences are operably linked to the nucleotide sequence, and preferably a promoter sequence is included as a regulatory sequence.

A polynucleotide comprising a nucleotide sequence encoding an enzyme herein can be a vector or construct useful for transferring a nucleotide sequence into a cell, for example (e.g., expression vector). Examples of a suitable vector/construct can be selected from a plasmid, yeast artificial chromosome (YAC), cosmid, phagemid, bacterial artificial chromosome (BAC), virus, or linear DNA (e.g., linear PCR product). A polynucleotide sequence in some aspects can be capable of existing transiently (i.e., not integrated into the genome) or stably (i.e., integrated into the genome) in a cell. A polynucleotide sequence in some aspects can comprise, or lack, one or more suitable marker sequences (e.g., selection or phenotype marker).

A polynucleotide sequence in certain embodiments can comprise one or more regulatory sequences operably linked to the nucleotide sequence encoding an enzyme. For example, a nucleotide sequence encoding an enzyme may be in operable linkage with a promoter sequence (e.g., a heterologous promoter). A promoter sequence can be suitable for expression in a cell (e.g., bacterial cell such as E. coli; eukaryotic cell such as a fungus, yeast, insect, or mammalian cell) or in an in vitro protein expression system, for example. Examples of other suitable regulatory sequences are disclosed herein (e.g., transcription terminator sequences).

In some embodiments, a polynucleotide sequence does not comprise a regulatory sequence operably linked to a nucleotide encoding a glucosyltransferase. Such a polynucleotide could be a cloning vector (e.g., cloning plasmid), for example, used simply for sub-cloning or gene shuttling purposes.

Possible initiation control regions or promoters that can be included in an expression vector herein are numerous and familiar to those skilled in the art. Virtually any promoter capable of driving these genes is suitable, including but not limited to, CYC1, HIS3, GAL1, GAL10, ADH1, PGK, PHO5, GAPDH, ADC1, TRP1, URA3, LEU2, ENO, TPI (useful for expression in Saccharomyces); AOX1 (useful for expression in Pichia); and lac, araB, tet, trp, lP_{L}, lP_{R}, T7, tac, and trc (useful for expression in Escherichia coli) as well as the amy, apr, npr promoters and various phage promoters useful for expression in Bacillus.

DNA fragments which control transcriptional termination may also be derived from various genes native to a preferred host cell. In certain embodiments, the inclusion of a termination control region is optional. In certain embodiments, the expression vector includes a termination control region derived from the preferred host cell.

In certain embodiments, any polypeptide disclosed herein is in the form of a fusion protein. For example, a polypeptide may include one or more tag sequences that can aide in purification of the polypeptide. Exemplary tag sequences include: GST (glutathione-S-transferase), intein-CBD (chitin-binding domain), MBD (maltose binding domain), and histidine tags.

In certain embodiments, an expression vector is included in a host cell, particularly a microbial host cell. In some embodiments, a microbial host cell can be found within the fungal or bacterial families, and/or grow over a wide range of temperature, pH values, and solvent tolerances.

For example, it is contemplated that any of bacteria, yeast, and filamentous fungi may suitably host the expression vector. Inclusion of an expression vector in a host cell may be used for intracellular and/or extracellular expression of a polypeptide as disclosed herein. Transcription, translation and the protein biosynthetic apparatus can remain invariant relative to the cellular feedstock used to generate cellular biomass; functional genes typically can be expressed regardless. Examples of host cells include, but are not limited to, bacterial, fungal or yeast species such as Aspergillus, Trichoderma, Saccharomyces, Pichia, Phaffia, Kluyveromyces, Candida, Hansenula, Yarrowia, Salmonella, Bacillus, Acinetobacter, Zymomonas, Agrobacterium, Erythrobacter, Chlorobium, Chromatium, Flavobacterium, Cytophaga, Rhodobacter, Rhodococcus, Streptomyces, Brevibacterium, Corynebacteria, Mycobacterium, Deinococcus, Escherichia, Erwinia, Pantoea, Pseudomonas, Sphingomonas, Methylomonas, Methylobacter, Methylococcus, Methylosinus, Methylomicrobium, Methylocystis, Alcaligenes, Synechocystis, Synechococcus, Anabaena, Thiobacillus, Methanobacterium, Klebsiella, and Myxococcus. In certain embodiments, a fungal host cell is Trichoderma, such as a strain of Trichoderma reesei. In certain embodiments, bacterial host strains include Escherichia, Bacillus, Kluyveromyces, and Pseudomonas. In some embodiments, the bacterial host cell is Bacillus subtilis or Escherichia coli.

In certain embodiments, (i) a host cell includes more than one expression vector, and/or (ii) multiple polypeptides are expressed in the host cell. For example, in certain embodiments, a host cell includes an expression vector for the expression of an alpha-glucanohydrolase. It would be recognized that the expression vector for expressing an alpha-glucanohydrolase can be the same or different than the expression vector for expressing a polypeptide capable of forming at least one alpha-1,2 branch from an alpha-glucan substrate,

An alpha-glucan substrate/backbone in certain embodiments of the present disclosure has a degree of polymerization (DP) of at least 3, comprises at least (i) alpha-1,6 glycosidic linkages or (ii) alpha-1,3 and alpha-1,6 glycosidic linkages, and typically is water-soluble.

In some aspects, an alpha-glucan substrate can have a DP of about, or at least about, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 100, 105, 110 150, 200, 250, 300, 400, 500, 600, 700, 800, 900, 1000, 1100, 1200, 1300, 1400, 1500, 1600, 1700, 1800, 1900, or 2000. The DP of an alpha-glucan substrate can optionally be expressed as a range between any two of these values. Merely as examples, the DP can be about 8-20, 8-30, 8-100, or 8-500 (which happen to be examples of DP8+ alpha-glucan substrates), 3-4, 3-5, 3-6, 3-7, 3-8, 4-5, 4-6, 4-7, 4-8, 5-6, 5-7, 5-8, 6-7, 6-8, or 7-8. Merely as other examples, the DP can be 90-120, 95-120, 100-120, 105-120, 110-120, 115-120, 90-115, 95-115, 100-115, 105-115, 110-115, 90-110, 95-110, 100-110, 105-110, 90-105, 95-105, 100-105, 90-100, 95-100, or 90-95.

An alpha-glucan substrate in certain embodiments comprises at least (i) alpha-1,6 glycosidic linkages or (ii) alpha-1,3 and alpha-1,6 glycosidic linkages. For example, the percentage of alpha-1,6 linkages can be at least about 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 69%, 70%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. The linkage profile of an alpha-glucan substrate can optionally be expressed as having a range between any two of these values. The other substrate linkages in any of these embodiments can be alpha-1,3 (e.g., up to 80%), and/or not include any alpha-1,4 or alpha-1,2 linkages, for example. In some aspects, an alpha-glucan substrate comprises at least 50% alpha-1,6 glycosidic linkages. In some other embodiments, an alpha-glucan substrate comprises 1 to 50% alpha-1,3 glycosidic linkages.

In certain embodiments, an alpha-glucan substrate is prepared using an enzyme selected from glucosyltransferases (typically from the GH70 family of glycoside hydrolases), dextrin dextranases, 4,6-alpha-glucosyltransferases (“Gtf-B type” from family GH70), or combinations thereof. Optionally, at least one alpha-glucosidase is further included, such as a dextranase and/or mutanase. In some embodiments, an alpha-glucan substrate is a product of a glucosyltransferase comprising, or consisting of, an amino acid sequence that is 100% identical to, or at least about 90%, 91%, 92%, 93%, 94%, 95%, 96%, 97%, 98%, or 99% identical to, SEQ ID NO:30 (GTF8117), 32 (GTF6831), or 33 (GTF5604). An alpha-glucan substrate herein can be prepared by an appropriate glucosyltransferase reaction composition (e.g., comprising at least water and sucrose, in addition to at least one GTF enzyme).

An alpha-glucan substrate can be synthesized (and optionally isolated) before enzymatically introducing alpha-1,2 branching, or may be concomitantly synthesized in the presence of an alpha-1,2 branching enzyme (i.e., glucan substrate backbone synthesis can be conducted in the same reaction mixture with the polypeptide having alpha-1,2 branching activity in the presence of an effective amount of sucrose). An alpha-glucan substrate may be produced in a variety of ways including, but not limited to, (1) synthesis from at least one glucosyltransferase (using a polypeptide that is different from the polypeptide having alpha-1,2 branching activity) in the presence of sucrose, (2) synthesis from maltodextrin obtainable from starch or sucrose (e.g., maltodextrin substrate synthesized from sucrose using an amylosucrase) using a polypeptide having dextrin dextranase activity, a Gtf-B type GH70 glucosyltransferase, or a combination thereof, (3) synthesis using method (1) and/or (2) in the presence of at least one alpha-glucanohydrolase (e.g., dextranase, mutanase, or a combination thereof), and (4) any combination of (1), (2), or (3) so long as the alpha-glucan substrate is capable of being acted upon by the polypeptide having alpha-1,2 branching activity. In a further embodiment, an alpha-glucan substrate may be synthesized prior to an alpha-1,2 branching step or may be synthesized concomitant with alpha-1,2 branching (i.e., the polypeptide having alpha-1,2 branching activity and an effective amount of sucrose is present in the aqueous reaction mixture). In the context of synthesizing an alpha-glucan substrate using any of the above embodiments, reactants for synthesis may include sucrose and/or maltodextrin, and optionally be in the presence of one or more additional acceptors. In another embodiment, the reactants may further comprise one or more acceptors, such as maltose, isomaltose, isomaltotriose, and methyl-alpha-D-glucan, to name a few.

In certain embodiments, the alpha-glucan substrate is synthesized using a combination of at least one glucosyltransferase capable of forming glucose oligomers with at least one alpha-glucanohydrolase in the same reaction mixture (i.e., they are concomitantly present and active in the reaction composition). As such, the reactants for the alpha-glucanohydrolase are represented by the glucose oligomers concomitantly being synthesized in the reaction system by the glucosyltransferase from sucrose.

A glucan product of a 1,2-branching reaction herein (e.g., “glucan composition comprising alpha-1,2 linkages”) (such a product can be that of a reaction composition herein and/or a product of a method herein of producing a glucan composition) can be characterized in terms of (i) any alpha-glucan substrate herein plus (ii) any added 1,2-branching as disclosed herein, for example. The percent 1,2-branching of a glucan composition comprising alpha-1,2 linkages can be about, at least about., or less than about, 1%, 2%, 3%, 4%, 5%, 6%., 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, or 55%, for example. This 1,2-branching profile can optionally be expressed as having a range between any two of these values. Merely as examples, the percentage of 1,2-branches in a glucan composition can be 15-50%, 15-45%,15-40%, 15-35%, 15-30%, 15-25%, 15-20%, 20-50%, 20-45%, 20-40%, 20-35%, 20-30%, 20-25%, 25-50%, 25-45%, 25-40%, 25-350, 25-30%, 30-50, 30-45%, 30-40%, 30-35%, 35-50%, 35-45%, 35-40%, 40-50%, or 40-45% (some or all of such 1,2-branching profiles optionally are associated with glucan compositions herein that do not increase blood-glucose levels when enterally administered to a mammal [can optionally be referred to as a dietary glucan fiber]). Merely as additional examples, the percentage of 1,2-branches in a glucan composition can be less than about 10%, 9%, 8%, 7%, 6%, 5%, or 4%, or range from 2-10%, 4-10%, 6-10%, 2-8%, 4-8%, 6-8%, 2-6%, 4-6%, 4.5-6%, 5-6%, 4-7%, 4.5-7%, or 5-7% (such 1,2-branching profiles optionally are associated with glucan compositions herein that slowly increase blood-glucose levels when enterally administered to a mammal [can optionally be referred to as low glycemic index glucan). In some aspects, a glucan composition comprising alpha-1,2 linkages comprises only 1,6-linkages and 1,2-branches (1,2,6) (e.g., a 1,6-linked backbone decorated with 1,2-linked pendant glucoses), With no other linkage types present. In other embodiments, a glucan composition comprising alpha-1,2 linkages comprises any alpha-glucan substrate backbone as disclosed herein decorated with 1,2-linked branches. The percentage of 1,2-branch points (as well as other linkages types) herein can be determined using an ¹H NMR or GC/MS method, for example, such as disclosed in the below Examples.

In some aspects, a glucan composition comprising alpha-1,2 linkages can have a DP of about, or at least about, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 105, 110, 120, 130, 140, 150, 160, 170, 180, 190, 200, 210, 220, 230, 240, 250, 300, 400, 500, 600, 700, 800, 900, 1000, 1100, 1200, 1300, 1400, 1500, 1600, 1700, 1800, 1900, or 2000. The DP of a glucan composition comprising alpha-1,2 linkages can optionally be expressed as a range between any two of these values. Merely as examples, the DP can be about 120-160, 125-160, 130-160, 135-160, 140-160, 145-160, 150-160, 155-160, 120-155, 125-155, 130-155, 135-155, 140-155, 145-155, 150-155, 120-150, 125-150, 130-150, 135-150, 140-150, 145-150, 120-145, 125-145, 130-145, 135-145, 140-145, 120-140, 125-140, 130-140, 135-140, 120-135, 125-135, 130-135, 120-130, 125-130, or 120-125 (such DP profiles optionally are associated with glucan compositions herein that do not increase blood-glucose levels when enterally administered to a mammal), or about 100-110. Merely as additional examples, the DP can be 100-120, 105-120, 110-120, 115-120, 100-115, 105-115, 110-115, 100-110, 105-110, or 100-105 (such DP profiles optionally are associated with glucan compositions herein that slowly increase blood-glucose levels when enterally administered to a mammal).

In some aspects, a glucan composition comprising alpha-1,2 linkages (e.g., one that is water-soluble) slowly releases glucose when fed (enteral administration) to a mammal such as a human; this type of glucan composition can be characterized as a “slow glucose release glucan composition” (or like terms). Such slow glucose release corresponds with a slow increase in blood glucose levels (low glycemic response. In some other aspects, a glucan composition comprising alpha-1,2 linkages does not release glucose when fed to a mammal, and thereby does not result in any increase in blood glucose levels (no glycemic response). Such effects on blood glucose levels (slow effect or no effect) are believed to be specific to the glucan composition comprising alpha-1,2 linkages, and thus independent of the effects on blood glucose levels that may result from a blood glucose-increasing ingredient (e.g., sucrose, free glucose, starch) potentially included in a substance being fed to the mammal. In other words, if a blood glucose-increasing ingredient(s) is included in a food/beverage that contains a glucan composition comprising alpha-1,2 linkages herein, any significant or fast increase in blood glucose levels in a mammal fed the food/beverage are believed to be attributable to such other blood glucose-increasing ingredient(s).

Slow glucose release herein is believed to primarily occur in the gut of a mammal following enteral administration (ingestion) of certain glucan compositions herein comprising alpha-1,2 linkages (e.g., less than about 10% alpha-1,2 branches). Slow glucose release typically does not result in a blood glucose spike. The benefit of a slow glucose release glucan herein, in comparison with sucrose and other forms of readily digestible glucose-containing carbohydrates (e.g., free glucose), is that it is digested slowly and steadily by mammals. Although a slow glucose release glucan herein is a calorically available carbohydrate—meaning that it is digestible and absorbable (e.g., refer to Table 35, which shows that sample 105-3 [an example of a slow glucose release glucan herein] ingestion resulted in an AUC value fairly equal to the AUC value resulting from free glucose ingestion)—its digestion results in a slow and sustained release of glucose as compared to the digestion of free glucose (e.g., refer to Table 35, which shows that sample 105-3 ingestion results in a maximum blood glucose concentration of 334 mg/dL at 1 h, whereas free glucose ingestion results in a maximum of 401 mg/dL just after 20 min). Thus, a slow glucose release glucan herein, following ingestion in a mammal, typically results in the same or similar total glucose release as that resulting from a readily digestible glucose-containing carbohydrate (e.g., total glucose released over 2-2.5 hours post-ingestion) (e.g., when the same amount of slow glucose release glucan or readily digestible glucose-containing carbohydrate are ingested). However, a slow glucose release glucan herein, following ingestion in a mammal (e.g., as measured within 0.25-2.5 hours post-ingestion), results in: (i) a blood glucose peak that is lower (e.g., at least about 10%, 12.5%, 15%, 17.5%, 20%, 22.5%, 25%, 10-25%, 10-20%, 10-15%, 15-25%, or 15-20% lower) than the peak induced by a readily digestible glucose-containing carbohydrate, and/or (ii) a blood glucose curve that is stretched compared to that of a readily digestible glucose-containing carbohydrate. The results of (i) and/or (ii) can be observed, for example, when the same or similar amount of slow glucose release glucan or readily digestible glucose-containing carbohydrate are ingested. Levels of glucose release in a mammal can be determined, for example, by measuring blood glucose levels.

Slow digestion and absorption of glucose from a slow glucose release glucan herein contributes to the low blood glucose response (low glycemic response) following its ingestion. It is believed that such low glycemic response in turn leads to a low release of insulin. A low glycemic response has been considered a physiologically beneficial effect by the European Food Safety Authority. In the long-term, a diet including carbohydrates that reduce undesirably high concentrations of glucose in blood and thus a lower demand for insulin (possibly such as a slow glucose release glucan herein), is supportive for the prevention and management of diabetes mellitus, cardiovascular disease, and possibly obesity. In addition, slow release carbohydrates are of interest in physical endurance activity where an optimum use of limited carbohydrate sources might be advantageous. Carbohydrates and their supply of glucose to the brain play a central role in cognitive performance and mood. Hence a steady and sustained glucose supply may play a role in memory performance and mood.

Thus, further disclosed herein is a method comprising enterally administering (e.g., ingesting) a substance (e.g., food, beverage, supplement, or pharmaceutical) to a mammal, wherein the substance comprises a glucan composition comprising alpha-1,2 linkages, wherein the administering results in less or slower blood glucose elevation in the mammal as compared to a mammal that is enterally administered a substance that lacks the glucan composition but instead contains a same amount of a readily digestible glucose-containing carbohydrate (e.g., if 1 g of the glucan composition was used, then comparison is with 1 g of the readily digestible glucose-containing carbohydrate), wherein the glucan composition is produced by any branching method or reaction herein. Enteral administration can, for example, be via self-administration (e.g., any form of ingestion such as eating, drinking, taking medicine) or non-self-administration (e.g., oral gavage, feeding apparatus such as feeding tube).

Thus, further disclosed herein is a method of producing an ingestible product (e.g., food or beverage), the method comprising incorporating a glucan composition comprising alpha-1,2 linkages into the ingestible product, wherein the glycemic index of the resulting ingestible product is not increased, or only marginally increased, compared to an ingestible product that lacks the glucan composition, and wherein the glucan composition is produced by any branching method or reaction herein.

Compositions Comprising Alpha-1,2-branched Glucan Compositions Produced Herein

A composition comprising an alpha-1,2-branched glucan as presently disclosed can be an aqueous composition in certain embodiments.

It is believed that an aqueous composition comprising an alpha-1,2-branched glucan can, in some aspects, have a viscosity of about, or at least about, 3, 4, 5, 10, 15, 20, 25, 30, 35, 40, 45, or 50 cPs (centipoise). Viscosity can be measured with an aqueous composition herein at any temperature between about 3° C. to about 110° C. (or any integer between 3 and 110° C.), for example. Viscosity can be measured at atmospheric pressure (about 760 torr) or any suitable higher or lower pressure.

The pH of an aqueous composition herein can be between about 2.0 to about 12.0, for example. Alternatively, pH can be about 2.0, 3.0, 4.0, 5.0, 6.0, 7.0, 8.0, 9.0, 10.0, 11.0, 12.0; or between 5.0 to about 12.0; or between about 4.0 and 8.0; or between about 5.0 and 8.0, for example.

An aqueous composition herein can comprise a solvent having at least about 10 wt % water. In other embodiments, a solvent is at least about 20, 30, 40, 50, 60, 70, 80, 90, or 100 wt % water (or any integer value between 10 and 100 wt %), for example.

An alpha-1,2-branched glucan herein can be present in an aqueous composition at a wt % of about, or at least about, 0.01, 0.05, 0.1, 0.2, 0.3, 0.4, 0.5, 0.6, 0.7, 0.8, 0.9, 1.0, 1.2, 1.4, 1.6, 1.8, 2.0, 2.5, 3.0, 3.5, 4.0, 4.5, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 wt %, for example.

An aqueous composition herein can generally comprises other components in addition to an alpha-1,2-branched glucan. For example, an aqueous composition can comprise one or more salts such as a sodium salt (e.g., NaCl). Other non-limiting examples of salts include those having (i) an aluminum, ammonium, barium, calcium, chromium (II or III), copper (I or II), iron (II or III), hydrogen, lead (II), lithium, magnesium, manganese (II or III), mercury (I or II), potassium, silver, sodium strontium, tin (II or IV), or zinc cation, and (ii) an acetate, borate, bromate, bromide, carbonate, chlorate, chloride, chlorite, chromate, cyanamide, cyanide, dichromate, dihydrogen phosphate, ferricyanide, ferrocyanide, fluoride, hydrogen carbonate, hydrogen phosphate, hydrogen sulfate, hydrogen sulfide, hydrogen sulfite, hydride, hydroxide, hypochlorite, iodate, iodide, nitrate, nitride, nitrite, oxalate, oxide, perchlorate, permanganate, peroxide, phosphate, phosphide, phosphite, silicate, stannate, stannite, sulfate, sulfide, sulfite, tartrate, or thiocyanate anion. Thus, any salt having a cation from (i) above and an anion from (ii) above can be in an aqueous composition, for example. A salt can be present in an aqueous composition herein at a wt % of about 0.01 to about 10.00 (or any hundredth increment between 0.01 and 10.00), for example.

A composition comprising an alpha-1,2-branched glucan herein can be non-aqueous (e.g., a dry composition) in some aspects. Examples of such embodiments include powders, granules, microcapsules, flakes, or any other form of particulate matter. Other examples include larger compositions such as pellets, bars, kernels, beads, tablets, sticks, or other agglomerates. A non-aqueous or dry composition herein typically has less than 3, 2, 1, 0.5, or 0.1 wt % water comprised therein. The amount of alpha-1,2-branched glucan herein in a non-aqueous or dry composition can be about, or at least about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.5, or 99.9 wt %, for example.

A composition comprising an alpha-1,2-branched glucan herein may optionally contain one or more active enzymes. Non-limiting examples of suitable enzymes include proteases, cellulases, hemicellulases, peroxidases, lipolytic enzymes (e.g., metallolipolytic enzymes), xylanases, lipases, phospholipases, esterases (e.g., arylesterase, polyesterase), perhydrolases, cutinases, pectinases, pectate lyases, mannanases, keratinases, reductases, oxidases (e.g., choline oxidase), phenoloxidases, lipoxygenases, ligninases, pullulanases, tannases, pentosanases, malanases, beta-glucanases, arabinosidases, hyaluronidases, chondroitinases, laccases, metalloproteinases, amadoriases, glucoamylases, arabinofuranosidases, phytases, isomerases, transferases and amylases. If an enzyme(s) is included, it may be comprised in a composition herein at about 0.0001-0.1 wt % (e.g., 0.01-0.03 wt %) active enzyme (e.g., calculated as pure enzyme protein), for example.

At least one, two, or more cellulases may be included in a composition herein. A cellulase herein can have endocellulase activity (EC 3.2.1.4), exocellulase activity (EC 3.2.1.91), or cellobiase activity (EC 3.2.1.21). A cellulase herein is an “active cellulase” having activity under suitable conditions for maintaining cellulase activity; it is within the skill of the art to determine such suitable conditions.

A cellulase herein may be derived from any microbial source, such as a bacteria or fungus. Chemically-modified cellulases or protein-engineered mutant cellulases are included. Suitable cellulases include, but are not limited to, cellulases from the genera Bacillus, Pseudomonas, Streptomyces, Trichoderma, Humicola, Fusarium, Thielavia and Acremonium. As other examples, a cellulase may be derived from Humicola insolens, Myceliophthora thermophila or Fusarium oxysporum; these and other cellulases are disclosed in U.S. Pat. Nos. 4,435,307, 5,648,263, 5,691,178, 5,776,757 and 7,604,974, which are all incorporated herein by reference. Exemplary Trichoderma reesei cellulases are disclosed in U.S. Pat. Nos. 4,689,297, 5,814,501, 5,324,649, and International Patent Appl. Publ. Nos. WO92/06221 and WO92/06165, all of which are incorporated herein by reference. Exemplary Bacillus cellulases are disclosed in U.S. Pat. No. 6,562,612, which is incorporated herein by reference. A cellulase, such as any of the foregoing, preferably is in a mature form lacking an N-terminal signal peptide. Commercially available cellulases useful herein include CELLUZYME® and CAREZYME® (Novozymes A/S); CLAZINASE® and PURADAX® HA (DuPont Industrial Biosciences), and KAC-500(B)® (Kao Corporation).

One or more cellulases can be directly added as an ingredient when preparing a composition disclosed herein. Alternatively, one or more cellulases can be indirectly (inadvertently) provided in the disclosed composition. For example, cellulase can be provided in a composition herein by virtue of being present in a non-cellulase enzyme preparation used for preparing a composition. Cellulase in compositions in which cellulase is indirectly provided thereto can be present at about 0.1-10 ppb (e.g., less than 1 ppm), for example.

A cellulase in certain embodiments can be thermostable. Cellulase thermostability refers to the ability of the enzyme to retain activity after exposure to an elevated temperature (e.g. about 60-70° C.) for a period of time (e.g., about 30-60 minutes). The thermostability of a cellulase can be measured by its half-life (t½) given in minutes, hours, or days, during which time period half the cellulase activity is lost under defined conditions.

A cellulase in certain embodiments can be stable to a wide range of pH values (e.g. neutral or alkaline pH such as pH of ˜7.0 to ˜11.0). Such enzymes can remain stable for a predetermined period of time (e.g., at least about 15 min., 30 min., or 1 hour) under such pH conditions.

The effective concentration of cellulase in an aqueous composition in which a fabric is treated can be readily determined by a skilled artisan. In fabric care processes, cellulase can be present in an aqueous composition (e.g., wash liquor) in which a fabric is treated in a concentration that is minimally about 0.01-0.1 ppm total cellulase protein, or about 0.1-10 ppb total cellulase protein (e.g., less than 1 ppm), to maximally about 100, 200, 500, 1000, 2000, 3000, 4000, or 5000 ppm total cellulase protein, for example.

A composition comprising an alpha-1,2-branched glucan herein can be in the form of, and/or comprised in, a household care product, personal care product, industrial product, pharmaceutical product, or food product, for example, such as any of those products described below. Any of these compositions can be aqueous compositions, for example.

Personal care products herein are not particularly limited and include, for example, skin care compositions, cosmetic compositions, antifungal compositions, and antibacterial compositions.

Personal care products herein may be in the form of, for example, lotions, creams, pastes, balms, ointments, pomades, gels, liquids, combinations of these and the like. The personal care products disclosed herein can include at least one active ingredient, if desired. An active ingredient is generally recognized as an ingredient that causes an intended pharmacological effect. A personal care product herein can be used in personal care cleaning applications in certain embodiments.

A skin care product typically may include at least one active ingredient for the treatment or prevention of skin ailments, providing a cosmetic effect, or for providing a moisturizing benefit to skin, such as zinc oxide, petrolatum, white petrolatum, mineral oil, cod liver oil, lanolin, dimethicone, hard fat, vitamin A, allantoin, calamine, kaolin, glycerin, or colloidal oatmeal, and combinations of these. A skin care product may include one or more natural moisturizing factors such as ceramides, hyaluronic acid, glycerin, squalane, amino acids, cholesterol, fatty acids, triglycerides, phospholipids, glycosphingolipids, urea, linoleic acid, glycosaminoglycans, mucopolysaccharide, sodium lactate, or sodium pyrrolidone carboxylate, for example. Other ingredients that may be included in a skin care product include, without limitation, glycerides, apricot kernel oil, canola oil, squalane, squalene, coconut oil, corn oil, jojoba oil, jojoba wax, lecithin, olive oil, safflower oil, sesame oil, shea butter, soybean oil, sweet almond oil, sunflower oil, tea tree oil, shea butter, palm oil, cholesterol, cholesterol esters, wax esters, fatty acids, and orange oil.

A personal care product herein can also be in the form of makeup, lipstick, mascara, rouge, foundation, blush, eyeliner, lip liner, lip gloss, other cosmetics, sunscreen, sun block, nail polish, nail conditioner, bath gel, shower gel, body wash, face wash, lip balm, skin conditioner, cold cream, moisturizer, body spray, soap, body scrub, exfoliant, astringent, scruffing lotion, depilatory, permanent waving solution, antidandruff formulation, antiperspirant composition, deodorant, shaving product, pre-shaving product, after-shaving product, cleanser, skin gel, rinse, dentifrice composition, toothpaste, or mouthwash, for example.

A personal care product in some aspects can be a hair care product. Examples of hair care products herein include shampoo, hair conditioner (leave-in or rinse-out), cream rinse, hair dye, hair coloring product, hair shine product, hair serum, hair anti-frizz product, hair split-end repair product, mousse, hair spray, and styling gel. A hair care product can be in the form of a liquid, paste, gel, solid, or powder in some embodiments. A hair care product as presently disclosed typically comprises one or more of the following ingredients, which are generally used to formulate hair care products: anionic surfactants such as polyoxyethylenelauryl ether sodium sulfate; cationic surfactants such as stearyltrimethylammonium chloride and/or distearyltrimethylammonium chloride; nonionic surfactants such as glyceryl monostearate, sorbitan monopalmitate and/or polyoxyethylenecetyl ether; wetting agents such as propylene glycol, 1,3-butylene glycol, glycerin, sorbitol, pyroglutamic acid salts, amino acids and/or trimethylglycine; hydrocarbons such as liquid paraffins, petrolatum, solid paraffins, squalane and/or olefin oligomers; higher alcohols such as stearyl alcohol and/or cetyl alcohol; superfatting agents; antidandruff agents; disinfectants; anti-inflammatory agents; crude drugs; water-soluble polymers such as methylcellulose, hydroxycellulose and/or partially deacetylated chitin; antiseptics such as paraben; ultra-violet light absorbers; pearling agents; pH adjustors; perfumes; and pigments.

A pharmaceutical product herein can be in the form of an emulsion, liquid, elixir, gel, suspension, solution, cream, or ointment, for example. Also, a pharmaceutical product herein can be in the form of any of the personal care products disclosed herein, such as an antibacterial or antifungal composition. A pharmaceutical product can further comprise one or more pharmaceutically acceptable carriers, diluents, and/or pharmaceutically acceptable salts. An alpha-1,2-branched glucan disclosed herein can also be used in capsules, encapsulants, tablet coatings, and excipients for medicaments and drugs.

A household care and/or industrial product herein can be in the form of drywall tape-joint compounds; mortars; grouts; cement plasters; spray plasters; cement stucco; adhesives; pastes; wall/ceiling texturizers; binders and processing aids for tape casting, extrusion forming, injection molding and ceramics; spray adherents and suspending/dispersing aids for pesticides, herbicides, and fertilizers; fabric care products such as fabric softeners and laundry detergents; dishwashing detergents; hard surface cleaners; air fresheners; polymer emulsions; gels such as water-based gels; surfactant solutions; paints such as water-based paints; protective coatings; adhesives; sealants and caulks; inks such as water-based ink; metal-working fluids; or emulsion-based metal cleaning fluids used in electroplating, phosphatizing, galvanizing and/or general metal cleaning operations, for example. A household care product or industrial product herein can be used in cleaning applications in certain embodiments, and as such can be comprised in detergent compositions, for example.

Alpha-1,2-branched glucans disclosed herein are believed to be useful for providing one or more of the following physical properties to a personal care product, pharmaceutical product, household care product, industrial product, or food product: glycemic index modification, dietary fiber, thickening, freeze/thaw stability, lubricity, moisture retention and release, texture, consistency, shape retention, emulsification, binding, suspension, dispersion, gelation, reduced mineral hardness, for example. Examples of a concentration or amount of an alpha-1,2-branched glucan in a product can be any of the weight percentages provided above, for example.

An alpha-1,2-branched glucan as presently disclosed may be formulated (e.g., blended, mixed, incorporated into, etc.) with one or more other materials suitable for use in a food product and/or other ingestible products herein (e.g., nutritional supplements, pharmaceuticals). Optionally, an alpha-1,2-branched glucan herein can be provided in a syrup for use in preparing and/or modifying any food or other ingestible product as presently disclosed.

In some embodiments, an alpha-1,2-branched glucan herein may be included in a product with at least one of the following: monosaccharides, disaccharides, glucose, sucrose, fructose, leucrose, corn syrup, high fructose corn syrup, isomerized sugar, maltose, trehalose, panose, raffinose, cellobiose, isomaltose, honey, maple sugar, fruit-derived sweeteners, sorbitol, maltitol, isomaltitol, lactose, nigerose, kojibiose, xylitol, erythritol, dihydrochalcone, stevioside, alpha-glycosyl stevioside, acesulfame potassium, alitame, neotame, glycyrrhizin, thaumantin, sucralose, L-aspartyl-L-phenylalanine methyl ester, saccharine, maltodextrin, starch, potato starch, tapioca starch, dextran, soluble corn fiber, resistant maltodextrins, branched maltodextrins, inulin, polydextrose, fructo-oligosaccharides, galacto-oligosaccharides, xylo-oligosaccharides, arabinoxylo-oligosaccharides, nigero-oligosaccharides, gentio-oligosaccharides, hemicellulose, fructose oligomer syrup, isomalto-oligosaccharides, fillers, excipients, and binders.

In certain embodiments, an ingestible product comprises 0.01 to 99 wt %, 0.1 to 90 wt %, 1 to 90 wt %, or 5 to 80 wt % of alpha-1,2-branched glucan herein on a dry solids basis.

The term “food” is intended to encompass food for human consumption as well as for animal (e.g., mammal) consumption. By “functional food” it is meant any fresh or processed food claimed to have a health-promoting and/or disease-preventing and/or disease-(risk)-reducing property beyond the basic nutritional function of supplying nutrients. Functional food may include, for example, processed food or foods fortified with health-promoting additives. Examples of functional food are foods fortified with vitamins, or fermented foods with live cultures.

Other ingredients that can be included in an ingestible product herein are water or other aqueous solutions, fats, sugars, starch, binders, thickeners, colorants, flavorants, odorants, acidulants (such as lactic acid or malic acid, among others), stabilizers, high intensity sweeteners, and/or minerals, among others.

One or more alpha-1,2-branched glucan products herein may be provided in any of the food embodiments presently disclosed. Depending on the desired effect(s), (i) an alpha-1,2-branched glucan with dietary fiber qualities and/or (ii) an alpha-1,2-branched glucan that has a low glycemic index may be used as appropriate.

Examples of suitable food products include bread, breakfast cereals, biscuits, cakes, cookies, crackers, yogurt, kefir, miso, natto, tempeh, kimchee, sauerkraut, water, milk, fruit juice, vegetable juice, carbonated soft drinks, non-carbonated soft drinks, coffee, tea, beer, wine, liquor, alcoholic drink, snacks, soups, frozen desserts, fried foods, pizza, pasta products, potato products, rice products, corn products, wheat products, dairy products, hard candies, nutritional bars, cereals, dough, processed meats and cheeses, yoghurts, ice cream confections, milk-based drinks, salad dressings, sauces, toppings, desserts, confectionery products, cereal-based snack bars, prepared dishes, and the like.

In certain embodiments, an ingestible product herein can comprise at least one dietary fiber source. In those aspects in which an alpha-1,2-branched glucan herein is a dietary fiber itself (e.g., glucan having about 15-45% 1,2-branches in some cases), such glucan can be the sole fiber source or in addition to one or more other fiber sources. Suitable dietary fibers herein include oligo- or polysaccharides such as resistant/branched maltodextrins/fiber dextrins (e.g., NUTRIOSE® from Roquette Freres, Lestrem, France; FIBERSOL-2® from ADM-Matsutani LLC, Decatur, Illinois), polydextrose (e.g., LITESSE® or LITESSE® ULTRA from Danisco-DuPont Nutrition & Health, Wilmington, DE), soluble corn fiber (e.g., PROMITOR® from Tate & Lyle, London, UK), isomalto-oligosaccharides (IMOs), alternan and/or malto-alternan oligosaccharides (MAOs) (e.g., FIBERMALT™ from Aevotis GmbH, Potsdam, Germany; SUCROMALT™ from Cargill Inc., Minneapolis, MN), pullulan, resistant starch, inulin, fructo-oligosaccharides (FOS), galacto-oligosaccharides (GOS), xylo-oligosaccharides, arabinoxylo-oligosaccharides, nigero-oligosaccharides, gentio-oligosaccharides, hemicellulose and fructose oligomer syrup.

An alpha-1,2-branched glucan herein can be added to foods as a replacement or supplement for conventional carbohydrates, for example.

In certain embodiments, an ingestible product herein can comprise at least one artificial sweetener including, but not limited to, stevia, aspartame, sucralose, neotame, acesulfame potassium, saccharin, and any combination thereof. An ingestible product herein can comprise at least one sugar substitute, for example, such as brazzein, curculin, erythritol, glycerol, glycyrrhizin, hydrogenated starch hydrolysates, inulin, isomalt, lactitol, mabinlin, maltitol, malto-oligosaccharide, malto-alternan oligosaccharides (such as XTEND® SUCROMALT™, available from Cargill Inc., Minneapolis, MN), mannitol, miraculin, a mogroside mix, monatin, monellin, osladin, pentadin, sorbitol, stevia, tagatose, thaumatin, xylitol, and any combination thereof.

In certain embodiments, a food product containing an alpha-1,2-branched glucan herein will have a lower (e.g., at least about 5%, 10%, 15%, 20%, or 25% lower) glycemic response, glycemic index, and/or glycemic load than a similar food product in which a conventional carbohydrate (e.g., a readily digestible glucose-containing carbohydrate) is used (e.g., when used at same or similar amount). Further, because an alpha-1,2-branched glucan in some aspects is characterized to have low or no digestibility in the human stomach and/or small intestine, the caloric content of the food product is reduced (following the above comparison). An alpha-1,2-branched glucan can be used in food products alone or in combination with bulking agents, such as sugar alcohols or maltodextrins, to reduce caloric content, to enhance the food's nutritional profile, and/or as a partial replacement for fat.

It is contemplated that an alpha-1,2-branched glucan herein may be used in food products as a tenderizer or texturizer, to increase crispness or snap, to improve eye appeal, and/or to improve the rheology of dough, batter, or other food compositions. It is also contemplated that the glucan be used in food products as a humectant, to increase product shelf life, to produce a softer, moister texture, to reduce water activity, and/or to immobilize and manage water. Additional uses of the glucan may include: replacement of an egg wash and/or to enhance the surface sheen of a food product, to alter flour starch gelatinization temperature, to modify the texture of the product, and to enhance browning of the product.

An alpha-1,2-branched glucan can be used in a variety of types of food products. One type of food product in which the present glucan can be useful is bakery products (baked foods), such as cakes, brownies, cookies, cookie crisps, muffins, breads, and sweet doughs. There are two main categories of bakery products: yeast-raised and chemically-leavened. In yeast-raised products, like donuts, sweet doughs, and breads, a glucan herein can be used to replace sugars, but a small amount of sugar may still be desired due to the need for a fermentation substrate for the yeast or for crust browning. Alpha-1,2-branched glucan in a syrup, for example, can be added with other liquids as a direct replacement for non-fiber containing syrups or liquid sweeteners. The dough would then be processed under conditions commonly used in the baking industry including being mixed, fermented, divided, formed or extruded into loaves or shapes, proofed, and baked or fried. The product can be baked or fried using conditions similar to traditional products. Breads are commonly baked at temperatures ranging from 420° F. to 520° F. (216-271° C.)°. for 20 to 23 minutes and doughnuts can be fried at temperatures ranging from 400-415° F. (204-213° C.), although other temperatures and times could also be used.

Chemically leavened products typically have more sugar and may contain have a higher level of the carbohydrate compositions and/or edible syrups comprising alpha-glucan herein. A finished cookie can contain 30% sugar, which could be replaced, entirely or partially, with carbohydrate compositions and/or syrups comprising the present glucan composition. These products could have a pH of 4-9.5, for example. The moisture content can be between 2-40%, for example.

Glucan compositions herein (e.g., in a syrup) can be readily incorporated and may be added to the fat at the beginning of mixing during a creaming step or in any method similar to the syrup or dry sweetener that it is being used to replace. The product would be mixed and then formed, for example by being sheeted, rotary cut, wire cut, or through another forming process. The products would then be baked under typical baking conditions, for example at 200-450° F. (93-232° C.).

Another type of food product in which a glucan herein (e.g., in a syrup) can be used is breakfast cereal. For example, glucan-containing syrups could be used to replace all or part of the sugar in extruded cereal pieces and/or in the coating on the outside of those pieces. The coating is typically 30-60% of the total weight of the finished cereal piece. The syrup can be applied in a spray or drizzled on, for example.

Another type of food product in which an alpha-1,2-branched glucan herein can be used is dairy products. Examples of suitable dairy products include yogurt, yogurt drinks, milk drinks, flavored milks, smoothies, ice cream, shakes, cottage cheese, cottage cheese dressing, and dairy desserts, such as quarg and the whipped mousse-type products. This would include dairy products that are intended to be consumed directly (such as packaged smoothies) as well as those that are intended to be blended with other ingredients (such as blended smoothies). It can be used in pasteurized dairy products, such as ones that are pasteurized at a temperature from 160° F. to 285° F. (71-141° C.).

Another type of food product in which an alpha-1,2-branched glucan herein can be used is confections. Examples of suitable confections include hard candies, fondants, nougats and marshmallows, gelatin jelly candies or gummies, jellies, chocolate, licorice, chewing gum, caramels and toffees, chews, mints, tableted confections, and fruit snacks. In fruit snacks, a composition comprising a glucan herein could be used in combination with fruit juice. The fruit juice would provide the majority of the sweetness, and the composition comprising the glucan would reduce the total sugar content and possibly add fiber. Compositions comprising the alpha-glucan herein can be added to the initial candy slurry and heated to the finished solids content. The slurry could be heated from 200-305° F. (93-152° C.) to achieve the finished solids content. Acid could be added before or after heating to give a finished pH of 2-7. The composition comprising the glucan could be used as a replacement for 0-100% of the sugar and 1-100% of the corn syrup or other sweeteners present.

Another type of food product in which an alpha-1,2-branched glucan herein can be used is jams and jellies. Jams and jellies are made from fruit; jam contains fruit pieces, while jelly is made from fruit juice. The composition comprising the present glucan can be used in place of sugar or other sweeteners as follows: weigh fruit and juice into a tank; premix sugar, the alpha-glucan-containing composition and pectin; add the dry composition to the liquid and cook to a temperature of 214-220° F. (101-104° C.); hot fill into jars and retort for 5-30 minutes.

Another type of food product in which an alpha-1,2-branched glucan herein can be used are beverages. Examples of suitable beverages include carbonated beverages, fruit juices, concentrated juice mixes (e.g., margarita mix), clear waters, and beverage dry mixes. The use of an alpha-1,2-branched glucan dietary fiber herein may overcome clarity problems that result when other types of fiber are added to beverages. A complete replacement of sugars may be possible (which could be, for example, being up to 12% or more of the total formula).

Another suitable type of food product is high solids fillings. Examples of high solids fillings include fillings in snack bars, toaster pastries, donuts, and cookies. A high solids filling could be an acid/fruit filling or a savory filling, for example. An alpha-1,2-branched glucan herein could be added to products that would be consumed as is, or products that would undergo further processing, by a food processor (additional baking) or by a consumer (bake stable filling). In certain embodiments, the high solids fillings would have a solids concentration between 67-90%. The solids could be entirely replaced with a composition comprising the alpha-glucan or it could be used for a partial replacement of the other sweetener solids present (e.g., replacement of current solids from 5-100%). Typically fruit fillings would have a pH of 2-6, while savory fillings would be between 4-8 pH. Fillings could be prepared cold or heated at up to 2500 F (121° C.) to evaporate to the desired finished solids content.

Another suitable type of food product that can comprise glucan herein is represented by extruded and sheeted snacks. Examples of extruded and sheeted include puffed snacks, crackers, tortilla chips, and corn chips. In preparing an extruded piece, a composition comprising the present glucan would be added directly with the dry products. A small amount of water would be added in the extruder, and then it would pass through various zones ranging from 100° F. to 300° F. (38-149° C.). The dried product could be added at levels from 0-50% of the dry products mixture. A syrup comprising the glucan could also be added at one of the liquid ports along the extruder. The product would come out at either a low moisture content (5%) and then baked to remove the excess moisture, or at a slightly higher moisture content (10%) and then fried to remove moisture and cook out the product. Baking could be at temperatures up to 500° F. (260° C.) for 20 minutes. Baking would more typically be at 350° F. (177° C.) for 10 minutes. Frying would typically be at 350° F. (177° C.) for 2-5 minutes. In a sheeted snack, the glucan could be used as a partial replacement of the other dry ingredients (for example, flour). The glucan could be from 0-50% of the dry weight. The product would be dry mixed, and then water added to form cohesive dough. The product mix could have a pH from 5 to 8. The dough would then be sheeted and cut and then baked or fried. Baking could be at temperatures up to 5000 F (260° C.) for 20 minutes. Frying would typically be at 350° F. (177° C.) for 2-5 minutes. Another potential benefit from the use of a composition comprising the glucan is a reduction of the fat content of fried snacks by as much as 15% when it is added as an internal ingredient or as a coating on the outside of a fried food.

Another type of food product in which a glucan herein can be used is gelatin desserts. The ingredients for gelatin desserts are often sold as a dry mix with gelatin as a gelling agent. The sugar solids could be replaced partially or entirely with a composition comprising the present glucan in the dry mix. The dry mix can then be mixed with water and heated to 212° F. (100° C.) to dissolve the gelatin and then more water and/or fruit can be added to complete the gelatin dessert. The gelatin is then allowed to cool and set. Gelatin can also be sold in shelf stable packs. In that case the stabilizer is usually carrageenan-based. As stated above, a composition comprising the alpha-glucan could be used to replace up to 100% of the other sweetener solids. The dry ingredients are mixed into the liquids and then pasteurized and put into cups and allowed to cool and set.

Another type of food product in which a composition comprising glucan herein can be used is snack bars. Examples of snack bars in which it can be used include breakfast and meal replacement bars, nutrition bars, granola bars, protein bars, and cereal bars. It could be used in any part of the snack bars, such as in the high solids filling, the binding syrup or the particulate portion. A complete or partial replacement of sugar in the binding syrup may be possible. The binding syrup is typically from 50-90% solids and applied at a ratio ranging from 10% binding syrup to 90% particulates, to 70% binding syrup to 30% particulates. The binding syrup is made by heating a solution of sweeteners, bulking agents and other binders (like starch) to 160-230° F. (71-110° C.) (depending on the finished solids needed in the syrup). The syrup is then mixed with the particulates to coat the particulates, providing a coating throughout the matrix. A composition comprising the glucan could also be used in the particulates themselves. This could be an extruded piece, directly expanded or gun puffed. It could be used in combination with another grain ingredient, corn meal, rice flour or other similar ingredient.

Another type of food product in which a composition comprising a glucan herein can be used is cheese, cheese sauces, and other cheese products. Examples of cheese, cheese sauces, and other cheese products in which it can be used include lower milk solids cheese, lower fat cheese, and calorie reduced cheese. In block cheese, it can help to improve the melting characteristics, or to decrease the effect of the melt limitation added by other ingredients such as starch. It could also be used in cheese sauces, for example as a bulking agent, to replace fat, milk solids, or other typical bulking agents.

Another type of food product in which a glucan herein can be used is films that are edible and/or water soluble. Examples of films in which it can be used include films that are used to enclose dry mixes for a variety of foods and beverages that are intended to be dissolved in water, or films that are used to deliver color or flavors such as a spice film that is added to a food after cooking while still hot. Other film applications include, but are not limited to, fruit and vegetable leathers, and other flexible films.

In another embodiment, compositions comprising a glucan herein can be used is soups, syrups, sauces, and dressings. A typical dressing could be from 0-50% oil, with a pH range of 2-7. It could be cold processed or heat processed. It would be mixed, and then stabilizer would be added. The composition comprising the glucan could easily be added in liquid or dry form with the other ingredients as needed. The dressing composition may need to be heated to activate the stabilizer. Typical heating conditions would be from 170-200° F. (77-93° C.) for 1-30 minutes. After cooling, the oil is added to make a pre-emulsion. The product is then emulsified using a homogenizer, colloid mill, or other high shear process. Sauces can have from 0-10% oil and from 10-50% total solids, and can have a pH from 2-8. Sauces can be cold processed or heat processed. The ingredients are mixed and then heat processed. A glucan herein could easily be added in liquid or dry form with the other ingredients as needed. Typical heating would be from 170-200° F. (77-93° C.) for 1-30 minutes. Soups are more typically 20-50% solids and in a more neutral pH range (4-8). They can be a dry mix, to which a dry composition comprising glucan herein could be added, or a liquid soup which is canned and then retorted. In soups, resistant corn syrup could be used up to 50% solids, though a more typical usage would be to deliver 5 g of fiber/serving.

Another type of food product in which a glucan herein can be used is coffee creamers. Examples of coffee creamers in which it can be used include both liquid and dry creamers. A dry blended coffee creamer can be blended with commercial creamer powders of the following fat types: soybean, coconut, palm, sunflower, or canola oil, or butterfat. These fats can be non-hydrogenated or hydrogenated. The composition comprising the glucan can be added as a fiber source, optionally together with fructo-oligosaccharides, polydextrose, inulin, maltodextrin, resistant starch, sucrose, and/or conventional corn syrup solids. The composition can also contain high intensity sweeteners, such as sucralose, acesulfame potassium, aspartame, or combinations thereof. These ingredients can be dry blended to produce the desired composition. A spray dried creamer powder is a combination of fat, protein and carbohydrates, emulsifiers, emulsifying salts, sweeteners, and anti-caking agents. The fat source can be one or more of soybean, coconut, palm, sunflower, or canola oil, or butterfat. The protein can be sodium or calcium caseinates, milk proteins, whey proteins, wheat proteins, or soy proteins. The carbohydrate could be a composition comprising the glucan alone or in combination with fructo-oligosaccharides, polydextrose, inulin, resistant starch, maltodextrin, sucrose, corn syrup or any combination thereof. The emulsifiers can be mono- and diglycerides, acetylated mono- and diglycerides, or propylene glycol monoesters. The salts can be trisodium citrate, monosodium phosphate, disodium phosphate, trisodium phosphate, tetrasodium pyrophosphate, monopotassium phosphate, and/or dipotassium phosphate. The composition can also contain high intensity sweeteners, such as those describe above. Suitable anti-caking agents include sodium silico-aluminates or silica dioxides. The products are combined in slurry, optionally homogenized, and spray dried in either a granular or agglomerated form. Liquid coffee creamers are simply a homogenized and pasteurized emulsion of fat (either dairy fat or hydrogenated vegetable oil), some milk solids or caseinates, corn syrup, and vanilla or other flavors, as well as a stabilizing blend. The product is usually pasteurized via HTST (high temperature short time) at 1850 F (85° C.) for 30 seconds, or UHT (ultra-high temperature), at 285° F. (141° C.) for 4 seconds, and homogenized in a two stage homogenizer at 500-3000 psi (3.45-20.7 MPa) first stage, and 200-1000 psi (1.38-6.89 MPa) second stage. The coffee creamer is usually stabilized so that it does not break down when added to coffee.

Another type of food product in which a glucan herein can be used (e.g., as a syrup) is food coatings such as icings, frostings, and glazes. In icings and frostings, the glucan can be used as a sweetener replacement (complete or partial) to lower caloric content and increase fiber content. Glazes are typically about 70-90% sugar, with most of the rest being water, and the glucan can be used to entirely or partially replace the sugar. Frosting typically contains about 2-40% of a liquid/solid fat combination, about 20-75% sweetener solids, color, flavor, and water. The glucan can be used to replace all or part of the sweetener solids, or as a bulking agent in lower fat systems.

Another type of food product in which a glucan herein can be used is pet food, such as dry or moist dog food. Pet foods are made in a variety of ways, such as extrusion, forming, and formulating as gravies. The glucan could be used at levels of 0-50% in each of these types.

Another type of food product in which a glucan herein can be used is fish and meat. Conventional corn syrup is already used in some meats, so a glucan-containing syrup herein can be used as a partial or complete substitute. For example, the glucan syrup could be added to brine before it is vacuum tumbled or injected into the meat. It could be added with salt and phosphates, and optionally with water binding ingredients such as starch, carrageenan, or soy proteins.

### Beneficial Physiological Properties

### Gas Production

A rapid rate of gas production in the lower gastrointestinal tract gives rise to gastrointestinal discomfort such as flatulence and bloating, whereas if gas production is gradual and low, the body can more easily cope. For example, it is possible that inulin gives a boost of gas production that is rapid and high when compared to the disclosed glucan at an equivalent dosage, whereas, in some embodiments, the disclosed glucan has a rate of gas release that is lower than that of inulin at an equivalent dosage.

In one embodiment, consumption of food products containing the disclosed glucan might result in a rate of gas production that is well tolerated for food applications. In one embodiment, the relative rate of gas production is no more than the rate observed for inulin under similar conditions, such as the same or less than inulin, or less than inulin, or much less than inulin at an equivalent dosage. In another embodiment, the relative rate of gas formation is measured over 3 hours or 24 hours using the methods described herein. In some embodiments, the rate of gas formation is at least 1%, 2%, 3%, 4%, 5%, 6%, 7%, 8%, 9%, 10%, 15%, 20%, 25%, or 30% less than the rate observed for inulin under the same reaction conditions.

### Short Chain Fatty Acid Production

Use of a glucan herein may facilitate the production of energy yielding metabolites through colonic fermentation. Use of a glucan herein may facilitate the production of short chain fatty acids (SCFAs), such as propionate and/or butyrate. SCFAs are known to lower cholesterol. Consequently, a glucan herein may lower the risk of developing high cholesterol. As the production of SCFAs or the increased ratio of SCFA to acetate is beneficial for the control of cholesterol levels in a mammal in need thereof, the disclosed glucan composition may be of particular interest to nutritionists and consumers for the prevention and/or treatment of cardiovascular risks. Thus, in another aspect, the disclosure provides a method for improving the health of a subject comprising administering a composition comprising the disclosed glucan composition to a subject in an amount effective to exert a beneficial effect on the health of said subject, such as for treating cholesterol-related diseases. In addition, it is generally known that SCFAs lower the pH in the gut and this helps calcium absorption. Thus, compounds according to the present disclosure may also affect mineral absorption. This means that they may also improve bone health, or prevent or treat osteoporosis by lowering the pH due to SCFA increases in the gut. The production of SCFA may increase viscosity in small intestine which reduces the re-absorption of bile acids; increasing the synthesis of bile acids from cholesterol and reduces circulating low density lipoprotein (LDL) cholesterol.

An “effective amount” of a compound or composition as defined herein refers to an amount effective, at dosages and for periods of time necessary, to achieve a desired beneficial physiological effect, such as lowering of blood cholesterol, increasing SCFA production or preventing or treating a gastrointestinal disorder. For instance, the amount of a composition administered to a subject will vary depending upon factors such as the subject's condition, the subject's body weight, the age of the subject, and whether a composition is the sole source of nutrition. The effective amount may be readily set by a medical practitioner or dietician. In general, a sufficient amount of the composition is administered to provide the subject with up to about 50 g per day; or, for example, about 25 g to about 35 g per day. In some embodiments, the amount of the disclosed glucan composition that a subject receives is in the range of about 0.1 g to about 50 g per day, or in the range of 0.5 g to 20 g per day, or 1 g to 10 g per day. A compound or composition as defined herein may be taken in multiple doses, for example 1 to 5 times, spread out over the day or acutely, or may be taken in a single dose. A compound or composition as defined herein may also be fed continuously over a desired period. In certain embodiments, the desired period is at least one week or at least two weeks or at least three weeks or at least one month or at least six months.

In some embodiments, this disclosure provides a method for decreasing blood triglyceride levels in a subject in need thereof by administering a compound or a composition as defined herein to a subject in need thereof. In another embodiment, this disclosure provides a method for decreasing low density lipoprotein levels in a subject in need thereof by administering a compound or a composition as defined herein to a subject in need thereof. In another embodiment, the disclosure provides a method for increasing high density lipoprotein levels in a subject in need thereof by administering a compound or a composition as defined herein to a subject in need thereof.

### Attenuation of Postprandial Blood Glucose Concentrations Glycemic Response

The presence of bonds other than alpha-1,4 backbone linkages in the disclosed glucan composition provides improved digestion resistance as enzymes of the human digestion track may have difficultly hydrolyzing such bonds and/or branched linkages. The presence of branches provides partial or complete indigestibility to the glucan in some embodiments, and therefore virtually no or a slower absorption of glucose into the body, which results in a lower glycemic response. Accordingly, the disclosure provides a glucan composition for the manufacture of food and drink compositions resulting in a lower glycemic response. For example, these compounds can be used to replace sugar or other rapidly digestible carbohydrates, and thereby lower the glycemic load of foods, reduce calories, and/or lower the energy density of foods. Also, the stability of the disclosed glucan composition possessing these types of bonds allows them to be easily passed through into the large intestine where they may serve as a substrate specific for the colonic microbial flora.

### Improvement of Gut Health

In a further embodiment, compounds as disclosed herein may be used for the treatment and/or improvement of gut health. In some embodiments, the glucan composition is slowly fermented in the gut by the gut microflora. In some embodiments, the present compounds exhibit (in an in vitro gut model) a tolerance no worse than inulin or other commercially available fibers such as PROMITOR® (soluble corn fiber, Tate & Lyle), NUTRIOSE® (soluble corn fiber or dextrin, Roquette), or FIBERSOL®-2 (digestion-resistant maltodextrin, Archer Daniels Midland Company & Matsutani Chemical) (i.e., similar level of gas production), and in particular, provide for an improved tolerance over one or more of the commercially available fibers, i.e. the fermentation of the present glucan results in less gas production than inulin in 3 hours or 24 hours, thereby lowering discomfort, such as flatulence and bloating, due to gas formation. In one aspect, the disclosure also relates to a method for moderating gas formation in the gastrointestinal tract of a subject by administering a compound or a composition as disclosed herein to a subject in need thereof, so as to decrease gut pain or gut discomfort due to flatulence and bloating. In further embodiments, compositions as disclosed herein provide subjects with improved tolerance to food fermentation, and may be combined with fibers, such as inulin or FOS, GOS, or lactulose to improve tolerance by lowering gas production. In another embodiment, compounds as disclosed herein may be administered to improve laxation or improve regularity by increasing stool bulk.

### Prebiotics and Probiotics

A glucan herein may be useful as a prebiotic, or as a “synbiotic” when used in combination with a probiotic, as discussed below. By “prebiotic” it is meant a food ingredient that beneficially affects a subject by selectively stimulating the growth and/or activity of one or a limited number of bacteria in the gastrointestinal tract, particularly the colon, and thus improves the health of the subject. Examples of prebiotics include fructo-oligosaccharides, inulin, polydextrose, resistant starch, soluble corn fiber, gluco-oligosaccharides, galacto-oligosaccharides, arabinoxylan-oligosaccharides, lactitol, and lactulose.

In another embodiment, compositions comprising a glucan herein can further comprise at least one probiotic organism. By “probiotic organism”, it is meant living microbiological dietary supplements that provide beneficial effects to a subject through their function in the digestive tract. In order to be effective, a probiotic micro-organisms must be able to survive the digestive conditions, and must be able to colonize the gastrointestinal tract at least temporarily without any harm to the subject. Only certain strains of microorganisms have these properties. In some embodiments, the probiotic microorganism is selected from the group comprising Lactobacillus spp., Bidobacterium spp., Bacillus spp., Enterococcus spp., Escherichia spp., Streptococcus spp., and Saccharomyces spp. Specific microorganisms include, but are not limited to Bacillus subtilis, Bacillus cereus, Bifidobacterium animalis, Bifidobacterium bificum, Bifidobacterium breve, Bifidobacterium infantis, Bifidobacterium lactis, Bifidobacterium longum, Bifidobacterium thermophilum, Enterococcus faecium, Enterococcus faecium, Lactobacillus acidophilus, Lactobacillus bulgaricus, Lactobacillus casei, Lactobacillus lactis, Lactobacillus plantarum, Lactobacillus reuteri, Lactobacillus rhamnosus, Streptococcus faecium, Streptococcus mutans, Streptococcus thermophilus, Saccharomyces boulardii, Torulopsia, Aspergillus oryzae, and Streptomyces among others, including their vegetative spores, non-vegetative spores (Bacillus) and synthetic derivatives. In some embodiments, probiotic microorganisms include, but are not limited to, members of three bacterial genera: Lactobacillus, Bifidobacterium and Saccharomyces.

In some embodiments, the composition comprises a probiotic organism in an amount sufficient to delivery at least 1 to 200 billion, 1 to 100 billion, or 1 to 50 billion viable probiotic organisms. The amount of probiotic organisms delivered as described above may be per dosage and/or per day, where multiple dosages per day may be suitable for some applications. Two or more probiotic organisms may be used in a composition, for example.

Compositions disclosed herein can be in the form of a fabric care composition. A fabric care composition herein can be used for hand wash, machine wash and/or other purposes such as soaking and/or pretreatment of fabrics, for example. A fabric care composition may take the form of, for example, a laundry detergent; fabric conditioner; any wash-, rinse-, or dryer-added product; unit dose or spray. Fabric care compositions in a liquid form may be in the form of an aqueous composition as disclosed herein. In other aspects, a fabric care composition can be in a dry form such as a granular detergent or dryer-added fabric softener sheet. Other non-limiting examples of fabric care compositions herein include: granular or powder-form all-purpose or heavy-duty washing agents; liquid, gel or paste-form all-purpose or heavy-duty washing agents; liquid or dry fine-fabric (e.g., delicates) detergents; cleaning auxiliaries such as bleach additives, “stain-stick”, or pre-treatments; substrate-laden products such as dry and wetted wipes, pads, or sponges; sprays and mists.

A detergent composition herein may be in any useful form, e.g., as powders, granules, pastes, bars, unit dose, or liquid. A liquid detergent may be aqueous, typically containing up to about 70 wt % of water and 0 wt % to about 30 wt % of organic solvent. It may also be in the form of a compact gel type containing only about 30 wt % water.

A detergent composition herein typically comprises one or more surfactants, wherein the surfactant is selected from nonionic surfactants, anionic surfactants, cationic surfactants, ampholytic surfactants, zwitterionic surfactants, semi-polar nonionic surfactants and mixtures thereof. In some embodiments, the surfactant is present at a level of from about 0.1% to about 60%, while in alternative embodiments the level is from about 1% to about 50%, while in still further embodiments the level is from about 5% to about 40%, by weight of the detergent composition. A detergent will usually contain 0 wt % to about 50 wt % of an anionic surfactant such as linear alkylbenzenesulfonate (LAS), alpha-olefinsulfonate (AOS), alkyl sulfate (fatty alcohol sulfate) (AS), alcohol ethoxysulfate (AEOS or AES), secondary alkanesulfonates (SAS), alpha-sulfo fatty acid methyl esters, alkyl- or alkenylsuccinic acid, or soap. In addition, a detergent composition may optionally contain 0 wt % to about 40 wt % of a nonionic surfactant such as alcohol ethoxylate (AEO or AE), carboxylated alcohol ethoxylates, nonylphenol ethoxylate, alkylpolyglycoside, alkyldimethylamineoxide, ethoxylated fatty acid monoethanolamide, fatty acid monoethanolamide, or polyhydroxy alkyl fatty acid amide (as described for example in WO92/06154, which is incorporated herein by reference).

A detergent composition herein typically comprises one or more detergent builders or builder systems. In some embodiments incorporating at least one builder, the cleaning compositions comprise at least about 1%, from about 3% to about 60%, or even from about 5% to about 40%, builder by weight of the composition. Builders include, but are not limited to, alkali metal, ammonium and alkanolammonium salts of polyphosphates, alkali metal silicates, alkaline earth and alkali metal carbonates, aluminosilicates, polycarboxylate compounds, ether hydroxypolycarboxylates, copolymers of maleic anhydride with ethylene or vinyl methyl ether, 1,3,5-trihydroxy benzene-2,4,6-trisulphonic acid, and carboxymethyloxysuccinic acid, various alkali metal, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof. Indeed, it is contemplated that any suitable builder will find use in various embodiments of the present disclosure. Additional examples of a detergent builder or complexing agent include zeolite, diphosphate, triphosphate, phosphonate, citrate, nitrilotriacetic acid (NTA), ethylenediaminetetraacetic acid (EDTA), diethylenetriaminepentaacetic acid (DTMPA), alkyl- or alkenylsuccinic acid, soluble silicates or layered silicates (e.g., SKS-6 from Hoechst).

In some embodiments, builders form water-soluble hardness ion complexes (e.g., sequestering builders), such as citrates and polyphosphates (e.g., sodium tripolyphosphate and sodium tripolyphospate hexahydrate, potassium tripolyphosphate, and mixed sodium and potassium tripolyphosphate, etc.). It is contemplated that any suitable builder will find use in the present disclosure, including those known in the art (See, e.g., EP2100949).

In some embodiments, suitable builders can include phosphate builders and non-phosphate builders. In some embodiments, a builder is a phosphate builder. In some embodiments, a builder is anon-phosphate builder. A builder can be used in a level of from 0.1% to 80%, or from 5% to 60%, or from 10% to 50%, by weight of the composition. In some embodiments, the product comprises a mixture of phosphate and non-phosphate builders. Suitable phosphate builders include mono-phosphates, di-phosphates, tri-polyphosphates or oligomeric-polyphosphates, including the alkali metal salts of these compounds, including the sodium salts. In some embodiments, a builder can be sodium tripolyphosphate (STPP). Additionally, the composition can comprise carbonate and/or citrate, preferably citrate that helps to achieve a neutral pH composition. Other suitable non-phosphate builders include homopolymers and copolymers of polycarboxylic acids and their partially or completely neutralized salts, monomeric polycarboxylic acids and hydroxycarboxylic acids and their salts. In some embodiments, salts of the above mentioned compounds include ammonium and/or alkali metal salts, i.e., lithium, sodium, and potassium salts, including sodium salts. Suitable polycarboxylic acids include acyclic, alicyclic, hetero-cyclic and aromatic carboxylic acids, wherein in some embodiments, they can contain at least two carboxyl groups which are in each case separated from one another by, in some instances, no more than two carbon atoms.

A detergent composition herein can comprise at least one chelating agent. Suitable chelating agents include, but are not limited to copper, iron and/or manganese chelating agents and mixtures thereof. In embodiments in which at least one chelating agent is used, the composition comprises from about 0.1% to about 15%, or even from about 3.0% to about 10%, chelating agent by weight of the composition.

A detergent composition herein can comprise at least one deposition aid. Suitable deposition aids include, but are not limited to, polyethylene glycol, polypropylene glycol, polycarboxylate, soil release polymers such as polytelephthalic acid, clays such as kaolinite, montmorillonite, atapulgite, illite, bentonite, halloysite, and mixtures thereof.

A detergent composition herein can comprise one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. Additional dye transfer inhibiting agents include manganese phthalocyanine, peroxidases, polyvinylpyrrolidone polymers, polyamine N-oxide polymers, copolymers of N-vinylpyrrolidone and N-vinylimidazole, polyvinyloxazolidones and polyvinylimidazoles and/or mixtures thereof; chelating agents examples of which include ethylene-diamine-tetraacetic acid (EDTA); diethylene triamine penta methylene phosphonic acid (DTPMP); hydroxy-ethane diphosphonic acid (HEDP); ethylenediamine N,N′-disuccinic acid (EDDS); methyl glycine diacetic acid (MGDA); diethylene triamine penta acetic acid (DTPA); propylene diamine tetracetic acid (PDT A); 2-hydroxypyridine-N-oxide (HPNO); or methyl glycine diacetic acid (MGDA); glutamic acid N,N-diacetic acid (N,N-dicarboxymethyl glutamic acid tetrasodium salt (GLDA); nitrilotriacetic acid (NTA); 4,5-dihydroxy-m-benzenedisulfonic acid; citric acid and any salts thereof; N-hydroxyethyl ethylenediaminetri-acetic acid (HEDTA), triethylenetetraaminehexaacetic acid (TTHA), N-hydroxyethyliminodiacetic acid (HEIDA), dihydroxyethylglycine (DHEG), ethylenediaminetetrapropionic acid (EDTP) and derivatives thereof, which can be used alone or in combination with any of the above. In embodiments in which at least one dye transfer inhibiting agent is used, a composition herein may comprise from about 0.0001% to about 10%, from about 0.01% to about 5%, or even from about 0.1% to about 3%, by weight of the composition.

A detergent composition herein can comprise silicates. In some of these embodiments, sodium silicates (e.g., sodium disilicate, sodium metasilicate, and/or crystalline phyllosilicates) find use. In some embodiments, silicates are present at a level of from about 1% to about 20% by weight of the composition. In some embodiments, silicates are present at a level of from about 5% to about 15% by weight of the composition.

A detergent composition herein can comprise dispersants. Suitable water-soluble organic materials include, but are not limited to the homo- or co-polymeric acids or their salts, in which the polycarboxylic acid comprises at least two carboxyl radicals separated from each other by not more than two carbon atoms.

A detergent composition herein may additionally comprise one or more enzymes as delineated above.

In some embodiments, a detergent composition can comprise one or more enzymes (e.g., any disclosed herein), each at a level from about 0.00001% to about 10% by weight of the composition and the balance of cleaning adjunct materials by weight of composition. In some other embodiments, a detergent composition can also comprise each enzyme at a level of about 0.0001% to about 10%, about 0.001% to about 5%, about 0.001% to about 2%, or about 0.005% to about 0.5%, by weight of the composition.

Enzymes that may be comprised in a detergent composition herein may be stabilized using conventional stabilizing agents, e.g., a polyol such as propylene glycol or glycerol; a sugar or sugar alcohol; lactic acid; boric acid or a boric acid derivative (e.g., an aromatic borate ester).

A detergent composition in certain embodiments may comprise one or more polymers. Examples of suitable polymers include carboxymethyl cellulose (CMC), poly(vinylpyrrolidone) (PVP), polyethylene glycol (PEG), poly(vinyl alcohol) (PVA), polycarboxylates such as polyacrylates, maleic/acrylic acid copolymers and lauryl methacrylate/acrylic acid copolymers.

A detergent composition herein may contain a bleaching system. For example, a bleaching system can comprise an H₂O₂ source such as perborate or percarbonate, which may be combined with a peracid-forming bleach activator such as tetraacetylethylenediamine (TAED) or nonanoyloxybenzenesulfonate (NOBS). Alternatively, a bleaching system may comprise peroxyacids (e.g., amide, imide, or sulfone type peroxyacids). Alternatively still, a bleaching system can be an enzymatic bleaching system comprising perhydrolase, for example, such as the system described in WO2005/056783.

A detergent composition herein may also contain conventional detergent ingredients such as fabric conditioners, clays, foam boosters, suds suppressors, anti-corrosion agents, soil-suspending agents, anti-soil redeposition agents, dyes, bactericides, tarnish inhibiters, optical brighteners, or perfumes. The pH of a detergent composition herein (measured in aqueous solution at use concentration) is usually neutral or alkaline (e.g., pH of about 7.0 to about 11.0).

A detergent composition herein may also contain at least one anti-redeposition agent and/or clay soil removal agent (such agents can optionally be characterized as whiteness maintenance agents in certain aspects). Examples of suitable anti-redeposition and/or clay soil removal agents herein include polyethoxy zwitterionic surfactants, water-soluble copolymers of acrylic or methacrylic acid with acrylic or methacrylic acid-ethylene oxide condensates (e.g., U.S. Pat. No. 3,719,647), cellulose derivatives such as carboxymethylcellulose and hydroxypropylcellulose (e.g., U.S. Pat. Nos. 3,597,416 and 3,523,088), and mixtures comprising nonionic alkyl polyethoxy surfactant, polyethoxy alkyl quaternary cationic surfactant and fatty amide surfactant (e.g., U.S. Pat. No. 4,228,044). Non-limiting examples of other suitable anti-redeposition and clay soil removal agents are disclosed in U.S. Pat. Nos. 4,597,898 and 4,891,160, and Int. Pat. Appl. Publ. No. WO95/32272, all of which are incorporated herein by reference.

Particular forms of detergent compositions that can be adapted for purposes disclosed herein are disclosed in, for example, US20090209445A1, US20100081598A1, U.S. Pat. No. 7,001,878B2, EP1504994B1, WO2001085888A2, WO2003089562A1, WO2009098659A1, WO2009098660A1, WO2009112992A1, WO2009124160A1, WO2009152031A1, WO2010059483A1, WO2010088112A1, WO2010090915A1, WO2010135238A1, WO2011094687A1, WO2011094690A1, WO2011127102A1, WO2011163428A1, WO2008000567A1, WO2006045391A1, WO2006007911A1, WO2012027404A1, EP1740690B1, WO2012059336A1, U.S. Pat. No. 6,730,646B1, WO2008087426A1, WO2010116139A1, and WO2012104613A1, all of which are incorporated herein by reference.

Laundry detergent compositions herein can optionally be heavy duty (all purpose) laundry detergent compositions. Exemplary heavy duty laundry detergent compositions comprise a detersive surfactant (10%-40% wt/wt), including an anionic detersive surfactant (selected from a group of linear or branched or random chain, substituted or unsubstituted alkyl sulphates, alkyl sulphonates, alkyl alkoxylated sulphate, alkyl phosphates, alkyl phosphonates, alkyl carboxylates, and/or mixtures thereof), and optionally non-ionic surfactant (selected from a group of linear or branched or random chain, substituted or unsubstituted alkyl alkoxylated alcohol, e.g., C8-C18 alkyl ethoxylated alcohols and/or C6-C12 alkyl phenol alkoxylates), where the weight ratio of anionic detersive surfactant (with a hydrophilic index (HIc) of from 6.0 to 9) to non-ionic detersive surfactant is greater than 1:1. Suitable detersive surfactants also include cationic detersive surfactants (selected from a group of alkyl pyridinium compounds, alkyl quaternary ammonium compounds, alkyl quaternary phosphonium compounds, alkyl ternary sulphonium compounds, and/or mixtures thereof); zwitterionic and/or amphoteric detersive surfactants (selected from a group of alkanolamine sulpho-betaines); ampholytic surfactants; semi-polar non-ionic surfactants and mixtures thereof.

A detergent herein such as a heavy duty laundry detergent composition may optionally include, a surfactancy boosting polymer consisting of amphiphilic alkoxylated grease cleaning polymers (selected from a group of alkoxylated polymers having branched hydrophilic and hydrophobic properties, such as alkoxylated polyalkylenimines in the range of 0.05 wt %-10 wt %) and/or random graft polymers (typically comprising of hydrophilic backbone comprising monomers selected from the group consisting of: unsaturated C1-C6 carboxylic acids, ethers, alcohols, aldehydes, ketones, esters, sugar units, alkoxy units, maleic anhydride, saturated polyalcohols such as glycerol, and mixtures thereof; and hydrophobic side chain(s) selected from the group consisting of: C4-C25 alkyl group, polypropylene, polybutylene, vinyl ester of a saturated C1-C6 mono-carboxylic acid, C1-C6 alkyl ester of acrylic or methacrylic acid, and mixtures thereof.

A detergent herein such as a heavy duty laundry detergent composition may optionally include additional polymers such as soil release polymers (include anionically end-capped polyesters, for example SRP1, polymers comprising at least one monomer unit selected from saccharide, dicarboxylic acid, polyol and combinations thereof, in random or block configuration, ethylene terephthalate-based polymers and co-polymers thereof in random or block configuration, for example REPEL-O-TEX SF, SF-2 AND SRP6, TEXCARE SRA100, SRA300, SRN100, SRN170, SRN240, SRN300 AND SRN325, MARLOQUEST SL), anti-redeposition agent(s) herein (0.1 wt % to 10 wt %), include carboxylate polymers, such as polymers comprising at least one monomer selected from acrylic acid, maleic acid (or maleic anhydride), fumaric acid, itaconic acid, aconitic acid, mesaconic acid, citraconic acid, methylenemalonic acid, and any mixture thereof, vinylpyrrolidone homopolymer, and/or polyethylene glycol, molecular weight in the range of from 500 to 100,000 Da); and polymeric carboxylate (such as maleate/acrylate random copolymer or polyacrylate homopolymer).

A detergent herein such as a heavy duty laundry detergent composition may optionally further include saturated or unsaturated fatty acids, preferably saturated or unsaturated C12-C24 fatty acids (0 wt % to 10 wt %); deposition aids disclosed herein (examples for which include polysaccharides, cellulosic polymers, poly diallyl dimethyl ammonium halides (DADMAC), and co-polymers of DAD MAC with vinyl pyrrolidone, acrylamides, imidazoles, imidazolinium halides, and mixtures thereof, in random or block configuration, cationic guar gum, cationic starch, cationic polyacylamides, and mixtures thereof).

A detergent herein such as a heavy duty laundry detergent composition may optionally further include dye transfer inhibiting agents, examples of which include manganese phthalocyanine, peroxidases, polyvinylpyrrolidone polymers, polyamine N-oxide polymers, copolymers of N-vinylpyrrolidone and N-vinylimidazole, polyvinyloxazolidones and polyvinylimidazoles and/or mixtures thereof; chelating agents, examples of which include ethylene-diamine-tetraacetic acid (EDTA), diethylene triamine penta methylene phosphonic acid (DTPMP), hydroxy-ethane diphosphonic acid (HEDP), ethylenediamine N,N′-disuccinic acid (EDDS), methyl glycine diacetic acid (MGDA), diethylene triamine penta acetic acid (DTPA), propylene diamine tetracetic acid (PDTA), 2-hydroxypyridine-N-oxide (HPNO), or methyl glycine diacetic acid (MGDA), glutamic acid N,N-diacetic acid (N,N-dicarboxymethyl glutamic acid tetrasodium salt (GLDA), nitrilotriacetic acid (NTA), 4,5-dihydroxy-m-benzenedisulfonic acid, citric acid and any salts thereof, N-hydroxyethylethylenediaminetriacetic acid (HEDTA), triethylenetetraaminehexaacetic acid (TTHA), N-hydroxyethyliminodiacetic acid (HEIDA), dihydroxyethylglycine (DHEG), ethylenediaminetetrapropionic acid (EDTP), and derivatives thereof.

A detergent herein such as a heavy duty laundry detergent composition may optionally include silicone or fatty-acid based suds suppressors; hueing dyes, calcium and magnesium cations, visual signaling ingredients, anti-foam (0.001 wt % to about 4.0 wt %), and/or a structurant/thickener (0.01 wt % to 5 wt %) selected from the group consisting of diglycerides and triglycerides, ethylene glycol distearate, microcrystalline cellulose, microfiber cellulose, biopolymers, xanthan gum, gellan gum, and mixtures thereof). A structurant can also be referred to as a structural agent.

A detergent herein can be in the form of a heavy duty dry/solid laundry detergent composition, for example. Such a detergent may include: (i) a detersive surfactant, such as any anionic detersive surfactant disclosed herein, any non-ionic detersive surfactant disclosed herein, any cationic detersive surfactant disclosed herein, any zwitterionic and/or amphoteric detersive surfactant disclosed herein, any ampholytic surfactant, any semi-polar non-ionic surfactant, and mixtures thereof; (ii) a builder, such as any phosphate-free builder (e.g., zeolite builders in the range of 0 wt % to less than 10 wt %), any phosphate builder (e.g., sodium tri-polyphosphate in the range of 0 wt % to less than 10 wt %), citric acid, citrate salts and nitrilotriacetic acid, any silicate salt (e.g., sodium or potassium silicate or sodium meta-silicate in the range of 0 wt % to less than 10 wt %); any carbonate salt (e.g., sodium carbonate and/or sodium bicarbonate in the range of 0 wt % to less than 80 wt %), and mixtures thereof; (iii) a bleaching agent, such as any photobleach (e.g., sulfonated zinc phthalocyanines, sulfonated aluminum phthalocyanines, xanthenes dyes, and mixtures thereof), any hydrophobic or hydrophilic bleach activator (e.g., dodecanoyl oxybenzene sulfonate, decanoyl oxybenzene sulfonate, decanoyl oxybenzoic acid or salts thereof, 3,5,5-trimethy hexanoyl oxybenzene sulfonate, tetraacetyl ethylene diamine-TAED, nonanoyloxybenzene sulfonate-NOBS, nitrile quats, and mixtures thereof), any source of hydrogen peroxide (e.g., inorganic perhydrate salts, examples of which include mono or tetra hydrate sodium salt of perborate, percarbonate, persulfate, perphosphate, or persilicate), any preformed hydrophilic and/or hydrophobic peracids (e.g., percarboxylic acids and salts, percarbonic acids and salts, perimidic acids and salts, peroxymonosulfuric acids and salts, and mixtures thereof); and/or (iv) any other components such as a bleach catalyst (e.g., imine bleach boosters examples of which include iminium cations and polyions, iminium zwitterions, modified amines, modified amine oxides, N-sulphonyl imines, N-phosphonyl imines, N-acyl imines, thiadiazole dioxides, perfluoroimines, cyclic sugar ketones, and mixtures thereof), and a metal-containing bleach catalyst (e.g., copper, iron, titanium, ruthenium, tungsten, molybdenum, or manganese cations along with an auxiliary metal cations such as zinc or aluminum and a sequestrate such as EDTA, ethylenediaminetetra(methylenephosphonic acid).

Compositions disclosed herein can be in the form of a dishwashing detergent composition. Examples of dishwashing detergents include automatic dishwashing detergents (typically used in dishwasher machines) and hand-washing dish detergents. A dishwashing detergent composition can be in any dry or liquid/aqueous form as disclosed herein, for example. Components that may be included in certain embodiments of a dishwashing detergent composition include, for example, one or more of a phosphate; oxygen- or chlorine-based bleaching agent; non-ionic surfactant; alkaline salt (e.g., metasilicates, alkali metal hydroxides, sodium carbonate); any active enzyme disclosed herein; anti-corrosion agent (e.g., sodium silicate); anti-foaming agent; additives to slow down the removal of glaze and patterns from ceramics; perfume; anti-caking agent (in granular detergent); starch (in tablet-based detergents); gelling agent (in liquid/gel based detergents); and/or sand (powdered detergents).

Dishwashing detergents such as an automatic dishwasher detergent or liquid dishwashing detergent can comprise (i) a non-ionic surfactant, including any ethoxylated non-ionic surfactant, alcohol alkoxylated surfactant, epoxy-capped poly(oxyalkylated) alcohol, or amine oxide surfactant present in an amount from 0 to 10 wt %; (ii) a builder, in the range of about 5-60 wt %, including any phosphate builder (e.g., mono-phosphates, di-phosphates, tri-polyphosphates, other oligomeric-polyphosphates, sodium tripolyphosphate-STPP), any phosphate-free builder (e.g., amino acid-based compounds including methyl-glycine-diacetic acid [MGDA] and salts or derivatives thereof, glutamic-N,N-diacetic acid [GLDA] and salts or derivatives thereof, iminodisuccinic acid (IDS) and salts or derivatives thereof, carboxy methyl inulin and salts or derivatives thereof, nitrilotriacetic acid [NTA], diethylene triamine penta acetic acid [DTPA], B-alaninediacetic acid [B-ADA] and salts thereof), homopolymers and copolymers of poly-carboxylic acids and partially or completely neutralized salts thereof, monomeric polycarboxylic acids and hydroxycarboxylic acids and salts thereof in the range of 0.5 wt % to 50 wt %, or sulfonated/carboxylated polymers in the range of about 0.1 wt % to about 50 wt %; (iii) a drying aid in the range of about 0.1 wt % to about 10 wt % (e.g., polyesters, especially anionic polyesters, optionally together with further monomers with 3 to 6 functionalities—typically acid, alcohol or ester functionalities which are conducive to polycondensation, polycarbonate-, polyurethane- and/or polyurea-polyorganosiloxane compounds or precursor compounds thereof, particularly of the reactive cyclic carbonate and urea type); (iv) a silicate in the range from about 1 wt % to about 20 wt % (e.g., sodium or potassium silicates such as sodium disilicate, sodium meta-silicate and crystalline phyllosilicates); (v) an inorganic bleach (e.g., perhydrate salts such as perborate, percarbonate, perphosphate, persulfate and persilicate salts) and/or an organic bleach (e.g., organic peroxyacids such as diacyl- and tetraacylperoxides, especially diperoxydodecanedioic acid, diperoxytetradecanedioic acid, and diperoxyhexadecanedioic acid); (vi) a bleach activator (e.g., organic peracid precursors in the range from about 0.1 wt % to about 10 wt %) and/or bleach catalyst (e.g., manganese triazacyclononane and related complexes; Co, Cu, Mn, and Fe bispyridylamine and related complexes; and pentamine acetate cobalt(III) and related complexes); (vii) a metal care agent in the range from about 0.1 wt % to 5 wt % (e.g., benzatriazoles, metal salts and complexes, and/or silicates); and/or (viii) any active enzyme disclosed herein in the range from about 0.01 to 5.0 mg of active enzyme per gram of automatic dishwashing detergent composition, and an enzyme stabilizer component (e.g., oligosaccharides, polysaccharides, and inorganic divalent metal salts).

It is believed that numerous commercially available detergent formulations can be adapted to include an alpha-1,2-branched glucan as disclosed herein. Examples include PUREX® ULTRAPACKS (Henkel), FINISH® QUANTUM (Reckitt Benckiser), CLOROX™ 2 PACKS (Clorox), OXICLEAN MAX FORCE POWER PAKS (Church & Dwight), TIDE® STAIN RELEASE, CASCADE® ACTIONPACS, and TIDE® PODS™ (Procter & Gamble).

Compositions disclosed herein can be in the form of an oral care composition, for example. Examples of oral care compositions include dentifrices, toothpaste, mouth wash, mouth rinse, chewing gum, edible strips, and tooth cream/gel that provide some form of oral care (e.g., treatment or prevention of cavities [dental caries], gingivitis, plaque, tartar, and/or periodontal disease). An oral care composition can also be for treating an “oral surface”, which encompasses any soft or hard surface within the oral cavity including surfaces of the tongue, hard and soft palate, buccal mucosa, gums and dental surfaces. A “dental surface” herein is a surface of a natural tooth or a hard surface of artificial dentition including a crown, cap, filling, bridge, denture, or dental implant, for example.

An oral care composition herein can comprise about 0.01-15.0 wt % (e.g., ˜0.1-10 wt % or ˜0.1-5.0 wt %, ˜0.1-2.0 wt %) of one or more alpha-1,2-branched glucans as disclosed herein, for example. One or more other thickening or dispersion agents can also be provided in an oral care composition herein, such as a carboxyvinyl polymer, carrageenan (e.g., L-carrageenan), natural gum (e.g., karaya, xanthan, gum arabic, tragacanth), colloidal magnesium aluminum silicate, or colloidal silica, for example.

An oral care composition herein may be a toothpaste or other dentifrice, for example. Such compositions, as well as any other oral care composition herein, can additionally comprise, without limitation, one or more of an anticaries agent, antimicrobial or antibacterial agent, anticalculus or tartar control agent, surfactant, abrasive, pH-modifying agent, foam modulator, humectant, flavorant, sweetener, pigment/colorant, whitening agent, and/or other suitable components. Examples of oral care compositions to which one or more alpha-1,2-branched glucans herein can be added are disclosed in U.S. Patent Appl. Publ. Nos. 2006/0134025, 2002/0022006 and 2008/0057007, which are incorporated herein by reference.

An anticaries agent herein can be an orally acceptable source of fluoride ions. Suitable sources of fluoride ions include fluoride, monofluorophosphate and fluorosilicate salts as well as amine fluorides, including olaflur (N′-octadecyltrimethylendiamine-N,N,N′-tris(2-ethanol)-dihydrofluoride), for example. An anticaries agent can be present in an amount providing a total of about 100-20000 ppm, about 200-5000 ppm, or about 500-2500 ppm, fluoride ions to the composition, for example. In oral care compositions in which sodium fluoride is the sole source of fluoride ions, an amount of about 0.01-5.0 wt %, about 0.05-1.0 wt %, or about 0.1-0.5 wt %, sodium fluoride can be present in the composition, for example.

An antimicrobial or antibacterial agent suitable for use in an oral care composition herein includes, for example, phenolic compounds (e.g., 4-allylcatechol; p-hydroxybenzoic acid esters such as benzylparaben, butylparaben, ethylparaben, methylparaben and propylparaben; 2-benzylphenol; butylated hydroxyanisole; butylated hydroxytoluene; capsaicin; carvacrol; creosol; eugenol; guaiacol; halogenated bisphenolics such as hexachlorophene and bromochlorophene; 4-hexylresorcinol; 8-hydroxyquinoline and salts thereof; salicylic acid esters such as menthyl salicylate, methyl salicylate and phenyl salicylate; phenol; pyrocatechol; salicylanilide; thymol; halogenated diphenylether compounds such as triclosan and triclosan monophosphate), copper (II) compounds (e.g., copper (II) chloride, fluoride, sulfate and hydroxide), zinc ion sources (e.g., zinc acetate, citrate, gluconate, glycinate, oxide, and sulfate), phthalic acid and salts thereof (e.g., magnesium monopotassium phthalate), hexetidine, octenidine, sanguinarine, benzalkonium chloride, domiphen bromide, alkylpyridinium chlorides (e.g. cetylpyridinium chloride, tetradecylpyridinium chloride, N-tetradecyl-4-ethylpyridinium chloride), iodine, sulfonamides, bisbiguanides (e.g., alexidine, chlorhexidine, chlorhexidine digluconate), piperidino derivatives (e.g., delmopinol, octapinol), magnolia extract, grapeseed extract, rosemary extract, menthol, geraniol, citral, eucalyptol, antibiotics (e.g., augmentin, amoxicillin, tetracycline, doxycycline, minocycline, metronidazole, neomycin, kanamycin, clindamycin), and/or any antibacterial agents disclosed in U.S. Pat. No. 5,776,435, which is incorporated herein by reference. One or more antimicrobial agents can optionally be present at about 0.01-10 wt % (e.g., 0.1-3 wt %), for example, in the disclosed oral care composition.

An anticalculus or tartar control agent suitable for use in an oral care composition herein includes, for example, phosphates and polyphosphates (e.g., pyrophosphates), polyaminopropanesulfonic acid (AMPS), zinc citrate trihydrate, polypeptides (e.g., polyaspartic and polyglutamic acids), polyolefin sulfonates, polyolefin phosphates, diphosphonates (e.g., azacycloalkane-2,2-diphosphonates such as azacycloheptane-2,2-diphosphonic acid), N-methyl azacyclopentane-2,3-diphosphonic acid, ethane-1-hydroxy-1,1-diphosphonic acid (EHDP), ethane-1-amino-1,1-diphosphonate, and/or phosphonoalkane carboxylic acids and salts thereof (e.g., their alkali metal and ammonium salts). Useful inorganic phosphate and polyphosphate salts include, for example, monobasic, dibasic and tribasic sodium phosphates, sodium tripolyphosphate, tetrapolyphosphate, mono-, di-, tri- and tetra-sodium pyrophosphates, disodium dihydrogen pyrophosphate, sodium trimetaphosphate, sodium hexametaphosphate, or any of these in which sodium is replaced by potassium or ammonium. Other useful anticalculus agents in certain embodiments include anionic polycarboxylate polymers (e.g., polymers or copolymers of acrylic acid, methacrylic, and maleic anhydride such as polyvinyl methyl ether/maleic anhydride copolymers). Still other useful anticalculus agents include sequestering agents such as hydroxycarboxylic acids (e.g., citric, fumaric, malic, glutaric and oxalic acids and salts thereof) and aminopolycarboxylic acids (e.g., EDTA). One or more anticalculus or tartar control agents can optionally be present at about 0.01-50 wt % (e.g., about 0.05-25 wt % or about 0.1-15 wt %), for example, in the disclosed oral care composition.

A surfactant suitable for use in an oral care composition herein may be anionic, non-ionic, or amphoteric, for example. Suitable anionic surfactants include, without limitation, water-soluble salts of C₈₋₂₀ alkyl sulfates, sulfonated monoglycerides of C₈₋₂₀ fatty acids, sarcosinates, and taurates. Examples of anionic surfactants include sodium lauryl sulfate, sodium coconut monoglyceride sulfonate, sodium lauryl sarcosinate, sodium lauryl isoethionate, sodium laureth carboxylate and sodium dodecyl benzenesulfonate. Suitable non-ionic surfactants include, without limitation, poloxamers, polyoxyethylene sorbitan esters, fatty alcohol ethoxylates, alkylphenol ethoxylates, tertiary amine oxides, tertiary phosphine oxides, and dialkyl sulfoxides. Suitable amphoteric surfactants include, without limitation, derivatives of C₈₋₂₀ aliphatic secondary and tertiary amines having an anionic group such as a carboxylate, sulfate, sulfonate, phosphate or phosphonate. An example of a suitable amphoteric surfactant is cocoamidopropyl betaine. One or more surfactants are optionally present in a total amount of about 0.01-10 wt % (e.g., about 0.05-5.0 wt % or about 0.1-2.0 wt %), for example, in the disclosed oral care composition.

An abrasive suitable for use in an oral care composition herein may include, for example, silica (e.g., silica gel, hydrated silica, precipitated silica), alumina, insoluble phosphates, calcium carbonate, and resinous abrasives (e.g., a urea-formaldehyde condensation product). Examples of insoluble phosphates useful as abrasives herein are orthophosphates, polymetaphosphates and pyrophosphates, and include dicalcium orthophosphate dihydrate, calcium pyrophosphate, beta-calcium pyrophosphate, tricalcium phosphate, calcium polymetaphosphate and insoluble sodium polymetaphosphate. One or more abrasives are optionally present in a total amount of about 5-70 wt % (e.g., about 10-56 wt % or about 15-30 wt %), for example, in the disclosed oral care composition. The average particle size of an abrasive in certain embodiments is about 0.1-30 microns (e.g., about 1-20 microns or about 5-15 microns).

An oral care composition in certain embodiments may comprise at least one pH-modifying agent. Such agents may be selected to acidify, make more basic, or buffer the pH of a composition to a pH range of about 2-10 (e.g., pH ranging from about 2-8, 3-9, 4-8, 5-7, 6-10, or 7-9). Examples of pH-modifying agents useful herein include, without limitation, carboxylic, phosphoric and sulfonic acids; acid salts (e.g., monosodium citrate, disodium citrate, monosodium malate); alkali metal hydroxides (e.g. sodium hydroxide, carbonates such as sodium carbonate, bicarbonates, sesquicarbonates); borates; silicates; phosphates (e.g., monosodium phosphate, trisodium phosphate, pyrophosphate salts); and imidazole.

A foam modulator suitable for use in an oral care composition herein may be a polyethylene glycol (PEG), for example. High molecular weight PEGs are suitable, including those having an average molecular weight of about 200000-7000000 (e.g., about 500000-5000000 or about 1000000-2500000), for example. One or more PEGs are optionally present in a total amount of about 0.1-10 wt % (e.g. about 0.2-5.0 wt % or about 0.25-2.0 wt %), for example, in the disclosed oral care composition.

An oral care composition in certain embodiments may comprise at least one humectant. A humectant in certain embodiments may be a polyhydric alcohol such as glycerin, sorbitol, xylitol, or a low molecular weight PEG. Most suitable humectants also may function as a sweetener herein. One or more humectants are optionally present in a total amount of about 1.0-70 wt % (e.g., about 1.0-50 wt %, about 2-25 wt %, or about 5-15 wt %), for example, in the disclosed oral care composition.

A natural or artificial sweetener may optionally be comprised in an oral care composition herein. Examples of suitable sweeteners include dextrose, sucrose, maltose, dextrin, invert sugar, mannose, xylose, ribose, fructose, levulose, galactose, corn syrup (e.g., high fructose corn syrup or corn syrup solids), partially hydrolyzed starch, hydrogenated starch hydrolysate, sorbitol, mannitol, xylitol, maltitol, isomalt, aspartame, neotame, saccharin and salts thereof, dipeptide-based intense sweeteners, and cyclamates. One or more sweeteners are optionally present in a total amount of about 0.005-5.0 wt %, for example, in the disclosed oral care composition.

A natural or artificial flavorant may optionally be comprised in an oral care composition herein. Examples of suitable flavorants include vanillin; sage; marjoram; parsley oil; spearmint oil; cinnamon oil; oil of wintergreen (methylsalicylate); peppermint oil; clove oil; bay oil; anise oil; eucalyptus oil; citrus oils; fruit oils; essences such as those derived from lemon, orange, lime, grapefruit, apricot, banana, grape, apple, strawberry, cherry, or pineapple; bean- and nut-derived flavors such as coffee, cocoa, cola, peanut, or almond; and adsorbed and encapsulated flavorants. Also encompassed within flavorants herein are ingredients that provide fragrance and/or other sensory effect in the mouth, including cooling or warming effects. Such ingredients include, without limitation, menthol, menthyl acetate, menthyl lactate, camphor, eucalyptus oil, eucalyptol, anethole, eugenol, cassia, oxanone, Irisone®, propenyl guaiethol, thymol, linalool, benzaldehyde, cinnamaldehyde, N-ethyl-p-menthan-3-carboxamine, N,2,3-trimethyl-2-isopropylbutanamide, 3-(1-menthoxy)-propane-1,2-diol, cinnamaldehyde glycerol acetal (CGA), and menthone glycerol acetal (MGA). One or more flavorants are optionally present in a total amount of about 0.01-5.0 wt % (e.g., about 0.1-2.5 wt %), for example, in the disclosed oral care composition.

An oral care composition in certain embodiments may comprise at least one bicarbonate salt. Any orally acceptable bicarbonate can be used, including alkali metal bicarbonates such as sodium or potassium bicarbonate, and ammonium bicarbonate, for example. One or more bicarbonate salts are optionally present in a total amount of about 0.1-50 wt % (e.g., about 1-20 wt %), for example, in the disclosed oral care composition.

An oral care composition in certain embodiments may comprise at least one whitening agent and/or colorant. A suitable whitening agent is a peroxide compound such as any of those disclosed in U.S. Pat. No. 8,540,971, which is incorporated herein by reference. Suitable colorants herein include pigments, dyes, lakes and agents imparting a particular luster or reflectivity such as pearling agents, for example. Specific examples of colorants useful herein include talc; mica; magnesium carbonate; calcium carbonate; magnesium silicate; magnesium aluminum silicate; silica; titanium dioxide; zinc oxide; red, yellow, brown and black iron oxides; ferric ammonium ferrocyanide; manganese violet; ultramarine; titaniated mica; and bismuth oxychloride. One or more colorants are optionally present in a total amount of about 0.001-20 wt % (e.g., about 0.01-10 wt % or about 0.1-5.0 wt %), for example, in the disclosed oral care composition.

Additional components that can optionally be included in an oral composition herein include one or more enzymes (above), vitamins, and anti-adhesion agents, for example. Examples of vitamins useful herein include vitamin C, vitamin E, vitamin B5, and folic acid. Examples of suitable anti-adhesion agents include solbrol, ficin, and quorum-sensing inhibitors. A composition comprising an alpha-1,2-branched glucan herein can be an ether derivative of the glucan in some embodiments (i.e., an alpha-1,2-branched glucan herein can be derivatized to be ether-linked to one or more different organic groups). The degree of substitution (DoS) of an alpha-1,2-branched glucan with one or more etherified organic groups can be about 0.0025 to about 3.0, for example. Alternatively, the DoS can be about, or at least about, 0.0025, 0.005, 0.01, 0.025, 0.05, 0.075, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0, for example (can optionally be expressed as a range between any two of these values).

An organic group etherified to an alpha-1,2-branched glucan herein can be an alkyl group such as a methyl, ethyl, propyl, butyl, pentyl, hexyl, heptyl, octyl, nonyl, or decyl group, for example. In some aspects, an organic group etherified to an alpha-1,2-branched glucan can be a substituted alkyl group in which there is a substitution on one or more carbons of the alkyl group. The substitution(s) may be one or more hydroxyl, aldehyde, ketone, and/or carboxyl groups. For example, a substituted alkyl group may be a hydroxy alkyl group, dihydroxy alkyl group, or carboxy alkyl group. Examples of suitable hydroxy alkyl groups are hydroxymethyl, hydroxyethyl, hydroxypropyl, hydroxybutyl and hydroxypentyl groups. Other examples include dihydroxy alkyl groups (diols) such as dihydroxymethyl, dihydroxyethyl, dihydroxypropyl, dihydroxybutyl and dihydroxypentyl groups. Examples of suitable carboxy alkyl groups are carboxymethyl (—CH₂COOH), carboxyethyl, carboxypropyl, carboxybutyl and carboxypentyl groups.

An organic group etherified to an alpha-1,2-branched glucan can be a positively charged organic group in some aspects. A positively charged group herein can be a substituted ammonium group, for example. Examples of substituted ammonium groups are primary, secondary, tertiary and quaternary ammonium groups. Further examples of suitable positively charged groups are disclosed in U.S. Pat. Appl. Publ. No. 2016/0311935, which is incorporated herein by reference.

An alpha-1,2-branched glucan ether compound in certain embodiments can contain one type of organic group. A specific non-limiting example of such a compound is carboxymethyl alpha-1,2-branched glucan. Alternatively, an alpha-1,2-branched glucan ether compound can contain two or more different types of organic groups. In some aspects, a glucan ether compound herein can comprise at least one nonionic organic group and at least one anionic group as ether groups. In some aspects, a glucan ether compound herein can comprise at least one nonionic organic group and at least one positively charged organic group as ether groups.

The percentage of the monosaccharide units of an alpha-1,2-branched glucan ether compound herein that are ether-linked to an organic group (i.e., where one or more hydroxyl groups of a monosaccharide monomeric unit have been etherified) can vary depending on the degree to which an alpha-1,2-branched glucan is etherified in an etherification reaction. This percentage can be at least about 30%, 40%, 50%, 60%, 70%, 80%, 90%, or 100% (or any integer value between 30% and 100%), for example.

Any alpha-1,2-branched glucan as presently disclosed can be used to prepare a corresponding ether compound. Any suitable process for ether-derivatizing oligosaccharides and/or polysaccharides can be employed, such as disclosed in U.S. Pat. Nos. 2,961,439, 2,344,179, 2,203,703, 2,203,704, 2,380,879, and 2,974,134, U.S. Pat. Appl. Publ. Nos. 2014/179913, 2016/0304629, 2016/0311935, 2015/0232785, and 20150239995, and Int. Pat. Appl. Publ. No. WO16/160738, all of which are incorporated herein by reference.

Solely as an example, an ether compound herein can be prepared by (i) adding about 35-45% (e.g., 40%) alpha-1,2 branches to a glucan backbone with at least 95% (e.g., 100%) alpha-1,6 linkages and a molecular weight of about 15-20 kD (e.g. ˜17 kD), and (ii) etherifying the alpha-1,2-branched glucan product with any organic group disclosed above (e.g., carboxymethyl group).

Another aspect of the present disclosure regards a method of producing a glucan composition that comprises alpha-1,2 linkages. Such a method can comprise the steps of:
(a) providing at least the following reaction components: water, sucrose, an alpha-glucan substrate, and a polypeptide that is capable of forming at least one alpha-1,2 branch from the alpha-glucan substrate, wherein the polypeptide comprises an amino acid sequence that is at least 90% identical to:
   (i) the mature form of a sequence selected from the group consisting of SEQ ID NOs:4, 1, 2, 3, 5, 6, 7, 8, 9, 10, 11, 12, and 13;
   (ii) SEQ ID NO:27 or a sub-sequence within any one of SEQ ID NOs:4, 2, 3, 5, 6, 7, 8, 9, 10, 11, 12, or 13 that aligns with SEQ ID NO:27; and/or
   (iii) a sequence selected from the group consisting of SEQ ID NOs:4, 1, 2, 3, 5, 6, 7, 8, 9, 10, 11, 12, and 13;
(b) combining the reaction components under suitable conditions whereby the polypeptide catalyzes the synthesis of at least one alpha-1,2 branch from the alpha-glucan substrate, thereby forming a glucan composition comprising one or more alpha-1,2 linkages; and
(c) optionally isolating the glucan composition comprising one or more alpha-1,2 linkages. Any of the features disclosed herein (e.g., above and in the below Examples) regarding a reaction composition (1,2-branching reaction) can characterize appropriate aspects of such a glucan production method, and vice versa.

The production of a glucan composition comprising alpha-1,2 linkages can be carried out by combining the reaction components under any suitable reaction conditions, such as those disclosed herein. A reaction may be carried out in an aqueous solution, and/or in certain embodiments, it can be carried out in situ within a product (e.g., a food, pharmaceutical, personal care, household care, or industrial product) following any known methodology. In certain embodiments, 1,2-branching enzyme(s) is added to a sucrose-containing liquid food product. The enzyme can reduce the amount of sucrose in the liquid food product while increasing the amount of fructose and alpha-1,2-branched glucan. Suitable methodology for in situ production of glucan within a food product can be found in WO2013/182686, for example, which is incorporated herein by reference.

The concentration of a 1,2-branching enzyme herein can depend on its specific catalytic activity, and typically is chosen to obtain the desired overall rate of reaction. The enzyme concentration typically ranges from 0.0001 mg to 20 mg per mL of total reaction volume, or from 0.001 mg to 10 mg per mL. The 1,2-branching enzyme may also be immobilized on a soluble or insoluble support using known methods; see for example, Immobilization of Enzymes and Cells; Gordon F. Bickerstaff, Editor; Humana Press, Totowa, NJ, USA; 1997. 1,2-branching enzyme may be provided in whole microbial cells, as microbial cell surface-displayed enzyme, permeabilized microbial cells, microbial cell extracts, partially purified form or purified form, or any mixture thereof.

In certain embodiments, a method to produce an alpha-1,2-branched glucan composition further includes step (d) of concentrating the glucan composition.

In certain embodiments, the alpha-glucan substrate concentration at the initiation of alpha-1,2 branching is at least about 10 g/L, or 50 g/L to 500 g/L, or 100 g/L to 500 g/L, or 150 g/L to 450 g/L, or 250 g/L to 450 g/L, or 250 g/L to 600 g/L.

The sucrose concentration used during the reaction may vary. In certain embodiments, the sucrose concentration initially present when the reaction components are combined is at least about 50 g/L, or 50 g/L to 600 g/L, or 100 g/L to 500 g/L, or 100 g/L to 200 g/L, or 150 g/L to 450 g/L, or 200 g/L to 450 g/L, or 250 g/L to 600 g/L. In particular embodiments, the sucrose concentration is about 200 g/L or 100 g/L. Higher concentrations of sucrose may be used if the reaction occurs concomitantly with an alpha-glucan substrate preparation reaction.

The weight ratio of sucrose to alpha-glucan substrate backbone during a 1,2 branching reaction herein may vary. In one embodiment, the weight ratio of sucrose to alpha-glucan substrate backbone may range from 0.01:1.0 to 1.0:0.01, inclusive. In certain embodiments, the method is carried out at a pH between 3 and 8, or between 4 and 8, or between 5 and 8, or between 5.5 and 7.5, or between 5.5 to about 6.5. In certain embodiments, the set of reaction components includes a suitable buffer including, but not limited to, phosphate, pyrophosphate, bicarbonate, acetate, or citrate. The concentration of buffer, when employed, is typically from 0.1 mM to 1.0 M, or from 1 mM to 300 mM, or from 10 mM to 100 mM. The method may optionally utilize pH control to maintain an optimum pH over the course of the reaction, by incremental or continuous addition of a suitable acid or base to maintain pH in the desired range for optimal enzyme activity.

The duration of a 1,2-branching reaction herein may vary and can often be determined by the amount of time it takes to use all of the available sucrose substrate. In certain embodiments, the reaction is conducted until at least 90%, or at least 95%, or at least 99% of the sucrose initially present in the reaction mixture is consumed. In certain embodiments, the reaction time is about 1 hour to 168 hours, 1 hour to 72 hours, 1 hour to 24 hours, or 1 hour to 2 hours.

The temperature of a 1,2-branching reaction herein may be chosen to control both the reaction rate and the stability of the enzymes(s) used, as desired. The temperature of the reaction may range from just above the freezing point of the reaction formulation (approximately 0° C.) to about 60° C., or from 5° C. to about 47° C., or a range of about 20° C. to about 37° C., for example.

In certain embodiments, the set of reaction components can further include an alpha-glucanohydrolase (exo- and/or endo-glucanohydrolase). In certain embodiments, the alpha-glucanohydrolase is a dextranase or mutanase, such as an endomutanase or endodextranase. In certain embodiments, the alpha-glucanohydrolase is a dextranase (EC 2.1.1.11), mutanase (EC 3.1.1.59) or combination thereof. In certain embodiments, the dextranase is a food grade dextranase from Chaetomium erraticum. In certain embodiments, the dextranase from Chaetomium erraticum is DEXTRANASE® PLUS L, available from Novozymes A/S, Denmark.

In certain embodiments, isolating a 1,2-branched glucan product composition includes at least one of centrifugation, filtration, fractionation, chromatographic separation, dialysis, evaporation, and dilution.

Any of the foregoing conditions herein for synthesizing a glucan composition with alpha-1,2 branches, such as the foregoing or those described in the below Examples, can be applied to practicing a reaction composition as presently disclosed.

Non-limiting examples of compositions and methods disclosed herein include:

1. A reaction composition comprising at least water, sucrose, an alpha-glucan substrate, and a polypeptide that is capable of forming at least one alpha-1,2 branch from the alpha-glucan substrate, wherein the polypeptide comprises an amino acid sequence that is at least 90% identical to: (i) the mature form of a sequence selected from the group consisting of SEQ ID NOs:4, 1, 2, 3, 5, 6, 7, 8, 9, 10, 11, 12, and 13; (ii) SEQ ID NO:27 or a sub-sequence within any one of SEQ ID NOs:4, 2, 3, 5, 6, 7, 8, 9, 10, 11, 12, or 13 that aligns with SEQ ID NO:27; and/or (iii) a sequence selected from the group consisting of SEQ ID NOs:4, 1, 2, 3, 5, 6, 7, 8, 9, 10, 11, 12, and 13.
2. The reaction composition of embodiment 1, wherein the sequence of (i) comprises: positions 36 to 1672 of SEQ ID NO:4, positions 21 to 2771 of SEQ ID NO:1, positions 21 to 2821 of SEQ ID NO:2, positions 41 to 2844 of SEQ ID NO:3, positions 51 to 1632 of SEQ ID NO:5, positions 51 to 1318 of SEQ ID NO:6, positions 51 to 1139 of SEQ ID NO:7, positions 51 to 1463 of SEQ ID NO:8, positions 41 to 2841 of SEQ ID NO:9, positions 46 to 2580 of SEQ ID NO:10, positions 51 to 1463 of SEQ ID NO:11, positions 21 to 2824 of SEQ ID NO:12, or positions 21 to 2771 of SEQ ID NO:13.
3. The reaction composition of embodiment 1, wherein the sequence of (ii) comprises: positions 36 to 1115 of SEQ ID NO:4, positions 1715 to 2821 of SEQ ID NO:2, positions 1735 to 2834 of SEQ ID NO:3, positions 51 to 1167 of SEQ ID NO:5, positions 93 to 1178 of SEQ ID NO:6, positions 51 to 1130 of SEQ ID NO:7, positions 51 to 1158 of SEQ ID NO:8, positions 1735 to 2841 of SEQ ID NO:9, positions 1274 to 2413 of SEQ ID NO:10, positions 51 to 1158 of SEQ ID NO: 11, positions 1715 to 2821 of SEQ ID NO: 12., or positions 1665 to 2771 of SEQ U) NO: 13.
4. The reaction composition of any of embodiments 1-3, wherein the alpha-glucan substrate has a degree of polymerization of at least 3, and comprises at least (i) alpha-1,6 glycosidic linkages or (ii) alpha-1,6 and alpha-1,3 glycosidic linkages.
5. A method of producing a glucan composition that comprises alpha-1,2 linkages, the method comprising: (a) providing at least the following reaction components: water, sucrose, an alpha-glucan substrate, and a polypeptide that is capable of forming at least one alpha-1,2 branch from the alpha-glucan substrate, wherein the polypeptide comprises an amino acid sequence that is at least 90% identical to: (i) the mature form of a sequence selected from the group consisting of SEQ ID NOs:4, 1, 2, 3, 5, 6, 7, 8, 9, 10, 11, 12, and 13; (ii) SEQ ID NO:27 or a sub-sequence within any one of SEQ ID NOs:4, 2, 3, 5, 6, 7, 8, 9, 10, 11, 12, or 13 that aligns with SEQ ID NO:27; and/or (iii) a sequence selected from the group consisting of SEQ ID NOs:4, 1, 2, 3, 5, 6, 7, 8, 9, 10, 11, 12, and 13; (b) combining the reaction components under suitable conditions whereby the polypeptide catalyzes the synthesis of at least one alpha-1,2 branch from the alpha-glucan substrate, thereby forming a glucan composition comprising one or more alpha-1,2 linkages; and (c) optionally isolating the glucan composition comprising one or more alpha-1,2 linkages.
6. The method of embodiment 5, wherein the alpha-glucan substrate (i) has a degree of polymerization of at least 3, and comprises at least (i) alpha-1,6 glycosidic linkages or (ii) alpha-1,6 and alpha-1,3 glycosidic linkages.
7. The method of any of embodiments 5-6, wherein the alpha-glucan substrate and sucrose are present in (b) in a ratio between 0.01:1 and 1:0.01, inclusive.
8. The method of any of embodiments 5-7, wherein the reaction components further comprise an alpha-glucanohydrolase.
9. A composition that comprises a glucan composition comprising one or more alpha-1,2 linkages produced by the method of any of embodiments 5-8 or as produced by the reaction composition of any of embodiments 1-4, preferably wherein the composition is in the form of a food product, pharmaceutical product, personal care product, household care product, or industrial product, optionally wherein the composition comprises about 0.01 to 99 wt % (dry solids basis) of the glucan composition.
10. The composition of embodiment 9, wherein the glucan composition comprising alpha-1,2 linkages is water-soluble and slowly releases glucose when fed to a mammal, wherein the mammal is preferably a human.
11. The composition of any of embodiments 9-10, wherein the glucan composition comprising alpha-1,2 linkages has 10% or less of alpha-1,2 branching.
12. The composition of embodiment 9, wherein the glucan composition comprising alpha-1,2 linkages is water-soluble and acts as a dietary fiber when fed to a mammal, wherein said mammal is preferably a human.
13. The composition of any of embodiments 9 or 12, wherein the glucan composition comprising alpha-1,2 linkages has at least about 15% N) alpha-1,2 branching.
14. The composition of any of embodiments 9-13, further comprising at least one ingredient selected from the group consisting of: synbiotics, peptides, peptide hydrolysates, proteins, protein hydrolysates, soy proteins, dairy proteins, amino acids, polyols, polyphenols, vitamins, minerals, herbals, herbal extracts, fatty acids, polyunsaturated fatty acids (PUFAs), phytosteroids, betaine, carotenoids, digestive enzymes, and probiotic organisms; preferably wherein the composition is in the form of a food product or pharmaceutical product.
15. The composition of any of embodiments 9-14, wherein the composition is in the form of a food product or pharmaceutical product, and further comprising at least one ingredient selected from the group consisting of: monosaccharides, disaccharides, glucose, sucrose, fructose, leucrose, corn syrup, high fructose corn syrup, isomerized sugar, maltose, trehalose, panose, raffinose, cellobiose, isomaltose, honey, maple sugar, fruit-derived sweeteners, sorbitol, maltitol, isomaltitol, lactose, nigerose, kojibiose, xylitol, erythritol, dihydrochalcone, stevioside, alpha-glycosyl stevioside, acesulfame potassium, alitame, neotame, glycyrrhizin, thaumantin, sucralose, L-aspartyl-L-phenylalanine methyl ester, saccharine, maltodextrin, starch, potato starch, tapioca starch, dextran, soluble corn fiber, resistant maltodextrins, branched maltodextrins, inulin, polydextrose, fructo-oligosaccharides, galacto-oligosaccharides, xylo-oligosaccharides, arabinoxylo-oligosaccharides, nigero-oligosaccharides, gentio-oligosaccharides, hemicellulose, fructose oligomer syrup, isomalto-oligosaccharides, fillers, excipients, and binders.
16. The composition of embodiment 9, wherein the composition is a detergent composition, and wherein the composition is preferably a household product.
17. A method comprising enterally administering a substance to a mammal, wherein the substance comprises a glucan composition comprising alpha-1,2 linkages, wherein the administering results in less or slower blood glucose elevation in the mammal as compared to a mammal that is enterally administered a substance that lacks the glucan composition but instead contains a same amount of a readily digestible glucose-containing carbohydrate, wherein the glucan composition is produced by the method of any of embodiments 5-8 or as produced by the reaction composition of any of embodiments 1-4, optionally wherein the mammal is a human, and optionally wherein the readily digestible glucose-containing carbohydrate is sucrose, free glucose, or starch.
18. A method of producing an ingestible product, the method comprising incorporating a glucan composition comprising alpha-1,2 linkages into the ingestible product, wherein the glycemic index of the resulting ingestible product is not increased, or only marginally increased, compared to an ingestible product that lacks the glucan composition (but is otherwise the same), and wherein the glucan composition is produced by the method of any of embodiments 5-8 or as produced by the reaction composition of any of embodiments 1-4.

### EXAMPLES

The disclosure is further defined in the following Examples. It should be understood that the Examples, while indicating certain embodiments, is given by way of illustration only. From the above discussion and the Examples, one skilled in the art can ascertain essential characteristics of this disclosure, and without departing from the spirit and scope thereof, can make various changes and modifications to adapt to various uses and conditions.

The meaning of abbreviations is as follows: “sec” or “s” means second(s), “ms” mean milliseconds, “min” means minute(s), “h” or “hr” means hour(s), “μL” means microliter(s), “mL” means milliliter(s), “L” means liter(s); “mL/min” is milliliters per minute; “μg/mL” is microgram(s) per milliliter(s); “LB” is Luria broth; “m” is micrometers, “nm” is nanometers; “OD” is optical density; “IPTG” is isopropyl-β-D-thio-galactoside; “g” is gravitational force; “mM” is millimolar; “SDS-PAGE” is sodium dodecyl sulfate polyacrylamide; “mg/mL” is milligrams per milliliters; “N” is normal; “w/v” is weight for volume; “DTT” is dithiothreitol; “BCA” is bicinchoninic acid; “DMAc” is N,N′-dimethyl acetamide; “LiCl” is Lithium chloride’ “NMR” is nuclear magnetic resonance; “DMSO” is dimethylsulfoxide; “SEC” is size exclusion chromatography; “GI” or “gi” means GenInfo Identifier, a system used by GENBANK® and other sequence databases to uniquely identify polynucleotide and/or polypeptide sequences within the respective databases; “DPx” means glucan degree of polymerization having “x” units in length; “ATCC” means American Type Culture Collection (Manassas, VA), “DSMZ” and “DSM” refer to Leibniz Institute DSMZ-German Collection of Microorganisms and Cell Cultures, (Braunschweig, Germany); “EELA” is the Finish Food Safety Authority (Helsinki, Finland;) “CCUG” refer to the Culture Collection, University of Goteborg, Sweden; “Suc.” means sucrose; “Gluc.” means glucose; “Fruc.” means fructose; “Leuc.” means leucrose; and “Rxn” means reaction.

### General Methods

All reagents, restriction enzymes and bacterial culture materials were obtained from BD Diagnostic Systems (Sparks, MD), Invitrogen/Life Technologies Corp. (Carlsbad, CA), Life Technologies (Rockville, MD), QIAGEN (Valencia, CA), Sigma-Aldrich Chemical Company (St. Louis, MO), or Pierce Chemical Co. (a division of Thermo Fisher Scientific Inc., Rockford, IL) unless otherwise specified. IPTG, (cat #16758), triphenyltetrazolium chloride, and BCA protein assay reagents were obtained from the Sigma Co., (St. Louis, MO). BELLCO spin flasks were obtained from the Bellco Co., (Vineland, NJ). LB medium was obtained from Becton, Dickinson and Company (Franklin Lakes, New Jersey).

Reducing sugars were determined using the PAHBAH assay (Lever, Anal. Biochem. 47, 273-279, 1972).

### Example 1

### Identification of Polypeptides Capable of Forming Glucans Having Alpha (1,2) Linkages

The GTF-J18 sequence (from Leuconostoc mesenteroides subsp. mesenteroides J18) (SEQ ID NO:1) contains three distinct regions: catalytic domain 1 (“CD1”), a glucan binding domain, and CD2. CD1 and CD2 are GH70 domains, responsible for different catalytic activity.

We have shown that an N-terminally truncated version of GTFJ18, designated GTFJ18T1 (SEQ ID NO:27), can produce alpha-1,2-branching when an alpha-1,6-linked polysaccharide is provided in the reaction (see Example 1 of International Pat. Appl. Publ. No. WO2015/183714, which is incorporated herein by reference) (also refer to Example 6 below). GTF-J18T1 (SEQ ID NO:27) only includes a portion of the glucan binding domain and CD2. This suggests that CD2 from GTF-J18 (“GTF-J18-CD2”) can catalyze alpha-1,2-branching on an alpha-1,6-linked polysaccharide substrate.

The GTF-J18-CD2 sequence (SEQ ID NO:28) was used as a query to BLAST against the non-redundant version of the GENBANK Protein database. Forty sequences were reported by BLAST as more than 50% identical to the GTF-J18-CD2 sequence (SEQ ID NO:28) in the BLAST High Scoring Segment. The forty sequences were aligned using the TARGET2K program from the SAM suite from UCSC (SAM: Package of HMM tools released by the Karplus group at UC Santa Cruz; the algorithms in SAM are described in Karplus et al., Bioinformatics 14:846-856, 1998). Thirteen sequences (SEQ ID NOs:1-13) that were at least 60% identical to the GTF-J18-CD2 sequence (SEQ ID NO:28) in the aligned region were selected as polypeptides possibly capable of forming glucans having alpha (1,2) linkages and are disclosed herein.

### Example 2

### Expression of Lactobacillus animalis KCTC 3501 Glucosyltransferase GTF8117

A putative glucosyltransferase gene, LanGtf1, with GI no. 335358117 (old) (new GI no. 948839227; GENBANK Acc. No. KRM57462.1) was identified from Lactobacillus animalis KCTC 3501 (“GTF8117” herein). GTF8117 protein has a 37 amino acid signal peptide predicted by SignalP4.0 (Petersen et al., Nature Methods 8:785-786, 2011). This indicates that GTF8117 is a secreted protein. The gene sequence encoding the mature protein of GTF8117 was codon-optimized for expression in Bacillus subtilis. The gene was synthesized by Generay (Shanghai, China), and inserted into the p2JM103BBI plasmid (Vogtentanz, Protein Expr. Purif 55:40-52, 2007), resulting in the pZZH561 plasmid. pZZHB561 contains (in 5′ to 3′ direction) an aprE promoter, a sequence encoding an aprE signal sequence used to direct protein secretion in Bacillus subtilis, an oligonucleotide that encodes Ala-Gly-Lys to facilitate secretion of the target protein, and the synthetic gene encoding the target protein.

Another expression plasmid, pDCQ1004, was constructed for expressing GTF8117 without a signal peptide. Plasmid pZZHB561 was used as the template for PCR amplification of the GTF8117 coding sequence together with the Tlat terminator. The PCR product was cloned into the SpeI and BamH sites of the B. subtilis integrative expression plasmid, p4JH, having an aprE promoter, but no signal sequence-coding region. The resulting pDCQ1004 plasmid contains SEQ ID NO:29, which encodes mature GTF8117 with an added N-terminal methionine (SEQ ID NO:30). pDCQ1004 was used to transform B. subtilis BG6006 cells to express GTF8117 (SEQ ID NO:30). The B. subtilis host BG6006 strain contains nine protease deletions (amyE::xylRPxylAcomK-ennC, degUHy32, oppA, ΔspoHE3501, ΔaprE, ΔnprE, Δepr, ΔispA, Δbpr, Δvpr, ΔwprA, Δmpr-ybfJ, ΔnprB). The transformed cells were spread on LB plates supplemented with 5 μg/mL chloramphenicol. The colonies grown on these plates were streaked several times onto LB plates with 25 μg/mL chloramphenicol. The resulting amplified colonies were grown in LB containing 25 g/mL chloramphenicol for 6-8 hours, and then subcultured into Grant's II medium and grown at 30° C. for 2-3 days. The cultures were spun at 15,000 g for 30 min at 4° C. and the collected supernatants, which were expected to contain soluble intracellular proteins due to cell autolysis, were filtered through 0.22-μm filters. The filtered supernatants were aliquoted and frozen at −80° C., and were used later in a p-hydroxybenzoic acid hydrazine (PAHBAH) assay to determine enzyme activity. The clone with supernatant with the highest activity in the PAHBAH assay (named as strain SG1024) was used to make seed vials for fermentation.

Further production of GTF8117 (SEQ ID NO:30) was conducted in Example 4.

### Example 3

### Expression of Streptococcus salivarius M18 Glucosyltransferase GTF6831

A putative glucosyltransferase (old GI no. 345526831, new GI no. 490287001; GENBANK Acc. No. WP_004182667.1) was identified from Streptococcus salivarius M18. This enzyme is herein referred to as “GTF6831” and has 1600 amino acid residues with the first 42 residues predicted to be the native signal peptide by the SignalP4.1 program. The gene nucleotide sequence (SEQ ID NO:31) encoding the mature protein of GTF6831 (SEQ ID NO:32) was synthesized by GenScript USA Inc. (Piscataway, NJ). The synthesized sequence (SEQ ID NO:31) was cloned into the NheI and HindIII sites of the Bacillus subtilis integrative expression plasmid p4JH under the aprE promoter and sequence encoding a B. subtilis AprE signal peptide. The construct was first transformed into E. coli DH10B and selected on LB with ampicillin (100 μg/mL) plates. The confirmed construct, pDCQ990-5, allowed expression of mature GTF6831 glucosyltransferase (SEQ ID NO:32). pDCQ990-5 was then transformed into B. subtilis BG6006, which contains nine protease deletions (amyE::xylRPxylAcomK-ennC, degUHy32, oppA, ΔspoIIE3501, ΔaprE, ΔnprE, Δepr, ΔispA, Δbpr, Δvpr, ΔwprA, Δmpr-ybfJ, ΔnprB), and selected on LB plates with chloramphenicol (5 μg/mL). The colonies grown on these plates were streaked several times onto LB plates with 25 μg/mL chloramphenicol. The multiple colonies of the resulting B. subtilis expression strain, SG1190, were grown in LB medium with 25 μg/mL chloramphenicol for 6-8 hours first, and then subcultured into Grant's II medium and grown at 30° C. for 2-3 days. The cultures were spun at 15,000 g for 30 min at 4° C. and the supernatants were filtered through 0.22-μm filters. The filtered supernatants were aliquoted and frozen at −80° C., and later used in a PAHBAH assay to determine enzyme activity. The clone with the highest PAHBAH activity was chosen to make the seed vial for fermentation.

Further production of GTF6831 (SEQ ID NO:32) was conducted in Example 5.

### Example 4

### Production of GTF8117 Glucosyltransferase in B. subtilis

The GTF8117 glucosyltransferase (SEQ ID NO:30) was produced using the SG1204 strain, which is a nine protease-knockout B. subtilis, comK strain, containing the pDCQ1004 plasmid. Initially, a frozen vial of SG1204 was grown into 40 mL seed medium containing 10 g/L soytone, 5 g/L yeast extract, 10 g/L NaCl, 10 g/L glucose and 10 mg/L chloramphenicol antibiotic. The growth temperature was 30° C. and an initial pH of 7.2. A 250-mL seed flask containing 40 mL of seed medium was placed in a shaker-incubator at 300 C and mixed at 300 rpm for 2.83 hours. After that time the inoculum grew to an OD (600 nm) value of 0.33 units. 30 mL of the grown seed medium was used to inoculate a production vessel containing 7 liters of fermentation medium.

The fermentation medium contained 5 g/L corn steep solids, 8 g/L sodium phosphate monobasic monohydrate, 8 g/L potassium phosphate monohydrate, 4.2 g/L magnesium sulfate heptahydrate, 0.3 g/L ferrous sulfate heptahydrate, 0.2 g/L manganese chloride tetrahydrate, 0.1 g/L calcium chloride dehydrate and 2.06 mL/L of the antifoam agent, FOAM BLAST 882.

The following operating conditions were set: 30° C., pH 7.2, and 25% dissolved oxygen. The fermentation ran initially in batch mode, starting with a residual glucose concentration of 10 g/L. As the residual glucose was almost depleted at 15.3 hours of elapsed fermentation time, we commenced continuous glucose feeding by initiating a fed-batch mode. The pH was controlled using a 69% v/v NH₄OH solution (20% NH₃ content). Glucose was fed until 40.6 hours of elapsed fermentation time, delivering approximately 1593 grams at 650 g/L glucose concentration. At 40.6 hours, the glucose feed stopped while maintaining the other operating conditions for approximately one-half hour in order to allow complete consumption of the remaining residual glucose. Then, at 41.1 hours of elapsed fermentation time, the lysis portion of the run was initiated by complementing the glucose cessation with an increase of the operating fermentation temperature to 33° C., a complete stop of the air flow, reduction of the agitation speed to 200 rpm, and a lift of the pH control conditions, resulting in complete, in situ cell lysis within the ensuing 8-hour period. The fermentation run ended 49.1 hours after onset.

The supernatant of the end-of-run broth was collected and analyzed by HPLC for the presence of GTF8117 (SEQ ID NO:30) by measuring the consumption rate of the sucrose and the corresponding production rate of fructose. This biochemical assay measured 4.64 mg GTF8117/mL (472 U/mL, based on sucrose consumption) and 4.25 mg GTF8117/mL (based on fructose production). The supernatant was stored at −80° C.

GTF8117 (SEQ ID NO:30) produced in this Example was used in one or more of the following Examples to synthesize glucan to serve as a substrate for an alpha-1,2-branching enzyme.

### Example 5

### Production of GTF6831 Glucosyltransferase in B. subtilis

The GTF6831 glucosyltransferase (SEQ ID NO:32) was produced using the SG1190 strain, which is a nine protease-knockout B. subtilis, comK strain, containing the pDCQ990-5 plasmid. The seed flask medium, fermentation medium, and all operating conditions were the same as outlined in Example 4. The fermentation run lasted 53 hours. Cell lysis was initiated at 41 hours elapsed fermentation time and lasted 12 hours. The total amount of glucose feed delivered was 2147 grams of 50% w/w solution.

After lysis, the supernatant was collected and analyzed by HPLC for the presence of enzyme GTF6831 (SEQ ID NO:32) by measuring the consumption rate of the residual sucrose and the corresponding production rate of fructose. This biochemical assay measured 108.7 U/mL supernatant. Centrifugation of the lysate produced a supernatant having 114.5 U/mL of activity; the supernatant was stored at −80° C.

GTF6831 (SEQ ID NO:32) produced in this Example was used in one or more of the following Examples to synthesize glucan to serve as a substrate for an alpha-1,2-branching enzyme.

### Example 6

### Expression of the Truncated Glucosyltransferase, GTFJ18T1, and Testing its Alpha-1,2 Branching Activity

The following example describes expression of a full length glucosyltransferase and a truncated version of this enzyme in E. coli, and testing their respective alpha-1,2 branching activity on a glucan backbone. The full length glucosyltransferase produced glucan with little alpha-1,2 branching, whereas the truncated version of the glucosyltransferase produced glucan with a significant amount of alpha-1,2 branching.

The putative glucosyltransferase (old GENBANK® gi: 356644413, new gi: 504090610, Acc. No. WP_014324604.1) from Leuconostoc mesenteroides subsp. mesenteroides J18 (designated as GTFJ18) has 2771 amino acids (SEQ ID NO:1). It was identified as a glycosyl hydrolase from complete genome sequencing of the J18 strain isolated from Kimchi (Jung et al., J Bacteriol. 194:730, 2012). The full length sequence of GTFJ18 has 68.6% amino acid identity to the DsrE protein (2835 amino acids in length) from Leuconostoc mesenteroides NRRL B-1299 (GENBANK® gi: 23320943, Acc. No. CAD22883.1). The DsrE protein was shown to be a bifunctional protein with two catalytic domains (Bozonnet et al., J. Bacteriol. 184:5763,2002). The first catalytic domain “CD1” catalyzes the synthesis of alpha-1,6 linkages and the second catalytic domain “CD2” catalyzes the synthesis of alpha-1,2 linkages. The CD1 and CD2 domains are separated by a glucan binding domain “GBD” (Fabre et al., J Bacteriol. 187:296, 2005). The CD1 domains of the DsrE and GTFJ18 proteins share 79.3% amino acid identity and the CD2 domains of the two proteins share 76.6% amino acid identity.

The N-terminal 20 amino acids of GTFJ18 (SEQ ID NO:1) was deduced as the signal peptide by the SignalP 4.0 program (Petersen et al., ibid.). To construct the full length GTFJ18 expression plasmid, DNA encoding the mature protein without the signal peptide was synthesized by GenScript USA Inc. The synthesized gene was subcloned into the NheI and HindIII sites of the pET23D+ vector (NOVAGEN®; Merck KGaA, Darmstadt, Germany). A polynucleotide encoding a truncated version of GTFJ18, termed GTF18T1 (SEQ ID NO:27), containing the C-terminal CD2 domain and part of a GBD domain (in total containing amino acid residues 1665-2771 of SEQ ID NO: 1) was also subcloned into the pET23D+vector. The plasmids carrying gene sequences encoding either the full length (SEQ ID NO:1) or truncated (SEQ ID NO:27) GTFJ18 protein were transformed into E. coli BL21 DE3 host cells resulting in strains EC0059 and EC0059T1, respectively. Cells of EC0059 and EC0059T1 were grown to OD ˜0.5 and induced with 1 mM IPTG at 37° C. for 3 hours or alternatively they were induced at 23° C. overnight. The cells were collected by centrifugation at 4000×g for 10 min and resuspended in PBS buffer pH 6.8. The cells were broken by passing through a French Press at 14,000 psig (˜96.53 MPa) twice and the cell debris was pelleted by centrifugation at 15,000×g for 20 min. The supernatant of each crude enzyme extract was aliquoted and frozen at −80° C.

The alpha-1,2 branching activities of each enzyme (GTFJ18 or GTFJ18T1) were individually tested on the glucan product of the glucosyltransferase, GTF5604, which is derived from Streptococcus criceti HS-6 (GENBANK® Acc. No. WP_004226213.1, old gi: 357235604) and disclosed in International Pat. Appl. Publ. No. WO2015/183714 (also referred to therein as SG1018 glucosyltransferase or GtfHS6). GTF5604 (SEQ ID NO:33) has 1338 amino acids with the N-terminal 36 amino acids deduced as its signal peptide by the SignalP 4.0 program. The native nucleotide sequence (positions 1289627-1293643 of GENBANK Acc. No. NZ_AEUV02000002.1) encoding full length GTF5604 (SEQ ID NO:33) including its native signal peptide was synthesized by GenScript and cloned into the SpeI and HindIII sites of the replicative Bacillus expression plasmid pHYT (Takara Bio Inc., Otsu, Japan) under the B. subtilis aprE promoter. The construct was first transformed into E. coli DH10B and selected on ampicillin (100 μg/mL) plates. The confirmed clone, pDCQ918, was then transformed into Bacillus subtilis strain BG6006 (amyE::xylRPxylAcomK-ermC, degUHy32, oppA, ΔspoIIE3501, ΔaprE, ΔnprE, Δepr, ΔispA, Δbpr, Δvpr, ΔwprA, Δmpr-ybfJ, ΔnprB) and selected on tetracycline (12.5 pg/mL) plates, after which the transformed strain, termed as SG1018, was grown in LB containing 10 μg/mL tetracycline first, followed by subculturing into Grant's II medium containing 12.5 pg/mL tetracycline and growth at 37° C. for 2-3 days. The cultures were spun at 15,000×g for 30 min at 4° C. and the supernatant was filtered through 0.22-μm filters. A glucan synthesis reaction was set up containing 10% (v/v) of the SG1018 supernatant with 100 g/L sucrose, 10 mM sodium citrate pH 5 and 1 mM CaCl₂). All sucrose was consumed in the reaction after 6 hours at 37° C.; the glucan product had a molecular weight of about 3000 and consisted of almost 100% alpha-1,6 linkages. The glucan synthesis reaction was subjected to heat inactivation at 95° C. for 30 min.

A branching reaction was set up with 70% (v/v) of the heat-inactivated glucan synthesis reaction. The enzyme being tested for branching activity off of the above glucan product, GTFJ18 or GTFJ18T1, was provided as 10% (v/v) of the above-prepared crude cell extract from EC0059 or EC0059T1, respectively, with 40 g/L sucrose. Each branching reaction was incubated at 37° C. or 30° C. for 22 hours and the products were analyzed by HPLC for sucrose consumption and NMR for linkage profile.

NMR data were acquired on an Agilent DD2 spectrometer (Agilent Technologies, Inc., Santa Clara, CA) operating at 500 MHz for ¹H using a 5-mm cryogenic triple-resonance pulsed-field gradient probe. Water suppression was obtained by carefully placing the observe transmitter frequency on resonance for the residual water signal in a “presat” experiment, and then using the first slice of a NOESY experiment with a full phase cycle (multiple of 32) and a mix time of 10 ms. One-dimensional ¹H spectra were acquired with a spectral width of 6410 Hz, acquisition time of 5.1 s, 65536 data points, 4 s pre-saturation and a 90-degree observe pulse. Signal averaging typically involved accumulation of 64 scans. Sample temperature was maintained at 25° C.

Liquid samples were prepared by adding either 50 or 100 μL of completed branching reactions to a 5-mm NMR tube along with 60 μL of D2O containing 12.4 mM 4,4-dimethyl-4-silapentane-1-sulfonic acid sodium salt (DSS) as an internal chemical shift reference, and the balance (450 or 400 μL) of D₂O for a total volume of 560 μL. The DSS methyl resonance was set to 0 ppm.

Chemical shift assignments for different anomeric linkages were taken from Goffin et al. (Bull Korean Chem. Soc. 30:2535, 2009). Assignments specific to alpha-1,2 branching on an alpha-1,6 backbone were taken from Maina et al. (Carb. Res. 343:1446,2008). Alpha-1,2 substitution on the 1,6 backbone (i.e., alpha 1-2,6 linkage) leads to a characteristic chemical shift (5.18 ppm) for the anomeric H adjacent the substitution site. The anomeric H of the 1,2-linked sugar (5.10 ppm) is obscured by leucrose in reaction mixtures but is directly observed in purified samples.

The product of the branching reaction comprising GTFJ18 contained 97% alpha-1,6 linkages and only 0.6% alpha-1,2 linkages. The product of the branching reaction comprising GTFJ18T1 (SEQ ID NO:27) contained 82% alpha-1,6 linkages and 18% alpha-1,2 linkages. Thus, the truncated enzyme, GTFJ18T1 (SEQ ID NO:27) showed much higher alpha-1,2 branching activity compared to its full length counterpart, GTFJ18. Although not intending to be bound by any theory herein, this result may be due to that the CD1 domain in full length GTFJ18 was very active and competed with the CD2 branching domain for the sucrose substrate.

Thus, GTFJ18T1 (SEQ ID NO:27) has significant alpha-1,2 branching activity and can be used to modify the structure of a glucan substrate. Further production of this enzyme was conducted in Example 7.

### Example 7

### Production of the GTFJ18T1 1,2-Branching Enzyme Using E. coli

The GTFJ18T1 1,2-branching enzyme (SEQ ID NO:27) disclosed in Example 6 was produced using the EC0059T1 strain, which is a BL21 DE3 E. coli strain containing the targeted enzyme genetic information in a plasmid. Initially, a frozen vial of EC0059T1 was grown into 260 mL seed medium containing 10 g/L yeast extract, 16 g/L tryptone, 5 g/L NaCl, no glucose, and 100 mg/L ampicillin antibiotic. The growth temperature was 30° C. and the initial pH was 6.8. A 1-L seed flask containing 260 mL of seed medium for inoculating two fermentation vessels was placed in a shaker-incubator and mixed at 250 rpm for 3.7 hours. After that time the inoculum grew to an OD (600 nm) value of 4.0 units. 125 mL of the grown seed medium was used to inoculate each of the production vessels containing 7 liters of fermentation medium.

The fermentation medium contained 5 g/L yeast extract, 5 g/L potassium phosphate monobasic, 1.9 g/L NaCl, 1.0 g/L TWEEN-80 and 0.1 mL/L BIOSPUMEX 153K antifoam agent. These components were added prior to vessel/broth sterilization. After the sterilization cycle was completed and the operating temperature leveled and its set value, we added via sterile technique the following compounds: 2/4 g/L magnesium sulfate heptahydrate, 50 mg/L ferrous sulfate heptahydrate, 100 mg/L calcium chloride dehydrate, 10 mL/L NIT trace elements cocktail, 100 mg/L ampicillin antibiotic and 10 g/L glucose. The NIT trace elements cocktail solution contained: 10 g/L citric acid monohydrate, 2 g/L manganese sulfate monohydrate, 2 g/L sodium chloride, 0.5 g/L ferrous sulfate heptahydrate, 0.2 g/L zinc sulfate heptahydrate, 20 mg/L copper sulfate pentahydrate, 20 mg/L sodium molybdate dehydrate and 2.94 mg/L calcium chloride dihydrate.

The fermentation run was controlled at 30° C., pH 6.8, and 25% dissolved oxygen. Initially, for the first 5 hours the fermentation ran in a batch mode, starting with a residual glucose concentration of 10 g/L. After 5 hours of elapsed fermentation time, we commenced with continuous glucose feeding and initiated a fed-batch mode. Glucose was carefully fed aiming to maintain a very low, residual glucose concentration (<0.1 g/L). At 17 hours of elapsed fermentation time, we induced production of GTFJ18T1 protein (SEQ ID NO:27) by adding IPTG to the broth, achieving a residual concentration of 0.5 mM. Induction lasted 7 hours in one experiment and 16 hours in the other. During this time we continued the feed of glucose while maintaining a very low residual concentration (<0.1 g/L).

At the end of the two fermentations (24 and 33 hours, respectively) the cell pellet was recovered from approximately 9 kg of fermentation broth in an RC-12 centrifuge using 2-liter bottles. A set of two plastic bags was placed within the bottles for easy recovery of the cell paste. The centrifugation was done at 6728 RCF for 20 minutes. After the liquid was decanted from the bags, the cell pellet was frozen at −80° C.; a total of 1943 grams of frozen paste was recovered. The frozen paste was subsequently added to 3.2 liters of buffer (50 mM potassium phosphate, pH 6.0), and after the paste had thawed, the resulting cell suspension was homogenized using an APV-100 homogenizer operated at a pressure of approximately 850 bar. The homogenate was cooled over ice, and then decanted into six 1-liter centrifuge bottles. The bottles were spun for over an hour in an RC-3 centrifuge at 5890 RCF. The supernatant was decanted into bottles and stored frozen at −80° C.; aliquots of the two fermentation run lysate supernatants were analyzed for the presence of the target enzyme, GTFJ18T1 (SEQ ID NO:27), by measuring the rate of conversion of sucrose to fructose. The biochemical assays measured 5.8 U/mL and 9.90 U/mL of active enzyme for the two fermentations, respectively. A second centrifugation of the 5.8 U/ml supernatant at 12,000 rpm using an SS34 rotor produced a clarified supernatant with 5.0 U/mL GTFJ18T1 activity (stored frozen at −80° C.).

GTFJ18T1 (SEQ ID NO:27) produced in this Example was used in one or more of the following Examples.

### Example 8

### Sequences of Fructobacillus tropaeoli F214-1 Glucosyltransferase, FtrGtf1 (GTF9905)

A glucosyltransferase gene, FtrGtf1, was identified from Fructobacillus tropaeoli F214-1. The nucleic acid sequence for the FtrGtf1 gene (GENBANK Acc. No. DF968096.1, SEQ ID NO:17), and the amino acid sequence of the hypothetical protein encoded by the FtrGtf1 gene (GENBANK Acc. No. GAP05007.1, gi 902949905, SEQ ID NO:4, “GTF9905” herein) were found in the NCBI database. A portion of SEQ ID NO:4 aligned with SEQ ID NO:28 in the analysis disclosed above in Example 1, thereby identifying GTF9905 as a putative alpha-1,2 branching enzyme. The mature, secreted form GTF9905 corresponds with positions 36-1672 of SEQ ID NO:4.

### Example 9

### Sequences for Expressing Mature GTF9905 (FtrGtf1)

A nucleotide sequence encoding the mature version of GTF9905 (Gtf1 from Fructobacillus tropaeoli F214-1) was codon-optimized for expression in Bacillus subtilis, resulting in SEQ ID NO:34. This sequence was synthesized by Generay (Shanghai, China) and inserted into the p2JM103BBI plasmid (Vogtentanz, Protein Expr. Purif 55:40-52, 2007), resulting in pZQ2-CRC08152-FtrGtf1 plasmid. pZQ2-CRC08152-FtrGtf1 contains (in 5′ to 3′ direction) an aprE promoter, a sequence encoding an aprE signal sequence used to direct protein secretion in Bacillus subtilis, an oligonucleotide that encodes Ala-Gly-Lys to facilitate the secretion of the target protein, and the synthetic sequence (SEQ ID NO:34) encoding the mature form of GTF9905 (i.e., positions 36-1672 of SEQ ID NO:4)

Plasmid pZQ2-CRC08152-FtrGtf1 was used to transform B. subtilis cells, and the transformed cells were spread on Luria Agar plates supplemented with 5 ppm chloramphenicol. A colony with correct insertion, as confirmed by PCR and sequencing, was selected and subjected to fermentation for GTF9905 production.

### Example 10

### Production of GTF9905 (FtrGtf1)

The B. subtilis transformant colony with correct insertion was picked into 5 mL Luria broth medium supplemented with 5 ppm chloramphenicol and grown overnight. 30 μL of this culture was then inoculated into a 250-mL shake flask containing 30 mL Grant's II medium supplemented with 5 ppm chloramphenicol, which was then incubated at 30° C. with 250 rpm shaking for 48 hours. The resulting culture supernatant was collected by centrifugation at 24,000×g for 1 h at 4° C. and filtered with 0.22-μm filter.

The supernatant, which contained GTF9905, was first dialyzed with 50 mM K₂HPO₄ pH 6.8 buffer in dialysis tubing (Thermo product #68100), after which the dialyzed supernatant was lyophilized using the FREEZONE 6 freeze-dry system (Labconco) and stored at −80° C. (22.5 U/mL).

GTF9905 produced in this Example was used in one or more of the following Examples.

### Example 11

### GTF8117 and GTF6831 Enzyme Activity Assay

Glucosyltransferase activity assays for GTF8117 and GTF6831 were performed by incubating each GTF enzyme with 200 g/L sucrose in 25 mM acetate buffer at pH 5.5 in the presence of 25 g/L dextran (MW ˜1500, Sigma-Aldrich, Cat. #31394) at 37° C. and 125 rpm orbital shaking. One aliquot of reaction mixture was withdrawn at 1 h, 2 h and 3 h incubation periods and heated at 90° C. for 5 min to inactivate the GTF. Insoluble material was removed by centrifugation at 13,000×g for 5 min, followed by filtration through 0.22-μm nylon membrane. The resulting filtrate was analyzed by HPLC with AMINEX HPX-87C columns series at 85° C. (Bio-Rad, Hercules, CA) to quantify sucrose concentration. The sucrose concentration at each time point was plotted against the reaction time and the initial reaction rate was determined from the slope of the linear plot. One unit of GTF activity was defined as the amount of enzyme needed to consume one micromole of sucrose in one minute under the assay conditions.

### Example 12

### GTF9905 and GTFJ18T1 Enzyme 1,2-Branching Activity Assay

Glucosyltransferase activity assays for 1,2-branching enzymes GTF9905 and GTFJ18T1 were performed by incubating each enzyme with 100 g/L sucrose in 50 mM acetate buffer at pH 5.5 in the presence of dextran 40K (Sigma-Aldrich Cat #D1662-100G) at 30° C. and 125 rpm orbital shaking. One aliquot of reaction mixture was withdrawn at 1 h, 2 h and 3 h incubation periods and heated at 90° C. for 5 min to inactivate the enzyme. Insoluble material was removed by centrifugation at 13,000×g for 5 min, followed by filtration through 0.2-μm nylon membrane. The resulting filtrate was analyzed by HPLC with AMINEX HPX-87C columns series at 85° C. (Bio-Rad, Hercules, CA) to quantify sucrose concentration. The sucrose concentration at each time point was plotted against the reaction time and the initial reaction rate was determined from the slope of the linear plot. One unit of GTF 1,2-branching enzyme activity was defined as the amount of enzyme needed to consume one micromole of sucrose in one minute under the assay conditions.

### Example 13

### Production of Soluble Alpha-1,2-Branched Polysaccharides by Stepwise Combination of Glucosyltransferase GTF8117 and Alpha-1,2 Branching Enzyme GTF9905

First, a reaction was performed to prepare glucan that was used in the second part of this Example as a substrate for 1,2-branching enzyme. A reaction mixture (20 mL) comprised of sucrose (488 g/L) and GTF8117 (Example 4, 9.4 U/mL) was adjusted to pH 5.5 with 6.0 N hydrochloric acid and stirred at 47° C. Aliquots were withdrawn at predetermined times and quenched by heating at 90° C. for 20 min. The resulting heat-treated aliquots were centrifuged and the supernatants analyzed by HPLC to determine the concentration of sucrose, glucose, fructose, leucrose, oligosaccharides and polysaccharides. After 6 h, the reaction mixture was heated to 90° C. for 30 minutes, and an aliquot of the heat-treated reaction mixture was centrifuged and the resulting supernatant analyzed for soluble monosaccharides, oligosaccharides and polysaccharides (DP8+) (Table 1). The DP8+ polysaccharides appeared to contain about 100% alpha-1,6 linkages.

TABLE 1
HPLC analysis of soluble monosaccharides, oligosaccharides and
polysaccharides produced by glucosyltransferase GTF8117.

| DP8 + | DP7 | DP6 | DP5 | DP4 | DP3 | DP2 | Sucrose | Leucrose | Glucose | Fructose |
|---|---|---|---|---|---|---|---|---|---|---|
| g/L | g/L | g/L | g/L | g/L | g/L | g/L | g/L | g/L | g/L | g/L |
| 241 | 0.0 | 0.0 | 0.0 | 0.0 | 0.0 | 5.5 | 0.1 | 20.1 | 3.4 | 240 |

A second reaction was then performed to conduct alpha-1,2 branching from the products of the first reaction. The second reaction mixture was prepared by mixing 5.84 mL of the heat-treated first reaction mixture prepared above with 2.92 mL of sucrose solution (600 g/L sucrose in deionized water; final sucrose concentration 175 g/L), 0.67 mL of 0.75 M sodium acetate buffer (pH 5.5, 50 mM final concentration), 0.070 mL deionized water and 0.50 mL of a centrifuged cell lysate containing alpha-1,2-branching enzyme GTF9905 (Example 10; final GTF9905 concentration 1.13 U/mL), followed by stirring at 30° C. Aliquots were withdrawn at predetermined times and quenched by heating at 90° C. for 20 min. The resulting heat-treated aliquots were centrifuged and the supernatants analyzed by HPLC to determine the concentration of sucrose, glucose, fructose, leucrose, oligosaccharides and polysaccharides. After 76.2 h, the reaction mixture was heated to 90° C. for 20 minutes, and an aliquot of the heat-treated reaction mixture was centrifuged. The resulting supernatant was analyzed for soluble monosaccharides, oligosaccharides and polysaccharides (Table 2), and analyzed by ¹H NMR spectroscopy to determine the anomeric linkages of the oligosaccharides and polysaccharides (Table 3).

TABLE 2
HPLC analysis of soluble monosaccharides, oligosaccharides and
polysaccharides produced by an alpha-1,2 branching reaction.

| DP8 + | DP7 | DP6 | DP5 | DP4 | DP3 | DP2 | Sucrose | Leucrose | Glucose | Fructose |
|---|---|---|---|---|---|---|---|---|---|---|
| g/L | g/L | g/L | g/L | g/L | g/L | g/L | g/L | g/L | g/L | g/L |
| 239 | 0.0 | 0.0 | 0.0 | 0.0 | 0 | 5.4 | 12 | 33 | 15 | 209 |

TABLE 3
Anomeric linkage analysis of soluble oligosaccharides and
polysaccharides by ¹H NMR spectroscopy.

| % | % | % | % | % | % alpha-1,2 |
|---|---|---|---|---|---|
| alpha-1,4 | alpha-1,3 | alpha-1,3,6 | alpha-1,2,6 | alpha-1,6 | branching |
| 0 | 0 | 0 | 51.7 | 48.3 | 51.7 |

Thus, GTF9905 has significant alpha-1,2 branching activity and can be used to modify the structure of a glucan substrate.

### Example 14

### Production of Soluble Alpha-1,2-Branched Polysaccharides by Stepwise Combination of Glucosyltransferase GTF6831 and Alpha-1,2 Branching Enzyme GTF9905

First, a reaction was performed to prepare glucan that was used in the second part of this Example as a substrate for 1,2-branching enzyme. A reaction mixture (20 mL) comprised of sucrose (488 g/L) and GTF6831 (Example 5, 4.6 U/mL) was adjusted to pH 5.5 with 6.0 N hydrochloric acid and stirred at 47° C. Aliquots were withdrawn at predetermined times and quenched by heating at 90° C. for 20 min. The resulting heat-treated aliquots were centrifuged and the supernatants analyzed by HPLC to determine the concentration of sucrose, glucose, fructose, leucrose, oligosaccharides and polysaccharides. After 24.5 h, the reaction mixture was heated to 90° C. for 30 minutes, and an aliquot of the heat-treated reaction mixture was centrifuged and the resulting supernatant analyzed for soluble monosaccharides, oligosaccharides and polysaccharides (DP8+) (Table 4).

TABLE 4
HPLC analysis of soluble monosaccharides, oligosaccharides and
polysaccharides produced by glucosyltransferase GTF6831.

| DP8 + | DP7 | DP6 | DP5 | DP4 | DP3 | DP2 | Sucrose | Leucrose | Glucose | Fructose |
|---|---|---|---|---|---|---|---|---|---|---|
| g/L | g/L | g/L | g/L | g/L | g/L | g/L | g/L | g/L | g/L | g/L |
| 263 | 0.1 | 1.5 | 1.3 | 0.0 | 0.0 | 4.8 | 2.8 | 36 | 4.3 | 221 |

A second reaction was then performed to conduct alpha-1,2 branching from the products of the first reaction. The second reaction mixture was prepared by mixing 5.84 mL of the heat-treated first reaction mixture prepared above with 2.92 mL of sucrose solution (600 g/L sucrose in deionized water; final sucrose concentration 175 g/L), 0.67 mL of 0.75 M sodium acetate buffer (pH 5.5, 50 mM final concentration), 0.070 mL deionized water and 0.50 mL of a centrifuged cell lysate containing alpha-1,2-branching enzyme GTF9905 (Example 10, final GTF9905 concentration 1.13 U/mL), followed by stirring at 30° C. Aliquots were withdrawn at predetermined times and quenched by heating at 90° C. for 20 min. The resulting heat-treated aliquots were centrifuged and the supernatants analyzed by HPLC to determine the concentration of sucrose, glucose, fructose, leucrose, oligosaccharides and polysaccharides. After 76.2 h, the reaction mixture was heated to 90° C. for 20 minutes, and an aliquot of the heat-treated reaction mixture was centrifuged. The resulting supernatant was analyzed for soluble monosaccharides, oligosaccharides and polysaccharides (Table 5), and analyzed by ¹H NMR spectroscopy to determine the anomeric linkages of the oligosaccharides and polysaccharides (Table 6).

TABLE 5
HPLC analysis of soluble monosaccharides, oligosaccharides and
polysaccharides produced by an alpha-1,2 branching reaction.

| DP8 + | DP7 | DP6 | DP5 | DP4 | DP3 | DP2 | Sucrose | Leucrose | Glucose | Fructose |
|---|---|---|---|---|---|---|---|---|---|---|
| g/L | g/L | g/L | g/L | g/L | g/L | g/L | g/L | g/L | g/L | g/L |
| 231 | 0.0 | 0.0 | 0.0 | 0.0 | 0 | 5.0 | 23 | 38 | 16 | 196 |

TABLE 6
Anomeric linkage analysis of soluble oligosaccharides and
polysaccharides by ¹H NMR spectroscopy.

| % | % | % | % | % | % alpha-1,2 |
|---|---|---|---|---|---|
| alpha-1,4 | alpha-1,3 | alpha-1,3,6 | alpha-1,2,6 | alpha-1,6 | branching |
| 0 | 0 | 0 | 50.8 | 49.2 | 50.8 |

Thus, it was further shown that GTF9905 has significant alpha-1,2 branching activity and can be used to modify the structure of a glucan substrate.

### Example 15

### Production of Soluble Alpha-1,2-Branched Polysaccharides by Stepwise Combination of Glucosyltransferase GTF6831 and Alpha-1,2 Branching Enzyme GTFJ18T1

First, a reaction was performed to prepare glucan that was used in the second part of this Example as a substrate for 1,2-branching enzyme. A reaction mixture (20 mL) comprised of sucrose (488 g/L) and GTF6831 (Example 5, 4.6 U/mL) was adjusted to pH 5.5 with 6.0 N hydrochloric acid and stirred at 47° C. Aliquots were withdrawn at predetermined times and quenched by heating at 90° C. for 20 min. The resulting heat-treated aliquots were centrifuged and the supernatants analyzed by HPLC to determine the concentration of sucrose, glucose, fructose, leucrose, oligosaccharides and polysaccharides. After 22.5 h, the reaction mixture was heated to 90° C. for 30 minutes, and an aliquot of the heat-treated reaction mixture was centrifuged and the resulting supernatant analyzed for soluble monosaccharides, oligosaccharides and polysaccharides (DP8+) (Table 7).

TABLE 7
HPLC analysis of soluble monosaccharides, oligosaccharides and
polysaccharides produced by glucosyltransferase GTF6831.

| DP8 + | DP7 | DP6 | DP5 | DP4 | DP3 | DP2 | Sucrose | Leucrose | Glucose | Fructose |
|---|---|---|---|---|---|---|---|---|---|---|
| g/L | g/L | g/L | g/L | g/L | g/L | g/L | g/L | g/L | g/L | g/L |
| 273 | 0.0 | 1.0 | 1.0 | 0.7 | 0.0 | 5.5 | 2.6 | 33.4 | 3.7 | 212 |

A second reaction was then performed to conduct alpha-1,2 branching from the products of the first reaction. The second reaction mixture was prepared by mixing 5.84 mL of the heat-treated first reaction mixture prepared above with 2.09 mL of sucrose solution (600 g/L sucrose in deionized water; final sucrose concentration 125 g/L), 0.67 mL of 0.75 M sodium acetate buffer (pH 5.5, 50 mM final concentration) and 1.40 mL of a centrifuged cell lysate containing alpha-1,2-branching enzyme GTFJ18T1 (Example 7, final GTFJ18T1 concentration 0.70 U/mL), followed by stirring at 30° C. Aliquots were withdrawn at predetermined times and quenched by heating at 90° C. for 20 min. The resulting heat-treated aliquots were centrifuged and the supernatants analyzed by HPLC to determine the concentration of sucrose, glucose, fructose, leucrose, oligosaccharides and polysaccharides. After 51.5 h, the reaction mixture was heated to 90° C. for 30 minutes, and an aliquot of the heat-treated reaction mixture was centrifuged. The resulting supernatant was analyzed for soluble monosaccharides, oligosaccharides and polysaccharides (Table 8), and analyzed by ¹H NMR spectroscopy to determine the anomeric linkages of the oligosaccharides and polysaccharides (Table 9).

TABLE 8
HPLC analysis of soluble monosaccharides, oligosaccharides
and polysaccharides produced by an alpha-1,2 branching reaction.

| DP8+ | DP7 | DP6 | DP5 | DP4 | DP3 | DP2 | Sucrose | Leucrose | Glucose | Fructose |
|---|---|---|---|---|---|---|---|---|---|---|
| g/L | g/L | g/L | g/L | g/L | g/L | g/L | g/L | g/L | g/L | g/L |
| 204 | 0.0 | 0.0 | 0.0 | 0.0 | 0 | 3.2 | 17.1 | 38.6 | 7.2 | 173 |

TABLE 9
Anomeric linkage analysis of soluble oligosaccharides and
polysaccharides by ¹H NMR spectroscopy.

| % | % | % | % | % | % alpha-1,2 |
|---|---|---|---|---|---|
| alpha-1,4 | alpha-1,3 | alpha-1,3,6 | alpha-1,2,6 | alpha-1,6 | branching |
| 0 | 0 | 0 | 37.1 | 62.9 | 37.1 |

Thus, it was further shown that GTFJ18T1 has significant alpha-1,2 branching activity and can be used to modify the structure of a glucan substrate.

### Example 16

### Production of Soluble Alpha-1,2-Branched Polysaccharides by Simultaneous Combination of Glucosyltransferase GTF8117 and Alpha-1,2 Branching Enzyme GTF9905 (1)

A reaction mixture comprising sucrose (450 g/L), GTF8117 (Example 4, 0.944 U/mL;), and alpha-1,2-branching enzyme GTF9905 (Example 10, 1.06 U/mL) in 54 mM acetate buffer, pH 5.5 was stirred at 30° C. Aliquots were withdrawn at predetermined times and quenched by heating at 90° C. for 20 min. Heat-treated aliquots were centrifuged and the supernatants were analyzed by HPLC to determine the concentration of sucrose, glucose, fructose, leucrose, oligosaccharides and polysaccharides. After 74 h, the reaction mixture was heated to 90° C. for 20 minutes and centrifuged. The resulting supernatant was analyzed for soluble monosaccharides, oligosaccharides and polysaccharides (DP8+) (Table 10), and analyzed by ¹H NMR spectroscopy to determine the anomeric linkages of the oligosaccharides and polysaccharides (Table 11).

TABLE 10
HPLC analysis of soluble monosaccharides, oligosaccharides and polysaccharides
produced by simultaneous glucan synthesis and alpha-1,2 branching reactions.

| DP8+ | DP7 | DP6 | DP5 | DP4 | DP3 | DP2 | Sucrose | Leucrose | Glucose | Fructose |
|---|---|---|---|---|---|---|---|---|---|---|
| g/L | g/L | g/L | g/L | g/L | g/L | g/L | g/L | g/L | g/L | g/L |
| 192 | 0.0 | 0.0 | 0.0 | 0.0 | 1.4 | 8.7 | 4.7 | 44.1 | 23.4 | 199 |

TABLE 11
Anomeric linkage analysis of soluble oligosaccharides and
polysaccharides by ¹H NMR spectroscopy.

| % | % | % | % | % | % alpha-1,2 |
|---|---|---|---|---|---|
| alpha-1,4 | alpha-1,3 | alpha-1,3,6 | alpha-1,2,6 | alpha-1,6 | branching |
| 0 | 0 | 0 | 39.3 | 60.7 | 39.3 |

Thus, it was further shown that GTF9905 has significant alpha-1,2 branching activity and can be used to modify the structure of a glucan substrate.

### Example 17

### Production of Soluble Alpha-1,2-Branched Polysaccharides by Simultaneous Combination of Glucosyltransferase GTF8117 and Alpha-1,2 Branching Enzyme GTF9905 (2)

A reaction mixture comprised of sucrose (500 g/L), GTF8117 (Example 4, 2.83 U/mL), and alpha-1,2-branching enzyme GTF9905 (Example 10, 3.17 U/mL) in 54 mM acetate buffer, pH 5.5 was stirred at 30° C. Aliquots were withdrawn at predetermined times and quenched by heating at 90° C. for 20 min. Heat-treated aliquots were centrifuged and the supernatants were analyzed by HPLC to determine the concentration of sucrose, glucose, fructose, leucrose, oligosaccharides and polysaccharides. After 28.5 h, the reaction mixture was heated to 90° C. for 20 minutes and centrifuged. The resulting supernatant was analyzed for soluble monosaccharides, oligosaccharides and polysaccharides (DP8+) (Table 12), and analyzed by ¹H NMR spectroscopy to determine the anomeric linkages of the oligosaccharides and polysaccharides (Table 13).

TABLE 12
HPLC analysis of soluble monosaccharides, oligosaccharides and polysaccharides
produced by simultaneous glucan synthesis and alpha-1,2 branching reactions.

| DP8+ | DP7 | DP6 | DP5 | DP4 | DP3 | DP2 | Sucrose | Leucrose | Glucose | Fructose |
|---|---|---|---|---|---|---|---|---|---|---|
| g/L | g/L | g/L | g/L | g/L | g/L | g/L | g/L | g/L | g/L | g/L |
| 215 | 0.0 | 0.0 | 0.0 | 0.0 | 1.4 | 8.5 | 2.2 | 46.3 | 23.8 | 234 |

TABLE 13
Anomeric linkage analysis of soluble polysaccharides
by ¹H NMR spectroscopy.

| % | % | % | % | % | % alpha-1,2 |
|---|---|---|---|---|---|
| alpha-1,4 | alpha-1,3 | alpha-1,3,6 | alpha-1,2,6 | alpha-1,6 | branching |
| 0 | 0 | 0 | 34.3 | 65.7 | 34.3 |

Thus, it was further shown that GTF9905 has significant alpha-1,2 branching activity and can be used to modify the structure of a glucan substrate.

### Example 18

### Production of Soluble Alpha-1,2-Branched Polysaccharides by Simultaneous Combination of Glucosyltransferase GTF8117 and Alpha-1,2 Branching Enzyme GTFJ18T1 (1)

A reaction mixture comprised of sucrose (450 g/L), GTF8117 (Example 4, 0.944 U/mL), and alpha-1,2-branching enzyme GTFJ18T1 (Example 7, 1.06 U/mL) in 54 mM acetate buffer, pH 5.5 was stirred at 30° C. Aliquots were withdrawn at predetermined times and quenched by heating at 90° C. for 20 min. Heat-treated aliquots were centrifuged and the supernatants were analyzed by HPLC to determine the concentration of sucrose, glucose, fructose, leucrose, oligosaccharides and polysaccharides. After 71 h, the reaction mixture was heated to 90° C. for 20 minutes and centrifuged. The resulting supernatant was analyzed for soluble monosaccharides, oligosaccharides and polysaccharides (DP8+) (Table 14), and analyzed by ¹H NMR spectroscopy to determine the anomeric linkages of the oligosaccharides and polysaccharides (Table 15).

TABLE 14
HPLC analysis of soluble monosaccharides, oligosaccharides and polysaccharides
produced by simultaneous glucan synthesis and alpha-1,2 branching reactions.

| DP8+ | DP7 | DP6 | DP5 | DP4 | DP3 | DP2 | Sucrose | Leucrose | Glucose | Fructose |
|---|---|---|---|---|---|---|---|---|---|---|
| g/L | g/L | g/L | g/L | g/L | g/L | g/L | g/L | g/L | g/L | g/L |
| 194 | 0.0 | 0.0 | 0.0 | 0.0 | 0.0 | 8.0 | 1.3 | 70.4 | 14.3 | 201 |

TABLE 15
Anomeric linkage analysis of soluble oligosaccharide and
polysaccharide by ¹H NMR spectroscopy.

| % | % | % | % | % | % alpha 1,2 |
|---|---|---|---|---|---|
| alpha-1,4 | alpha-1,3 | alpha-1,3,6 | alpha-1,2,6 | alpha-1,6 | branching |
| 0 | 0 | 0 | 34.0 | 66.0 | 34.0 |

Thus, it was further shown that GTFJ18T1 has significant alpha-1,2 branching activity and can be used to modify the structure of a glucan substrate.

### Example 19

### Production of Soluble Alpha-1,2-Branched Polysaccharides by Simultaneous Combination of Glucosyltransferase GTF8117 and Alpha-1,2 Branching Enzyme GTFJ18T1 (2)

A reaction mixture comprised of sucrose (450 g/L), GTF8117 (Example 4, 0.472 U/mL), and alpha-1,2-branching enzyme GTFJ18T1 (Example 7, 1.06 U/mL) in 54 mM acetate buffer, pH 5.5 was stirred at 30° C. Aliquots were withdrawn at predetermined times and quenched by heating at 90° C. for 20 min. Heat-treated aliquots were centrifuged and the supernatants were analyzed by HPLC to determine the concentration of sucrose, glucose, fructose, leucrose, oligosaccharides and polysaccharides. After 131 h, the reaction mixture was heated to 90° C. for 20 minutes and centrifuged. The resulting supernatant was analyzed for soluble monosaccharides, oligosaccharides and polysaccharides (DP8+) (Table 16), and analyzed by ¹H NMR spectroscopy to determine the anomeric linkages of the oligosaccharides and polysaccharides (Table 17).

TABLE 16
HPLC analysis of soluble monosaccharides, oligosaccharides and polysaccharides
produced by simultaneous glucan synthesis and alpha-1,2 branching reactions.

| DP8+ | DP7 | DP6 | DP5 | DP4 | DP3 | DP2 | Sucrose | Leucrose | Glucose | Fructose |
|---|---|---|---|---|---|---|---|---|---|---|
| g/L | g/L | g/L | g/L | g/L | g/L | g/L | g/L | g/L | g/L | g/L |
| 131 | 0.0 | 0.0 | 0.0 | 0.0 | 2.5 | 11.2 | 0.4 | 107 | 20.5 | 182 |

TABLE 17
Anomeric linkage analysis of soluble oligosaccharide and
polysaccharide by ¹H NMR spectroscopy.

| % | % | % | % | % | % alpha-1,2 |
|---|---|---|---|---|---|
| alpha-1,4 | alpha-1,3 | alpha-1,3,6 | alpha-1,2,6 | alpha-1,6 | branching |
| 0 | 0 | 0 | 39.1 | 60.9 | 39.1 |

Thus, it was further shown that GTFJ18T1 has significant alpha-1,2 branching activity and can be used to modify the structure of a glucan substrate.

### Example 20

### Isolation and Characterization of Alpha-1,2-Branched Polysaccharides

Four 500-mL reactions (1-4) containing 200 g/L sucrose and 9.44 U/mL of GTF8117 (Example 4) were adjusted to pH 5.5 and mixed at 47° C. on a rotary shaker for 18 h. Aliquots of each product mixture were removed at predetermined times, heated at 90° C. for 20 min, cooled to ca. 25° C., and then centrifuged; the resulting supernatants were analyzed by HPLC to determine the concentration of sucrose, glucose, fructose, and leucrose present during conversion of sucrose to a linear alpha-1,6-dextran polysaccharide (Table 18). After 18 h, the four reaction mixtures were heated at 90° C. for 20 min, cooled to ca. 25° C., and then centrifuged; the resulting supernatants were combined and stored at 5° C. prior to use in a subsequent reaction (below) that added alpha-1,2-glucosyl branching to the alpha-1,6-linked dextran polysaccharide reaction product.

TABLE 18
HPLC analysis of soluble monosaccharides and disaccharides
produced by conversion of sucrose to polysaccharide using GTF8117.

| Reaction | Sucrose | Leucrose | Glucose | Fructose |
|---|---|---|---|---|
| no. | g/L | g/L | g/L | g/L |
| 1 | 0.92 | 7.0 | 2.3 | 103 |
| 2 | 0.89 | 6.6 | 2.2 | 97.3 |
| 3 | 0.95 | 6.9 | 2.4 | 103 |
| 4 | 0.95 | 6.9 | 2.3 | 103 |

Two 250-mL jacketed resin kettles were each charged with 100 mL of the GTF8117 reaction product supernatant from above (final concentration: 80 g/L of total dissolved solids derived from sucrose), 33.3 mL of a 600 g/L sucrose stock solution (final concentration of 80 g/L sucrose), and 91.7 mL of distilled water. The mixtures were heated in situ at 80° C. for 30 minutes, then cooled to 30° C., after which 25 mL of GTFJ18T1 enzyme solution (Example 7, 0.5 U/mL final GTFJ18T1 concentration) was added and the pH immediately adjusted to 5.5 with 0.5% sodium hydroxide (these two reactions are referred to below as Reactions 1 and 2). The reaction pH was continuously controlled at 5.5 using a pH electrode connected to a peristaltic pump that fed 0.5% sodium hydroxide into the reaction mixture as needed. Aliquots were withdrawn at predetermined times and quenched by heating at 90° C. for 20 min. The resulting heat-treated aliquots were centrifuged and the supernatants analyzed by HPLC to determine the concentration of sucrose, glucose, fructose, leucrose, oligosaccharides and polysaccharides. After 44 h, each reaction mixture (Reactions 1 and 2) was heated to 90° C. for 20 minutes and centrifuged. The resulting supernatants were analyzed by HPLC for soluble monosaccharides, oligosaccharides and polysaccharides (Table 19); by ¹H NMR spectroscopy to determine the anomeric linkages of the oligosaccharides and polysaccharides (Table 20); and by size-exclusion chromatography for molecular weight (Table 21).

TABLE 19
HPLC analysis of soluble monosaccharides and disaccharides
produced in Reactions 1 and 2.

| Reaction | Sucrose | Leucrose | Glucose | Fructose |
|---|---|---|---|---|
| no. | g/L | g/L | g/L | g/L |
| 1 | 27.1 | 7.4 | 0 | 50.0 |
| 2 | 34.4 | 5.8 | 0 | 46.9 |

TABLE 20
Anomeric linkage analysis of soluble alpha-1,2-
branched polysaccharides produced in Reactions 1 and 1.

| Reaction | % | % | % | % | % | % alpha-1,2 |
|---|---|---|---|---|---|---|
| no. | alpha-1,4 | alpha-1,3 | alpha-1,3,6 | alpha-1,2,6 | alpha-1,6 | branching |
| 1 | 0 | 0 | 0 | 46.7 | 53.3 | 46.7 |
| 2 | 0 | 0 | 0 | 41.4 | 58.6 | 41.4 |

TABLE 21
Molecular weight analysis of alpha-1,2-branched
polysaccharides produced in Reactions 1 and 2.

| Reaction | | | | | |
|---|---|---|---|---|---|
| no. | Mn | Mp | Mw | Mz | PDI |
| 1 | 11348 | 29189 | 25460 | 35600 | 2.244 |
| 2 | 11023 | 27148 | 23868 | 33166 | 2.165 |

Thus, it was further shown that GTFJ18T1 has significant alpha-1,2 branching activity and can be used to modify the structure of a glucan substrate.

The supernatants from Reactions 1 and 2 were combined, and the alpha-1,2-branched polysaccharides therein were purified and isolated by ultrafiltration (UF) using a 5-kDa molecular weight cutoff (MWCO) polyethersulfone (PES) membrane (Pall Centramate™ LV). HPLC analysis of the UF retentate indicated no detectable monosaccharides, disaccharides or DP2-DP8 oligosaccharides. The UF retentate was adjusted to ca. 5 wt % dissolved solids and the resulting solution lyophilized to produce the soluble alpha-1,2-branched polysaccharides as a dry solid. The solid polysaccharide product was analyzed by ¹H NMR spectroscopy (Table 22) and by GC/MS to determine the anomeric linkages of the polysaccharides (Table 23).

TABLE 22
Anomeric linkage analysis (¹H NMR) of soluble alpha-1,2-
branched polysaccharides produced in Reactions 1 and 2 (combined).

| sample | % | % | % | % | % | % | % alpha-1,2 |
|---|---|---|---|---|---|---|---|
| no. | alpha-1,4 | alpha-1,3 | alpha-1,3,6 | alpha-1,2 | alpha-1,2,6 | alpha-1,6 | branching |
| 105-1 | 0 | 0 | 0 | 31.1 | 31.1 | 37.8 | 45.2 |

TABLE 23
Anomeric linkage analysis (GC/MS) of soluble alpha-1,2-branched
polysaccharides produced using GTFJ18Tl in Reactions 1 and 2 (combined).

| sample | % | % | % | % | % | % |
|---|---|---|---|---|---|---|
| no. | alpha-1,3 | alpha-1,2 | alpha-1,6 | alpha-1,4 | alpha-1,3,6 | alpha-1,2,6 |
| 105-1 | 0.3 | 0.8 | 57.5 | 0.2 | 0.5 | 40.7 |

### Example 21

Isolation and characterization of alpha-1,2-branched polysaccharides Two 250-mL jacketed resin kettles were each charged with 100 mL of the GTF8117 reaction product supernatant produced in the first part of Example 20 above (final concentration: 80 g/L of total dissolved solids derived from sucrose), 33.3 mL of a 600 g/L sucrose stock solution (final concentration of 80 g/L sucrose), and 91.7 mL of distilled water. The mixtures were heated in situ at 80° C. for 30 minutes, then cooled to 30° C., after which 25 mL of GTFJ18T1 enzyme solution (Example 7, 0.5 U/mL final GTFJ18T1 concentration) was added and the pH immediately adjusted to 5.5 with 0.5% sodium hydroxide (these two reactions are referred to below as Reactions 3 and 4). The reaction pH was continuously controlled at 5.5 using a pH electrode connected to a peristaltic pump that fed 0.5% sodium hydroxide into the reaction mixture as needed. Aliquots were withdrawn at predetermined times and quenched by heating at 90° C. for 20 min. The resulting heat-treated aliquots were centrifuged and the supernatants analyzed by HPLC to determine the concentration of sucrose, glucose, fructose, leucrose, oligosaccharides and polysaccharides. After 4 h, each reaction mixture (Reactions 3 and 4) was heated to 90° C. for 20 minutes and centrifuged. The resulting supernatants were analyzed by HPLC for soluble monosaccharides, oligosaccharides and polysaccharides (Table 24), by ¹H NMR spectroscopy to determine the anomeric linkages of the oligosaccharides and polysaccharides (Table 25), and by size-exclusion chromatography for molecular weight (Table 26).

TABLE 24
HPLC analysis of soluble monosaccharides and
disaccharides produced in Reactions 3 and 4.

| Reaction | Sucrose | Leucrose | Glucose | Fructose |
|---|---|---|---|---|
| no. | g/L | g/L | g/L | g/L |
| 3 | 64.7 | 3.9 | 1.6 | 47.0 |
| 4 | 65.9 | 4.0 | 1.3 | 46.4 |

TABLE 25
Anomeric linkage analysis of soluble alpha-1,2-
branched polysaccharides produced in Reactions 3 and 4.

| Reaction | % | % | % | % | % | % alpha-1,2 |
|---|---|---|---|---|---|---|
| no. | alpha-1,4 | alpha-1,3 | alpha-1,3,6 | alpha-1,2,6 | alpha-1,6 | branching |
| 3 | 0 | 0 | 0 | 21.4 | 78.6 | 21.4 |
| 4 | 0 | 0 | 0 | 20.5 | 79.5 | 20.5 |

TABLE 26
Molecular weight analysis of alpha-1,2-branched polysaccharides
produced in Reactions 3 and 4.

| Reaction | | | | | |
|---|---|---|---|---|---|
| no. | Mn | Mp | Mw | Mz | PDI |
| 3 | 10395 | 23593 | 20458 | 28218 | 1.968 |
| 4 | 10282 | 23121 | 20092 | 27639 | 1.954 |

Thus, it was further shown that GTFJ18T1 has significant alpha-1,2 branching activity and can be used to modify the structure of a glucan substrate.

The supernatants from Reactions 3 and 4 were combined, and the alpha-1,2-branched polysaccharides therein were purified and isolated by ultrafiltration (UF) using a 5-kDa molecular weight cutoff (MWCO) polyethersulfone (PES) membrane (Pall Centramate™ LV). HPLC analysis of the UF retentate indicated no detectable monosaccharides, disaccharides or DP2-DP8 oligosaccharides. The UF retentate was adjusted to ca. 5 wt % dissolved solids and the resulting solution lyophilized to produce the soluble alpha-1,2-branched polysaccharides as a dry solid. The solid polysaccharide product was analyzed by ¹H NMR spectroscopy (Table 27) and by GC/MS to determine the anomeric linkages of the polysaccharides (Table 28).

TABLE 27
Anomeric linkage analysis (¹H NMR) of soluble alpha-1,2-
branched polysaccharides produced in Reactions 3 and 4 (combined).

| sample | % | % | % | % | % | % | % alpha-1,2 |
|---|---|---|---|---|---|---|---|
| no. | alpha-1,4 | alpha-1,3 | alpha-1,3,6 | alpha-1,2 | alpha-1,2,6 | alpha-1,6 | branching |
| 105-2 | 0 | 0 | 0 | 17.7 | 17.3 | 65 | 21.0 |

TABLE 28
Anomeric linkage analysis (GC/MS) of soluble alpha-1,2-branched
polysaccharides produced using GTFJ18T1 in Reactions 3 and 4 (combined).

| sample | % | % | % | % | % | % |
|---|---|---|---|---|---|---|
| no. | alpha-1,3 | alpha-1,2 | alpha-1,6 | alpha-1,4 | alpha-1,3,6 | alpha-1,2,6 |
| 105-2 | 0.2 | 0.4 | 78.1 | 0.1 | 0.4 | 20.8 |

### Example 22

### Isolation and Characterization of Alpha-1,2-Branched Polysaccharides

Two 250-mL jacketed resin kettles were each charged with 175 mL of the GTF8117 reaction product supernatant produced in the first part of Example 20 above (final concentration: 140 g/L of total dissolved solids derived from sucrose), 4.2 mL of a 600 g/L sucrose stock solution (final concentration of 10 g/L sucrose), and 45.8 mL of distilled water. The mixtures were heated in situ at 80° C. for 30 minutes, then cooled to 30° C., after which 25 mL of GTFJ18T1 enzyme solution (Example 7, 0.5 U/mL final GTFJ18T1 concentration) was added and the pH immediately adjusted to 5.5 with 0.5% sodium hydroxide (these two reactions are referred to below as Reactions 5 and 6). The reaction pH was continuously controlled at 5.5 using a pH electrode connected to a peristaltic pump that fed 0.5% sodium hydroxide into the reaction mixture as needed. Aliquots were withdrawn at predetermined times and quenched by heating at 90° C. for 20 min. The resulting heat-treated aliquots were centrifuged and the supernatants analyzed by HPLC to determine the concentration of sucrose, glucose, fructose, leucrose, oligosaccharides and polysaccharides. After 20 h, each reaction mixture (Reactions 5 and 6) was heated to 90° C. for 20 minutes and centrifuged. The resulting supernatants were analyzed by HPLC for soluble monosaccharides, oligosaccharides and polysaccharides (Table 29), by ¹H NMR spectroscopy to determine the anomeric linkages of the oligosaccharides and polysaccharides (Table 30), and by size-exclusion chromatography for molecular weight (Table 31).

TABLE 29
HPLC analysis of soluble monosaccharides and disaccharides
produced in Reactions 5 and 6.

| Reaction | Sucrose | Leucrose | Glucose | Fructose |
|---|---|---|---|---|
| no. | g/L | g/L | g/L | g/L |
| 5 | 4.2 | 4.2 | 0 | 58.5 |
| 6 | 0.4 | 3.7 | 0 | 53.8 |

TABLE 30
Anomeric linkage analysis of soluble alpha-1,2-
branched polysaccharides produced in Reactions 5 and 6.

| Reaction | % | % | % | % | % | % alpha-1,2 |
|---|---|---|---|---|---|---|
| no. | alpha-1,4 | alpha-1,3 | alpha-1,3,6 | alpha-1,2,6 | alpha 1,6 | branching |
| 5 | 0 | 0 | 0 | 4.6 | 95.4 | 4.6 |
| 6 | 0 | 0 | 0 | 5.8 | 94.2 | 5.8 |

TABLE 31
Molecular weight analysis of alpha-1,2-branched polysaccharides
produced in Reactions 5 and 6.

| Reaction | | | | | |
|---|---|---|---|---|---|
| no. | Mn | MP | Mw | Mz | PDI |
| 5 | 9032 | 20263 | 17725 | 24229 | 1.962 |
| 6 | 9492 | 20425 | 17851 | 24297 | 1.881 |

Thus, it was further shown that GTFJ18T1 has significant alpha-1,2 branching activity and can be used to modify the structure of a glucan substrate.

The supernatants from Reactions 5 and 6 were combined, and the alpha-1,2-branched polysaccharides therein were purified and isolated by ultrafiltration (UF) using a 5-kDa molecular weight cutoff (MWCO) polyethersulfone (PES) membrane (Pall Centramate™ LV). HPLC analysis of the UF retentate indicated no detectable monosaccharides, disaccharides or DP2-DP8 oligosaccharides. The UF retentate was adjusted to ca. 5 wt % dissolved solids and the resulting solution lyophilized to produce the soluble alpha-1,2-branched polysaccharides as a dry solid. The solid polysaccharide product was analyzed by ¹H NMR spectroscopy (Table 32) and by GC/MS to determine the anomeric linkages of the polysaccharides (Table 33).

TABLE 32
Anomeric linkage analysis of soluble alpha-1,2-
branched polysaccharides produced in Reactions 5 and 6.

| sample | % | % | % | % | % | % | % alpha-1,2 |
|---|---|---|---|---|---|---|---|
| no. | alpha-1,4 | alpha-1,3 | alpha-1,3,6 | alpha-1,2 | alpha-1,2,6 | alpha-1,6 | branching |
| 105-3 | 0 | 0 | 0 | 5.1 | 5.1 | 89.8 | 5.4 |

TABLE 33
Anomeric linkage analysis of soluble alpha-1,2-
branched polysaccharides produced in Reactions 5 and 6.

| sample | % | % | % | % | % | % |
|---|---|---|---|---|---|---|
| no. | alpha-1,3 | alpha-1,2 | alpha-1,6 | alpha-1,4 | alpha-1,3,6 | alpha-1,2,6 |
| 105-3 | 0.2 | 0.5 | 90.1 | 0.4 | 0.5 | 8.3 |

### Example 23

### Glycemic Response in Mice

The objective of the screening study was to assess the digestibility of sample 105-1 (Example 20), sample 105-2 (Example 21), and sample 105-3 (Example 22) that were each produced using GTF8117 and GTFJ18T1, by evaluating the glycemic response of male mice following a single dose. Seven groups of male C57Bl/6 mice each received a single dose of one of the three test substances (sample 105-1, -2, or -3), positive or negative control substances, or a vehicle control. Blood was obtained via the tail vein in order to measure glucose levels using a glucometer. Multiple readings were obtained on the day of test substance exposure.

The oral route of administration was selected because it is the intended route of human exposure and the most efficient way to deliver an accurate dose. Groups of 12 young adult male C57Bl/6J mice were dosed by oral gavage with one of the following treatments: a test substance (sample 105-1, sample 105-2, or sample 105-3), a positive control substance (glucose [data shown], or an alpha-1,3-linked glucan having no alpha-1,2 branches [data not shown]), a negative control substance (Litesse® Ultra), or a vehicle control (deionized water). The dose level was 2000 mg/kg for all treatments except for the water control, which was administered at the same dose volume (10 mL/kg) as the other treatments (Table 34).

The experiment was conducted after habituating the animals to the testing procedure, including daily gavage with water and multiple baseline glucometer readings prior to dosing. Three baseline glucometer readings were performed within approximately 1 hour prior to dosing (pre-dose measurements 1-3), with approximately 20 minutes between each of the three readings and between the third reading and dosing. Post-dose glucometer readings were performed after approximately 20, 40, 60, and 120 minutes. The ALPHA TRAK 2 glucometer was used to measure the glucose in at least 0.3 microliter of blood obtained by pricking the tail vein with a sterile needle. The area under the curve (AUC) was calculated for 0 to 2 hours. The AUC was calculated by adding together a series of trapezoids rather than a prediction model of a smooth curve. The final baseline value was designated as “0 minutes post-dosing” for the purpose of the calculation.

Under the conditions of the study, administration of sample 105-1 or sample 105-2 at 2000 mg/kg by oral gavage did not result in any increase in blood glucose; the glycemic response was similar to that of the Litesse® Ultra dietary fiber or deionized water (Table 35). Administration of sample 105-3 at 2000 mg/kg by oral gavage resulted in a glycemic response with a slower onset and lower peak magnitude than that induced by free glucose (a positive control) (the alpha-1,3-linked glucan having no alpha-1,2 branches induced a glycemic response very similar to that induced by glucose [data not shown]). A glycemic response (i.e., a statistically significant increase in blood glucose levels) was considered to be a potential indicator of the digestibility of these test substances at a maximal dose of 2000 mg/kg.

TABLE 34
Study design.

| Group | Number and Sex | Treatment | Description | Dose Level^{a} |
|---|---|---|---|---|
| 1 | 12 male | Water | Vehicle | n/a |
| | | | Control | |
| 2 | 12 male | Litesse ® Ultra | Negative | 2000 mg/kg |
| | | | Control | |
| 3 | 12 male | Sample 105-1 | Test | 2000 mg/kg |
| | | | Substance | |
| 4 | 12 male | Sample 105-2 | Test | 2000 mg/kg |
| | | | Substance | |
| 5 | 12 male | Sample 105-3 | Test | 2000 mg/kg |
| | | | Substance | |
| 6 | 12 male | Glucose | Positive | 2000 mg/kg |
| | | | Control | |

^{a}Weight of test substance/kg animal body weight.

TABLE 35
Glucometer readings* (mg/dL).

| | Pre-Dose Measurements | | | Post-Dose Measurements | | | | |
|---|---|---|---|---|---|---|---|---|
| | 1 | 2 | 3 | 20 min | 40 min | 1 hr | 2 hr | AUC, 0-2 hrª |
| Water | 194 | 250 | 266 | 264 | 292 | 263 | 249 | 31780 |
| | (24) | (53) | (51) | (29) | (56) | (59) | (39) | (3875) |
| Litesse ® | 186 | 208 | 273 | 288 | 268 | 299 | 247 | 33233 |
| Ultra | (27) | (28) | (45) | (70) | (34) | (47) | (38) | (3917) |
| 105-1 | 195 | 218 | 265 | 275 | 306 | 276 | 231 | 32229 |
| | (29) | (25) | (39) | (62) | (63) | (40) | (40) | (3494) |
| 105-2 | 192 | 215 | 243 | 294 | 290 | 280 | 250 | 32793 |
| | (25) | (29) | (34) | (42) | (63) | (43) | (55) | (2331) |
| 105-3 | 200 | 212 | 289 | 301 | 331 | 334 | 273 | 37072 |
| | (31) | (18) | (40) | (41) | (61) | (42)^{#} | (62) | (3611)^{#} |
| Glucose | 204 | 206 | 274 | 401 | 374 | 308 | 245 | 37876 |
| | (35) | (27) | (50) | (77)^{#} | (73)^{#} | (44) | (53) | (3974)^{#} |

*Data (each measurement reading) are presented as “mean (SD)”.
^{#}Statistically significant at p < 0.05 by One Way ANOVA followed by Dunnett's test, compared with the water control.
ªThe final baseline value was designated as “0 minutes post-dosing” for the purpose of the AUC calculation.

Thus, sample 105-3 can be characterized as a slow glucose release glucan composition herein, and samples 105-1 and 105-2 can be characterized as having dietary fiber qualities. It is noteworthy that sample 105-3 has less than 10% alpha-1,2 branching (Example 22), whereas samples 105-1 and 105-2 each have greater than 15% alpha-1,2 branching (Examples 20-21).

### Example 24

### Preparation of a Yogurt/Drinkable Smoothie

The following example describes the preparation of a yogurt/drinkable smoothie containing the present alpha-1,2-branched glucan.

TABLE 36

| Ingredients | wt % |
|---|---|
| Distilled Water | 49.00 |
| SUPRO XT40 Soy Protein Isolate | 6.50 |
| Fructose | 1.00 |
| GRINDSTED ASD525, Danisco | 0.30 |
| Apple Juice Concentrate (70 Brix) | 14.79 |
| Strawberry Puree, Single Strength | 4.00 |
| Banana Puree, Single Strength | 6.00 |
| Plain Lowfat Yogurt-Greek Style, Cabot | 9.00 |
| 1% Red 40 Solution | 0.17 |
| Strawberry Flavor (DD-148-459-6) | 0.65 |
| Banana Flavor (#29513) | 0.20 |
| 75/25 Malic/Citric Blend | 0.40 |
| Alpha-1,2-branched glucan (presently disclosed) | 8.00 |
| TOTAL | 100.00 |

Step No. Procedure

### Pectin Solution Formation

1 Heat 50% of the formula water to 160° F. (˜71.1° C.).
2 Disperse the pectin with high shear; mix for 10 minutes.
3 Add the juice concentrates and yogurt; mix for 5-10 minutes until the yogurt is dispersed.

### Protein Slurry

1 Into 50% of the batch water at 140° F. (60° C.), add the SUPRO XT40 and mix well.
2 Heat to 170° F. (˜76.7° C.) and hold for 15 minutes.
3 Add the pectin/juice/yogurt slurry to the protein solution; mix for 5 minutes.
4 Add the fructose, glucan, flavors and colors; mix for 3 minutes.
5 Adjust the pH using phosphoric acid to the desired range (pH range 4.0-4.1).
6 Ultra High Temperature (UHT) processing at 224° F. (˜106.7° C.) for 7 seconds with UHT homogenization after heating at 2500/500 psig (17.24/3.45 MPa) using an indirect steam (IDS) unit.
7 Collect product in bottles and cool in ice bath.
8 Store product in refrigerated conditions.

### Example 25

### Preparation of a Water Preparation Comprising Alpha-1,2-Branched Glucan

The following example describes the preparation of an enhanced water composition.

TABLE 37

| Ingredient | wt % |
|---|---|
| Water, deionized | 86.41 |
| Pistachio Green #06509 | 0.00 |
| Alpha-1,2-branched glucan (presently | 8.00 |
| disclosed herein) | |
| Sucrose | 5.28 |
| Citric Acid | 0.08 |
| Flavor (M748699M) | 0.20 |
| Vitamin C, ascorbic acid | 0.02 |
| TOTAL | 100.00 |

Step No. Procedure
1 Add dry ingredients and mix for 15 minutes.
2 Add remaining dry ingredients; mix for 3 minutes.
3 Adjust pH to 3.0+/−0.05 using citric acid as shown in formulation.
4 Ultra High Temperature (UHT) processing at 224° F. (˜106.7° C.) for 7 seconds with homogenization at 2500/500 psig (17.24/3.45 MPa).
5 Collect product in bottles and cool in ice bath.
6 Store product in refrigerated conditions.

### Example 26

### Preparation of a Spoonable Yogurt Formulation

The following example describes the preparation of a spoonable yogurt containing alpha-1,2-branched glucan.

TABLE 38

| Ingredient | wt % |
|---|---|
| Skim Milk | 84.00 |
| Sugar | 5.00 |
| Yogurt (6051) | 3.00 |
| Cultures (add to pH break point) | |
| Alpha-1,2-branched glucan (presently | 8.00 |
| disclosed herein) | |
| TOTAL | 100.00 |

Step No. Procedure
1 Add dry ingredients to base milk liquid; mix for 5 min.
2 Pasteurize at 195° F. (˜90.6° C.) for 30 seconds, homogenize at 2500 psig (˜17.24 MPa), and cool to 105-110° F. (˜40.6-43.3° C.).
3 Inoculate with culture; mix gently and add to water batch or hot box at 108° F. (˜42.2° C.) until pH reaches 4.5-4.6.

### Fruit Prep Procedure

1 Add water to batch tank; heat to 140° F. (˜60° C.).
2 Pre-blend carbohydrates and stabilizers. Add to batch tank and mix well.
3 Add acid to reduce the pH to the desired range (target pH 3.5-4.0).
4 Add flavor.
5 Cool and refrigerate.

### Example 27

### Preparation of a Model Snack Bar Formulation

The following example describes the preparation of a model snack bar containing alpha-1,2-branched glucan as presently disclosed.

TABLE 39

| Ingredients | wt % |
|---|---|
| Corn Syrup 63 DE | 15.30 |
| Alpha-1,2-branched glucan (presently | 16.60 |
| disclosed herein) solution (70 Brix) | |
| Sunflower Oil | 1.00 |
| Coconut Oil | 1.00 |
| Vanilla Flavor | 0.40 |
| Chocolate Chips | 7.55 |
| SUPRO ® Nugget 309 | 22.10 |
| Rolled Oats | 18.00 |
| Arabic Gum | 2.55 |
| Alkalized Cocoa Powder | 1.00 |
| Milk Chocolate Coating Compound | 14.50 |
| TOTAL | 100.00 |

Step No. Procedure
1 Combine corn syrup with liquid glucan solution. Warm syrup in microwave for 10 seconds.
2 Combine syrup with oils and liquid flavor in mixing bowl. Mix for 1 minute at speed 2.
3 Add all dry ingredients in bowl and mix for 45 seconds at speed 1.
4 Scrape and mix for another 30 seconds or until dough is mixed.
5 Melt chocolate coating.
6 Fully coat the bar with chocolate coating.

### Example 28

### Preparation of a Wafer

The following example describes the preparation of a wafer containing alpha-1,2-branched glucan as presently disclosed.

TABLE 40

| Ingredients | wt % |
|---|---|
| Flour, white plain | 38.17 |
| Alpha-1,2-branched glucan (presently | 2.67 |
| disclosed herein) | |
| Oil, vegetable | 0.84 |
| GRINSTED ® CITREM 2-in-1 | 0.61 |
| (Danisco) citric acid ester made from | |
| sunflower or palm oil (emulsifier) | |
| Salt | 0.27 |
| Sodium bicarbonate | 0.11 |
| Water | 57.33 |

Step No. Procedure
1. High shear the water, oil and CITREM for 20 seconds.
2. Add dry ingredients slowly, high shear for 2-4 minutes.
3. Rest batter for 60 minutes.
4. Deposit batter onto hot plate set at 200° C. top and bottom, bake for 1 minute 30 seconds.
5. Allow cooling pack as soon as possible.

### Example 29

### Preparation of a Soft Chocolate Chip Cookie

The following example describes the preparation of a soft chocolate chip cookie containing alpha-1,2-branched glucan as presently disclosed.

TABLE 41

| Ingredients | wt % |
|---|---|
| Stage 1 | |
| Lactitol, C | 16.00 |
| Cake margarine | 17.70 |
| Salt | 0.30 |
| Baking powder | 0.80 |
| Eggs, dried whole | 0.80 |
| Bicarbonate of soda | 0.20 |
| Vanilla flavor | 0.26 |
| Caramel flavor | 0.03 |
| Sucralose powder | 0.01 |
| Stage 2 | |
| Alpha-1,2-branched glucan (presently | 9.50 |
| disclosed herein) Solution (70 brix) | |
| Water | 4.30 |
| Stage 3 | |
| Flour, pastry | 21.30 |
| Flour, high ratio cake | 13.70 |
| Stage 4 | |
| Chocolate chips, 100% lactitol, sugar- | 15.10 |
| free | |

Step No. Procedure
1. Cream together Stage 1 ingredients under fast speed for 1 minute.
2. Blend with Stage 2 under slow speed for 2 minutes.
3. Add Stage 3 under slow speed for 20 seconds.
4. Scrape down bowl; add Stage 4 under slow speed for 20 seconds.
5. Divide into 30 g pieces, flatten, and place onto silicone-lined baking trays.
6. Bake at 190 C for 10 minutes approximately.

### Example 3 0

### Preparation of a Reduced Fat Short-Crust Pastry Dough

The following example describes the preparation of a reduced fat short-crust pastry dough containing alpha-1,2-branched glucan as presently disclosed.

TABLE 42

| Ingredients | wt % |
|---|---|
| Flour, plain white | 56.6 |
| Water | 15.1 |
| Margarine | 11.0 |
| Shortening | 11.0 |
| alpha-1,2-branched glucan (presently | 6.0 |
| disclosed herein) | |
| Salt | 0.3 |

Step No. Procedure
1. Dry blend the flour, salt and glucan (dry).
2. Gently rub in the fat until the mixture resembles fine breadcrumbs.
3. Add enough water to make a smooth dough.

### Example 31

### Preparation of a Low Sugar Cereal Cluster

The following example describes the preparation of a low sugar cereal cluster containing alpha-1,2-branched glucan as presently disclosed.

TABLE 43

| Ingredients | wt % |
|---|---|
| Syrup Binder | 30.0 |
| Lactitol, MC 50% | |
| Alpha-1,2-branched glucan (presently | |
| disclosed herein) Solution (70 brix) 25% | |
| Water 25% | |
| Cereal Mix | 60.0 |
| Rolled Oats 70% | |
| Flaked Oats 10% | |
| Crisp Rice 10% | |
| Rolled Oats 10% | |
| Vegetable oil | 10.0 |

Step No. Procedure
1. Chop the fines.
2. Weigh the cereal mix and add fines.
3. Add vegetable oil on the cereals and mix well.
4. Prepare the syrup by dissolving the ingredients.
5. Allow the syrup to cool down.
6. Add the desired amount of syrup to the cereal mix.
7. Blend well to ensure even coating of the cereals.
8. Spread onto a tray.
9. Place in a dryer/oven and allow to dry out.
10. Leave to cool down completely before breaking into clusters.

### Example 32

### Preparation of a Pectin Jelly

The following example describes the preparation of a pectin jelly containing alpha-1,2-branched glucan as presently disclosed.

TABLE 44

| Ingredients | wt % |
|---|---|
| Component A | |
| Xylitol | 4.4 |
| Pectin | 1.3 |
| Component B | |
| Water | 13.75 |
| Sodium citrate | 0.3 |
| Citric Acid, anhydrous | 0.3 |
| Component C | |
| Alpha-1,2-branched glucan (presently | 58.1 |
| disclosed herein) Solution (70 brix) | |
| Xylitol | 21.5 |
| Component D | |
| Citric acid | 0.35 |
| Flavor, Color | q.s. |

Step No. Procedure
1. Dry blend the pectin with the xylitol (Component A).
2. Heat Component B until solution starts to boil.
3. Add Component A gradually, and then boil until completely dissolved.
4. Add Component C gradually to avoid excessive cooling of the batch.
5. Boil to 113° C.
6. Allow to cool to <100° C. and then add color, flavor and acid (Component D). Deposit immediately into starch molds.
7. Leave until firm, then de-starch.

### Example 33

### Preparation of a Chewy Candy

The following example describes the preparation of a chewy candy containing alpha-1,2-branched glucan as presently disclosed.

TABLE 45

| Ingredients | wt % |
|---|---|
| Alpha-1,2-branched glucan (presently | 35 |
| disclosed herein) | |
| Xylitol | 35 |
| Water | 10 |
| Vegetable fat | 4.0 |
| Glycerol Monostearate (GMS) | 0.5 |
| Lecithin | 0.5 |
| Gelatin 180 bloom (40% solution) | 4.0 |
| Xylitol, CM50 | 10.0 |
| Flavor, color & acid | q.s. |

Step No. Procedure
1. Mix the glucan, xylitol, water, fat, GMS and lecithin together, and then cook gently to 158° C.
2. Cool the mass to below 90° C. and then add the gelatin solution, flavor, color and acid.
3. Cool further and then add the xylitol CM50. Pull the mass immediately for 5 minutes.
4. Allow the mass to cool again before processing (cut and wrap or drop rolling).

### Example 34

### Preparation of a Coffee-Cherry Ice Cream

The following example describes the preparation of a coffee-cherry ice cream containing alpha-1,2-branched glucan as presently disclosed.

TABLE 46

| Ingredients | wt % |
|---|---|
| Fructose, C | 8.00 |
| Alpha-1,2-branched glucan (presently | 10.00 |
| disclosed herein) | |
| Skimmed milk powder | 9.40 |
| Anhydrous Milk Fat (AMF) | 4.00 |
| CREMODAN ® SE 709 | 0.65 |
| Emulsifier & Stabilizer System (Danisco) | |
| Cherry Flavoring U35814 (Danisco) | 0.15 |
| Instant coffee | 0.50 |
| Tri-sodium citrate | 0.20 |
| Water | 67.10 |

Step No. Procedure
1. Add the dry ingredients to the water while agitating vigorously.
2. Melt the fat.
3. Add the fat to the mix at 40° C.
4. Homogenize at 200 bar/70-75° C.
5. Pasteurize at 80-85° C./20-40 seconds.
6. Cool to ageing temperature (5° C.).
7. Age for minimum 4 hours.
8. Add flavor to the mix.
9. Freeze in continuous freezer to desired overrun (100% is recommended).
10. Harden and store at −25° C.

1-17. (canceled)

18. A composition comprising an ether derivative of an alpha-glucan, wherein the alpha-glucan comprises (i) a backbone and (ii) one or more alpha-1,2 linkages that branch from the backbone,
wherein the backbone has a degree of polymerization of 3 to 2000, and 100% of the glycosidic linkages of the backbone are alpha-1,6 linkages,
and wherein the alpha-glucan does not contain any glycosidic linkages aside from alpha-1,6 and alpha-1,2 glycosidic linkages,
and wherein the composition is in the form of a unit dose.

19. The composition of claim 18, wherein the degree of polymerization is 10 to 1300.

20. The composition of claim 18, wherein the degree of polymerization is 1100 to 1400.

21. The composition of claim 18, wherein the degree of polymerization is 10 to 150.

22. The composition of claim 18, wherein the alpha-glucan has about 3% to 40% alpha-1,2 branching.

23. The composition of claim 18, wherein the alpha-glucan has about 3% to 20% alpha-1,2 branching.

24. The composition of claim 18, wherein the alpha-glucan has about 1% to 10% alpha-1,2 branching.

25. The composition of claim 18, wherein the alpha-glucan has about 3% to 7% alpha-1,2 branching.

26. The composition of claim 18, wherein the alpha-glucan has a degree of substitution of about 0.0025 to about 3.0 with at least one organic group in ether linkage to the alpha-glucan.

27. The composition of claim 26, wherein the degree of substitution is about 0.05 to about 0.4.

28. The composition of claim 26, wherein the organic group is carboxy alkyl, hydroxy alkyl, or alkyl.

29. The composition of claim 26, wherein the organic group is carboxymethyl, hydroxypropyl, dihydroxypropyl, hydroxyethyl, methyl, or ethyl.

30. The composition of claim 26, wherein the organic group is a positively charged organic group.

31. The composition of claim 30, wherein the positively charged organic group is a substituted ammonium group.

32. The composition of claim 31, wherein the substituted ammonium group is a quaternary ammonium group.

33. The composition of claim 18, further comprising at least one enzyme selected from a protease, cellulase, polyesterase, amylase, cutinase, lipase, pectate lyase, perhydrolase, xylanase, peroxidase, mannanase, or laccase.

34. The composition of claim 18, further comprising at least one surfactant selected from an anionic surfactant, nonionic surfactant, cationic surfactant, ampholytic surfactant, or zwitterionic surfactant.

35. The composition of claim 18, further comprising at least one of a fabric conditioner, clay, foam booster, suds suppressor, anti-corrosion agent, soil-suspending agent, anti-soil redeposition agent, dye, bactericide, tarnish inhibiter, optical brightener, or perfume.

36. The composition of claim 18, wherein the unit dose comprises a liquid, solution, gel, paste, dispersion, suspension, or emulsion.

37. The composition of claim 18, wherein the unit dose comprises a powder, granule, microcapsule, flake, pellet, bar, kernel, bead, tablet, stick, pad, or sponge.


SEQUENCE LISTING (28 sequences)

SEQ ID NO: 1 - protein, 2771 aa, Leuconostoc mesenteroides
MTAGIFSAVIFGVSTTNVSADSTNNTGVTVSQATDKVADTTATTDKVADTTATTDKVADTAATTDKVADTTATTDKVADTAATTDKVADTTATTDKVADTAAATDKAADTAATTDKVADTAAATDKVADTVAATDKVADTTATTDKAADTAATTDKVTDTTAATDKAADTTATTDKVADTTATTSEKSKSIKQIDGKTYFIGNDGQPKKNFTAIVDGQVLYFDKDTGALTSNSSQYTDGLANIGNEHNAAYSLSSDSFTQVDGYLTANSWYRPKDILKNGTTWTAATANDFRPLLMSWWPDKDTQVSYLKYMQSAGLLSDDVALSNNDSMNSLTDTAMTVQKKIEEKIGLLGSTDWLKADMNQMVDSQSNWNISSESKGTDHLQGGALLYVNSDLTPNANSDYRLLNRTPTNQKGQITTNGNQGGYEMLLANDVDNSNPIVQAEQLNWLYYMMNIGSIAQNDPTANFDGYRVDAVDNVNADLLQIAGDYFKAAYGTNQSDANANNHISILEDWDNNDPAYVKAQGNNQLTMDFPMHLALKYSLNMPSSARSGLEPAISTSLVNRAADATENEAQPNYSFIRAHDSEVQTVIAQIIKDKINPSSDGLTVSTDEIAKAFEIYNADELKADKEYTAYNIPSSYALMLTNKDTIPRVYYGDLFTDDGQYMSAKSPYYDALTSLLQSRVKYVSGGQSMNMTYLHNNQGLLTSVRYGKDAMTANDTGTSETRTQGIGLIVGNKTDLNLNNDEQIVLNMGAAHKNQAYRALMLSTKDGLKIYNNDDEAPVSYTDDQGRLIFKSDVVYGVSDAQVSGYLAAWVPVGANDSQDARTESSTTASTDGNTYHSNSALDSQLIYEGFSNFQAMPTQADEYTNIKIAENAQLFKSLGITSFELAPQYRSSTDNSFLDSVIQNGYAFTDRYDIGYNTPTKYGTVDQLLDALRALHAQGIQAINDWVPDQIYNLPGEEIVTASRTNGSGKVNESSVINNTLYDSRTVGGGEYQAIYGGAFLDKLKQDYPELFETKQISTGEAMNPDVKITEWSAKYFNGSNIQGRGAWYVLKDWSTNQYFNVSSGSEFLPKQLLGEKTSTGFTNVDNGKTEFYSTSGYQAKNTFIQDNDNWYYFDNDGYMVVGGQEINGKKYYFLPNGVELQDAYLSDGTSEYYYSSDGRQISNQYYQGSDNNWRYFFADGHMAVGLATITTENGTTNQQYFDANGVQLKGVAIKDTDGNVHYFDGKTGNMVINSWGKISDGSWLYLNDSGVAVTGPQNINGQNLYFNEDGIQVKGEAITDNSGNIHYYDRSTGNMVVNSWGETNNGSWLYLNDKGDAVTGEQVIDGQKLYFSSNGIQLKNTFKKLSDGSWLYLNDKGLPVTGAQVIDGQNLYFDQDGKQVKGDVATDGQGNTHYYDGNTGNMVTNSWAELPDGSWMYLDNDGNPLTGQQKIDGQSLYFNDAGKQIKNALVKLDDGSTIYLDDKGVSSTGIQRIDDKIYYFDPDGKQVVCRFEELPDGSWMYLDDDGVAATGAQKINGQELYFDNSGKQVKNDKVINDDGTINYYTGMSGEKLKNDFGELPDGSWMYLDNQGNAVIGAQKINGQNLYFKTDGRQVKGEANVDSSGEMHFYDPDSGELITNRFEQVASGVWAYFDAKGVAVTGEQRIGKQNLFFDPTGYQVKGDKRTIDGVLYTFDKESGERKGLDSISVLPTNGQYTTDKAQNWYYQVDGENVKGLYTNNDGQLRYFDLTTGVQTKGNFVTIGNDTYYFTKEQGDGQIVSEVVSGHYGTVQLSDNSSAWVYRGANDQILKGLQNINGRLQYFDLTTGAQLKGGAANYDGNLYYFESSDGNLVSKIQQSYSTGNYVTDGDKVTYADEQNNQVTGLALIDDQLQYFDPSDGRQVKNEQVIVDGVTYYFDKNGNGQYLFTNTATMSTNEFAKHSAAYSNDSSSFKNTIDGFLTADTWYRPKDILENGQTWVVSSTNDVRPLITVWWLNKDVQVNYSNFMKQNGLLDTSSQFNLQSDQYDLNVAAQKVQVAIEKRISKEKSTDWLKDLLFEAHEDTPSFVKQQFIWNKDSEYQGQGDAWFQGGYLKYGNNELTPTTNSDYRESGNTLDFLLANDVDNSNPAVQAENLNWLHYLMNFGTITANDDDANFDSIRIDAVDFIDNDAIQRTYDYMRDAYKVDASEDNANKHISLVEAGLDAGTSTIKSDALVESNFREAATLSLANQSGENSSLTNMLQDIDGGQIIADHANNATENESTPNYSIIHAHDKGIQEKVGAAITDVTGADWTNFTDDQLKEGLAAYYQDQRSTNKKYNIYNLPSIYALMLTNKDTVPRVYYGDMYQDDGQYMEKQSIYYDAIVSLMNTRKSYVSGGQTMDVDEHGLLKSVRFGKDAMTASDLGTNETRTEGVGVLVGNDSSLKLNDSDTVTLEMGAAHKNQKYRAALLTTSDGIVTYDADNDAPTIWTDDRGTLTFSNKEIAGQDYTSVQGFANSQVSGYLAVWVPVGASDDQDVRTAALTDANLDDKVLHSNAALDSNLIYEGFSNFQPKATTNDELTNVVIAKNANLFEKWGITSFEMAPQYRSSGDHTFLDSTIDNGYAFTDRYDLGFETPTKYGTDKDLRTAIKALHQSNMQVMADVVDNQVYNLSGQEVVSASRAGVYGNDVSTGFGTQLYAVNSVGGGKYQAQYGGEYLNELKQQYPDLFEAKTYDYWVKNYSNDGSDPYYTLSQNTRKDMPSSEVIKQWSAKYMNGTNVLGNGMGYVLKDWNTGEYFKIGEKNADFITN

SEQ ID NO: 2 - protein, 2821 aa, Leuconostoc mesenteroides
MTAGIFSAVIFGVSTTNVSADSTNNTGVTVSQAPDKVADTTATTDKVADTTATTDKAADTTATTDKVADTAAATDKVADTTATTDKVADTTATTDKVADTAAATDKVADTTATTDKVADTTATTDKVADTTATTDKVADTAAATDKAADTTATTDKAADTTATTDKVADTAATTDKAADTTATTDKVADTAATTDKAADTAATTDKVTDTTVATNKAVDTTATTDKVDDTTATTSEKSKSIKQIDGKTYFIGDDGQPKKNFTAIVDGQVLYFDKDTGALTSNSSQYTDGLVNIGNEHNAAYSLSSDSFTQVDGYLTANSWYRPKDILKNGTTWTAATANDFRPLLMSWWPDKDTQVSYLKYMQSAGLLSDDVALSNNDSMNSLTDTAMTVQKNIEEKIGLLGSTDWLKADMNQMVDSQSNWNISSESKGTDHLQGGALLYVNSDLTPNANSDYRLLNRTPTNQKGQITTNGNQGGYEMLLANDVDNSNPIVQAEQLNWLYYMMNIGSIAQNDPTANFDGYRVDAVDNVNADLLQIAGDYFKAAYGTNQSDANANNHISILEDWDNNDPAYVKAQGNNQLTMDFPMHLALKYSLNMPSSARSGLEPAISTSLVNRAADATENEAQPNYSFIRAHDSEVQTVIAQIIKDKINPSSDGLTVSTDEIAKAFEIYNADELKADKEYTAYNIPSSYALMLTNKDTIPRVYYGDLFTDDGQYMSAKSPYYDALTSLLQSRVKYVSGGQSMNMTYLHNNQGLLTSVRYGKDAMTANDTGTSETRTQGIGLIVGNKTDLNLNNDEQIVLNMGAAHKNQAYRALMLSTKDGLKIYNSDDEAPVSYTDDQGRLIFKSDVVYGVSDAQVSGYLAAWVPVGANDSQDARTESSTTASTDGNTYHSNSALDSQVIYEGFSNFQAMPTQADEYTNIKIAENAQLFKSLGITSFELAPQYRSSTDNSFLDSVIQNGYAFTDRYDIGYNTPTKYGTVDQLLDALRALHAQGIQAINDWVPDQIYNLPGEEIVTASRTNGSGKVNESSVINNTLYDSRTVGGGEYQAIYGGAFLDKLKQDYPELFETKQISTGEAMNPDVKITEWSAKYFNGSNIQGRGAWYVLKDWSTNQYFNVSSGSEFLPKQLLGEKTSTGFTNVDNGKTEFYSTSGYQAKNTFIQDNDNWYYFDNDGYMVVGGQEINGKKYYFLPNGVELQDAYLSDGTSEYYYSSDGRQISNQYYQGSDNNWRYFFADGHMAVGLATITTENGTTNQQYFDANGMQLKGVAIKDTDGNVHYFDGKTGNMVINSWGKISDGSWLYLNDSGVAVTGPQNINGQNLYFNEDGIQVKGEAITDNSGNIHYYDRSTGNMVVNSWGETNNGSWLYLNDKGDAVTGEQVIDGQKLYFSSNGIQLKNTFKKLSDGSWLYLNDKGLPVTGAQVIDGQNLYFDQDGKQVKGDVATDGQGNTHYYDGNTGNMVTNSWAELADGSWMYLDNDGNPLTGPQKIDGQSLYFNDAGKQIKNALVKLDDGSTIYLDDKGVSSTGIQRIDDKIYYFDPDGKQVVCRFEELPDGSWMYLDDDGVAATGAQKINGQELYFDNNGKQVKNDKVINDDGTINYYTGMSGEKLKNDFGELPDGSWMYLDNQGNAVIGAQKINGQNLYFKTDGRQVKGEANVDSSGEMHFYDPDSGELITNRFEQVASGVWAYFDANGVAVTGEQRIGKQNLFFDPTGYQVKGDKRTIDGVLYTFDKESGERKGLDSISVLPTNGQYTTDKAQNWYYQVDGENVKGLYTNNDGQLRYFDLTTGVQTKGNFVTIGNDTYYFTKEQGDGQIVSEVVSGHYGTVQLSDNSSAWVYRGANDQILKGLQNINGRLQYFDLTTGAQLKGGAANYDGNLYYFESSDGNLVSKIQQSYSTGNYVTDGDKVTYVDEQNNQVTGLALIDDQLQYFNPSDGSQVKNEQVIVDGVTYYFDKNGNGQYLFTNTATMSTNEFAKHSAAYSNDSSSFKNTIDGFLTADTWYRPKDILENGQTWVVSSTNDVRPLITVWWPNKDVQVNYLNFMKKNGLLDTSSQFNLQFDQYDLNVAAQKVQVAIEKRISKEKSTDWLKDLLFEAHEDTPSFVKQQFIWNKDSEYQGQGDAWFQGGYLKYDNSELTPTTNSDYRESGNTLDFLLANDVDNSNPAVQAENLNWLHYLMNFGTITANDDDANFDSIRIDAVDFIDNDAIQRTYDYMRDAYKVDASEDNANKHISLVEAGLDAGTSTIKNNALVESNFREAATLSLANQSGKNSSLTNMLQDIDGGQIIADHANNATENEATPNYSIIHAHDKGIQEKVGAAITDVTGADWTNFTDDQLKEGLAAYYQDQRSTNKKYNIYNLPSIYALMLTNKDTVPRVYYGDMYQDDGQYMEKQSIYYDAIVSLMNTRKSYVSGGQTMDVDEHGLLKSVRFGKDAMTASELGTNETRTEGVGVLVGNDSSLKLNDSDTVTLEMGAAHKNQEYRAALLTTSDGIVTYDADNDAPTIWTDDRGTLTFSNKEIAGQDYTSVQGFANPQVSGYLAVWVPVGASDDQDARTAASTDKNTDDKVLHSNAALDSNLIYEGFSNFQPKATTNDELTNVVIAKNANLFEKWGITSFEMAPQYRSSGDHTFLDSTIDNGYVFTDRYDLGFETPTKYGTDKDLRTAIKALHQSNMQVMADVVDNQVYNLSGQEVVSASRAGVYGNDVSTGFGTQLYAVNSVGGGKYQAQYGGEYLNELKQQYPDLFEAKTYDYWVKNYSNDGSDPYYTLSQNTRKDMPSSEVIKQWSAKYMNGTNVLGNGMGYVLKDWNTGEYFKIGEKNADFITN

SEQ ID NO: 3 - protein, 2844 aa, Leuconostoc carnosum
MRDKNVICDRKKLYKSGKLLVTAGIFSTVVFGMAVSDVSANDTDNTVLTSNSGFLDKVVDTTSTDKAATPDKVVDTTSTDKAVTPDKVADTTSTDKAVTPDKVADTTSTDKAATPDKVVDTTSTDKAATPDKVVDTTSTDKAATPDKVVDTTSTDKAATPDKVVDTTSTDKAATPDKVADTTSTDKAATPDKVVDTTSTDKAATPDKVVDTTSTDKAATPDKVVDTTSTDKAATPDKVVDTTSTDKAADRTISISGKTVKNIEEIGGKTYFVGDDGKVKKNFTVIVDGQVMYFDKESGALTSNHKQYKEGLSDITNEHNAAYSLENDNFTQIDSYLTANSWYRPKDILKSGTTWTASTDKDSRPLLMSWWPDQQTELSYLKYMQSAGFLAEDVNLSENNSIDDLTAAAMDVQKNVEAKISLSGNTDWLKEDMNQFVDSQSNWNISSESKGTDHLQGGALLYGNSDMTPDANSDYRLLNRTPKNQTGQISATNDQGGYEMLLANDVDNSNPIVQAEQLNWLHYMMNIGSITKNDSTANFDGYRVDAVDNVNADLLQIAGDYFKAAYGTDKSDANANNHISILEDWDNSDPDYVKKHGNEQLTMDFPMHLALKYALNMPIDMRSGLEPAIKTSLVNRSQDATENEAQPNYSFIRAHDSEVQTVIAQIIKDKINPKSDGLTVTPDEIAKAFEIYNADELKADKAYTAFNIPSSYALLLTNKDTVPRVYYGDLFTDDGQYMSDHSPYYDAITTLLASRIKYAAGGQSMGMTYLHDNQEVLTSVRYGKGALTADDLGNVDTRTQGIGLVISNKTDLSLKSDESVVLNMGVAHKNQAYRPAMLTTKSGLKIYDTDDGAPIVYTNNLGQLIFNADTVYGVSDPQVSGYLAAWVPVGATEDQDARTKGSHDGTTDGNVYHSNAALDSQVIYEGFSNFQAMPTTTDEYTNVKIAQNAQWFKKLGLTSFELAPQYRSSTDSSFLDSVIQNGYAFTDRYDVGYNTPTKYGTVDQLLDALRALHSQDIQAINDWVPDQIYNLPGEQIVTASRTNGSGKYDDDSVISNTLYDSRTIGGGEYQAMYGGAFLDQLKQAYPGLFETKQLSTGVAMDPDVKIKEWSAKYFNGSNIQGRGAWYVLKDWATNKYFSVSSNNTFLPKQLLGEKASTGFITNDGKTEFYSTSGYQAKNTFIEDNGNWYYFDNDGYSVVGKQVIDNKHYYFLPNGVELQDAYLSDGDKQYYYKKTGRQIVNQYYRDEQGDWRYFFADGHMALGLTDIVSNDGTHATQYFDNNGVQVKGTSERDKDGNIHYFDGTSGNLVVSSWGQLSDGSWLYLNDKGIAVTGAQQIDGQSLYFNEDGKEVKGDAVTDNQGNIRYFDGESGHMVVNSWGKLPDGSWMYLNDKGIAVTGQQKINNEVLYFNADGKQIKSAFKELVDGSWLYLNDKGIAVTGAQQIDGQSLYFNEDGKEVKGDAVTDNQGNIRYFDGESGHMVVNSWGKLPDGSWMYLNDKGIAVTGQQKINNEILYFDADGKQLKNTLKTLSDGSRIYLDGKGVSATGVQKINGKVSYFDVNGKQVSNHIQELPDGSWMYLDNDGLALIGNQDVDGKQLYFDVDGKQIKNDKVKNSDGTINYYTGTVGEKLKHDFGQLSDGSWMYLDENGNAVTGEQNINGQHLYFKDDGQQVKGDVFEDDLGRMRYYSANSGEMVVNQFEQISDGAWAYFGDDGVAVTGEQHINGQDLFFDATGQQVKGESRTINGIPYTFEKESGEKRSVNIAPLLAMGNYVTNNGTDWQYEVQGNPVKGLYSTSDNKLRYFDLTTGVQIKGNFVTIGHNTYYFNPANGDGELLPDVSDGHYGTIQVKDANTNEKTVWVYRNQSNTILKGIQNIHGNIQYFDLSTGEQIKGGIANYDGNDYYFESAKGNLTSKIKQVYTDGQYVTKDGKSIYEDAQQQSVSGLVSINGQLQYFNPQDGVQVKNQQIIVDGVTYYFDENGNGQYLFTNTTVMPMDDFTKHNTVYSDNDNNFKNNVDGFLTADTWYRPKEILKAGTTWTTTSESDMRPLITTWWPNKNVQVNYLNFMKQNNLLHTNVEYSLLSDQYDLNIAAQAVQTAIEKRIAQENSTDWLQNLLFTAQDDQPSFVKQQFIWNKDSEYQGKGDAWFQGGYLKYGNNKLTPNTNSNYRKTDNAFEFLLANDIDNSNPVVQAENLNWLEYLMNFGTITGKDDDANFDSIRVDAVDFISNDTIQRTYDYLRDAYQVDQSEAKANQHISLVEAGLDAGTSTVKNDALIESNLREAATLSLANASGKNSALTNMLQDVDGGTLIADHTHNSTENEATPNYSIIHAHDKGIQEKVGAAISDATGADWTNFTDTQLKSGLDLYYKDQRATDKKYNIYNLPSIYALMLTNKDTVPRVYYGDMYQDNGQYMAEKSIYYNALESLMSARKSYVSGGQTMDVDSHGLLKSVRFGKGAMTADTVGNEETRTEGIGVLVGNDASLKLNDSDTVTLDMGAAHKNQKYRAAILTTNNGLSTFDSDKDAPIAWTNDKGILTFSNKNVSGQDNTNVHGVANPQVSGYLAVWVPVGAKDDQNAGTSASTVVNTDGKVLHSNASLDSNLIFEGFSNFQPRATTNDELTNVVIAKNADLFQKWGITSFEMAPQYRSSQDHTFLDSTIDNGYAFTDRYDLGFNTPTKYGTDSDLRLAIKSLHKAGMQVMADVVDNQVYNLPDQEVVSASRAGVYGNDVATGFDTQLYAVNSVGGGKYQAQYGGQYLSELKNKYPDLFEAKAYDYWTKNYANDGSNPYYTLSQQTRDDIPSDEKIKQWSAKYMNGTNVLGHGMGYVLKDWNTGQYFKINKDGDSNLPV

SEQ ID NO: 4 - protein, 1672 aa, Fructobacillus tropaeoli
MRKKLYKSGKMWVAASVAISFSALSISVGNNGAKADDSQQSSTQIQSTQVTTALPAGGQYSTTNGGQSWNYLVNGVAIKGMYQDGQGQLRYFNFIDGTQVKGEFLSINGTYYYFDQNSGEGHLVPTQSNGHYTEIGNTGAWGYQNSNGELVKGIQNIDGQLRYFDENTGNQVKGGSATIGNKSYYFEPSQGTLTTTIDQVSDAQNANIRGLATVNGQLNYFDPTTGEQAKHKQVATNGATYYFNDSGVGTYLFTNVQNTPANDVSQHNAVNSTDTKDYTNTVDGFLTADTWYRPKYILDNGENWRASNEGEYRPFIMNWWPNKNVEVNYLKLMQNNNLLSSTVQYDLFTDQAILNQAAYQAQIAIEKRIKSEGSTDWLNTLLFGGDDSHPSFVKQQFIWNSDSESPWQGDAWFQGGYLKYGNSVMTPTSNSNYRQAGNAFDFLLANDVDNQNPIVQAEDLNWLYYLMNFGSITTNGLDNDSNFDSIRLDAVDFIHNDAIQRTYDYLRQAFNLTKNEATANQHLSLVEAGVDAGTTTYNSDGLIESNIRPLATDSLTNAPGKNASLSNLIKDVDSGEVIADHANFSTDDGIPNYSIIHAHDKGIQENVGAAITAATGADWTNFTTEQLEQGLDLYYQDQRSTNKKYNIYNLPSIYALMLTNKGTVPRVYYGDMYQDNGQYMQQKSLYYDAISSLMTARKQYVAGGQTMSVDENGLLKSVRFGKNAMTAQDTGDAETRTEGVGVIIGNDPSVKVADGQTVTLDMGAAHKNQAYRPLILTTSDGIQTYDSDENAPVVYTDDNGILTFSNQDINGQANTKIVGTLNPQVSGYLAVWVPVGASADQDARTAPSTQSTNDGKVLHTGAALDSNLIFEGFSNFQPMPTTHDEMTNVVISQNASQFAKWGITSFEMAPQYRSSEDHSFLDSTIDNGYAFSDRYDLGFGTPTKYGTDEDLRNAIKALHQNGMQVMADVVMNQLYSLNGKEVVSASRAGVYGNDVDLPFGTQLYVVNTTGGGEYQKKYGGAFLNIIKEKYPTLFDSESYDYYLKNYSDNGHGPAYMTTATATREAIPSDQPLKEWSAKYMNGTNILGLGMGYVLKDWNNGAYFKLSGTDTTLPQSLVALTGWNQNDDGTWSYYSTDTDDRVTGKQVIDGRTLLFDNQGNQIKGGWGENPDGTWSYYNADTGDRVIGEQVIDGRTLFFDNQGVQVKGGWGENYDGTWSYYNADTGDRVTGKQVIDGRTLLFDNRGVQVKGGWGENSDGTWSYYNADTGDRVTGNQLIGGRNLLFDNQGNQIKGGWDENPDGTWSYYNADTGDRVTGVQVIDGKQLLFDSNGIQVKNSWQKNANGTWSYYDANDGHLVPANSSNDGTSSSTQDSGNKSNQNPSSSSNAVNKTTGWIKNSDGTWSYLSAKSGQKVTGSQTIDGKQLLFDDHGVQIKGGWGKNADGTWSYYDANSGELTSTSDMSNVNPQQTTTTTNEQSTTNQPTDITKNSDGVYVYKNDSNKKAQGYLNDGSSWKWFNDGQLYTGFQNYMGAYYYFINGIRQQNQWENIWGLKYYVGDDGRTVEGIHAIDGHAYDFGTDGTFNVKGSASGYLNDGKSWMWYEGGNPYTGFRYYMDTYYWFENGVRQDNAWHQAWGLTYYTGADGRAVQGVQNINGKLYYFGNDGTFFMRTNQEV

SEQ ID NO: 5 - protein, 1632 aa, Lactobacillus kunkeei
MNNNNSEQKLRFKMYKSGKHWVVAGLTTAVVSVAIYSGSSIINGGVTVKADVQSTTQATTSVSDNSKDSSNSISNNNSNDDNQIATLPQDGTYSTGDNGQTWKYVEQNKNIQGLYKDSDNQLRYFNEYDGTQAKGDIVNVNNNNYYFDSGSGQGHKIDNYSGGNYVESNVNNENGWIYKATDNTEVKGIATIDGNVQYFDQSTGLQLKGGAVQVGGADYYFDPNKGNLVGKVDQVVNSNNYSDNKLLDSNKNVVKGLTVNNGTLQYYDPSTGDQVKNKQVIANGVTYYFDASGNGTYLFTNTGNSAVNDFSQRNAANSVNPSDYKNVVDGFFTADTWYRPKQIIDNGTTWRNSNSNDLRPMITAWWPNKDVQVNYLKLMQDNGLLDKSDTYTLQSDQQVLNQAAQSAQVNIEKKISQTGSTDWLNDLLFASHGNNPSFVKQQYVWNSDSESPWQGDAWFQGGYLKYGNSVMTPNTNSNYRDSNNLFDFLLANDVDNSNPAVQAEDLNWLYYLTNFGTITANDSNANFDSIRIDAVDFISNDIIQRSYDYLRQKFNLTQSDANADSHISLVEGGVDAGTTSYSNDGLVEAPFRLGAYPLLHKQGGDVFKDLINEEDSGIDISNHNGETNTTNTIGGLTLSGGKPNYSIVHAHDKDVQEKVGQAIVDTTGIKDWTDFTPSQLAQGLETFYNDQRQTDKKYNDYNVPSAYAIMLTNKGTVPRIYYGDMYQDDGQFMQKKSLYYDDIANLMTARKKYVSGGQYMVDNDGILTSVRFGKGANTVNDSGTSDTRNQGIGLIVGSDPKKVLNDGDTIVLHMGAAHKNQKYRALMLTTENGVQNYSSDDNAPVAETDDNGDLVFSNKDINGQDNTAIKQVANPEVNGYLAAWVPFGASDDQDARTSPSTSQNNDGNVLHENDALDSNLIFEGFSNFQPTPTNHDEYANVVIAKNASLFKDWGVTSFEMAPQYRSSQDHTFVDSTIDNGYAFSDRYDLGFGTPTKYGTDEDLRNAIKSLHANGMQVMADVVYNQLYNLPGEEVVSATRAGVTGNDNALPFGTQLYVVNTVGGGDYQKKYGGAFLNQLQEQYPSLFQSQKYKYYYKNYANNGAGPGYLTVTDGERSAIPSDQPITAWSAKYMNGTNILGRGMGYVLKDWNTGAYFKISGDDSTLPTSLTYRSGWVENPDSTWSYYSKDSIAKLTGAQIVNDQRVFFDNNGIQVKGGWVENPDGTYSYYDKNSGELLVGSQLVDGRHVFFDNVGVQVKGGWVANTDGSYSYYNANDGTMLTGAQFIDGQNVYFDADGKQVKGNWVQNNDGSWAYYDANLGHLVKDAKHVDSQPSTQQVNNKQSEEKSNSPLVEAQNNKDSASVESQNHQNSVSVESKNENKNQSNDVRNPSEKTDTKTSNEKSKEVSKEDAAYDNAKKSLVEAKKLVDKKPNKTNINKYKKALKSYENAQKKMNKSVISSYKKAAKELNAAKKNLSKKNNKVNMKKYSIALNKYRNAKKSYVVIKKKDLDKSKSALNTAKKALAKKKTKQSQKKYNDALKKYYDAEKTYLNLTGNYTKKYYYDFDKVGKKVKVIKSTSVYNSLKPNSKKVVKKIKKGSVIKVKKLILSNKKSYFDLGYNRFVIASKSSIKKAK

SEQ ID NO: 6 - protein, 1318 aa, Lactobacillus kunkeei
MKSKDNNSKIHFKMYKSGKQWVIAGLTTAVAAVSIYGGTTIFNDSVVAKADTAVKSSQSSNADTTQSSSAVVDSSSANSSSPSSTEVSVNVKNTSNADTQGIQALPDGGNYTSEDNGQTWKYSYQNNDVKGLYQENNNIRYFSENDGSQVKGNILNVNNKNYYFDSNNGDGHLIKDYSGGNYVEQSQDKWTYKTSDNNLVKGIATIDGQIKYYDPTTGIQLKGGAFEIGGAAYYVDPAQGNVVGRVNSIINSGNYVNKNNNTTFVDDNNNTVKGLNVVRGNLQYFNSTTGYQAKSEQVVANGATYYFDKDGNGTFLFNNTGSFSVTDFAKHNVANSNQPSDFTNTVDGFLTAETWYRPKQILDNGTKWRNSNKDDLRPLITVWWPNKDVQVNYLKLMQDNGLLDNSVKYTVFSDQQTLNEAAQSAQVNIEKRITQQKASSWLYDLLFKGDSSHPSFMNQQYIWNMGSESQWQGDAWFQGGYLKYGNSPLTPTTNSKYRKTDNQFDFLLANDVDNSNPAVQAEDINWLYYLTHFGTITGKNDKANFDSIRLDAVDFVSNEIIERSNDYLRDLYKLTKSDANADKHISLVEAGVDAGTSSTNGDSLVEAPFRLSAYGLLHNSGKVDALQDLVKEVDSGVLISDHSKNSKDGGVPNYSIVHAHDKDVQERVGQAIVDSTGIKDWTNFTPAQLAKGLSVYYADQRKTVKKYNDYNMPSAYAIMLTNKGTVPRIYYGDMYQDDGQFMQKKSFYYDDIVKLMTARLKYVAGGQTMSVDSNGFLKSVRFGKGAKTVNSKGTKETRHEGIGLIVGNDAKKVLSNGQKVTLNMGAAHKNQKYRALMLTTNKGVQTFASDKNAPVVKTDGNGVLTFTNKDIKGQANTSVRGVLNPKVSGYLAVWVPVGAKDNQDARTQPSKKNRNDGKVLHSNDALDSNLIYEGFSNFQPQPKKRSQYANVVIAKNAKLFKKWGITSFEMAPQYRSSNDKTFVDSTINNGYAFSDRYDLGFGKPTKYGTDKDLRKAIESLHDQGMQVMADAVLNQLYNLPGKQVVSAQRAGVTGNIADLPFGKQLYVVNTIGGGKYQKKYGGKFLKALKAKYPNIFKGKTYKYNYKNYSPTGEGYLTLNTGKTVSIPSNQPITEWSAKYMNGTNILGRGMGYVLKDWNTGTYFKLNGLKTVLPSELTDKSNWVNEVSTSSKDVYNKSVSNLKDAKKKLAAKKSKENTQLYNNALNSYYKAEKEYLSSAKLYNKKYYYDFDKLPSKVKVAKVTYSYKSTDFSKDNRIKKLKKGTVLNVKGLVLNGKVTRINIGNGKFVTASKDFIKAIR

SEQ ID NO: 7 - protein, 1139 aa, Lactobacillus kunkeei
MKAEHNNLKLHFKMYKSGKQWVIAGLTTVVASIAIFSGNQLFSGNVVAKADGVTTTQVSQNSNSSNGISTLPKDGKYVSSDNGKTWKYVSQNNAAKGLYSDGSNIFYFNESDGTQVKGNIISVGKNTYYFDSKTGQGTKISDYTGGKYTNPSTDNKAAWTYQDKSNNTVKGVATIGDSVQYYDEKDGFQLKGGSINLGNTNYYVDSNQGNVTSKVNRVVSSNDGTLKGLNVVNGNLQYLDYKTGKQVKNKQVAVNGVTYYFDDNGNGSYLFTNLGKSVRTNFDKYNAVNGVNKSNFTNTVDGFLTADSWYRPKQVLANGKKWRKSTSKDFRPLVTVAWPNKDVQVNYLALMQMNGLLSNKEKYTVFSDQQTLNAAAQKAQVNIEKRISKKKSTKWLDKLLFQGTTTEPSFIKRQYVWSSASEYPGQGDAQWFQGGYLKYGNNRLTPKTNSKYRQIGNAFDFLLANDVDNSNPSVQAEDINWLHYLTHFGSITAKDPKANFDSIRIDAVDFISNDIVQRSNDYLRDLYKLTKNDSNADKHISLVEGSLEAATSYSHSDSLIESPYRNDIDGLLGNSGNSADLSKLIKEYDSGIIISDHSGTTNNSSIPTYSIVHAHDKGVQERVGQAIMDNSGITDWANFTPKQLRDGLKLYYDDQRKTVKKYNDYNVPSAYAIMLTNNNTVPRIYYGDMYQDDGQFMQKKSLYYDDIVSLMLARTKYVAGGQSMSMDSNGFLTSVRYGKGANNVNSQGTSETRNEGIGVIVGNDTNKVLNDGQTVTLNMGAAHKNQQYRPVLLTTKDGIKTYSSDKNAPVVETDSNGVLTFSNKDVNGQSDTSVRGTLNPQVSGYLAVWVPVGAKNGQDARTKPSKKARNDGKVLHLNDALASNLIFEGFSNFQPMPKNKSQYTNVVIAKNAKTFKKWGITSFEMAPQYRSTNDKSFVDSTIKNGYAFSDRYDLGFGKPTKYGTDKDLRKAIKSLHAQGMQVMADVVYNQLYNLPGKEVVSTSRASAYGNNVDVPFGNQLYVVNTIGGGKYQTKFGGKYLKELKKKYPSLFKAKTYKYYYKDNQKDGSVKLALTSSKRSSIPANKPIKEWSAKYMNGTNVLGLGMGYVLKDWNNGKYFKINGTKTSLPSSIYYKSTKNK

SEQ ID NO: 8 - protein, 1463 aa, Lactobacillus kunkeei
MNINSNERKVRFKMYKSGKQWIVAGLTTAVISIAVYGGSSIANGGIEAKADAQNAATSSIVNTNNSTNSSNANSIASLPQNGTYSTNDNGQTWKYVSQNKDIQGLYKDNNDQLRYFNEYDGTQAKGDIVNVNNDNYYFDKDSGQGHKIDSYTGGSYSESKVNNQDGWIYKSSDNNDVKGVATVDGNIQYFDQNTGLQLKGGSAQIGGVDYYFDPNKGNLVGKVDQVVNSNDYSDNKLLDSNKNVVKGLVVNNGQLQFFDTSNGNQAKNKQVIANGITYYFDTNGNGQYLFTNTGKSAVDDFTQRNAANSVNPSDYKNVVDGFFTADTWYRPKQILDNGTTWRNSNSNELRPMITAWWPNKDVQVNYLKLMQNNGLLDKSNSYSIQSDQQTLNQAAQKAQVNIEKKISQTGNTDWLNDLLFKGNGDNPSFVKQQYIWSSDSESPWQGDAWFQGGYLKYGNSVMTPNTNSNYRDSNNLFDFLLANDVDNSNPAVQAEDLNWLYYLTNFGTITANDSNANFDSIRIDAVDFISNDIIQRSYDYLRQKFNLMQSDANADSHISLVEGGVDAGTTSYSNDGLVEAPFRLDAYPLLHKQDGDVFKNLIDEEDSGIDISNHNGETNTNNTIGGITLSGGKPNYSIVHAHDKDVQEKVGQAIIDTTGIKDWTDFTPSQLAQGLETFYNDQRQTVKKYNDYNVPSAYAIMLTNKGTVPRIYYGDMYQDDGQFMQKKSLYYDDIANLMTARKKYVSGGQSMVDNNGILTSVRFGKGANTVSDSGTEDTRNQGIGLIVGSDPKKVLNDGDTVVLHMGAAHKNQKYRALMLTTENGIQNYNSDDNAPVAETDDNGDLVFSNKDINGQANTAIKQVANPEVNGYLAAWVPVGASDDQDSRTAPSTSQNNDGNVLHENDALDSNLIFEGFSNFQPTPTNHDEYANVVIAKNASLFKDWGVTSFEMAPQYRSSQDHTFVDSTIDNGYAFSDRYDLGFGTPTKYGTDEDLRNAIKSLHDNGMQVMADVVYNQLYNLPGQEVVSATRAGVTGNTNALPFGTQLYVVNTIGGGDYQKKYGGAFLNELQEQYPSLFKSQKYKYYYKNYANNGAGPGYLTVNDAERSDIPYNQPITEWSAKYMNGTNILGRGMGYVLKDWNTGDYFKLSGSDSTLPSSLTYKSGWVENPDSTWSYYEKNNIDKLTGSQVINEERVFFDNNGIQVKGGWVKNSNGTYSYYDKNSGNILTGDQLIDGEHFFFDNNGVQVKGKWIKNSDGSKSYYDSHLGKLIKTDKKVSSNARKKKSKEELLYENALKVLRKDKKRLDKNKTKANIRKYNKSLKKYRKAKKKLLAITKNRVANARKAIKIAKKVLSKRKNINNEKRYYKALKEYYVAEKSYLKITGNYNKKYYYEFDKLTPKVKVVKNIYSYKSRHFTKKNRVKKIKKGTLVRVKSIVRSGKVARINIGNGHFITSSKDFIKMFK

SEQ ID NO: 9 - protein, 2841 aa, Leuconostoc mesenteroides
MRDKRIICDRKKLYKSGKLLVTAGIFSAVIFGVSTTNVSADSTNNTGVTVSQAPDKVADTTATTDKVADTTATTDKAADTTATTDKVADTAAATDKVADTTATTDKVADTTATTDKVADTAAATDKVADTTATTDKVADTTATTDKVADTTATTDKVADTAAATDKAADTTATTDKAADTTATTDKVADTAATTDKAADTTATTDKVADTAATTDKAADTAATTDKVTDTTVATNKAVDTTATTDKVDDTTATTSEKSKSIKQIDGKTYFIGDDGQPKKNFTAIVDGQVLYFDKDTGALTSNSSQYTDGLVNIGNEHNAAYSLSSDSFTQVDGYLTANSWYRPKDILKNGTTWTAATANDFRPLLMSWWPDKDTQVSYLKYMQSAGLLSDDVALSNNDSMNSLTDTAMTVQKNIEEKIGLLGSTDWLKADMNQMVDSQSNWNISSESKGTDHLQGGALLYVNSDLTPNANSDYRLLNRTPTNQKGQITTNGNQGGYEMLLANDVDNSNPIVQAEQLNWLYYMMNIGSIAQNDPTANFDGYRVDAVDNVNADLLQIAGDYFKAAYGTNQSDANANNHISILEDWDNNDPAYVKAQGNNQLTMDFPMHLALKYSLNMPSSARSGLEPAISTSLVNRAADATENEAQPNYSFIRAHDSEVQTVIAQIIKDKINPSSDGLTVSTDEIAKAFEIYNADELKADKEYTAYNIPSSYALMLTNKDTIPRVYYGDLFTDDGQYMSAKSPYYDALTSLLQSRVKYVSGGQSMNMTYLHNNQGLLTSVRYGKDAMTANDTGTSETRTQGIGLIVGNKTDLNLNNDEQIVLNMGAAHKNQAYRALMLSTKDGLKIYNSDDEAPVSYTDDQGRLIFKSDVVYGVSDAQVSGYLAAWVPVGANDSQDARTESSTTASTDGNTYHSNSALDSQVIYEGFSNFQAMPTQADEYTNIKIAENAQLFKSLGITSFELAPQYRSSTDNSFLDSVIQNGYAFTDRYDIGYNTPTKYGTVDQLLDALRALHAQGIQAINDWVPDQIYNLPGEEIVTASRTNGSGKVNESSVINNTLYDSRTVGGGEYQAIYGGAFLDKLKQDYPELFETKQISTGEAMNPDVKITEWSAKYFNGSNIQGRGAWYVLKDWSTNQYFNVSSGSEFLPKQLLGEKTSTGFTNVDNGKTEFYSTSGYQAKNTFIQDNDNWYYFDNDGYMVVGGQEINGKKYYFLPNGVELQDAYLSDGTSEYYYSSDGRQISNQYYQGSDNNWRYFFADGHMAVGLATITTENGTTNQQYFDANGMQLKGVAIKDTDGNVHYFDGKTGNMVINSWGKISDGSWLYLNDSGVAVTGPQNINGQNLYFNEDGIQVKGEAITDNSGNIHYYDRSTGNMVVNSWGETNNGSWLYLNDKGDAVTGEQVIDGQKLYFSSNGIQLKNTFKKLSDGSWLYLNDKGLPVTGAQVIDGQNLYFDQDGKQVKGDVATDGQGNTHYYDGNTGNMVTNSWAELADGSWMYLDNDGNPLTGPQKIDGQSLYFNDAGKQIKNALVKLDDGSTIYLDDKGVSSTGIQRIDDKIYYFDPDGKQVVCRFEELPDGSWMYLDDDGVAATGAQKINGQELYFDNNGKQVKNDKVINDDGTINYYTGMSGEKLKNDFGELPDGSWMYLDNQGNAVIGAQKINGQNLYFKTDGRQVKGEANVDSSGEMHFYDPDSGELITNRFEQVASGVWAYFDANGVAVTGEQRIGKQNLFFDPTGYQVKGDKRTIDGVLYTFDKESGERKGLDSISVLPTNGQYTTDKAQNWYYQVDGENVKGLYTNNDGQLRYFDLTTGVQTKGNFVTIGNDTYYFTKEQGDGQIVSEVVSGHYGTVQLSDNSSAWVYRGANDQILKGLQNINGRLQYFDLTTGAQLKGGAANYDGNLYYFESSDGNLVSKIQQSYSTGNYVTDGDKVTYVDEQNNQVTGLALIDDQLQYFNPSDGSQVKNEQVIVDGVTYYFDKNGNGQYLFTNTATMSTNEFAKHSAAYSNDSSSFKNTIDGFLTADTWYRPKDILENGQTWVVSSTNDVRPLITVWWPNKDVQVNYLNFMKKNGLLDTSSQFNLQFDQYDLNVAAQKVQVAIEKRISKEKSTDWLKDLLFEAHEDTPSFVKQQFIWNKDSEYQGQGDAWFQGGYLKYDNSELTPTTNSDYRESGNTLDFLLANDVDNSNPAVQAENLNWLHYLMNFGTITANDDDANFDSIRIDAVDFIDNDAIQRTYDYMRDAYKVDASEDNANKHISLVEAGLDAGTSTIKNNALVESNFREAATLSLANQSGKNSSLTNMLQDIDGGQIIADHANNATENEATPNYSIIHAHDKGIQEKVGAAITDVTGADWTNFTDDQLKEGLAAYYQDQRSTNKKYNIYNLPSIYALMLTNKDTVPRVYYGDMYQDDGQYMEKQSIYYDAIVSLMNTRKSYVSGGQTMDVDEHGLLKSVRFGKDAMTASELGTNETRTEGVGVLVGNDSSLKLNDSDTVTLEMGAAHKNQEYRAALLTTSDGIVTYDADNDAPTIWTDDRGTLTFSNKEIAGQDYTSVQGFANPQVSGYLAVWVPVGASDDQDARTAASTDKNTDDKVLHSNAALDSNLIYEGFSNFQPKATTNDELTNVVIAKNANLFEKWGITSFEMAPQYRSSGDHTFLDSTIDNGYVFTDRYDLGFETPTKYGTDKDLRTAIKALHQSNMQVMADVVDNQVYNLSGQEVVSASRAGVYGNDVSTGFGTQLYAVNSVGGGKYQAQYGGEYLNELKQQYPDLFEAKTYDYWVKNYSNDGSDPYYTLSQNTRKDMPSSEVIKQWSAKYMNGTNVLGNGMGYVLKDWNTGEYFKIGEKNADFITN

SEQ ID NO: 10 - protein, 2580 aa, Lactobacillus kunkeei
MNNYQDRKLHYKMYKSGKNWVVAGIVSATMSIVLFQNINDVTAKASQNQITYSGSYASTDSGSAKSAYSASSYINGSSSLSVPSTDNSNKSSLASSTNSSVVTSSLSSNVMNQSSNSHASSISSDVSNQSQSSKSASTSSNSSEKSDSASQSNNSVQSYESVSLSVSLASNASQNLKSHTKNIDGKTYFYDLNNNEVKSALIDDNNTYYYFGPNGYLTTFDDSKFSDGSINSSDQLSIYTPDGKSITNVDGFLTADSWYRPKQIQVSDTQWRNSTNADFRPLLSVWWPNKTVEINYLNYMSKNGLVNGHFDNNSSANDINQAAATVRLSIENKIKADNGDLSAIRSLFTTFINSQDEWNIDSEDYNSGDGLQGGSLLFGNNSETKDANSNYRLLNRNPTQQDGKIDYTHTQDPGFEFLLANDVDNSNPVVQAETLNWLHYLMNFGSIVNKDSSANFDGVRVDAVDNMDADVLNIISQYFRDAYKINANDVNSNNHLSILEDWSGNDPYYQKDHGTNQMTLDSDYLSSLRSILMYNPSVRSDMSTIINAGIVDRADDNTTNKAIPNYEIVRAHDAGVQDIISQIIIDNIDPHASASNPTWEQMREAFKIYDADENSTVKKYTQYNIPASYALTLTNKDTTPRVYYGDLYTDDGQFMEQKTPYYDAIAEMLKSRVKYVAGGQHMEAVKVNNGEDTILTSVRYGKDALNADDLGDSLTRTSGIAVVESNNPTLSLSDKDKVVIHMGAAHKNQEYRQLLTASNSGIDSFDSDSSKGYVVVRTDDNGDLTLDGNIIKGYANPQVSGYLSMWVPLGASDNQDIRTAPSSESSTDGQSIHSNEASDSNVILEAFSNFQNFPQTTDQYENVVIQKNAEDFKNLGFTYLELPPQYKSTKDGSFIDSVVQNGYSFNDRYDLGFDTPTKYGTAEQLIDAIKSLHAQGIKVLADIVPDQIYSLPNQQIVNATRTNPYGDANFNSNLINVLYDAFSKGSGTDYQYKYGGEFLEQLKKLYPDLFTTKQISTGQPIDASKKLKIWTAEYLNGSNIQGRGSGYVLSSTPNSNYYTVVDDGNTNIKSSNLPKGLLGDNVEYGLKLIDGKMKYVSTGGFIAKNTFIQDDNHNWYYVDNNGDFVTSPQVINGNKYFFLSNGVNLRDFISVNSDGTMNYYQSNGILANNPGYYYSNNINQMVHVNNNGVLDTGIVNVNGYVQYFDDNGYQVKGDIVNFNGRKMYFDDGSGNLVYNRFVSYNGNWYYAGSNGLLVNGLQNINNQSLYFDDNGKQVKGGMISLDNHKMYFDANSGNLYKNSFVLLDGDVYYANNDGYIVNGYQNIGGRNLYFDSDGKQVKDQFVNINGNKVYFNGTDGSEVKDDFIIHDDKEYYADNQGHLLTGYNFVNGQNMYFNEDGSQVKNSIVSLDGKLMYFDENSGNQVKNGFILHNGNVYYSNKDGILVTGYQNIDGQDLFFNADGTQIKGGTAEIDGVNYYFENGEGHLVGKVDQVINSNRFSDNKLLDANNNVVKGLIFNNGQLQYFDSLTGDQAKNKQIIADGNTYYFDNSGNGTYLFTNIGESETNDFSQRNAANSVNLSDYKNVVDGFFTADTWYRPKQILDNGTTWRNSNSNDFRPMITVWWPNKNVQVNYLKLMQNNNLLDKSTNYTLQSNQQMLNQAAQNAQVNIEKKIAQTGNTDWLNDLLFKGNGDTPSFVKQQYIWNILSESPGQDDALLQNGYFKYVNSELTPNTNSSYRISNNLCDFLLANDVDNSNPAVQAEDLNWLYYLTNFGTITANDPNANFDSIRIDAYAFINNDIVQRSNDYMKQKYKLTESSNNANSHLSIVEAGVDAGTTSTNNDELVESPFRTISYGLIHKDRNPQDMNNFIKEVDTGVVIADHENNSQEIGQPNYSIVHAHDKDIQDKVGEAIVDATGIKDWTNFTPSELSAGLKLYYDDQRSSEKKYNDYNIPSAYAIMLTNKGTVPRIYYGDMYQDDGQFMQKQSVYYDDIVSLLTARKKYVSGGQSMSVDHNNFLESVRFGKGAGSESDSGNAETRNEGIGLIVGNDQNKKLNDGDTVVLHMGAAHRNQKYRALMLTTNDGIKNYDSDENAPIAETDSNGDLVFSNKDINGQANTSVRGVLNPEVAGYVAAWVPLGASDDQDSRTLSSNKSYNDGKVLHSGDDLDSNVIFEAFSNFQPEPTNENEYENVVIPQKASLFKDWGITSFELPPQYRSSNDHTFVDATINNGYAFSDRYDLGFGQPTKYGTDVDLRNTIKSLHDNGMQVMADVVYNQLYNLPGQEVVSAVRAGFTGNTVSLPFGNQLYVVNTVGGGDYQKKYGGAFLQKLYQEYPSLFDSEKYQYNSKNYVTDLLVMTDGERSAIPSDQPITEWSAKYMNGTNILGRGMGYVLKDWNTGTYFKLNGNNSVLPDVLTYRNSFDDNTGTQSSSSAQSSASNHETQISSSAQSSASNHEAQSSSSAQSSVSSHEVQSSSSAQSSASSHEVQSSSSAQSSASSHEVQSSSSAQSSASSYEAQSSSSAQSSASSHEVQSSSSAQSSVSSHETQSSSSAQSSASSHEVQSSSSAQSSASSNEAQSSSSAQ

SEQ ID NO: 11 - protein, 1463 aa, Lactobacillus kunkeei
MNINSNERKVRFKMYKSGKQWIVAGLTTAVISIAVYGGSSIANGGIEAKADAQNAATSSIVNTNNSTNSSNANSIASLPQNGTYSTNDNGQTWKYVSQNKDIQGLYKDNNDQLRYFNEYDGTQAKGDIVNVNNDNYYFDKDSGQGHKIDSYTGGSYSESKVNNQDGWIYKSSDNNDVKGVATVDGNIQYFDQNTGLQLKGGSAQIGGVDYYFDPNKGNLVGKVDQVVNSNDYSDNKLLDSNKNVVKGLVVNNGQLQFFDTSNGNQAKNKQVIANGITYYFDTNGNGQYLFTNTGKSAVDDFTQRNAANSVNPSDYKNVVDGFFTADTWYRPKQILDNGTTWRNSNSNELRPMITAWWPNKDVQVNYLKLMQNNGLLDKSNSYSIQSDQQTLNQAAQKAQVNIEKKISQTGNTDWLNDLLFKGNGDNPSFVKQQYIWSSDSESPWQGDAWFQGGYLKYGNSVMTPNTNSNYRDSNNLFDFLLANDVDNSNPAVQAEDLNWLYYLTNFGTITANDSNANFDSIRIDAVDFISNDIIQRSYDYLRQKFNLMQSDANADSHISLVEGGVDAGTTSYSNDGLVEAPFRLDAYPLLHKQDGDVFKNLIDEEDSGIDISNHNGETNTNNTIGGITLSGGKPNYSIVHAHDKDVQEKVGQAIIDTTGIKDWTDFTPSQLAQGLETFYNDQRQTVKKYNDYNVPSAYAIMLTNKGTVPRIYYGDMYQDDGQFMQKKSLYYDDIANLMTARKKYVSGGQSMVDNNGILTSVRFGKGANTVSDSGTEDTRNQGIGLIVGSAPKKVLNDGDTVVLHMGAAHKNQKYRALMLTTENGIQNYNSDDNAPVAETDDNGDLVFSNKDINGQANTAIKQVANPEVNGYLAAWVPVGASDDQDSRTAPSTSQNNDGNVLHENDALDSNLIFEGFSNFQPTPTNHDEYANVVIAKNASLFKDWGVTSFEMAPQYRSSQDHTFVDSTIDNGYAFSDRYDLGFGTPTKYGTDEDLRNAIKSLHDNGMQVMADVVYNQLYNLPGQEVVSATRAGVTGNTNALPFGTQLYVVNTIGGGDYQKKYGGAFLNELQEQYPSLFKSQKYKYYYKNYANNGAGPGYLTVNDAERSDIPYNQPITEWSAKYMNGTNILGRGMGYVLKDWNTGDYFKLSGSDSTLPSSLTYKSGWVENPDSTWSYYEKNNIDKLTGSQVINEERVFFDNNGIQVKGGWVKNSNGTYSYYDKNSGNILTGDQLIDGEHFFFDNNGVQVKGKWIKNSDGSKSYYDSHLGKLIKTDKKVSSNARKKKSKEELLYENALKVLRKDKKRLDKNKTKANIRKYNKSLKKYRKAKKKLLAITKNRVANARKAIKIAKKVLSKRKNINNEKRYYKALKEYYVAEKSYLKITGNYNKKYYYEFDKLTPKVKVVKNIYSYKSRHFTKKNRVKKIKKGTLVRVKSIVRSGKVARINIGNGHFITSSKDFIKMFK

SEQ ID NO: 12 - protein, 2824 aa, Leuconostoc carnosum
MTAGIFSTVVFGMAVSDVSANDTDNTVLTSNSGFLDKVVDTTSTDKAATPDKVVDTTSTDKAVTPDKVADTTSTDKAVTPDKVADTTSTDKAATPDKVVDTTSTDKAATPDKVVDTTSTDKAATPDKVVDTTSTDKAATPDKVVDTTSTDKAATPDKVADTTSTDKAATPDKVVDTTSTDKAATPDKVVDTTSTDKAATPDKVVDTTSTDKAATPDKVVDTTSTDKAADRTISISGKTVKNIEEIGGKTYFVGDDGKVKKNFTVIVDGQVMYFDKESGALTSNHKQYKEGLSDITNEHNAAYSLENDNFTQIDSYLTANSWYRPKDILKSGTTWTASTDKDSRPLLMSWWPDQQTELSYLKYMQSAGFLAEDVNLSENNSIDDLTAAAMDVQKNVEAKISLSGNTDWLKEDMNQFVDSQSNWNISSESKGTDHLQGGALLYGNSDMTPDANSDYRLLNRTPKNQTGQISATNDQGGYEMLLANDVDNSNPIVQAEQLNWLHYMMNIGSITKNDSTANFDGYRVDAVDNVNADLLQIAGDYFKAAYGTDKSDANANNHISILEDWDNSDPDYVKKHGNEQLTMDFPMHLALKYALNMPIDMRSGLEPAIKTSLVNRSQDATENEAQPNYSFIRAHDSEVQTVIAQIIKDKINPKSDGLTVTPDEIAKAFEIYNADELKADKAYTAFNIPSSYALLLTNKDTVPRVYYGDLFTDDGQYMSDHSPYYDAITTLLASRIKYAAGGQSMGMTYLHDNQEVLTSVRYGKGALTADDLGNVDTRTQGIGLVISNKTDLSLKSDESVVLNMGVAHKNQAYRPAMLTTKSGLKIYDTDDGAPIVYTNNLGQLIFNADTVYGVSDPQVSGYLAAWVPVGATEDQDARTKGSHDGTTDGNVYHSNAALDSQVIYEGFSNFQAMPTTTDEYTNVKIAQNAQWFKKLGLTSFELAPQYRSSTDSSFLDSVIQNGYAFTDRYDVGYNTPTKYGTVDQLLDALRALHSQDIQAINDWVPDQIYNLPGEQIVTASRTNGSGKYDDDSVISNTLYDSRTIGGGEYQAMYGGAFLDQLKQAYPGLFETKQLSTGVAMDPDVKIKEWSAKYFNGSNIQGRGAWYVLKDWATNKYFSVSSNNTFLPKQLLGEKASTGFITNDGKTEFYSTSGYQAKNTFIEDNGNWYYFDNDGYSVVGKQVIDNKHYYFLPNGVELQDAYLSDGDKQYYYKKTGRQIVNQYYRDEQGDWRYFFADGHMALGLTDIVSNDGTHATQYFDNNGVQVKGTSERDKDGNIHYFDGTSGNLVVSSWGQLSDGSWLYLNDKGIAVTGAQQIDGQSLYFNEDGKEVKGDAVTDNQGNIRYFDGESGHMVVNSWGKLPDGSWMYLNDKGIAVTGQQKINNEVLYFNADGKQIKSAFKELVDGSWLYLNDKGIAVTGAQQIDGQSLYFNEDGKEVKGDAVTDNQGNIRYFDGESGHMVVNSWGKLPDGSWMYLNDKGIAVTGQQKINNEILYFDADGKQLKNTLKTLSDGSRIYLDGKGVSATGVQKINGKVSYFDVNGKQVSNHIQELPDGSWMYLDNDGLALIGNQDVDGKQLYFDVDGKQIKNDKVKNSDGTINYYTGTVGEKLKHDFGQLSDGSWMYLDENGNAVTGEQNINGQHLYFKDDGQQVKGDVFEDDLGRMRYYSANSGEMVVNQFEQISDGAWAYFGDDGVAVTGEQHINGQDLFFDATGQQVKGESRTINGIPYTFEKESGEKRSVNIAPLLAMGNYVTNNGTDWQYEVQGNPVKGLYSTSDNKLRYFDLTTGVQIKGNFVTIGHNTYYFNPANGDGELLPDVSDGHYGTIQVKDANTNEKTVWVYRNQSNTILKGIQNIHGNIQYFDLSTGEQIKGGIANYDGNDYYFESAKGNLTSKIKQVYTDGQYVTKDGKSIYEDAQQQSVSGLVSINGQLQYFNPQDGVQVKNQQIIVDGVTYYFDENGNGQYLFTNTTVMPMDDFTKHNTVYSDNDNNFKNNVDGFLTADTWYRPKEILKAGTTWTTTSESDMRPLITTWWPNKNVQVNYLNFMKQNNLLHTNVEYSLLSDQYDLNIAAQAVQTAIEKRIAQENSTDWLQNLLFTAQDDQPSFVKQQFIWNKDSEYQGKGDAWFQGGYLKYGNNKLTPNTNSNYRKTDNAFEFLLANDIDNSNPVVQAENLNWLEYLMNFGTITGKDDDANFDSIRVDAVDFISNDTIQRTYDYLRDAYQVDQSEAKANQHISLVEAGLDAGTSTVKNDALIESNLREAATLSLANASGKNSALTNMLQDVDGGTLIADHTHNSTENEATPNYSIIHAHDKGIQEKVGAAISDATGADWTNFTDTQLKSGLDLYYKDQRATDKKYNIYNLPSIYALMLTNKDTVPRVYYGDMYQDNGQYMAEKSIYYNALESLMSARKSYVSGGQTMDVDSHGLLKSVRFGKGAMTADTVGNEETRTEGIGVLVGNDASLKLNDSDTVTLDMGAAHKNQKYRAAILTTNNGLSTFDSDKDAPIAWTNDKGILTFSNKNVSGQDNTNVHGVANPQVSGYLAVWVPVGAKDDQNAGTSASTVVNTDGKVLHSNASLDSNLIFEGFSNFQPRATTNDELTNVVIAKNADLFQKWGITSFEMAPQYRSSQDHTFLDSTIDNGYAFTDRYDLGFNTPTKYGTDSDLRLAIKSLHKAGMQVMADVVDNQVYNLPDQEVVSASRAGVYGNDVATGFDTQLYAVNSVGGGKYQAQYGGQYLSELKNKYPDLFEAKAYDYWTKNYANDGSNPYYTLSQQTRDDIPSDEKIKQWSAKYMNGTNVLGHGMGYVLKDWNTGQYFKINKDGDSNLPV

SEQ ID NO: 13 - protein, 2771 aa, Leuconostoc mesenteroides; identical to SEQ ID NO: 1

SEQ ID NO: 14 - DNA, 8316 bp, Leuconostoc mesenteroides
  source 1..8316: mol_type = unassigned DNA
gtgacagccggtattttttctgctgtgatatttggcgtttccacaactaacgtaagcgctgatagcactaacaacactggtgttacggtgtcacaagcaacagataaagtagcagacacgacggctacaacggacaaagtagcggacacaacagctacaacagataaagtggccgacacagcagctacaacggacaaagtagcggacacaacagctacaacagataaagtggccgacacagcagctacaacggacaaagtagcggacacaacagctacaacagataaagtggccgacacagcagctgcaacagataaagcagccgatacagcggctacaacggataaagtggccgacacagcagctgcaacggataaagtagcggacacagtagctgcaacggacaaagtagcggacacaacagctacaacagataaagcagccgatacagcggctacaacggataaagtaaccgacacaacagctgcaacggataaagcagcggacacgacggctacaacagataaagtggccgacacaacagccacaacatcagaaaaatcaaaaagtattaaacaaattgatggtaaaacttatttcattggtaatgatggtcagcctaagaaaaattttacagccattgttgacggtcaagtattatatttcgacaaagatactggtgctttgacatcaaatagtagtcaatataccgatggtttagccaatataggaaatgagcataatgcggcttattcattgtcttcggatagttttacacaagttgatggctatctaacggctaacagttggtaccgacctaaagatatattaaaaaatggtacaacttggacggctgcaacagcaaacgattttcgaccattgttaatgtcttggtggccagataaagatacccaagtttcatacttaaaatatatgcaatctgcgggattattatcagatgacgttgcattatcaaacaatgatagtatgaacagtttgacggatacggctatgactgttcaaaaaaaaattgaagaaaaaattggcttattgggcagtactgactggcttaaggccgatatgaaccaaatggttgattcacaatcgaattggaacattagtagtgagtctaaaggaacagatcatttgcaaggtggtgcgctcctatacgttaatagtgatttaacaccaaatgccaattctgattatcgtttattaaatcgaacgccaactaaccaaaaaggtcaaattacaacaaatggtaatcaaggtggctatgagatgctgttggccaacgatgttgataattctaacccaattgttcaagctgaacaattaaattggttatactacatgatgaatatcggtagcatcgcccaaaatgatccaacagcaaattttgacggttacagagttgatgctgttgataacgtgaatgctgacttgttgcaaattgctggagattattttaaagcagcatatggaacaaatcaaagtgacgctaatgcaaacaatcacatttccatcttagaagactgggacaataatgatccagcgtatgtaaaagcacaggggaacaaccaattaaccatggattttccaatgcatttggcattgaagtattcattgaatatgccaagtagtgctcgtagcggtttggaaccagcaatttcaacaagcctagtaaatcgtgcagcagacgccacagaaaatgaagcccaaccgaactattcatttattcgtgcacatgatagtgaagtacaaacggttattgctcaaattattaaagataaaattaatcctagttccgatggattgactgtctcaacagatgaaattgccaaagcattcgaaatatataatgctgacgaattaaaggctgataaagagtataccgcatataatataccctcatcatatgcattgatgttaactaacaaagatacaattcctcgtgtgtattatggtgatttgtttacggatgatggacaatatatgtctgcaaaatcaccatattatgatgcacttacttcattgcttcaatcgcgagtaaaatatgtttcaggtggtcaatctatgaatatgacttaccttcataataatcaaggccttttgacgtcagtccgctatggaaaagacgccatgacagctaacgacactggtacaagtgaaacgcgcacacaaggtattggattaattgtcggcaacaaaactgatttaaacctgaataatgatgagcaaattgtacttaacatgggggccgcacacaaaaatcaagcttaccgtgcattaatgttaagtactaaagatggcttgaaaatttataataatgatgacgaggcaccggtatcgtatacagatgaccaaggccgtttgatttttaaatctgatgtggtttatggtgtgagtgatgctcaggtttctggttatttagcagcttgggtgccagtcggtgcaaacgatagccaagatgctagaacagaaagtagtacaacagcgtcaacagatggtaatacctatcattcaaatagtgccttagattctcaacttatatatgaaggcttttcaaacttccaagccatgccaacacaggccgatgagtataccaatatcaagattgccgaaaatgcacaattattcaagagccttgggataacaagctttgaattggcacctcaataccgttcaagtacggataacagtttcttagattcagttattcaaaatggctatgccttcacggatcgctacgacattggttataatacgcctacaaagtacggtacagttgaccaactattagatgctttaagagcattgcatgctcaaggcattcaggctatcaatgattgggtcccagaccaaatttataatttgcctggggaagaaatagtgacagccagtcgaacaaacggttcgggaaaggtgaatgaaagttcagttattaataatacgctatatgattctcgtactgttggtggcggagagtatcaagcaatatatggaggtgctttcttagataagttaaaacaagattatcctgagttatttgaaacaaaacaaatttcaacaggtgaagcaatgaaccctgatgtcaaaatcacagaatggtcagctaagtattttaatggctcaaacattcaaggacgtggtgcatggtatgttctcaaggattggtcaacaaatcaatactttaatgtttcaagtggtagtgaatttttacctaagcaactgttaggcgaaaaaacaagtacagggtttaccaacgtggacaatggcaagactgagttttattctacgagtggctaccaagcaaagaatacatttattcaagataatgacaattggtattattttgataatgatggctatatggttgttggcggtcaagaaattaatggtaaaaaatattatttcctaccaaatggtgtagagttacaagatgcttatttgtctgatgggactagtgagtattactacagtagtgatggtcgtcaaatttctaatcaatattatcaaggatcagacaacaactggcgttatttctttgcagatggtcatatggctgtagggttagcaacaattactacagaaaatggtacaacaaatcaacaatatttcgatgcaaatggtgtgcaacttaagggcgtagctataaaggatactgatggcaatgtgcactattttgatggcaagacaggaaacatggttataaattcctggggtaaaataagcgatggttcatggttatacttaaatgatagcggtgtagcggtcacaggaccgcaaaatattaacggccaaaatctttacttcaacgaagacggtattcaagtaaagggtgaagccattactgataatagtggaaacatacattattatgatcgcagcacaggaaatatggttgtgaactcatggggtgaaacgaataatggttcatggctatacttgaacgacaagggtgatgccgttacaggagaacaagttattgacggtcaaaaactatatttcagtagtaatggaatccaacttaaaaatacattcaagaagctatccgatggttcatggctatatttgaacgataaaggtcttccagtgacaggagcacaggtcattgatggacaaaacttgtatttcgaccaagatgggaagcaagtcaagggtgacgttgctacagatggacaaggtaacactcattattatgatggcaacacaggaaatatggttactaattcatgggcagagttaccggacggttcatggatgtatctagacaatgatggcaatcctttaacaggacagcaaaagattgatggccagtcactctactttaatgatgctggtaagcaaatcaaaaacgcattggttaaactagatgatgggtcaacaatttacctcgatgataaaggtgtttcatcaactggtattcaaagaattgatgataagatatattattttgatcctgatggtaaacaagtagtatgtcgttttgaagaattaccagatggttcatggatgtatctagatgatgacggtgttgctgctacgggcgctcaaaaaattaatggccaggaattatatttcgacaatagcgggaaacaagtcaagaacgacaaagtaattaatgacgatggaacaataaactattacacaggtatgagcggtgaaaaactaaaaaatgattttggtgaattaccagacggttcatggatgtacttggataatcaaggtaatgctgtaataggtgcccaaaaaattaatggccagaatctttacttcaagacagacggacgacaggttaagggtgaagcaaatgttgattcatcaggtgaaatgcacttctatgatcctgattctggcgagctaattacaaatagatttgaacaagttgctagtggtgtatgggcttactttgatgccaaaggtgttgctgtaactggtgagcaacgcattggtaagcaaaatttattttttgatccaactggttatcaagttaaaggcgacaaacgaacaattgacggcgttctctatacctttgataaagaaagtggtgagagaaagggtttagattctatatcggtattacccaccaatggacaatacacaaccgataaggcccaaaattggtattaccaagtcgatggtgaaaatgtaaaagggctatatacaaataatgatggtcaattacgttacttcgatttgacaactggcgtgcagactaaaggtaattttgtgacaattggcaatgatacctactatttcaccaaggaacaaggggatggacagatagtttctgaggttgtgtcaggacactatggtactgtccagttgagtgacaattcgtctgcatgggtttatcgcggtgcaaatgatcaaattttgaaaggcctacagaatataaacggtcgtctgcaatattttgatctaaccaccggtgcgcaattaaaaggcggtgctgcaaactatgatggcaacctttattattttgaatcatcagatggtaacctagtcagtaaaattcagcaatcttattctactgggaattatgtgaccgatggtgataaagtaacatatgctgatgagcaaaacaaccaagtcacgggattagcgttgattgatgatcaactacaatacttcgatccaagtgacggtcgtcaagtcaagaatgagcaggttatcgttgatggcgtcacatactactttgataaaaatggtaatggacaatacttgtttacaaatactgcaacgatgtcaactaatgaatttgccaaacatagtgctgcttatagcaatgatagttctagcttcaagaatacgatagatggtttcttgacggccgatacctggtatcgccctaaagatatcttggaaaacggacaaacgtgggtagtttcttcaacaaatgacgtgcgaccactgataacagtttggtggctaaataaagatgttcaagttaattattcaaattttatgaagcaaaatggtttgctagatacaagtagtcaatttaatctacaatctgatcaatatgacttgaatgtcgccgcgcaaaaagttcaagtggctattgaaaaacgcatttcgaaagaaaagagtacagattggttgaaagatcttttgtttgaagctcatgaagatacgccttcatttgtgaaacaacaatttatttggaataaagattctgaatatcaaggtcaaggggatgcgtggttccaaggtggttatctgaaatatggtaacaatgaattaaccccaacaacgaactcagattatcgtgaatccggtaatacattagacttcttgcttgctaatgatgtcgacaattctaacccagcggttcaagctgaaaatttgaattggttacattatttaatgaactttggcacgattacagctaatgatgatgatgctaattttgacagtattcgtattgatgccgttgactttattgataatgatgccattcagagaacctacgattacatgcgtgatgcttataaagttgatgcaagtgaagacaacgctaataagcatatttcactagtggaagctggattagatgctggtacctctacaattaagagtgatgctttagttgaatctaactttagagaggcagctacactatcgctagcaaatcaatcaggggaaaatagttccttgactaatatgttgcaagacattgatggtggccagattatagctgatcacgccaacaatgcaacagaaaatgaatcaacgccaaattattcaattattcatgctcatgataaggggattcaagaaaaggttggtgcagcaattaccgacgttactggtgcagactggacgaatttcaccgatgaccaattaaaagaaggattagcagcttattatcaagatcaacgttcaacgaataaaaagtataacatctataacttacctagtatctatgctttaatgttgaccaataaggacacagttcctcgtgtttattacggtgatatgtatcaagatgatggccagtatatggaaaagcaaagtatttattatgatgccattgtttccttgatgaacactagaaagagttatgtgagtggtgggcaaactatggatgtagatgaacatggtttgttgaagagtgttcgttttggtaaagacgcaatgacagctagtgaccttggtacgaatgaaacacgcactgaaggtgttggtgtgctggtcggcaatgattcttcactaaaactaaatgattcagatacagttactttagagatgggggcggctcataaaaaccaaaagtaccgagctgcattgttgacaactagtgatggtattgttacgtatgatgctgataatgatgcaccaacaatctggacagatgaccgtggtacattgacgttctcaaataaggagattgctggtcaagattatactagtgtgcaaggattcgctaattcacaagtatcaggttacttagcagtttgggtgcccgtaggagctagtgacgatcaagatgtccgaacagcagcattaacagatgcaaatcttgatgacaaagtactgcattctaatgctgcattagattcgaacttgatttacgaaggcttttctaactttcaacctaaagcaaccaccaatgatgaattgactaacgtagtaattgctaaaaatgctaatttatttgaaaagtggggaatcacaagttttgagatggcaccacaatatcgttcaagtggggaccacacgttcttagattcaacgattgataatggttatgcatttactgaccgatacgatttgggatttgaaacaccaactaagtacggtactgataaggatttgcgtactgcaattaaagcattgcaccaatcaaatatgcaggttatggctgatgtagttgataaccaagtttataatttatctggacaggaggtcgtatcagcttcacgtgccggtgtttacggcaatgatgtgtcaactggatttgggacacaactctatgcggttaatagtgttggtggtggtaaatatcaagcccaatacggtggtgaatatttgaatgaattgaagcaacaatacccagatttgttcgaagctaagacgtatgactattgggttaaaaattattcaaatgacggatcggatccgtattacacactgtcgcaaaacacacgaaaagatatgccaagtagtgaggtcattaaacaatggtcagctaaatatatgaatggtactaatgtattaggaaacggtatgggatatgttttgaaagattggaatacaggtgagtatttcaaaattggagaaaagaatgctgattttataacaaattaa

SEQ ID NO: 15 - DNA, 8466 bp, Leuconostoc mesenteroides
  source 1..8466: mol_type = unassigned DNA
gtgacagccggtattttttctgctgtgatatttggcgtttccacaactaacgtaagcgctgatagcactaacaacactggtgttacggtgtcacaagcaccagataaagtagcggacacaacagctacaacagataaagtagcagacacgacggctacaacggataaagcagcggacacaacagctacaacagataaagtggccgacacagcagctgcaacggacaaagtagcggacacaacagctacaacggacaaagtagcggacacaacagctacaacagataaagtggccgacacagcagctgcaacggacaaagtagcggacacaacagctacaacagataaagtagcggacacaacagctacaacggacaaagtagcggacacaacagctacaacagataaagtagcggacacagcagctgcaacggataaagcagcggacacgacggctacaacagataaagcagcggacacgacggctacaacagataaagtggccgacacagcagctacaacagataaagcagcggacacgacggctacaacagataaagtggccgacacagcagctacaacagataaagcagccgatacagcggctacaacggataaagtaaccgacacaacagttgcaacgaataaagcagtggacacaacagctacaacagataaagtggacgacacaacagccacaacgtcagaaaaatcaaaaagtattaaacaaattgatggtaaaacttatttcattggtgatgatggtcagcctaagaaaaattttacagccattgttgacggtcaagtattatatttcgacaaagatactggtgctttgacatcaaatagtagtcaatataccgatggtttagtcaatataggaaatgagcataatgcggcttattcattgtcttcggatagttttacacaagttgatggctatctaacggctaacagttggtaccgacctaaagatatattaaaaaatggtacaacttggacggctgcaacagcaaacgattttcgaccattgttaatgtcttggtggccagataaagatacccaagtttcatacttaaaatatatgcaatctgcgggattattatcagatgacgttgcattatcaaacaatgatagtatgaacagtttgacggatacggctatgactgttcaaaaaaacattgaagaaaaaattggcctattgggcagtactgactggcttaaggccgatatgaaccaaatggttgattcacaatcgaattggaacattagtagtgagtctaaaggaacagatcatttgcaaggtggtgcgctcctatacgttaatagtgatttaacaccaaatgccaattctgattatcgtttattaaatcgaacgccaactaaccaaaaaggtcaaattacaacaaatggtaatcaaggtggctatgagatgctgttggccaacgatgttgataattctaacccaattgttcaagctgaacaattgaattggttatactacatgatgaatatcggtagcatcgcccaaaatgatccaacagcaaattttgacggttacagagttgatgctgttgataacgtgaatgctgacttgttgcaaattgctggagattattttaaagcagcatatggaacaaatcaaagtgacgctaatgcaaacaatcacatttccatcttagaagactgggacaataatgatccagcgtatgtaaaagcacaggggaacaaccaattaaccatggattttccaatgcatttggcattgaagtattcattgaatatgccaagtagtgctcgtagcggtttggaaccagcaatttcaacaagcctagtaaatcgtgcagcagacgccacagaaaatgaagcccaaccgaactattcatttattcgtgcacatgatagtgaagtacaaacggttattgctcaaattattaaagataaaattaatcctagttccgatggattgactgtctcaacagatgaaattgccaaagcattcgaaatatataatgctgacgaattaaaggctgataaagagtataccgcatataatataccctcatcatatgcattgatgttaactaacaaagatacaattcctcgtgtgtattatggtgatttgtttacggatgatggacaatatatgtctgcaaaatcaccatattatgatgcacttacttcattgcttcaatcgcgagtaaaatatgtttcaggtggtcaatctatgaatatgacttaccttcataataatcaaggccttttgacgtcagtccgctatggaaaagacgccatgacagctaacgacactggtacaagtgaaacgcgcacacaaggtattggattaattgtcggcaacaaaactgatttaaacctgaataatgatgagcaaattgtgcttaacatgggcgccgcacacaaaaatcaagcttaccgtgcattaatgttaagtactaaagatggcttgaaaatttataatagtgatgacgaggcaccggtatcgtatacagatgatcaaggccgtttgatttttaaatctgatgtggtttatggtgtgagtgatgctcaggtttctggttatttagcagcttgggtgccagtcggtgcaaacgatagccaagatgctagaacagaaagtagtacaacagcgtcaacagatggtaatacctatcattcaaatagtgccttagattctcaagttatatatgaaggtttttcaaacttccaagccatgccaacacaggccgatgagtataccaatatcaagattgccgaaaatgcacaattattcaagagccttgggataacaagctttgaattggcacctcaataccgttcaagtacggataacagtttcttagattcagttattcaaaatggctatgccttcacggatcgctacgacattggttataatacgcctacaaagtacggtacagttgaccaactattagatgctttaagagcattgcatgcccaaggcattcaggctatcaatgattgggtcccagaccaaatttataatttgcctggcgaagaaatagtgacagccagtcgaacaaacggttcgggaaaggtgaatgaaagttcagttattaataatacgctatatgattctcgtactgttggtggcggagagtatcaagcaatatatggaggtgctttcttagataagttaaaacaagattatcctgagttatttgaaacaaaacaaatttcaacaggtgaagcaatgaaccctgatgtcaaaatcacagaatggtcagctaagtattttaatggctcaaacattcaaggacgtggtgcatggtatgttctcaaggattggtcaacaaatcaatactttaatgtttcaagtggtagtgaatttttacctaagcaactgttaggcgaaaaaacaagtacagggtttaccaacgtggacaatggcaagactgagttttattctacgagtggctaccaagcaaagaatacatttattcaagataatgacaattggtattattttgataatgatggctatatggttgttggcggtcaagaaattaatggtaaaaaatattatttcctaccaaatggtgtagagttacaagatgcttatttgtctgatgggactagtgagtattactacagtagtgatggtcgtcaaatttctaatcaatattatcaaggatcagacaacaactggcgttatttctttgcagatggtcatatggctgtagggttagcaacaattactacagaaaatggtacaacaaatcaacaatatttcgatgcaaatggtatgcaacttaagggcgtagctataaaggatactgatggcaatgtgcactattttgatggtaagacaggaaacatggttataaattcctggggcaaaataagcgatggttcatggttatacttaaatgatagcggtgtagcggtcacaggaccgcaaaatattaacggtcaaaatctttacttcaacgaagacggtattcaagtaaagggtgaagccattactgataatagtggaaacatacattattatgatcgcagcacaggaaatatggttgtgaactcatggggtgaaacgaataatggttcatggctatacttgaacgacaagggtgatgccgttacaggagaacaagttattgacggtcaaaaactatatttcagtagtaatggaatccaacttaaaaatacattcaagaagctatccgatggttcatggctatatttgaacgataaaggtcttccagtgacaggagcacaggtcattgatggacaaaacttgtatttcgaccaagatgggaagcaagtcaaaggtgacgttgctacagatggacaaggtaacactcattattatgacggcaacacaggaaatatggttactaattcatgggcagagttagcggacggttcatggatgtatctagataatgatggcaatcctttaacaggaccgcaaaagattgatggccagtcactctactttaatgatgctggtaagcaaatcaaaaacgcattggttaaactagatgatgggtcaacaatttacctcgatgataaaggtgtttcatcaaccggtattcaaagaattgatgataagatatattattttgatcctgatggtaaacaagtagtatgtcgttttgaagaattaccagatggttcatggatgtatctagatgatgacggtgttgctgctacgggcgctcaaaaaattaatggccaggaattatatttcgacaataacgggaaacaagtcaaaaatgacaaagtaattaatgacgatggaacaataaactattacacaggtatgagcggtgaaaaactaaaaaatgattttggtgaattaccagacggttcatggatgtacttggataatcaaggtaatgctgtaataggtgcccaaaaaattaatggccagaatctttacttcaagacagacggacgacaggttaagggtgaagcaaatgttgattcatcgggtgaaatgcacttctatgatcctgattctggcgagctaattacaaatagatttgaacaagttgctagtggtgtatgggcttactttgatgccaacggtgttgctgtaactggtgagcaacgcattggtaagcaaaatttattttttgatccaactggttatcaagttaaaggcgacaaacgaacaattgacggcgttctctatacctttgataaagaaagtggtgagagaaagggtttagattctatatcggtattacccaccaatggacaatacacaaccgataaggcccaaaattggtattaccaagtcgatggtgaaaatgtaaaagggctatatacaaataatgatggtcaattacgttacttcgatttgacaactggcgtgcagactaaaggtaattttgtgacaattggcaatgatacctactatttcaccaaggaacaaggggatggacagatagtttctgaggttgtgtcaggacactatggtactgtccagttgagtgacaattcgtctgcatgggtttatcgcggtgcaaatgatcaaattttgaaaggcctacagaatataaacggtcgtctgcaatattttgatctaaccaccggtgcgcaattaaaaggcggtgctgcaaactatgatggcaacctttattattttgaatcatcagatggtaacctagtcagtaaaattcagcaatcttattctactgggaattatgtgaccgatggtgataaagtaacatatgttgatgagcaaaacaaccaagtcacgggattagcgttgattgatgatcaactacaatacttcaatccaagtgacggtagtcaagtcaagaatgagcaggttatcgttgatggcgtcacatactactttgataaaaatggtaatggacaatacttgtttacaaatactgcaacgatgtctactaatgaatttgccaaacatagtgctgcttatagcaatgatagttctagcttcaagaatacgatagatggtttcttgacggccgatacctggtatcgccctaaagatatcttggaaaacggacaaacgtgggtagtttcttcaacaaatgacgtgcgaccactgataacagtttggtggccaaataaagatgttcaagttaattatttaaattttatgaagaaaaatggtttgctagatacaagtagtcaatttaatctacaatttgatcaatatgacttgaatgtcgccgcgcaaaaagttcaagtggctattgaaaaacgcatttcgaaagaaaagagtacagattggttgaaagatcttttgtttgaagctcatgaagatacgccttcatttgtgaaacaacaatttatttggaataaagattctgaatatcaaggtcaaggggatgcgtggttccaaggtggttatctgaaatatgataatagtgaattaaccccaacaacgaactcagattatcgtgaatccggtaatacattagacttcttgcttgctaatgatgtcgacaattctaacccagcggttcaagctgaaaatttgaattggttacattatttaatgaactttggcacgattacagctaatgatgatgatgccaactttgacagtattcgtattgatgccgttgactttattgataatgatgccattcagagaacctacgattacatgcgtgatgcttataaagttgatgcaagtgaagacaacgctaataagcatatttcactagttgaagctggattagatgctggtacctctacaattaagaataatgctttagttgaatctaactttagagaggcagctacactatcgctagcaaatcaatcagggaaaaatagttccttgactaatatgttgcaagacattgatggtggccagattatagctgatcacgccaacaatgcaacagaaaatgaagcaacgccaaattattcaattattcatgctcatgataaggggattcaagaaaaggttggtgcagcaattaccgacgttactggtgcagactggacgaatttcaccgatgaccaattaaaagaaggattagcagcttattatcaagatcaacgttcaacgaataaaaagtataacatctataacttacctagtatctatgctttgatgttgaccaataaggacacagttcctcgtgtttattacggtgatatgtatcaagatgatggccagtatatggaaaagcaaagtatttattatgatgccattgtttccttgatgaacactagaaagagttatgtgagcggtggacaaaccatggatgtagatgaacatggtttgttgaagagcgttcgttttggtaaagacgcaatgacagctagtgaacttggtacgaatgaaacacgcactgaaggtgttggtgtgctggtcggtaatgattcttcactaaaactaaatgattcagatacagttactttagagatgggggcggctcataaaaaccaagagtaccgagctgcattgttgacaactagtgatggtattgttacgtatgatgctgataatgatgcaccaacgatctggacagatgaccggggtacattaacgttctcaaataaggagattgctggtcaagattatactagtgtgcaaggattcgctaatccacaagtatcaggttacttagcagtttgggtgcccgtaggagctagtgacgatcaagatgcccgaacagcagcatcaacagataaaaatactgatgacaaagtactgcattctaatgctgcattagattcgaacttgatttacgaaggtttttcgaactttcaacctaaagcaaccaccaatgatgaactgactaacgtagtaattgctaaaaatgctaatttatttgaaaagtggggaatcacaagttttgagatggcaccacaatatcgttcaagtggggaccacacgttcttagattcaacgattgataatggttatgtatttactgaccgatacgatttgggatttgaaacaccaactaagtacggtactgataaggatttgcgtactgcaattaaagcattgcaccaatcaaatatgcaggttatggctgatgtagttgataaccaagtttataatttatctggacaagaggtcgtatcagcttcacgtgctggtgtttacggcaatgatgtgtcaactggatttgggacacaactctatgcggttaatagtgttggtggtggtaaatatcaagcccaatacggtggtgaatatttgaatgaattgaagcaacaatacccagatttgttcgaagctaagacgtatgactattgggttaaaaattattcaaatgacggatcggatccgtattacacactgtcgcaaaacacacgaaaagatatgccaagtagtgaggtcattaaacaatggtcagctaaatatatgaatggtactaatgtattaggaaacggtatgggatatgttttgaaagattggaatacaggtgagtacttcaaaattggagaaaagaatgctgattttataacaaattaa

SEQ ID NO: 16 - DNA, 8535 bp, Leuconostoc carnosum
  source 1..8535: mol_type = unassigned DNA
atgagagacaagaatgtaatatgcgatcgaaaaaaattgtataaatccggaaaattgttagtgacagcaggaattttctctacggttgtgtttggtatggccgtatctgacgttagtgctaatgacactgacaacactgtgttaacttcgaacagtggatttttagataaagtagtagatacgacaagtacggataaggcagccacaccagataaagtagtagatacgacaagtacggataaggcagtcacaccagataaagtagcagatacgacaagtacggataaggcagtcacaccagataaagtagcagatacgacaagtacggataaggcagccacaccggataaagtagtagatacgacaagtacggataaggcagccacaccggataaagtagtagatacgacaagtacggataaggcagccacaccggataaagtagtagatacgacaagtacggataaggcagccacaccagataaagtagtagatacgacgagtacggataaggcagccacaccggataaagtagcagatacaacgagtacggataaggcagccacaccagataaagtagtagatacgacgagtacagataaggcagccacaccagataaagtagtagatacgacgagtacagataaggcagccacaccagataaagtagtagatacgacaagtacagataaggcagccacaccggataaagtggtagatacgacaagtacggataaggcagcagatagaaccattagtatttctggtaaaacagttaaaaatattgaggaaattggtgggaaaacttattttgtaggggatgatggcaaagtcaaaaaaaacttcacggttattgttgatgggcaagtaatgtattttgacaaagagtctggagcattgacttcgaaccataagcaatataaagaaggtctttcagatataacaaatgaacataatgctgcctattcattagaaaacgacaactttacacaaattgatagttacttaactgccaatagttggtaccgtccaaaagatatattaaaaagtggcacaacatggacagcctcaaccgataaagactctcgcccattacttatgtcatggtggccagatcaacaaacagaactatcttatttaaagtatatgcaatcagcaggtttcttagcagaggatgttaatttatcagaaaataatagcattgatgatctaacagctgcagcaatggatgttcagaaaaatgttgaagcaaagattagcctatcaggtaatacagattggttgaaagaagatatgaatcagtttgttgattcacagtcaaattggaatatcagcagtgagtcaaaaggcactgatcatcttcagggcggcgctttattgtatggtaacagtgatatgacaccagatgctaattctgattatcggttactgaatcgcacaccaaaaaaccaaaccggacaaatcagtgctacgaatgatcagggtggttatgagatgttattggctaatgatgtcgataattctaatccaattgttcaagccgaacaattaaattggttacactacatgatgaacattggtagtattacaaaaaatgattcaacggctaattttgatggttatagagttgatgcggtagataacgtgaacgctgatttgttacaaattgctggtgactacttcaaggccgcttatgggacggacaaaagtgatgcaaatgctaataatcatatttctattcttgaagattgggataatagtgatcctgactacgtcaaaaagcacgggaatgaacaattaactatggattttccgatgcatttggcattaaaatatgctttaaatatgcctattgatatgcgtagtgggcttgaaccggcaattaagacaagtctggtgaaccgatcacaagatgcaacagaaaatgaggcacagccaaactactcgtttatacgtgcccatgatagtgaagtacagactgttattgctcaaattattaaagataaaattaatccaaagtcagatggtttgacagttacacctgatgaaattgcgaaagcttttgaaatctataatgcagatgaactaaaagctgataaggcatacacagcttttaatatcccgtcatcttatgcacttttgttgactaataaagatacggtgcctcgtgtttactatggcgatttatttacagatgatggacaatatatgtctgatcattcaccatattatgatgctattacgactttattagcgtcacgtattaaatatgcggctggtggtcaaagcatgggtatgacgtatttacacgataatcaagaagttttgacatctgtacgttatggtaaaggggctctgacagcagatgatttgggtaatgttgacacacgtacacaaggtattggacttgttatcagtaataaaacggatttgagtttaaagagtgatgagagcgttgtgctgaatatgggtgtcgcacataaaaatcaagcatatcgcccggcaatgttgactactaagtcaggattaaaaatttacgatactgatgatggtgcaccaattgtttatacaaataatttaggacagctaatttttaacgcagatacagtctatggtgtgagtgatccacaagtttcagggtaccttgctgcgtgggttcctgttggcgcaactgaagatcaagatgctagaactaaaggcagtcatgatggaacaactgatgggaatgtctatcattccaacgctgcattagattctcaggtcatatatgaaggtttctctaatttccaggcaatgccaacgaccactgatgaatatactaacgtaaagattgcacaaaatgcacagtggtttaaaaaattaggactgacaagttttgaactggcaccgcaatatcgttctagcacagatagtagctttttagactctgttattcaaaatggatacgcttttactgatcgatatgatgtgggttataatacaccaacaaaatatggtacagtcgatcaattattagatgcattgagagcattacattcacaagatatccaagcaattaatgattgggtacctgatcaaatttataacttacccggggagcaaattgtaacagcgagtcgtaccaatggatccggaaaatacgatgatgattcagtgattagtaatactttatatgattcacgtactatcggtggtggagaatatcaagcgatgtatggcggggctttcttagatcaattgaagcaagcatatcctggtttgtttgaaacaaaacagctttctactggtgttgcaatggatcctgatgtaaaaataaaagaatggtctgctaagtattttaatggttctaacattcaagggcgtggtgcttggtatgtactaaaagactgggcaactaataagtacttcagtgtctcgagtaataatacatttttacctaagcaattattgggtgaaaaagctagtacaggatttattacgaacgatggaaaaacagaattttattcaacaagtggctaccaggcaaagaatacatttattgaagacaacggtaactggtattacttcgataatgatggttatagcgtagttggaaagcaagtgattgataataaacactactatttcttgcctaatggtgtcgaattgcaagatgcctatttatcagatggggataagcaatattattataaaaaaactggtcgacaaatagtaaatcagtattatagagatgagcaaggtgattggaggtatttctttgcagatgggcatatggctcttgggttaactgacattgtttcaaatgatggaactcatgcgacacagtattttgataacaatggtgttcaagtgaaaggaacatcagaacgagataaagatggtaatattcactattttgatggtacatctggtaatttagttgttagttcatggggacaattgtcggacggatcatggctttacctgaatgataaaggcattgctgttacgggtgctcaacaaattgatggtcaatcactatactttaatgaagatggtaaagaagtaaagggtgatgcggtaacagataatcaagggaatatacgttattttgatggtgaatctggtcacatggtggttaattcatggggtaaattacctgatggttcatggatgtatttgaatgataagggtattgctgttaccgggcaacaaaaaatcaacaatgaagtcttatatttcaatgctgatggtaagcaaatcaaaagtgcatttaaagaactagtggacggatcatggctttacctgaatgataaaggcattgctgttacgggtgctcaacaaattgatggtcaatcactatactttaatgaagatggtaaagaagtaaagggtgatgcggtaacagataatcaagggaatatacgttattttgatggtgaatctggtcacatggtggttaattcatggggtaaattacccgatggttcatggatgtatttgaatgataagggtattgctgttaccgggcaacaaaaaatcaacaatgaaatcttatattttgatgctgatggtaagcagttaaagaatacactgaaaacattatccgatggttcacgtatttatttggatggtaaaggtgtttctgcaacaggtgttcaaaaaattaatggtaaagtgtcatactttgatgttaatggcaaacaagttagcaatcatattcaagaactccctgatggttcatggatgtacctagataatgatggattagctctaattggtaatcaagacgttgatggcaaacaactttactttgatgttgatggtaagcaaataaaaaatgacaaagtcaaaaatagtgatggaacaattaattattacacgggtactgttggtgaaaaactaaagcatgattttggtcagttatccgatggttcgtggatgtatttagatgaaaatggtaatgctgttactggtgaacaaaatattaatgggcaacatttatactttaaagatgatggacagcaagttaaaggagatgtttttgaggatgacttaggccgtatgcgttattatagtgctaactcaggtgagatggttgtcaatcaatttgaacaaatttctgatggtgcctgggcttactttggtgatgatggtgtggcggtaactggtgaacaacatatcaatggacaagatcttttctttgatgcgacaggtcaacaggttaaaggtgaaagtcgaaccattaatggtattccttatacctttgaaaaggagagtggtgagaaacggtctgttaatatagcaccattactagccatgggaaattatgttacaaataatggtacagattggcaatatgaagtgcaaggcaatcctgttaaagggttatatagcacttctgataataagttgcgttattttgatctgacaacaggtgtacagattaaaggcaactttgttactattggacataacacatactatttcaatccagctaatggagacggtgaattattgccagatgtttctgatggccattatggtacgatccaagttaaagatgcgaatacaaatgaaaagacagtttgggtttaccgtaatcaaagtaatactattttgaaaggtattcaaaatattcatggtaatatacaatactttgacttatcaactggggagcaaataaagggtggtattgcaaactatgatggcaatgattattactttgaatcagcaaagggtaacctcacaagtaaaattaagcaagtctatacagatggtcaatatgtaactaaagatggaaaaagcatttatgaagacgcacaacaacaaagtgtcagtgggttagtgtctattaatggtcagttacaatacttcaatccacaagatggcgtacaagttaaaaatcaacaaattattgttgatggcgttacttactactttgatgaaaatggcaatggtcagtatttgtttacgaatacaacggtcatgccaatggatgattttacaaagcacaatacagtttatagtgataacgacaacaactttaaaaataatgttgatgggttcttaactgctgatacatggtatcgtccaaaggaaatcttaaaagcaggaacaacttggacaacaacatcggaaagtgacatgagaccactgatcacgacatggtggccaaataaaaatgtgcaagttaactatctaaactttatgaaacaaaataatctattgcacacaaatgtggagtatagcttgctatctgaccaatatgatttgaatattgcagctcaagcagtacaaacagctattgaaaagcgaattgctcaagaaaatagcactgattggttgcaaaatcttctattcacagctcaagacgatcaaccatcttttgttaagcaacagtttatttggaataaagattctgaataccaaggaaaaggtgatgcttggttccagggtggttacctgaaatacgggaacaataaattgacacctaataccaactcaaattatcgtaaaactgacaacgcgtttgaatttttgttagcaaatgatatcgataattcgaatccggtcgttcaagctgaaaatcttaattggttagaatatctgatgaattttggtactataactggtaaggatgacgatgcaaactttgatagtattcgtgttgatgctgttgattttattagtaatgatacaattcaacgaacatatgattatttacgtgatgcctatcaagtagatcagagtgaagccaaggctaatcaacatatttcattagtagaggctggattagatgcaggaacatcaacagttaaaaatgatgcgcttattgaatctaatttgcgtgaagcggctacgttatctttggccaatgcatctggtaaaaatagtgcgttaaccaatatgttgcaagatgttgatggtggaacattgattgcagatcatacccataattcaaccgaaaatgaagccacaccaaattattcaattattcatgctcatgacaaaggtattcaagagaaagtgggggctgcaatttccgatgctactggggccgactggactaattttacagatacacaattaaaatctggtttggatctttattataaggatcaacgtgctacggataagaaatataatatttataacttgccaagtatttatgcattaatgctcaccaataaagacaccgtaccacgtgtttattatggtgatatgtatcaagataatggtcagtatatggcagagaaaagtatctattataatgctttagaatcattaatgtcagcacgtaaaagctatgtaagtggtggacaaactatggatgttgatagccacggtttgctaaaaagtgttcgttttggtaaaggggctatgactgctgacacggtaggtaatgaagaaacaagaactgaaggtatcggtgtgttagttggtaacgatgcttcattgaaactcaatgattcagatacagtgaccttagatatgggcgcagcacacaaaaatcaaaagtatcgtgcagcaattttgactactaataatggtctatcaacgttcgattctgataaagatgcaccgattgcttggactaatgataaggggatactaacattttcaaataaaaatgttagtggacaagataatactaatgtccatggtgttgctaatccacaggtatctggttatttggcagtatgggttccagttggtgctaaagatgatcaaaatgctgggacaagcgcatctacagtagtcaacacagatggtaaagtattacactctaatgcatcattagattccaacttaatttttgaaggattctctaacttccaacctagagcgacaacaaatgacgaattaaccaatgtcgttatagctaaaaatgccgatttattccagaaatggggtattactagctttgaaatggcaccgcaatatcgctcaagtcaggaccacacatttttagattcaaccattgataacggttatgccttcacagatcgttatgacttaggatttaacacaccaacgaaatatggaacagatagtgatttgcgattggcaatcaaatcattacataaagctggcatgcaagtaatggctgatgttgttgataatcaagtctataacttaccagaccaagaagttgtttctgcttcacgcgcaggtgtatatggtaatgatgtcgcgaccggatttgatacacaattgtatgctgttaattctgttggcggcggtaaatatcaagcacagtatggtggacagtatttgagtgagttaaagaataagtatccagatttgtttgaagctaaagcttatgattattggacaaaaaattatgctaatgatggatcaaacccgtactacacattatcacaacagactcgagatgatataccatcagacgagaaaattaagcaatggtcagcgaaatatatgaacgggaccaatgttttgggacatggtatgggttacgtacttaaggattggaatacgggtcaatattttaagattaataaagatggcgattccaacttaccagtttaa

SEQ ID NO: 17 - DNA, 5016 bp, Fructobacillus tropaeoli
  source 1..5016: mol_type = unassigned DNA
atgagaaagaaattgtataagtcagggaaaatgtgggttgccgcttcagttgccatctccttttccgcgttatctattagcgtggggaataatggtgctaaagcagatgattcacaacaatcatcaactcagattcagtcaacgcaagtaacaactgcgttgccagctggtggacaatattcaactacaaatggtgggcaaagctggaattatttagtcaatggtgttgcaattaaagggatgtaccaagatggccaaggtcagctgcgctattttaatttcattgatggtactcaagttaaaggtgaatttttaagtattaatggtacttattattattttgatcaaaatagtggcgagggacatttggttccaactcaaagtaacggccattatacggaaattggcaatactggtgcttggggctatcagaatagtaatggggaacttgttaagggaattcaaaatatcgatggtcaacttaggtattttgatgagaatactggtaaccaagttaaaggtggctcggctactattggtaataagagttattattttgaaccaagtcaaggaacacttacaacaacaattgatcaagtatccgatgctcaaaatgctaatattcgaggattggcaaccgttaatggtcagttaaattactttgatcctacaactggtgaacaagctaagcataagcaagtggctacaaatggtgcaacgtattatttcaatgatagtggtgttggaacatatttgttcaccaatgtacaaaatacaccggctaatgatgtttcacaacacaatgctgtaaatagtactgatactaaggattatacgaatactgttgatggcttcttaacggctgatacttggtatcgaccaaagtatattttggataatggtgaaaattggcgcgcttcaaacgaaggggagtatcgccctttcattatgaattggtggcctaataaaaacgttgaagttaattatctgaaattaatgcaaaataataatttgctatcaagtacggttcaatatgatcttttcacagatcaggctattttgaatcaagccgcttatcaggctcaaattgcaattgaaaagcgaattaagtcagagggtagcacagattggttaaatacgctgttatttggtggtgatgatagccatccttcatttgttaagcaacaatttatttggaattctgattctgaatctccatggcagggtgacgcttggttccagggtgggtatttgaaatacgggaatagtgttatgacgcctacaagtaattcaaattaccgccaggcaggaaatgcttttgatttcttgttagctaatgatgttgataatcaaaatccaattgtgcaggctgaggatttgaattggctatattatttgatgaattttggttctattacgactaatggtttagataatgattccaattttgatagtattcgattagatgctgttgattttattcataatgacgctattcaaagaacatacgattatttgagacaggcattcaatttaacgaaaaatgaagctactgcaaatcaacatttatcattagtggaagcaggggttgatgctggaacaaccacttataatagcgatggcttaattgaatcaaatattcgtccgttagcaactgattctttaactaacgctccaggtaaaaatgcatcactgtcaaacttaataaaggatgtcgatagtggtgaagtgattgcggatcatgctaatttttctactgatgatggtattccaaattattctattattcatgcacatgataaaggaattcaggaaaacgttggtgcagcgattactgctgcaacaggggctgattggactaattttactaccgaacaattagaacaaggccttgatttatattatcaagatcaacggtcaaccaacaaaaagtataatatttataatttaccaagtatatatgctttaatgctgacaaataagggtactgttccaagagtatattatggagatatgtatcaagataatggtcagtatatgcaacaaaaaagcctatattatgatgccattagttcactaatgactgctagaaagcaatacgtcgctggtggtcaaacaatgagcgttgatgaaaatgggttattaaagagtgtacgttttggaaaaaacgcaatgactgctcaagatacaggtgatgctgaaacaagaacagaaggtgttggtgtcattattggtaatgatccatcagtgaaggttgcggatggtcaaacagttaccttggatatgggtgctgctcataaaaaccaagcttatcgtccattaatcttgacgacaagcgatggcattcaaacttacgatagtgatgaaaatgcaccagttgtatatacagatgacaatggaatcttgactttttcaaatcaagatattaatggtcaagctaacacaaaaattgttggaactttgaatccacaggtttcgggttaccttgctgtttgggttccggttggtgccagtgctgatcaagatgctagaacagcaccatctactcaaagtactaatgatgggaaagtattgcatacgggagctgcgttggattcaaaccttatttttgagggattttctaatttccaaccaatgccaactacacatgatgagatgacaaatgttgtgattagtcaaaatgctagtcaattcgctaaatggggtattactagttttgaaatggcaccgcagtatcgctcatcagaggatcatagtttcttagattcaacaattgataatggatatgctttttcagatcgctatgatttgggatttggaacgcctactaaatatggtactgatgaagatttaaggaacgcaatcaaggctcttcatcaaaatggcatgcaggtgatggctgatgtcgtaatgaatcagttatattccttaaacggaaaggaagttgtttcagctagtcgtgcgggtgtttacggaaatgatgtagatttgccttttggaacacagctatacgtggtcaatactaccggtggtggtgagtatcagaagaaatatggtggtgctttcttaaacattattaaagaaaaatatccaactttatttgattcagagtcttacgattattatttgaaaaattattctgataatggccacggaccagcatatatgacaacagctacagcaactcgagaggcgattccatctgatcagccgcttaaagaatggtcggctaaatatatgaatggtacgaacattttaggacttggtatgggatatgttcttaaagattggaataatggtgcctatttcaaattatcaggaactgatacaacgcttccacaatcattggttgccttgactgggtggaatcaaaacgatgatggcacttggtcatattacagcactgacactgatgatcgagtaacagggaaacaagtgattgatggaagaaccctacttttcgataatcaaggaaaccagattaagggtggttggggtgaaaaccctgatggtacatggtcgtattacaatgctgacactggtgatcgagtaataggtgagcaagtaattgatggaagaactctattttttgataatcagggagttcaagttaaaggcggttggggtgaaaactatgatggtacatggtcatattacaacgctgataccggtgatcgagtaacgggaaagcaagtaattgatggaagaacattattgtttgataatcggggagttcaagtgaaaggcggttggggtgaaaactctgatggtacatggtcatattacaacgctgacaccggtgaccgagtaacaggcaatcaattgattgggggaagaaatttacttttcgataatcaaggaaatcaaatcaagggcggttgggatgaaaaccctgatggaacttggtcatattacaatgccgatactggtgatcgagtaaccggggtgcaagtgattgatggaaagcagttgctattcgatagtaatggtattcaggttaaaaattcatggcagaaaaatgccaatggaacttggtcatattatgatgctaatgacggacatttagtccctgcaaatagctcaaatgatggaacttcatcatctactcaagatagtgggaataagagcaatcaaaatccttcaagtagtagcaatgctgtaaataagactactggttggattaagaatagtgatggaacctggtcttatctatctgcaaaaagtggtcaaaaggtcactggaagtcaaactatcgatggcaagcaattgttgtttgatgaccatggcgtccaaattaaaggtggctggggtaaaaatgctgatggtacatggtcatattatgatgcgaactcaggggaactgacttcaactagtgatatgagtaatgtaaatccacagcaaacaacaactactacgaatgaacagagtacaaccaatcagccaacagatatcacaaaaaatagtgatggtgtttatgtatataagaatgattccaataagaaggcgcaaggctacttgaatgatggatcatcatggaagtggtttaatgatggtcaactttacaccggtttccaaaattacatgggtgcttactattactttattaatggtattcgtcaacaaaaccagtgggaaaatatctggggattgaagtattatgttggtgatgatggtcgaacagttgaaggaattcatgctatcgacggtcatgcttatgactttggtaccgatgggactttcaatgtgaagggatcggctagtggctacttgaatgatggcaagagctggatgtggtacgaaggtggtaacccatatactggtttccgctactacatggatacttactattggttcgaaaatggtgtgcgtcaagataatgcttggcatcaagcttggggtctgacatactacacaggtgcagacggtcgagcagttcagggtgttcaaaacatcaatggtaagctttactacttcggtaatgacggcaccttctttatgcggacaaaccaagaagta

SEQ ID NO: 18 - DNA, 4899 bp, Lactobacillus kunkeei
  source 1..4899: mol_type = unassigned DNA
atgaataataataatagtgaacaaaaacttcgttttaaaatgtataaatcgggaaagcattgggttgttgcaggtttaacaacagctgttgtatcagttgcaatatacagtggctcttcaatcattaatggtggagtaactgtaaaggcagatgtgcaaagcactacacaagcaactacatcagtttctgataattcaaaagattcatcaaatagtattagtaataataattcaaatgatgacaatcaaattgcaacacttcctcaagatggtacatattcaactggagataacggtcaaacttggaagtatgttgaacaaaataaaaacatacagggactttataaagatagtgacaaccaactaagatactttaatgaatatgatggaactcaagcaaagggtgatatcgtaaatgttaataataataactattattttgatagtggtagtggtcaaggacataagatagataactactcaggtggaaattatgttgaatctaacgtcaataatgaaaatggttggatttataaagctactgacaatactgaagtaaaagggattgccactatcgatggtaatgttcaatattttgatcaatcaactggacttcaactaaagggtggagcggttcaagtaggaggagccgactactattttgatccaaacaagggaaacttagttggtaaagttgatcaagttgtaaattcaaacaattatagtgataataaattactagatagtaataaaaatgtagtaaaaggattaacagttaataatggaacattacaatactatgatccttctactggtgatcaagttaaaaataaacaagtgattgctaatggcgtcacatattattttgatgcaagtggtaacggaacatacttgttcacaaatacaggcaatagtgccgttaatgatttctcacaacgtaacgctgcaaatagtgtaaatccaagtgattacaaaaatgtagtagatggatttttcaccgcagatacttggtatcgaccaaaacaaattatcgataacggtacaacatggagaaattccaatagtaatgacttacgtccaatgattaccgcttggtggccaaataaggatgttcaagttaattatttgaaattgatgcaagacaatggacttttagataaaagtgatacttatacattgcaatcagatcaacaagttttaaatcaagccgcccagagcgctcaagtaaatattgaaaagaaaatatcacagacaggaagtacagattggttaaacgatttattgtttgcaagtcatggcaataatccttcatttgtaaaacaacaatatgtatggaattctgactctgaatctccatggcaaggtgatgcttggttccaaggaggatacttaaaatatgggaatagtgttatgacaccaaacactaattctaattacagagattcaaataatttatttgatttcttgctagcaaacgatgtagataattcaaatccagcagttcaagctgaggacttaaattggctatattacttaactaattttgggacaattactgctaatgattcaaatgctaattttgatagtattagaatcgatgctgttgattttattagtaacgatatcatacaaagaagttatgattaccttcgccaaaagtttaatttaacacaaagtgatgctaatgctgattcacatatttcattagttgaaggtggagtagatgcaggaaccacttcttacagtaatgatggattagttgaagcgccatttagactaggtgcctatccattgctacataagcaaggtggagacgtatttaaagatttaataaacgaagaagattctggaatcgatatttcaaatcataacggtgaaactaatacaactaacactattggtggattaactttatctggtggaaaacctaactactcaattgttcatgcacatgataaagatgttcaggaaaaagttgggcaagccattgttgataccacaggtattaaggattggactgattttactccaagtcagttagcacaaggtctagaaacattctataacgatcaaagacaaacagacaagaaatacaatgattataacgttcctagtgcatacgcaattatgttaactaataagggaacggttcctcgtatttactatggtgatatgtaccaagatgatggtcaattcatgcaaaagaagagtctttactatgatgatatagcaaacttgatgactgcaagaaagaaatatgtatctggtggtcaatatatggttgataacgatggaattctaactagtgtacgttttggtaagggcgcaaatacagtcaatgattctggtacatcagatactagaaaccaagggattggattaatagttggatcagatcctaaaaaggtattaaacgatggggataccattgttttacacatgggtgctgcacataaaaatcaaaagtatcgtgcattaatgctaacaactgaaaacggagttcaaaattatagttcagatgataacgctccagttgctgaaactgatgataacggtgatttagttttcagtaacaaagatattaatggacaagataatactgcaattaaacaagtcgctaatccagaagtaaatggttatttagctgcctgggttccatttggcgcttctgatgatcaagatgctagaacatcaccatcaactagccaaaataacgatggtaatgttttacatgaaaatgatgcattggactcaaatcttatttttgaaggattttcaaacttccaacctactccaacaaatcatgatgaatatgcaaatgtagtaattgcaaaaaatgcatcattatttaaagattggggtgtaacaagttttgaaatggcacctcaatatagatcaagccaagatcatactttcgttgactcaactatagataatggttacgctttttctgatagatacgatttaggattcgggacaccaactaaatatggaacagatgaagatctaagaaatgccattaaatcacttcatgcaaatggaatgcaagtaatggccgatgttgtttataatcaactatataacttaccaggagaagaagttgtttcggcaacaagagctggagtaactggaaatgataatgcacttccttttggaacacaactatatgttgttaatacagttggtggtggagactatcaaaagaaatacggaggggcattcttaaaccaattacaagaacaatacccttcattattccaatctcaaaaatacaagtattactacaagaattatgctaataatggtgctggaccaggttatttaactgtaactgatggagaacgttctgcaattccttctgatcaaccaattactgcatggtcagctaagtatatgaatggtaccaatattcttggtcgtggtatgggatatgttctaaaagactggaacactggcgcttacttcaagataagtggtgatgactctactcttcctacgagcttaacttatagaagcggatgggtagaaaatccagacagcacttggtcatactattcaaaggatagcattgccaaattaactggagcacaaattgttaatgatcaaagagtattctttgataacaacggtattcaggttaaaggtggatgggtagaaaatccagatggaacttattcatactatgacaaaaatagcggtgaacttttggtaggttctcaactagtagacggaagacatgtattctttgataatgttggagtgcaagttaaaggtggatgggtagctaacactgatggtagttattcatactacaatgctaatgatggtaccatgttaacgggtgctcaatttatcgatggacaaaatgtatactttgatgctgatggtaaacaggttaagggtaactgggtgcaaaataacgatggttcttgggcatattatgatgctaatttaggtcatcttgtaaaagatgctaagcatgttgattcacaaccaagcactcaacaagttaataataaacaatccgaggaaaaatcaaattcaccattagttgaagctcaaaacaataaagattcagcttcagtagaatctcaaaatcatcaaaattcagtgtcagtagaatctaagaatgaaaacaaaaatcaatcaaacgatgtaagaaatccttccgaaaaaacagacaccaaaacatctaatgaaaaatcaaaagaagtatccaaagaagatgctgcatatgacaatgccaaaaaatcattagtagaagctaaaaagttggtagataagaagccaaataaaactaatattaataaatataaaaaggcattgaagtcttatgaaaatgcacaaaagaagatgaataagtctgtgatttcatcatataaaaaggctgcaaaagaactaaacgctgctaagaaaaacctttctaagaaaaataataaggttaatatgaagaaatatagtattgctttaaacaaatatcgtaatgctaagaaatcttacgtggtaattaagaaaaaagatttagataaatctaaatctgcgttaaatactgctaaaaaagcacttgctaaaaagaagacaaagcaaagtcaaaagaagtataacgatgcattgaaaaaatattacgatgcagaaaaaacttatctaaacttgactggtaattacactaaaaaatattattatgatttcgataaagtagggaaaaaggttaaagttatcaaatcaacttccgtatataattcattgaaaccaaatagtaaaaaagtagtaaagaagattaaaaaaggatcagtaattaaagtgaagaaacttattttgtcaaataagaagtcatactttgatttaggatataaccgttttgtaatcgcaagtaaatcttctataaaaaaagctaagtaa

SEQ ID NO: 19 - DNA, 3957 bp, Lactobacillus kunkeei
  source 1..3957: mol_type = unassigned DNA
atgaaatcaaaagataacaattcaaaaatacatttcaaaatgtataaatctggaaaacaatgggttattgctggattaactacagcggttgctgctgtttctatttatggtggaacaactatatttaatgacagcgtggtagctaaggctgatacagcagttaaatcttcacaaagttccaacgctgatactactcaatcttcatctgctgttgttgattctagctctgcaaattcaagcagtccatcatcgactgaagtatcagtaaacgtaaaaaatacatcaaatgcagatacacaaggtattcaagcattaccagatggtggtaactacacatcagaagataatggtcaaacttggaaatactcatatcaaaacaatgatgttaaaggtctatatcaagaaaacaataacattcgttattttagtgaaaatgatggatcacaagtaaaaggaaacatcttaaatgtaaataacaaaaactattattttgatagtaataatggtgatggccatttaattaaagactattctggtggaaactatgttgaacaaagtcaagataaatggacttataagacatctgataataaccttgttaaaggtatcgcaactattgatggacaaattaaatattacgatcctactacaggtatccaattaaaaggtggagcttttgagattggtggggctgcttattacgttgaccccgctcaaggaaacgttgtcggaagagtaaattcaattattaactctggaaattatgtcaataagaacaacaatactacttttgttgatgataataacaatacagttaagggattgaatgtagtaaggggtaacttacaatactttaactcaacaacaggatatcaagctaagagtgaacaagttgttgctaacggagccacatattactttgataaagatggtaacggaactttcttatttaataacacaggttcattttcagtaactgattttgcaaaacacaacgttgctaacagtaatcaaccatctgattttaccaatactgtagatggtttcttgacagctgaaacatggtatcgtccaaaacaaattttagataatggtactaagtggcgcaactcaaataaagatgatcttagacctcttattacagtttggtggcctaacaaagacgtacaggttaattacttgaaactaatgcaagataatggactattggataatagtgttaaatatactgtattttctgaccaacaaactttaaatgaagcagctcaatcagctcaagttaatatcgaaaagagaatcactcaacaaaaagcatcatcatggttatatgatttattattcaagggagattcatctcatccatcatttatgaaccaacaatatatttggaatatgggttcagaatcacaatggcaaggggatgcttggttccaaggaggatacctaaaatatggtaatagtcctttaaccccaacaaccaactcaaaatatagaaaaacagataatcaatttgatttccttttggccaatgatgttgataattctaatccagcagttcaagctgaagatattaattggttatattacttaactcatttcggaacaattaccggtaaaaatgataaagccaattttgatagcattagattggatgcagtcgattttgtaagtaacgagattattgaacgaagcaatgattatctaagagatttatataagttaaccaagagtgatgcaaatgctgataagcatatatcacttgttgaagccggtgttgacgctggaactagttcaacaaatggtgattctttggttgaagcaccttttagattatcagcctatggattacttcataattctggtaaagttgatgcattgcaagacttagttaaagaagttgattcaggtgttcttatttccgatcacagtaaaaactctaaggatggtggggttcctaattattccatcgtgcatgctcatgataaagatgttcaagaaagagtaggacaagcaattgttgattccactggcattaaagattggacaaactttacacctgctcagttagctaaaggattaagcgtttactatgctgatcaaagaaagacagttaaaaaatataatgattacaatatgccaagtgcttatgcaattatgttgacaaacaagggtacagttccacgtatttattacggtgatatgtatcaagatgatggtcaatttatgcaaaagaagagcttctactatgatgatattgtcaaattgatgactgctagattgaaatacgttgcaggtggtcaaacaatgagcgttgattctaacggatttttgaagagtgttcgttttggtaaaggcgcaaagacagttaattctaagggaacaaaagaaacaagacacgaaggtattggtttaattgttggaaatgatgctaagaaagttctatcaaatggacaaaaagtaacattgaacatgggtgctgctcataagaaccaaaaatatcgtgcattaatgttaacaactaacaagggagttcaaacatttgcttcagataaaaatgctcctgtagttaagacagatggaaatggtgttttaacatttactaacaaggatattaaaggtcaagctaataccagtgttagaggagttttaaatcctaaggtttctggataccttgctgtatgggtaccagtaggcgctaaagataaccaagatgcccgtactcaaccatctaagaaaaatagaaatgatggtaaagtgcttcattcaaatgatgctttagattctaatttaatttacgaaggattctccaatttccaaccacaaccaaagaaacgcagccaatatgcaaatgttgtaattgccaaaaatgcaaagctattcaaaaaatggggaatcactagttttgaaatggctccacaatatcgttcaagtaatgataaaacttttgttgattcaacaatcaacaacggatatgcattctcagatagatatgacttaggtttcggtaagccaaccaagtatggtactgataaggacttaagaaaggctattgaatcactgcatgatcaaggcatgcaagttatggctgatgctgtattaaatcaactatacaacttacctggaaaacaagttgtttcagcacaaagagctggagtaacaggtaacatagctgatttgccatttggaaagcaattatacgttgttaatacaattggtggcggtaagtatcaaaagaaatatggtggaaaattcttaaaggctttaaaagctaaatatccaaacatatttaagggtaagacttataaatataactacaagaattattctccaactggtgaaggatatttaacacttaatacaggtaagacggtttcaattccatccaaccagcctattactgaatggtcagctaaatacatgaatggtactaatatccttggtagaggtatgggttatgtattgaaggattggaatacaggaacatactttaagttgaacggactaaagacagttcttccttctgaactaacagataagagtaactgggttaatgaagtttctacatcttcaaaagatgtatataataagtctgtttcaaatttaaaagacgctaaaaagaaattagctgcaaagaaatcaaaagaaaacacacaactatataacaatgctctaaattcttattacaaagcagaaaaagaatacttgagcagtgctaaactttacaataaaaagtactactatgattttgataaattaccttcaaaagttaaagtagctaaggtcacttactcatataaatcaactgatttcagcaaagataataggattaaaaagcttaaaaaaggaactgttttaaatgttaagggattagttcttaacggaaaagttacaagaattaatattggtaacggtaagtttgtaactgcatctaaagatttcataaaagcaattagatag

SEQ ID NO: 20 - DNA, 3420 bp, Lactobacillus kunkeei
  source 1..3420: mol_type = unassigned DNA
atgaaagcagaacataataatttaaaattacatttcaaaatgtataaatctggaaaacaatgggttattgcgggactaacaactgtggtagcttcaattgctatcttcagtggaaaccaactatttagtgggaatgttgtagcaaaggcagacggggtaacaacaacccaagtttctcaaaatagcaatagtagtaatggaatttctacacttccaaaagatggaaagtatgtatctagtgataatggaaaaacatggaagtatgtttctcaaaacaatgctgcaaaaggattatatagtgatggttcaaacattttctactttaatgaaagtgatggaactcaagtaaaaggtaacattattagtgttggaaaaaacacttattatttcgatagcaaaactggtcaaggaacaaaaatatcagattatactggtggaaagtacactaacccaagtactgataataaagctgcttggacctaccaagataagagcaataacactgtaaagggtgttgcaacaattggagattcagttcaatattacgatgaaaaagatggatttcagctaaaaggtggatctattaacttaggaaacacaaattattatgtagattcaaaccaaggaaatgtaactagtaaagtaaatagagttgtttcttctaatgacgggactttaaaaggattaaacgtcgtaaatggtaatttacagtaccttgattataaaactggaaaacaagtaaagaataaacaagtagcagttaatggtgttacatactactttgatgataatggaaatggttcatatttatttaccaatttaggtaaatctgttcgtactaattttgataaatataatgctgttaatggtgtaaataaatctaatttcacaaacacagttgatggattcttaacagcagattcatggtatcgtcctaaacaagttttggctaacggtaagaaatggcgtaagtctacttctaaagattttagaccattagtaacagtagcatggccaaacaaagatgttcaggttaattatttagctttaatgcaaatgaacggtttgttatctaataaggaaaagtacactgttttctctgatcaacaaacattaaatgctgctgctcaaaaagcccaagttaatattgagaaacgcattagtaaaaagaaaagtactaaatggttagacaagttgttattccaaggtacaactactgaaccatcatttataaagagacaatatgtttggagttctgcatctgaatatcctggacaaggggatgctcaatggttccaaggaggatacttgaagtatggtaataatcgtttaactccaaaaactaattcaaagtatcgtcaaattggtaatgcttttgactttttattagcaaatgatgttgataactctaacccatcagttcaagctgaagatattaattggctacattatttaactcactttggttcaataactgctaaagatcctaaagctaatttcgatagtatcagaattgatgctgtggactttatcagtaatgatatagttcaaagaagtaatgattacctaagggatttatacaaacttacaaagaatgatagcaatgctgataagcatatttcattggttgaaggtagtctagaagcggcaacaagttattcacattcagattctctaattgaatctccttatagaaatgatattgatggactattaggaaactcaggaaattctgcagacttaagtaaattaattaaagagtatgattctggaatcattatttcagatcattcaggaactactaacaatagttctattccaacttattctattgttcatgcccatgataagggagttcaagaaagagttggacaagcaattatggataattctggtataactgattgggcaaactttactcctaagcaattaagagatggattgaagttatactatgatgatcaaagaaaaactgttaagaaatacaatgactataacgttccaagtgcctatgcaataatgctaactaacaacaacactgttcctcgtatctactatggtgatatgtatcaagatgatggacaattcatgcaaaaaaagagtctatattatgatgacatcgttagtttgatgctggctagaaccaaatatgttgccggtggtcaaagtatgagcatggattcaaatggattcctaacaagtgttcgttatggaaaaggtgctaataatgttaattcacaaggtacatctgaaactagaaatgaaggaattggagtaattgttggtaacgacactaataaagttcttaatgatggacaaacagttactttaaatatgggtgcagctcataagaaccaacaatatcgtcctgttttattaacaaccaaagatggaatcaagacatattcttctgataaaaatgcgcctgttgtagaaactgattcaaatggggttcttacatttagcaataaagatgttaatggacaaagtgacacaagtgtaaggggaacattaaatccacaagtttccggatatttagctgtatgggtacctgttggtgctaagaatggacaagatgctagaacaaaaccatcaaagaaagccagaaatgatggtaaagtacttcatttaaatgatgctttagcttctaacttgatttttgaaggattctcaaactttcaacctatgccaaaaaataagagtcaatacactaacgttgtaattgccaaaaatgctaaaacatttaaaaaatggggtattacaagttttgaaatggcacctcaatatcgttctaccaacgataagagttttgtcgattcaactataaaaaacgggtatgcattctcagacagatatgatttaggtttcggtaagccaaccaaatacggtacagataaagatttaagaaaggccattaagtccctacatgctcaaggcatgcaagttatggctgatgttgtatataatcaactatataaccttcctggtaaagaagtagtatctacttctcgagctagtgcttatggtaataatgttgatgttccatttggaaaccaattatatgttgtaaatactattggtggtggaaagtatcaaactaagtttggtggaaaataccttaaagaactaaagaagaaatatcctagtttatttaaggcaaaaacatataaatactattacaaagacaatcaaaaagatggctcagttaaattagctcttacttcaagcaagagatcaagcattccagcaaacaaacctattaaggaatggtctgctaaatatatgaacggtacaaatgtattgggattaggaatgggatatgtcttaaaagattggaataatggtaaatactttaagattaatgggacaaagacttcgttaccaagttctatttattacaaaagtacaaaaaataagtaa

SEQ ID NO: 21 - DNA, 4392 bp, Lactobacillus kunkeei
  source 1..4392: mol_type = unassigned DNA
atgaatatcaatagtaatgaacgtaaggttcgatttaaaatgtataaatctggtaaacaatggattgtagctggattaactacagcagtaatatctattgctgtgtatggtggttcttcaattgccaatggtggaattgaagctaaagctgatgcacaaaacgcagctacttcttcaattgtgaatactaataattctacaaatagtagtaatgctaattcaattgcatctcttcctcaaaatggtacatattcaacaaatgataacggacaaacttggaagtatgtatcacaaaataaggacattcaaggtttatataaggataataatgatcagttaagatattttaacgaatatgatggcactcaagccaagggtgatattgtaaatgttaataatgacaattactattttgataaagacagtggtcagggacataagattgatagctacactgggggaagctactctgaaagtaaggtcaataaccaggatggttggatttataaatcaagtgataacaatgatgttaaaggagtcgctactgtcgacggtaatattcaatattttgatcaaaatacaggtctgcaattaaaaggtggatctgcgcaaattggtggagttgattactattttgacccaaacaagggtaatttagttggaaaagttgatcaagtagtaaactcaaatgattatagcgataataagttactggatagcaacaaaaatgtagttaaaggtttagtagtaaataatggtcagttacaattttttgatacatctaacggtaatcaggctaagaataagcaagtaatagctaatggaattacctactattttgatactaatggaaatggtcaatacttgtttactaatacaggtaagagtgctgttgatgactttacacaaagaaatgctgctaacagtgttaacccaagtgattacaaaaatgttgtagatggattttttacagcagatacttggtaccgtccaaaacaaatattagataatggtactacttggcgtaattcaaatagtaatgaactccgtcctatgattactgcttggtggccaaacaaggatgttcaagttaattatttgaaattaatgcaaaataacggactattagataaaagcaattcgtattcaatacaatctgatcaacagacattaaatcaagcagcacagaaagctcaagtaaatattgaaaagaaaatttcacaaacggggaatactgattggttaaatgatttgttatttaaaggaaacggcgataacccatcatttgtgaagcaacagtatatttggagttcagattcagaatcgccatggcaaggagatgcatggttccaaggtggatatttaaaatatggcaatagtgtcatgacaccaaatactaattctaattatagagattctaataacttgtttgatttcttgttagctaatgatgtagataattcaaaccctgcagttcaagcggaggacttaaattggctatactacttaactaacttcggaactattaccgctaatgattctaatgctaattttgatagtattagaattgatgccgttgattttattagtaacgatattatacaaagaagttatgattacttacgtcaaaagtttaatttaatgcaaagtgatgcaaatgctgattcgcatatttcgttagttgaaggtggagtggatgcaggaactacatcttatagcaatgacggattagttgaggcaccattcagactggatgcttatcctttgctacataagcaagatggagacgtatttaaaaacttaatagatgaagaagattctggaatcgacatttcaaatcataatggagaaaccaatacaaataatactattggtggaataactctatctggtggaaaacctaactattcaattgttcatgcccatgataaggatgttcaagaaaaagtaggtcaagctataatcgatacaactggaattaaggattggactgactttacaccaagtcaattagcacaaggattagaaactttctataacgatcaaagacaaacagtaaaaaaatacaatgattataatgtgcctagtgcatatgcaatcatgctaactaacaagggaacagttcctcgtatttattatggtgatatgtaccaagatgacggtcaatttatgcaaaagaagagtctttattatgatgatatagcaaacttgatgactgcgagaaagaaatatgtatctggtggtcaatctatggttgataataatggaattctaacaagcgtacgttttggtaaaggtgccaatacagttagtgattcaggaacagaagatacaaggaatcaaggtattggattaatagttggatctgaccccaaaaaggttttaaatgatggggataccgttgttttacacatgggtgcagcccataagaaccaaaaatatcgtgccttgatgctaactactgaaaatggaattcaaaattataattcagatgataatgcaccagttgctgaaacagatgataatggtgatttagttttcagtaacaaagatattaatggtcaagctaacactgcaattaaacaagttgccaatccagaggtgaatggttatctagcagcatgggttccggttggggcttccgatgaccaagattcaagaacagcaccatccactagtcaaaataatgatggtaatgttttgcacgaaaacgacgcactagattcaaatcttatttttgaaggattttctaacttccaaccaactccaacaaatcatgatgaatatgcaaacgttgtaattgctaagaatgcgtcactatttaaggattggggtgtaacaagtttcgaaatggcaccacaatacagatctagtcaagatcatacattcgttgattctacaatcgataacggttacgccttttcagatagatatgacttgggatttggtacaccaactaaatatggaacagatgaagacttaagaaatgcaataaaatctcttcatgataatggtatgcaagtaatggctgatgtagtatacaaccaattatataatttgccaggacaagaagttgtatctgcaaccagagcaggtgttactggtaatacaaatgcattaccttttggtactcaattgtatgtagttaatacaatcggtggtggagattatcaaaagaaatatggtggagcattcttaaatgaactgcaagaacaatatccatcattattcaaatctcaaaagtacaagtattactacaaaaattatgctaataatggtgctggaccaggttatttaacagttaatgatgcagaacgttcggatattccatataatcaaccaatcactgaatggtcagctaagtatatgaatggaactaatattcttggacgtggtatgggttatgtattaaaggactggaatacaggtgattatttcaagttaagcgggagcgattcaacactaccaagtagtttaacttacaagagcggttgggttgaaaatcctgatagtacttggtcatactatgaaaagaataatattgacaaattaactggatctcaagttattaatgaggaaagagttttctttgataacaacggtattcaagttaaaggaggatgggtaaaaaattcaaatggtacctattcttactatgacaagaatagtggaaacatcctaactggtgatcaattaatcgacggtgaacatttcttctttgataacaatggtgttcaagtaaaaggaaaatggataaagaatagtgatgggtctaagtcatattacgattctcacttaggaaaacttataaagacggataaaaaagtatcttctaatgctcgaaaaaagaaatctaaggaagaattattgtatgagaatgccttaaaagttttaagaaaagataagaaacgtctcgataaaaataagacaaaggcaaatatcagaaaatataataaatctctaaaaaaatatcgtaaagcaaagaagaaacttttagcgattactaagaatagagttgctaatgctagaaaagcgattaaaattgctaaaaaagttctttctaagagaaaaaatattaataatgaaaaaagatattataaagctctaaaagaatattatgtagcggaaaaatcatatttgaaaattactggaaattacaataagaagtattattacgaatttgataaacttactccaaaagtcaaagtggttaagaacatttattcctataagagtagacattttactaaaaagaatagagttaaaaagattaaaaaaggtactcttgttagagttaaaagtatagttcgaagtggtaaagtcgctcgaattaatatcggaaatggtcattttataacttcatctaaagattttattaagatgtttaaataa

SEQ ID NO: 22 - DNA, 8526 bp, Leuconostoc mesenteroides
  source 1..8526: mol_type = unassigned DNA
atgagagacaaaagaataatatgcgatcgtaaaaaattgtataaatctgggaaactgttagtgacagccggtattttttctgctgtgatatttggcgtttccacaactaacgtaagcgctgatagcactaacaacactggtgttacggtgtcacaagcaccagataaagtagcggacacaacagctacaacagataaagtagcagacacgacggctacaacggataaagcagcggacacaacagctacaacagataaagtggccgacacagcagctgcaacggacaaagtagcggacacaacagctacaacggacaaagtagcggacacaacagctacaacagataaagtggccgacacagcagctgcaacggacaaagtagcggacacaacagctacaacagataaagtagcggacacaacagctacaacggacaaagtagcggacacaacagctacaacagataaagtagcggacacagcagctgcaacggataaagcagcggacacgacggctacaacagataaagcagcggacacgacggctacaacagataaagtggccgacacagcagctacaacagataaagcagcggacacgacggctacaacagataaagtggccgacacagcagctacaacagataaagcagccgatacagcggctacaacggataaagtaaccgacacaacagttgcaacgaataaagcagtggacacaacagctacaacagataaagtggacgacacaacagccacaacgtcagaaaaatcaaaaagtattaaacaaattgatggtaaaacttatttcattggtgatgatggtcagcctaagaaaaattttacagccattgttgacggtcaagtattatatttcgacaaagatactggtgctttgacatcaaatagtagtcaatataccgatggtttagtcaatataggaaatgagcataatgcggcttattcattgtcttcggatagttttacacaagttgatggctatctaacggctaacagttggtaccgacctaaagatatattaaaaaatggtacaacttggacggctgcaacagcaaacgattttcgaccattgttaatgtcttggtggccagataaagatacccaagtttcatacttaaaatatatgcaatctgcgggattattatcagatgacgttgcattatcaaacaatgatagtatgaacagtttgacggatacggctatgactgttcaaaaaaacattgaagaaaaaattggcctattgggcagtactgactggcttaaggccgatatgaaccaaatggttgattcacaatcgaattggaacattagtagtgagtctaaaggaacagatcatttgcaaggtggtgcgctcctatacgttaatagtgatttaacaccaaatgccaattctgattatcgtttattaaatcgaacgccaactaaccaaaaaggtcaaattacaacaaatggtaatcaaggtggctatgagatgctgttggccaacgatgttgataattctaacccaattgttcaagctgaacaattgaattggttatactacatgatgaatatcggtagcatcgcccaaaatgatccaacagcaaattttgacggttacagagttgatgctgttgataacgtgaatgctgacttgttgcaaattgctggagattattttaaagcagcatatggaacaaatcaaagtgacgctaatgcaaacaatcacatttccatcttagaagactgggacaataatgatccagcgtatgtaaaagcacaggggaacaaccaattaaccatggattttccaatgcatttggcattgaagtattcattgaatatgccaagtagtgctcgtagcggtttggaaccagcaatttcaacaagcctagtaaatcgtgcagcagacgccacagaaaatgaagcccaaccgaactattcatttattcgtgcacatgatagtgaagtacaaacggttattgctcaaattattaaagataaaattaatcctagttccgatggattgactgtctcaacagatgaaattgccaaagcattcgaaatatataatgctgacgaattaaaggctgataaagagtataccgcatataatataccctcatcatatgcattgatgttaactaacaaagatacaattcctcgtgtgtattatggtgatttgtttacggatgatggacaatatatgtctgcaaaatcaccatattatgatgcacttacttcattgcttcaatcgcgagtaaaatatgtttcaggtggtcaatctatgaatatgacttaccttcataataatcaaggccttttgacgtcagtccgctatggaaaagacgccatgacagctaacgacactggtacaagtgaaacgcgcacacaaggtattggattaattgtcggcaacaaaactgatttaaacctgaataatgatgagcaaattgtgcttaacatgggcgccgcacacaaaaatcaagcttaccgtgcattaatgttaagtactaaagatggcttgaaaatttataatagtgatgacgaggcaccggtatcgtatacagatgatcaaggccgtttgatttttaaatctgatgtggtttatggtgtgagtgatgctcaggtttctggttatttagcagcttgggtgccagtcggtgcaaacgatagccaagatgctagaacagaaagtagtacaacagcgtcaacagatggtaatacctatcattcaaatagtgccttagattctcaagttatatatgaaggtttttcaaacttccaagccatgccaacacaggccgatgagtataccaatatcaagattgccgaaaatgcacaattattcaagagccttgggataacaagctttgaattggcacctcaataccgttcaagtacggataacagtttcttagattcagttattcaaaatggctatgccttcacggatcgctacgacattggttataatacgcctacaaagtacggtacagttgaccaactattagatgctttaagagcattgcatgcccaaggcattcaggctatcaatgattgggtcccagaccaaatttataatttgcctggcgaagaaatagtgacagccagtcgaacaaacggttcgggaaaggtgaatgaaagttcagttattaataatacgctatatgattctcgtactgttggtggcggagagtatcaagcaatatatggaggtgctttcttagataagttaaaacaagattatcctgagttatttgaaacaaaacaaatttcaacaggtgaagcaatgaaccctgatgtcaaaatcacagaatggtcagctaagtattttaatggctcaaacattcaaggacgtggtgcatggtatgttctcaaggattggtcaacaaatcaatactttaatgtttcaagtggtagtgaatttttacctaagcaactgttaggcgaaaaaacaagtacagggtttaccaacgtggacaatggcaagactgagttttattctacgagtggctaccaagcaaagaatacatttattcaagataatgacaattggtattattttgataatgatggctatatggttgttggcggtcaagaaattaatggtaaaaaatattatttcctaccaaatggtgtagagttacaagatgcttatttgtctgatgggactagtgagtattactacagtagtgatggtcgtcaaatttctaatcaatattatcaaggatcagacaacaactggcgttatttctttgcagatggtcatatggctgtagggttagcaacaattactacagaaaatggtacaacaaatcaacaatatttcgatgcaaatggtatgcaacttaagggcgtagctataaaggatactgatggcaatgtgcactattttgatggtaagacaggaaacatggttataaattcctggggcaaaataagcgatggttcatggttatacttaaatgatagcggtgtagcggtcacaggaccgcaaaatattaacggtcaaaatctttacttcaacgaagacggtattcaagtaaagggtgaagccattactgataatagtggaaacatacattattatgatcgcagcacaggaaatatggttgtgaactcatggggtgaaacgaataatggttcatggctatacttgaacgacaagggtgatgccgttacaggagaacaagttattgacggtcaaaaactatatttcagtagtaatggaatccaacttaaaaatacattcaagaagctatccgatggttcatggctatatttgaacgataaaggtcttccagtgacaggagcacaggtcattgatggacaaaacttgtatttcgaccaagatgggaagcaagtcaaaggtgacgttgctacagatggacaaggtaacactcattattatgacggcaacacaggaaatatggttactaattcatgggcagagttagcggacggttcatggatgtatctagataatgatggcaatcctttaacaggaccgcaaaagattgatggccagtcactctactttaatgatgctggtaagcaaatcaaaaacgcattggttaaactagatgatgggtcaacaatttacctcgatgataaaggtgtttcatcaaccggtattcaaagaattgatgataagatatattattttgatcctgatggtaaacaagtagtatgtcgttttgaagaattaccagatggttcatggatgtatctagatgatgacggtgttgctgctacgggcgctcaaaaaattaatggccaggaattatatttcgacaataacgggaaacaagtcaaaaatgacaaagtaattaatgacgatggaacaataaactattacacaggtatgagcggtgaaaaactaaaaaatgattttggtgaattaccagacggttcatggatgtacttggataatcaaggtaatgctgtaataggtgcccaaaaaattaatggccagaatctttacttcaagacagacggacgacaggttaagggtgaagcaaatgttgattcatcgggtgaaatgcacttctatgatcctgattctggcgagctaattacaaatagatttgaacaagttgctagtggtgtatgggcttactttgatgccaacggtgttgctgtaactggtgagcaacgcattggtaagcaaaatttattttttgatccaactggttatcaagttaaaggcgacaaacgaacaattgacggcgttctctatacctttgataaagaaagtggtgagagaaagggtttagattctatatcggtattacccaccaatggacaatacacaaccgataaggcccaaaattggtattaccaagtcgatggtgaaaatgtaaaagggctatatacaaataatgatggtcaattacgttacttcgatttgacaactggcgtgcagactaaaggtaattttgtgacaattggcaatgatacctactatttcaccaaggaacaaggggatggacagatagtttctgaggttgtgtcaggacactatggtactgtccagttgagtgacaattcgtctgcatgggtttatcgcggtgcaaatgatcaaattttgaaaggcctacagaatataaacggtcgtctgcaatattttgatctaaccaccggtgcgcaattaaaaggcggtgctgcaaactatgatggcaacctttattattttgaatcatcagatggtaacctagtcagtaaaattcagcaatcttattctactgggaattatgtgaccgatggtgataaagtaacatatgttgatgagcaaaacaaccaagtcacgggattagcgttgattgatgatcaactacaatacttcaatccaagtgacggtagtcaagtcaagaatgagcaggttatcgttgatggcgtcacatactactttgataaaaatggtaatggacaatacttgtttacaaatactgcaacgatgtctactaatgaatttgccaaacatagtgctgcttatagcaatgatagttctagcttcaagaatacgatagatggtttcttgacggccgatacctggtatcgccctaaagatatcttggaaaacggacaaacgtgggtagtttcttcaacaaatgacgtgcgaccactgataacagtttggtggccaaataaagatgttcaagttaattatttaaattttatgaagaaaaatggtttgctagatacaagtagtcaatttaatctacaatttgatcaatatgacttgaatgtcgccgcgcaaaaagttcaagtggctattgaaaaacgcatttcgaaagaaaagagtacagattggttgaaagatcttttgtttgaagctcatgaagatacgccttcatttgtgaaacaacaatttatttggaataaagattctgaatatcaaggtcaaggggatgcgtggttccaaggtggttatctgaaatatgataatagtgaattaaccccaacaacgaactcagattatcgtgaatccggtaatacattagacttcttgcttgctaatgatgtcgacaattctaacccagcggttcaagctgaaaatttgaattggttacattatttaatgaactttggcacgattacagctaatgatgatgatgccaactttgacagtattcgtattgatgccgttgactttattgataatgatgccattcagagaacctacgattacatgcgtgatgcttataaagttgatgcaagtgaagacaacgctaataagcatatttcactagttgaagctggattagatgctggtacctctacaattaagaataatgctttagttgaatctaactttagagaggcagctacactatcgctagcaaatcaatcagggaaaaatagttccttgactaatatgttgcaagacattgatggtggccagattatagctgatcacgccaacaatgcaacagaaaatgaagcaacgccaaattattcaattattcatgctcatgataaggggattcaagaaaaggttggtgcagcaattaccgacgttactggtgcagactggacgaatttcaccgatgaccaattaaaagaaggattagcagcttattatcaagatcaacgttcaacgaataaaaagtataacatctataacttacctagtatctatgctttgatgttgaccaataaggacacagttcctcgtgtttattacggtgatatgtatcaagatgatggccagtatatggaaaagcaaagtatttattatgatgccattgtttccttgatgaacactagaaagagttatgtgagcggtggacaaaccatggatgtagatgaacatggtttgttgaagagcgttcgttttggtaaagacgcaatgacagctagtgaacttggtacgaatgaaacacgcactgaaggtgttggtgtgctggtcggtaatgattcttcactaaaactaaatgattcagatacagttactttagagatgggggcggctcataaaaaccaagagtaccgagctgcattgttgacaactagtgatggtattgttacgtatgatgctgataatgatgcaccaacgatctggacagatgaccggggtacattaacgttctcaaataaggagattgctggtcaagattatactagtgtgcaaggattcgctaatccacaagtatcaggttacttagcagtttgggtgcccgtaggagctagtgacgatcaagatgcccgaacagcagcatcaacagataaaaatactgatgacaaagtactgcattctaatgctgcattagattcgaacttgatttacgaaggtttttcgaactttcaacctaaagcaaccaccaatgatgaactgactaacgtagtaattgctaaaaatgctaatttatttgaaaagtggggaatcacaagttttgagatggcaccacaatatcgttcaagtggggaccacacgttcttagattcaacgattgataatggttatgtatttactgaccgatacgatttgggatttgaaacaccaactaagtacggtactgataaggatttgcgtactgcaattaaagcattgcaccaatcaaatatgcaggttatggctgatgtagttgataaccaagtttataatttatctggacaagaggtcgtatcagcttcacgtgctggtgtttacggcaatgatgtgtcaactggatttgggacacaactctatgcggttaatagtgttggtggtggtaaatatcaagcccaatacggtggtgaatatttgaatgaattgaagcaacaatacccagatttgttcgaagctaagacgtatgactattgggttaaaaattattcaaatgacggatcggatccgtattacacactgtcgcaaaacacacgaaaagatatgccaagtagtgaggtcattaaacaatggtcagctaaatatatgaatggtactaatgtattaggaaacggtatgggatatgttttgaaagattggaatacaggtgagtacttcaaaattggagaaaagaatgctgattttataacaaattaa

SEQ ID NO: 23 - DNA, 7740 bp, Lactobacillus kunkeei
  source 1..7740: mol_type = unassigned DNA
atgaataattatcaagatagaaaattgcattataaaatgtataaaagtggaaaaaactgggttgttgcaggtattgtttcagcaacaatgtctattgtattattccaaaatattaatgatgttacagcaaaagctagtcaaaatcagattacatattcaggtagctatgcttcgacagattccggtagtgctaaaagtgcatattcagcgtcatcttacataaacggatcaagttctttgtctgtacctagcactgataattcaaacaaatcaagcttagcgtcttcaacaaactctagtgttgtgacttcttcattaagttcaaatgttatgaatcaatcatctaattcacatgcgtcatcaataagttcagacgtttcaaatcaatcacaaagttcaaaatcagcatcaacgagttcaaattcgtcagaaaaatctgattcagcatcacagtctaataattctgtgcaatcatatgaatcggtttctttaagtgtatctttagcatccaatgcttcacaaaacttaaaatcgcatacaaagaacattgatggaaagacatatttttatgatttaaataataatgaagttaagtctgctttaattgacgataacaacacatactattattttggacccaatggatatctgactacatttgatgattcaaagttttcggatggctcaatcaactcatcagatcagttatctatttataccccagatggtaaatcaattacaaatgttgatggctttttaacagctgactcatggtatcgtcctaagcaaattcaagtaagcgatactcaatggagaaattctactaatgcagattttcgaccactattgagtgtgtggtggcctaacaagacagttgaaattaattacttaaattatatgagcaaaaatggtctagttaatggtcattttgataataatagtagtgcaaacgatatcaaccaagcagctgcaactgtaagattaagcattgaaaacaaaattaaagcagataatggtgatttaagtgcgattagatctttatttactactttcataaattctcaggatgaatggaatatcgacagtgaagactataattctggagatggtttacaaggtgggtcacttctttttggaaacaatagtgaaactaaagatgccaactcaaactacagattacttaatagaaatccgactcaacaagatggaaaaattgattatacacatacacaagatccaggctttgaatttttgttggctaacgacgttgataattctaatcctgtcgtacaagctgaaacactcaattggttgcattacctaatgaattttgggtcaattgttaacaaagatagttcagcaaattttgatggcgtgcgtgttgatgctgttgataatatggacgccgatgtgctaaatatcattagccaatattttagagatgcatacaaaattaacgccaatgacgttaattctaataatcatctttctattctagaagactggagtggaaatgacccatattaccaaaaagatcatggaacaaatcaaatgactcttgatagtgactatttaagctcattacgatcgattttaatgtacaatccttctgttagatctgatatgtcaactattattaatgctggtattgtggatagagctgatgacaatacaactaacaaagctattccaaattatgaaattgtaagggctcatgatgcaggtgttcaagacatcatttcacaaattattattgacaacattgatccccatgcttcagcttctaatccaacgtgggaacaaatgagagaagctttcaagatatacgatgctgatgaaaatagcactgtaaaaaaatacacacaatataacattcccgcatcatatgcattgacccttacaaataaagatacaaccccaagagtttactatggtgacctatacactgacgacggtcaatttatggaacaaaagactccatattatgatgctattgctgaaatgcttaaatcaagagttaagtatgtagctggtggacaacatatggaagcggttaaggttaataatggtgaagatacgattcttacttctgttagatatgggaaggatgcattaaatgcagatgacttaggtgatagtctaactagaacatctggaatagctgttgttgaaagtaataatcctactttgtcactttcggataaagacaaagttgttattcatatgggggcagcccataagaatcaagaatatcgtcaattattaacggcttcaaattcaggaattgattcttttgacagtgatagttcaaaaggttatgttgtcgttcgtacagatgataatggtgacttaacattagatggaaatatcattaagggatatgctaacccgcaagtatctggatacttatcaatgtgggtaccactaggggcttctgataatcaagacattagaactgctccttcttcagaatcaagtactgatggtcaatccattcattctaatgaagcatctgactcgaacgttatattagaagcattttctaactttcaaaattttccacaaacaactgatcaatatgaaaatgttgttattcaaaaaaatgcagaagattttaaaaatttaggttttacttatttagaattaccaccacaatataagtcaacaaaagatggttcgtttattgattcggttgtacaaaatggttattcatttaatgatagatatgacctaggatttgatacgccaactaagtatggtacggctgaacaattgattgatgcaattaaatctctacatgcacaaggaataaaagtacttgctgatatcgttccagatcagatatattcattgccaaaccaacaaattgtaaacgcaactagaacaaatccttatggagatgcaaactttaattcaaatcttattaatgttttgtatgatgcgtttagtaagggttctggaactgattatcaatataagtatggtggtgagttcttagaacaactaaaaaaattatatcctgacttatttaccacaaaacaaatttcaacaggacaaccaatcgatgcttctaaaaagttaaagatttggactgctgaatatttaaatggttcaaatatccaaggtcgaggttcggggtatgtattaagtagtacaccaaattctaattactacactgttgtagatgatggaaacacaaacataaagagttctaatctacccaaaggattattaggtgataatgttgaatatggtttaaaacttattgatggcaagatgaagtacgtttccaccggtggatttattgctaaaaatacttttattcaagatgataatcacaattggtactatgttgataataacggtgattttgttacttctccacaagttatcaatggaaataagtatttcttcctatcaaacggtgttaatttaagagacttcatttcagttaatagtgatggaacgatgaattattatcaaagtaatggaattttagcaaataaccccgggtattattacagtaataatataaatcaaatggtacatgttaataataacggtgttttagatactgggattgttaatgttaatggttatgttcaatattttgatgataatggttaccaagttaagggagacatagttaattttaatggtcgaaaaatgtattttgacgatggatctggtaatttagtttacaatcgatttgtttcttacaatggaaattggtactatgcaggttccaacgggctattggtaaacggattacaaaatataaataaccaaagtctttacttcgatgataatggaaaacaagttaaaggtggaatgatttcattagataatcataaaatgtattttgatgctaatagtggaaacctttataaaaatagttttgtattgttagatggagatgtctattatgcaaataatgatggatatattgtaaatggatatcaaaacataggcggaagaaatctatattttgattcagacggaaaacaagtaaaagaccaatttgttaatattaacgggaataaagtatattttaatggtactgatggttctgaagtaaaagacgattttattattcatgatgacaaagaatattatgcggacaatcaaggacatcttttgacgggatataattttgttaatgggcaaaatatgtatttcaatgaggatggatcgcaagtaaaaaattcgattgttagtttagatggtaaattaatgtattttgacgaaaattcaggtaaccaggttaagaatggctttattttacacaatgggaatgtttactattctaacaaagacggcattttggttactggatatcagaatatagacgggcaagatttgttttttaatgcagatggtactcaaattaaaggtggtactgctgaaatagatggtgttaattattattttgaaaatggagaaggacatttagttggcaaagttgaccaagtaattaactcaaatagatttagtgacaataagcttttggatgctaataataatgttgttaaaggattaatttttaataatggtcaattgcaatacttcgattcattaactggagatcaagctaaaaataaacaaattattgctgatggcaatacttattattttgataattctggtaatggaacttatttatttaccaatattggtgaaagtgaaacaaatgatttttcacaaaggaatgcagctaatagtgttaacctaagtgattacaaaaatgttgtagatggattctttacagcagatacttggtaccgtccaaaacaaatattagataatggtactacttggcgtaattcaaatagtaatgatttccgaccaatgattacagtatggtggccaaataaaaacgttcaagttaattatttaaagttaatgcaaaataataatctcttagataagagcaccaattatacattacagtccaatcaacaaatgttaaatcaagctgctcaaaatgcgcaggttaacattgaaaagaaaattgcacaaacgggaaatactgattggttaaatgatctgttatttaaaggaaatggagatacaccatcatttgtaaagcaacaatatatttggaatattttatctgaatcaccaggacaagatgatgctttgttacaaaatggatattttaaatatgttaacagcgagctaacaccaaataccaattcttcatatagaatatcaaataatttatgtgatttcttacttgctaatgatgtggataattccaatccagctgttcaggctgaagacttaaactggttatattatttaactaattttggaactattaccgccaatgatccaaatgcaaactttgacagtattagaattgatgcgtatgcctttataaataatgatattgtccaaagaagtaatgactacatgaaacaaaaatataaattaactgaaagttctaataacgcaaatagccatttgtcaattgtggaggcaggtgtcgatgctggaacgacatcaactaataatgatgagttagtggaatcaccttttagaacaatatcgtatggattaatacacaaagatagaaatcctcaagatatgaataactttataaaagaagtggatacaggtgtcgttattgccgatcatgaaaataattcacaagaaattggacagcctaactattctatcgttcatgcgcatgataaggatatacaagacaaagttggcgaagcgattgttgatgcaaccggaatcaaggactggacgaattttactccttctgaattatctgcaggtttgaaattatattatgatgatcaacgaagttctgaaaaaaaatataatgattataatatacctagcgcttacgcaattatgttgacaaataaaggtacagtcccacgaatatattatggtgatatgtatcaggatgatggacaatttatgcaaaaacaaagcgtttattatgacgatattgtttcactattgacagctagaaaaaaatacgtttctggtggtcaatcaatgtctgtagatcataacaatttcttagaaagtgtacgttttggtaagggagctggtagtgaaagcgattctggtaatgcagaaacgcgtaatgaaggaatcggcttgattgttggaaatgaccagaataagaaattaaatgatggagacactgttgttttgcatatgggtgctgctcatagaaatcaaaagtatcgtgcattaatgttaacgacaaatgatggtataaaaaattacgattctgatgaaaatgctcctattgccgaaacagatagtaatggagatcttgtttttagtaataaggatattaacggacaagccaacacaagtgttagaggtgttttaaacccagaagttgccggatatgttgcagcatgggttcctttaggagcatctgatgatcaggattcgagaacattatctagtaataaatcttataatgatggaaaagtacttcattcaggagatgatttagattctaatgttatttttgaagccttttcaaattttcaaccagaaccaacaaatgaaaatgaatatgagaatgtagtaatcccccaaaaagcttcattatttaaagactggggtattactagttttgaacttcctccacaatacaggtctagtaatgaccatacttttgttgatgcaaccattaataatggttatgcgttctctgatagatatgacctgggatttggtcaacccactaaatatggaactgatgtagatttaagaaacacaattaaatcattgcatgataacggaatgcaggtcatggcagatgtagtttataatcaactatataatttgccggggcaagaagttgtttctgctgtaagagcaggatttactggtaacacagtttcattgccatttggaaatcaattatatgttgtgaatactgttggtggtggtgactatcaaaagaaatatggtggagcatttttgcagaagctataccaagaatatccaagcttgtttgattcagaaaaatatcaatataatagtaagaattatgttactgatttacttgttatgacagatggagaacgttcggcgattccttctgatcaaccaatcacagaatggtctgccaagtacatgaatggaactaacattcttggacgtggtatgggatatgtattaaaagattggaacacaggaacttactttaaactgaatggaaacaattctgtgcttccggatgttttaacttatagaaatagttttgatgataatacaggaactcaatcaagctcaagtgcgcaaagcagtgcatcaaaccatgaaacacaaattagctcaagtgcccaaagcagtgcatcaaaccatgaagcacaaagtagctcaagtgcgcaaagcagtgtatcaagtcatgaagtacaaagtagctcaagtgcgcaaagcagtgcttcaagtcatgaagtacaaagtagctcaagtgcgcaaagcagtgcttcaagccatgaagtacaaagtagctcaagtgcgcaaagcagtgcatcaagttatgaagcacaaagtagctcaagtgcgcaaagcagtgcttcaagccatgaagtacaaagtagctcaagtgcgcaaagcagtgtatcaagtcatgaaacacaaagtagctcaagtgcgcaaagcagtgcttcaagccatgaagtacaaagtagctcaagtgcgcaaagcagtgcatcaagtaatgaagcacaaagtagctcaagtgcccaa

SEQ ID NO: 24 - DNA, 4392 bp, Lactobacillus kunkeei
  source 1..4392: mol_type = unassigned DNA
atgaatatcaatagtaatgaacgtaaggttcgatttaaaatgtataaatctggtaaacaatggattgtagctggattaactacagcagtaatatctattgctgtgtatggtggttcttcaattgccaatggtggaattgaagctaaagctgatgcacaaaacgcagctacttcttcaattgtgaatactaataattctacaaatagtagtaatgctaattcaattgcatctcttcctcaaaatggtacatattcaacaaatgataacggacaaacttggaagtatgtatcacaaaataaggacattcaaggtttatataaggataataatgatcagttaagatattttaacgaatatgatggcactcaagccaagggtgatattgtaaatgttaataatgacaattactattttgataaagacagtggtcagggacataagattgatagctacactgggggaagctactctgaaagtaaggtcaataaccaggatggttggatttataaatcaagtgataacaatgatgttaaaggagtcgctactgtcgacggtaatattcaatattttgatcaaaatacaggtctgcaattaaaaggtggatctgcgcaaattggtggagttgattactattttgacccaaacaagggtaatttagttggaaaagttgatcaagtagtaaactcaaatgattatagcgataataagttactggatagcaacaaaaatgtagttaaaggtttagtagtaaataatggtcagttacaattttttgatacatctaacggtaatcaggctaagaataagcaagtaatagctaatggaattacctactattttgatactaatggaaatggtcaatacttgtttactaatacaggtaagagtgctgttgatgactttacacaaagaaatgctgctaacagtgttaacccaagtgattacaaaaatgttgtagatggattttttacagcagatacttggtaccgtccaaaacaaatattagataatggtactacttggcgtaattcaaatagtaatgaactccgtcctatgattactgcttggtggccaaacaaggatgttcaagttaattatttgaaattaatgcaaaataacggactattagataaaagcaattcgtattcaatacaatctgatcaacagacattaaatcaagcagcacagaaagctcaagtaaatattgaaaagaaaatttcacaaacggggaatactgattggttaaatgatttgttatttaaaggaaacggcgataacccatcatttgtgaagcaacagtatatttggagttcagattcagaatcgccatggcaaggagatgcatggttccaaggtggatatttaaaatatggcaatagtgtcatgacaccaaatactaattctaattatagagattctaataacttgtttgatttcttgttagctaatgatgtagataattcaaaccctgcagttcaagcggaggacttaaattggctatactacttaactaacttcggaactattaccgctaatgattctaatgctaattttgatagtattagaattgatgccgttgattttattagtaacgatattatacaaagaagttatgattacttacgtcaaaagtttaatttaatgcaaagtgatgcaaatgctgattcgcatatttcgttagttgaaggtggagtggatgcaggaactacatcttatagcaatgacggattagttgaggcaccattcagactggatgcttatcctttgctacataagcaagatggagacgtatttaaaaacttaatagatgaagaagattctggaatcgacatttcaaatcataatggagaaaccaatacaaataatactattggtggaataactctatctggtggaaaacctaactattcaattgttcatgcccatgataaggatgttcaagaaaaagtaggtcaagctataatcgatacaactggaattaaggattggactgactttacaccaagtcaattagcacaaggattagaaactttctataacgatcaaagacaaacagtaaaaaaatacaatgattataatgtgcctagtgcatatgcaatcatgctaactaacaagggaacagttcctcgtatttattatggtgatatgtaccaagatgacggtcaatttatgcaaaagaagagtctttattatgatgatatagcaaacttgatgactgcgagaaagaaatatgtatctggtggtcaatctatggttgataataatggaattctaacaagcgtacgttttggtaaaggtgccaatacagttagtgattcaggaacagaagatacaaggaatcaaggtattggattaatagttggatctgcccccaaaaaggttttaaatgatggggataccgttgttttacacatgggtgcagcccataagaaccaaaaatatcgtgccttgatgctaactactgaaaatggaattcaaaattataattcagatgataatgcaccagttgctgaaacagatgataatggtgatttagttttcagtaacaaagatattaatggtcaagctaacactgcaattaaacaagttgccaatccagaggtgaatggttatctagcggcatgggttccggttggggcttccgatgaccaagattcaagaacagcaccatccactagtcaaaataatgatggtaatgttttgcacgaaaacgacgcactagattcaaatcttatttttgaaggattttctaacttccaaccaactccaacaaatcatgatgaatatgcaaacgttgtaattgctaagaatgcgtcactatttaaggattggggtgtaacaagtttcgaaatggcaccacaatacagatctagtcaagatcatacattcgttgattctacaatcgataacggttacgccttttcagatagatatgacttgggatttggtacaccaactaaatatggaacagatgaagacttaagaaatgcaataaaatctcttcatgataatggtatgcaagtaatggctgatgtagtatacaaccaattatataatttgccaggacaagaagttgtatctgcaaccagagcaggtgttactggtaatacaaatgcattaccttttggtactcaattgtatgtagttaatacaatcggtggtggagattatcaaaagaaatatggtggagcattcttaaatgaactgcaagaacaatatccatcattattcaaatctcaaaagtacaagtattactacaaaaattatgctaataatggtgctggaccaggttatttaacagttaatgatgcagaacgttcggatattccatataatcaaccaatcactgaatggtcagctaagtatatgaatggaactaatattcttggacgtggtatgggttatgtattaaaggactggaatacaggtgattatttcaagttaagcgggagcgattcaacactaccaagtagtttaacttacaagagcggttgggttgaaaatcctgatagtacttggtcatactatgaaaagaataatattgacaaattaactggatctcaagttattaatgaggaaagagttttctttgataacaacggtattcaagttaaaggaggatgggtaaaaaattcaaatggtacctattcttactatgacaagaatagtggaaacatcctaactggtgatcaattaatcgacggtgaacatttcttctttgataacaatggtgttcaagtaaaaggaaaatggataaagaatagtgatgggtctaagtcatattacgattctcacttaggaaaacttataaagacggataaaaaagtatcttctaatgctcgaaaaaagaaatctaaggaagaattattgtatgagaatgccttaaaagttttaagaaaagataagaaacgtctcgataaaaataagacaaaggcaaatatcagaaaatataataaatctctaaaaaaatatcgtaaagcaaagaagaaacttttagcgattactaagaatagagttgctaatgctagaaaagcgattaaaattgctaaaaaagttctttctaagagaaaaaatattaataatgaaaaaagatattataaagctctaaaagaatattatgtagcggaaaaatcatatttgaaaattactggaaattacaataagaagtattattacgaatttgataaacttactccaaaagtcaaagtggttaagaacatttattcctataagagtagacattttactaaaaagaatagagttaaaaagattaaaaaaggtactcttgttagagttaaaagtatagttcgaagtggtaaagtcgctcgaattaatatcggaaatggtcattttataacttcatctaaagattttattaagatgtttaaataa

SEQ ID NO: 25 - DNA, 8475 bp, Leuconostoc carnosum
  source 1..8475: mol_type = unassigned DNA
gtgacagcaggaattttctctacggttgtgtttggtatggccgtatctgacgttagtgctaatgacactgacaacactgtgttaacttcgaacagtggatttttagataaagtagtagatacgacaagtacggataaggcagccacaccagataaagtagtagatacgacaagtacggataaggcagtcacaccagataaagtagcagatacgacaagtacggataaggcagtcacaccagataaagtagcagatacgacaagtacggataaggcagccacaccggataaagtagtagatacgacaagtacggataaggcagccacaccggataaagtagtagatacgacaagtacggataaggcagccacaccggataaagtagtagatacgacaagtacggataaggcagccacaccagataaagtagtagatacgacgagtacggataaggcagccacaccggataaagtagcagatacaacgagtacggataaggcagccacaccagataaagtagtagatacgacgagtacagataaggcagccacaccagataaagtagtagatacgacgagtacagataaggcagccacaccagataaagtagtagatacgacaagtacagataaggcagccacaccggataaagtggtagatacgacaagtacggataaggcagcagatagaaccattagtatttctggtaaaacagttaaaaatattgaggaaattggtgggaaaacttattttgtaggggatgatggcaaagtcaaaaaaaacttcacggttattgttgatgggcaagtaatgtattttgacaaagagtctggagcattgacttcgaaccataagcaatataaagaaggtctttcagatataacaaatgaacataatgctgcctattcattagaaaacgacaactttacacaaattgatagttacttaactgccaatagttggtaccgtccaaaagatatattaaaaagtggcacaacatggacagcctcaaccgataaagactctcgcccattacttatgtcatggtggccagatcaacaaacagaactatcttatttaaagtatatgcaatcagcaggtttcttagcagaggatgttaatttatcagaaaataatagcattgatgatctaacagctgcagcaatggatgttcagaaaaatgttgaagcaaagattagcctatcaggtaatacagattggttgaaagaagatatgaatcagtttgttgattcacagtcaaattggaatatcagcagtgagtcaaaaggcactgatcatcttcagggcggcgctttattgtatggtaacagtgatatgacaccagatgctaattctgattatcggttactgaatcgcacaccaaaaaaccaaaccggacaaatcagtgctacgaatgatcagggtggttatgagatgttattggctaatgatgtcgataattctaatccaattgttcaagccgaacaattaaattggttacactacatgatgaacattggtagtattacaaaaaatgattcaacggctaattttgatggttatagagttgatgcggtagataacgtgaacgctgatttgttacaaattgctggtgactacttcaaggccgcttatgggacggacaaaagtgatgcaaatgctaataatcatatttctattcttgaagattgggataatagtgatcctgactacgtcaaaaagcacgggaatgaacaattaactatggattttccgatgcatttggcattaaaatatgctttaaatatgcctattgatatgcgtagtgggcttgaaccggcaattaagacaagtctggtgaaccgatcacaagatgcaacagaaaatgaggcacagccaaactactcgtttatacgtgcccatgatagtgaagtacagactgttattgctcaaattattaaagataaaattaatccaaagtcagatggtttgacagttacacctgatgaaattgcgaaagcttttgaaatctataatgcagatgaactaaaagctgataaggcatacacagcttttaatatcccgtcatcttatgcacttttgttgactaataaagatacggtgcctcgtgtttactatggcgatttatttacagatgatggacaatatatgtctgatcattcaccatattatgatgctattacgactttattagcgtcacgtattaaatatgcggctggtggtcaaagcatgggtatgacgtatttacacgataatcaagaagttttgacatctgtacgttatggtaaaggggctctgacagcagatgatttgggtaatgttgacacacgtacacaaggtattggacttgttatcagtaataaaacggatttgagtttaaagagtgatgagagcgttgtgctgaatatgggtgtcgcacataaaaatcaagcatatcgcccggcaatgttgactactaagtcaggattaaaaatttacgatactgatgatggtgcaccaattgtttatacaaataatttaggacagctaatttttaacgcagatacagtctatggtgtgagtgatccacaagtttcagggtaccttgctgcgtgggttcctgttggcgcaactgaagatcaagatgctagaactaaaggcagtcatgatggaacaactgatgggaatgtctatcattccaacgctgcattagattctcaggtcatatatgaaggtttctctaatttccaggcaatgccaacgaccactgatgaatatactaacgtaaagattgcacaaaatgcacagtggtttaaaaaattaggactgacaagttttgaactggcaccgcaatatcgttctagcacagatagtagctttttagactctgttattcaaaatggatacgcttttactgatcgatatgatgtgggttataatacaccaacaaaatatggtacagtcgatcaattattagatgcattgagagcattacattcacaagatatccaagcaattaatgattgggtacctgatcaaatttataacttacccggggagcaaattgtaacagcgagtcgtaccaatggatccggaaaatacgatgatgattcagtgattagtaatactttatatgattcacgtactatcggtggtggagaatatcaagcgatgtatggcggggctttcttagatcaattgaagcaagcatatcctggtttgtttgaaacaaaacagctttctactggtgttgcaatggatcctgatgtaaaaataaaagaatggtctgctaagtattttaatggttctaacattcaagggcgtggtgcttggtatgtactaaaagactgggcaactaataagtacttcagtgtctcgagtaataatacatttttacctaagcaattattgggtgaaaaagctagtacaggatttattacgaacgatggaaaaacagaattttattcaacaagtggctaccaggcaaagaatacatttattgaagacaacggtaactggtattacttcgataatgatggttatagcgtagttggaaagcaagtgattgataataaacactactatttcttgcctaatggtgtcgaattgcaagatgcctatttatcagatggggataagcaatattattataaaaaaactggtcgacaaatagtaaatcagtattatagagatgagcaaggtgattggaggtatttctttgcagatgggcatatggctcttgggttaactgacattgtttcaaatgatggaactcatgcgacacagtattttgataacaatggtgttcaagtgaaaggaacatcagaacgagataaagatggtaatattcactattttgatggtacatctggtaatttagttgttagttcatggggacaattgtcggacggatcatggctttacctgaatgataaaggcattgctgttacgggtgctcaacaaattgatggtcaatcactatactttaatgaagatggtaaagaagtaaagggtgatgcggtaacagataatcaagggaatatacgttattttgatggtgaatctggtcacatggtggttaattcatggggtaaattacctgatggttcatggatgtatttgaatgataagggtattgctgttaccgggcaacaaaaaatcaacaatgaagtcttatatttcaatgctgatggtaagcaaatcaaaagtgcatttaaagaactagtggacggatcatggctttacctgaatgataaaggcattgctgttacgggtgctcaacaaattgatggtcaatcactatactttaatgaagatggtaaagaagtaaagggtgatgcggtaacagataatcaagggaatatacgttattttgatggtgaatctggtcacatggtggttaattcatggggtaaattacccgatggttcatggatgtatttgaatgataagggtattgctgttaccgggcaacaaaaaatcaacaatgaaatcttatattttgatgctgatggtaagcagttaaagaatacactgaaaacattatccgatggttcacgtatttatttggatggtaaaggtgtttctgcaacaggtgttcaaaaaattaatggtaaagtgtcatactttgatgttaatggcaaacaagttagcaatcatattcaagaactccctgatggttcatggatgtacctagataatgatggattagctctaattggtaatcaagacgttgatggcaaacaactttactttgatgttgatggtaagcaaataaaaaatgacaaagtcaaaaatagtgatggaacaattaattattacacgggtactgttggtgaaaaactaaagcatgattttggtcagttatccgatggttcgtggatgtatttagatgaaaatggtaatgctgttactggtgaacaaaatattaatgggcaacatttatactttaaagatgatggacagcaagttaaaggagatgtttttgaggatgacttaggccgtatgcgttattatagtgctaactcaggtgagatggttgtcaatcaatttgaacaaatttctgatggtgcctgggcttactttggtgatgatggtgtggcggtaactggtgaacaacatatcaatggacaagatcttttctttgatgcgacaggtcaacaggttaaaggtgaaagtcgaaccattaatggtattccttatacctttgaaaaggagagtggtgagaaacggtctgttaatatagcaccattactagccatgggaaattatgttacaaataatggtacagattggcaatatgaagtgcaaggcaatcctgttaaagggttatatagcacttctgataataagttgcgttattttgatctgacaacaggtgtacagattaaaggcaactttgttactattggacataacacatactatttcaatccagctaatggagacggtgaattattgccagatgtttctgatggccattatggtacgatccaagttaaagatgcgaatacaaatgaaaagacagtttgggtttaccgtaatcaaagtaatactattttgaaaggtattcaaaatattcatggtaatatacaatactttgacttatcaactggggagcaaataaagggtggtattgcaaactatgatggcaatgattattactttgaatcagcaaagggtaacctcacaagtaaaattaagcaagtctatacagatggtcaatatgtaactaaagatggaaaaagcatttatgaagacgcacaacaacaaagtgtcagtgggttagtgtctattaatggtcagttacaatacttcaatccacaagatggcgtacaagttaaaaatcaacaaattattgttgatggcgttacttactactttgatgaaaatggcaatggtcagtatttgtttacgaatacaacggtcatgccaatggatgattttacaaagcacaatacagtttatagtgataacgacaacaactttaaaaataatgttgatgggttcttaactgctgatacatggtatcgtccaaaggaaatcttaaaagcaggaacaacttggacaacaacatcggaaagtgacatgagaccactgatcacgacatggtggccaaataaaaatgtgcaagttaactatctaaactttatgaaacaaaataatctattgcacacaaatgtggagtatagcttgctatctgaccaatatgatttgaatattgcagctcaagcagtacaaacagctattgaaaagcgaattgctcaagaaaatagcactgattggttgcaaaatcttctattcacagctcaagacgatcaaccatcttttgttaagcaacagtttatttggaataaagattctgaataccaaggaaaaggtgatgcttggttccagggtggttacctgaaatacgggaacaataaattgacacctaataccaactcaaattatcgtaaaactgacaacgcgtttgaatttttgttagcaaatgatatcgataattcgaatccggtcgttcaagctgaaaatcttaattggttagaatatctgatgaattttggtactataactggtaaggatgacgatgcaaactttgatagtattcgtgttgatgctgttgattttattagtaatgatacaattcaacgaacatatgattatttacgtgatgcctatcaagtagatcagagtgaagccaaggctaatcaacatatttcattagtagaggctggattagatgcaggaacatcaacagttaaaaatgatgcgcttattgaatctaatttgcgtgaagcggctacgttatctttggccaatgcatctggtaaaaatagtgcgttaaccaatatgttgcaagatgttgatggtggaacattgattgcagatcatacccataattcaaccgaaaatgaagccacaccaaattattcaattattcatgctcatgacaaaggtattcaagagaaagtgggggctgcaatttccgatgctactggggccgactggactaattttacagatacacaattaaaatctggtttggatctttattataaggatcaacgtgctacggataagaaatataatatttataacttgccaagtatttatgcattaatgctcaccaataaagacaccgtaccacgtgtttattatggtgatatgtatcaagataatggtcagtatatggcagagaaaagtatctattataatgctttagaatcattaatgtcagcacgtaaaagctatgtaagtggtggacaaactatggatgttgatagccacggtttgctaaaaagtgttcgttttggtaaaggggctatgactgctgacacggtaggtaatgaagaaacaagaactgaaggtatcggtgtgttagttggtaacgatgcttcattgaaactcaatgattcagatacagtgaccttagatatgggcgcagcacacaaaaatcaaaagtatcgtgcagcaattttgactactaataatggtctatcaacgttcgattctgataaagatgcaccgattgcttggactaatgataaggggatactaacattttcaaataaaaatgttagtggacaagataatactaatgtccatggtgttgctaatccacaggtatctggttatttggcagtatgggttccagttggtgctaaagatgatcaaaatgctgggacaagcgcatctacagtagtcaacacagatggtaaagtattacactctaatgcatcattagattccaacttaatttttgaaggattctctaacttccaacctagagcgacaacaaatgacgaattaaccaatgtcgttatagctaaaaatgccgatttattccagaaatggggtattactagctttgaaatggcaccgcaatatcgctcaagtcaggaccacacatttttagattcaaccattgataacggttatgccttcacagatcgttatgacttaggatttaacacaccaacgaaatatggaacagatagtgatttgcgattggcaatcaaatcattacataaagctggcatgcaagtaatggctgatgttgttgataatcaagtctataacttaccagaccaagaagttgtttctgcttcacgcgcaggtgtatatggtaatgatgtcgcgaccggatttgatacacaattgtatgctgttaattctgttggcggcggtaaatatcaagcacagtatggtggacagtatttgagtgagttaaagaataagtatccagatttgtttgaagctaaagcttatgattattggacaaaaaattatgctaatgatggatcaaacccgtactacacattatcacaacagactcgagatgatataccatcagacgagaaaattaagcaatggtcagcgaaatatatgaacgggaccaatgttttgggacatggtatgggttacgtacttaaggattggaatacgggtcaatattttaagattaataaagatggcgattccaacttaccagtttaa

SEQ ID NO: 26 - DNA, 8436 bp, Leuconostoc mesenteroides
  source 1..8436: mol_type = unassigned DNA
atgagagacaaaagaataatatgcgatcgtaaaaaattgtataaatctgggaaactgttagtgacagccggtattttttctgctgtgatatttggcgtttccacaactaacgtaagcgctgatagcactaacaacactggtgttacggtgtcacaagcaacagataaagtagcagacacgacggctacaacggacaaagtagcggacacaacagctacaacagataaagtggccgacacagcagctacaacggacaaagtagcggacacaacagctacaacagataaagtggccgacacagcagctacaacggacaaagtagcggacacaacagctacaacagataaagtggccgacacgacggctacaacggacaaagtagcggacacaacagctacaacagataaagtggccgacacagcagctgcaacagataaagcagccgatacagcggctacaacggataaagtggccgacacagcagctgcaacggataaagtagcggacacagtagctgcaacggacaaagtagcggacacaacagctacaacagataaagcagccgatacagcggctacaacggataaagtaaccgacacaacagctgcaacggataaagcagcggacacgacggctacaacagataaagtggccgacacaacagccacaacatcagaaaaatcaaaaagtattaaacaaattgatggtaaaacttatttcattggtaatgatggtcagcctaagaaaaattttacagccattgttgacggtcaagtattatatttcgacaaagatactggtgctttgacatcaaatagtagtcaatataccgatggtttagccaatataggaaatgagcataatgcggcttattcattgtcttcggatagttttacacaagttgatggctatctaacggctaacagttggtaccgacctaaagatatattaaaaaatggtacaacttggacggctgcaacagcaaacgattttcgaccattgttaatgtcttggtggccagataaagatacccaagtttcatacttaaaatatatgcaatctgcgggattattatcagatgacgttgcattatcaaacaatgatagtatgaacagtttgacggatacggctatgactgttcaaaaaaaaattgaagaaaaaattggcttattgggcagtactgactggcttaaggccgatatgaaccaaatggttgattcacaatcgaattggaacattagtagtgagtctaaaggaacagatcatttgcaaggtggtgcgctcctatacgttaatagtgatttaacaccaaatgccaattctgattatcgtttattaaatcgaacgccaactaaccaaaaaggtcaaattacaacaaatggtaatcaaggtggctatgagatgctgttggccaacgatgttgataattctaacccaattgttcaagctgaacaattaaattggttatactacatgatgaatatcggtagcatcgcccaaaatgatccaacagcaaattttgacggttacagagttgatgctgttgataacgtgaatgctgacttgttgcaaattgctggagattattttaaagcagcatatggaacaaatcaaagtgacgctaatgcaaacaatcacatttccatcttagaagactgggacaataatgatccagcgtatgtaaaagcacaggggaacaaccaattaaccatggattttccaatgcatttggcattgaagtattcattgaatatgccaagtagtgctcgtagcggtttggaaccagcaatttcaacaagcctagtaaatcgtgcagcagacgccacagaaaatgaagcccaaccgaactattcatttattcgtgcacatgatagtgaagtacaaacggttattgctcaaattattaaagataaaattaatcctagttccgatggattgactgtctcaacagatgaaattgccaaagcattcgaaatatataatgctgacgaattaaaggctgataaagagtataccgcatataatataccctcatcatatgcattgatgttaactaacaaagatacaattcctcgtgtgtattatggtgatttgtttacggatgatggacaatatatgtctgcaaaatcaccatattatgatgcacttacttcattgcttcaatcgcgagtaaaatatgtttcaggtggtcaatctatgaatatgacttaccttcataataatcaaggccttttgacgtcagtccgctatggaaaagacgccatgacagctaacgacactggtacaagtgaaacgcgcacacaaggtattggattaattgtcggcaacaaaactgatttaaacctgaataatgatgagcaaattgtacttaacatgggggccgcacacaaaaatcaagcttaccgtgcattaatgttaagtactaaagatggcttgaaaatttataataatgatgacgaggcaccggtatcgtatacagatgaccaaggccgtttgatttttaaatctgatgtggtttatggtgtgagtgatgctcaggtttctggttatttagcagcttgggtgccagtcggtgcaaacgatagccaagatgctagaacagaaagtagtacaacagcgtcaacagatggtaatacctatcattcaaatagtgccttagattctcaacttatatatgaaggcttttcaaacttccaagccatgccaacacaggccgatgagtataccaatatcaagattgccgaaaatgcacaattattcaagagccttgggataacaagctttgaattggcacctcaataccgttcaagtacggataacagtttcttagattcagttattcaaaatggctatgccttcacggatcgctacgacattggttataatacgcctacaaagtacggtacagttgaccaactattagatgctttaagagcattgcatgctcaaggcattcaggctatcaatgattgggtcccagaccaaatttataatttgcctggggaagaaatagtgacagccagtcgaacaaacggttcgggaaaggtgaatgaaagttcagttattaataatacgctatatgattctcgtactgttggtggcggagagtatcaagcaatatatggaggtgctttcttagataagttaaaacaagattatcctgagttatttgaaacaaaacaaatttcaacaggtgaagcaatgaaccctgatgtcaaaatcacagaatggtcagctaagtattttaatggctcaaacattcaaggacgtggtgcatggtatgttctcaaggattggtcaacaaatcaatactttaatgtttcaagtggtagtgaatttttacctaagcaactgttaggcgaaaaaacaagtacagggtttaccaacgtggacaatggcaagactgagttttattctacgagtggctaccaagcaaagaatacatttattcaagataatgacaattggtattattttgataatgatggctatatggttgttggcggtcaagaaattaatggtaaaaaatattatttcctaccaaatggtgtagagttacaagatgcttatttgtctgatgggactagtgagtattactacagtagtgatggtcgtcaaatttctaatcaatattatcaaggatcagacaacaactggcgttatttctttgcagatggtcatatggctgtagggttagcaacaattactacagaaaatggtacaacaaatcaacaatatttcgatgcaaatggtgtgcaacttaagggcgtagctataaaggatactgatggcaatgtgcactattttgatggcaagacaggaaacatggttataaattcctggggtaaaataagcgatggttcatggttatacttaaatgatagcggtgtagcggtcacaggaccgcaaaatattaacggccaaaatctttacttcaacgaagacggtattcaagtaaagggtgaagccattactgataatagtggaaacatacattattatgatcgcagcacaggaaatatggttgtgaactcatggggtgaaacgaataatggttcatggctatacttgaacgacaagggtgatgccgttacaggagaacaagttattgacggtcaaaaactatatttcagtagtaatggaatccaacttaaaaatacattcaagaagctatccgatggttcatggctatatttgaacgataaaggtcttccagtgacaggagcacaggtcattgatggacaaaacttgtatttcgaccaagatgggaagcaagtcaagggtgacgttgctacagatggacaaggtaacactcattattatgatggcaacacaggaaatatggttactaattcatgggcagagttaccggacggttcatggatgtatctagacaatgatggcaatcctttaacaggacagcaaaagattgatggccagtcactctactttaatgatgctggtaagcaaatcaaaaacgcattggttaaactagatgatgggtcaacaatttacctcgatgataaaggtgtttcatcaactggtattcaaagaattgatgataagatatattattttgatcctgatggtaaacaagtagtatgtcgttttgaagaattaccagatggttcatggatgtatctagatgatgacggtgttgctgctacgggcgctcaaaaaattaatggccaggaattatatttcgacaatagcgggaaacaagtcaagaacgacaaagtaattaatgacgatggaacaataaactattacacaggtatgagcggtgaaaaactaaaaaatgattttggtgaattaccagacggttcatggatgtacttggataatcaaggtaatgctgtaataggtgcccaaaaaattaatggccagaatctttacttcaagacagacggacgacaggttaagggtgaagcaaatgttgattcatcaggtgaaatgcacttctatgatcctgattctggcgagctaattacaaatagatttgaacaagttgctagtggtgtatgggcttactttgatgccaaaggtgttgctgtaactggtgagcaacgcattggtaagcaaaatttattttttgatccaactggttatcaagttaaaggcgacaaacgaacaattgacggcgttctctatacctttgataaagaaagtggtgagagaaagggtttagattctatatcggtattacccaccaatggacaatacacaaccgataaggcccaaaattggtattaccaagtcgatggtgaaaatgtaaaagggctatatacaaataatgatggtcaattacgttacttcgatttgacaactggcgtgcagactaaaggtaattttgtgacaattggcaatgatacctactatttcaccaaggaacaaggggatggacagatagtttctgaggttgtgtcaggacactatggtactgtccagttgagtgacaattcgtctgcatgggtttatcgcggtgcaaatgatcaaattttgaaaggcctacagaatataaacggtcgtctgcaatattttgatctaaccaccggtgcgcaattaaaaggcggtgctgcaaactatgatggcaacctttattattttgaatcatcagatggtaacctagtcagtaaaattcagcaatcttattctactgggaattatgtgaccgatggtgataaagtaacatatgctgatgagcaaaacaaccaagtcacgggattagcgttgattgatgatcaactacaatacttcgatccaagtgacggtcgtcaagtcaagaatgagcaggttatcgttgatggcgtcacatactactttgataaaaatggtaatggacaatacttgtttacaaatactgcaacgatgtcaactaatgaatttgccaaacatagtgctgcttatagcaatgatagttctagcttcaagaatacgatagatggtttcttgacggccgatacctggtatcgccctaaagatatcttggaaaacggacaaacgtgggtagtttcttcaacaaatgacgtgcgaccactgataacagtttggtggctaaataaagatgttcaagttaattattcaaattttatgaagcaaaatggtttgctagatacaagtagtcaatttaatctacaatctgatcaatatgacttgaatgtcgccgcgcaaaaagttcaagtggctattgaaaaacgcatttcgaaagaaaagagtacagattggttgaaagatcttttgtttgaagctcatgaagatacgccttcatttgtgaaacaacaatttatttggaataaagattctgaatatcaaggtcaaggggatgcgtggttccaaggtggttatctgaaatatggtaacaatgaattaaccccaacaacgaactcagattatcgtgaatccggtaatacattagacttcttgcttgctaatgatgtcgacaattctaacccagcggttcaagctgaaaatttgaattggttacattatttaatgaactttggcacgattacagctaatgatgatgatgctaattttgacagtattcgtattgatgccgttgactttattgataatgatgccattcagagaacctacgattacatgcgtgatgcttataaagttgatgcaagtgaagacaacgctaataagcatatttcactagtggaagctggattagatgctggtacctctacaattaagagtgatgctttagttgaatctaactttagagaggcagctacactatcgctagcaaatcaatcaggggaaaatagttccttgactaatatgttgcaagacattgatggtggccagattatagctgatcacgccaacaatgcaacagaaaatgaatcaacgccaaattattcaattattcatgctcatgataaggggattcaagaaaaggttggtgcagcaattaccgacgttactggtgcagactggacgaatttcaccgatgaccaattaaaagaaggattagcagcttattatcaagatcaacgttcaacgaataaaaagtataacatctataacttacctagtatctatgctttaatgttgaccaataaggacacagttcctcgtgtttattacggtgatatgtatcaagatgatggccagtatatggaaaagcaaagtatttattatgatgccattgtttccttgatgaacactagaaagagttatgtgagtggtgggcaaactatggatgtagatgaacatggtttgttgaagagtgttcgttttggtaaagacgcaatgacagctagtgaccttggtacgaatgaaacacgcactgaaggtgttggtgtgctggtcggcaatgattcttcactaaaactaaatgattcagatacagttactttagagatgggggcggctcataaaaaccaaaagtaccgagctgcattgttgacaactagtgatggtattgttacgtatgatgctgataatgatgcaccaacaatctggacagatgaccgtggtacattgacgttctcaaataaggagattgctggtcaagattatactagtgtgcaaggattcgctaattcacaagtatcaggttacttagcagtttgggtgcccgtaggagctagtgacgatcaagatgtccgaacagcagcattaacagatgcaaatcttgatgacaaagtactgcattctaatgctgcattagattcgaacttgatttacgaaggcttttctaactttcaacctaaagcaaccaccaatgatgaattgactaacgtagtaattgctaaaaatgctaatttatttgaaaagtggggaatcacaagttttgagatggcaccacaatatcgttcaagtggggaccacacgttcttagattcaacgattgataatggttatgcatttactgaccgatacgatttgggatttgaaacaccaactaagtacggtactgataaggatttgcgtactgcaattaaagcattgcaccaatcaaatatgcaggttatggctgatgtagttgataaccaagtttataatttatctggacaggaggtcgtatcagcttcacgtgccggtgtttacggcaatgatgtgtcaactggatttgggacacaactctatgcggttaatagtgttggtggtggtaaatatcaagcccaatacggtggtgaatatttgaatgaattgaagcaacaatacccagatttgttcgaagctaagacgtatgactattgggttaaaaattattcaaatgacggatcggatccgtattacacactgtcgcaaaacacacgaaaagatatgccaagtagtgaggtcattaaacaatggtcagctaaatatatgaatggtactaatgtattaggaaacggtatgggatatgttttgaaagattggaatacaggtgagtatttcaaaattggagaaaagaatgctgattttataacaaattaa

SEQ ID NO: 27 - protein, 1107 aa, Leuconostoc mesenteroides
VKGDKRTIDGVLYTFDKESGERKGLDSISVLPTNGQYTTDKAQNWYYQVDGENVKGLYTNNDGQLRYFDLTTGVQTKGNFVTIGNDTYYFTKEQGDGQIVSEVVSGHYGTVQLSDNSSAWVYRGANDQILKGLQNINGRLQYFDLTTGAQLKGGAANYDGNLYYFESSDGNLVSKIQQSYSTGNYVTDGDKVTYADEQNNQVTGLALIDDQLQYFDPSDGRQVKNEQVIVDGVTYYFDKNGNGQYLFTNTATMSTNEFAKHSAAYSNDSSSFKNTIDGFLTADTWYRPKDILENGQTWVVSSTNDVRPLITVWWLNKDVQVNYSNFMKQNGLLDTSSQFNLQSDQYDLNVAAQKVQVAIEKRISKEKSTDWLKDLLFEAHEDTPSFVKQQFIWNKDSEYQGQGDAWFQGGYLKYGNNELTPTTNSDYRESGNTLDFLLANDVDNSNPAVQAENLNWLHYLMNFGTITANDDDANFDSIRIDAVDFIDNDAIQRTYDYMRDAYKVDASEDNANKHISLVEAGLDAGTSTIKSDALVESNFREAATLSLANQSGENSSLTNMLQDIDGGQIIADHANNATENESTPNYSIIHAHDKGIQEKVGAAITDVTGADWTNFTDDQLKEGLAAYYQDQRSTNKKYNIYNLPSIYALMLTNKDTVPRVYYGDMYQDDGQYMEKQSIYYDAIVSLMNTRKSYVSGGQTMDVDEHGLLKSVRFGKDAMTASDLGTNETRTEGVGVLVGNDSSLKLNDSDTVTLEMGAAHKNQKYRAALLTTSDGIVTYDADNDAPTIWTDDRGTLTFSNKEIAGQDYTSVQGFANSQVSGYLAVWVPVGASDDQDVRTAALTDANLDDKVLHSNAALDSNLIYEGFSNFQPKATTNDELTNVVIAKNANLFEKWGITSFEMAPQYRSSGDHTFLDSTIDNGYAFTDRYDLGFETPTKYGTDKDLRTAIKALHQSNMQVMADVVDNQVYNLSGQEVVSASRAGVYGNDVSTGFGTQLYAVNSVGGGKYQAQYGGEYLNELKQQYPDLFEAKTYDYWVKNYSNDGSDPYYTLSQNTRKDMPSSEVIKQWSAKYMNGTNVLGNGMGYVLKDWNTGEYFKIGEKNADFITN

SEQ ID NO: 28 - protein, 856 aa, Leuconostoc mesenteroides
TMSTNEFAKHSAAYSNDSSSFKNTIDGFLTADTWYRPKDILENGQTWVVSSTNDVRPLITVWWLNKDVQVNYSNFMKQNGLLDTSSQFNLQSDQYDLNVAAQKVQVAIEKRISKEKSTDWLKDLLFEAHEDTPSFVKQQFIWNKDSEYQGQGDAWFQGGYLKYGNNELTPTTNSDYRESGNTLDFLLANDVDNSNPAVQAENLNWLHYLMNFGTITANDDDANFDSIRIDAVDFIDNDAIQRTYDYMRDAYKVDASEDNANKHISLVEAGLDAGTSTIKSDALVESNFREAATLSLANQSGENSSLTNMLQDIDGGQIIADHANNATENESTPNYSIIHAHDKGIQEKVGAAITDVTGADWTNFTDDQLKEGLAAYYQDQRSTNKKYNIYNLPSIYALMLTNKDTVPRVYYGDMYQDDGQYMEKQSIYYDAIVSLMNTRKSYVSGGQTMDVDEHGLLKSVRFGKDAMTASDLGTNETRTEGVGVLVGNDSSLKLNDSDTVTLEMGAAHKNQKYRAALLTTSDGIVTYDADNDAPTIWTDDRGTLTFSNKEIAGQDYTSVQGFANSQVSGYLAVWVPVGASDDQDVRTAALTDANLDDKVLHSNAALDSNLIYEGFSNFQPKATTNDELTNVVIAKNANLFEKWGITSFEMAPQYRSSGDHTFLDSTIDNGYAFTDRYDLGFETPTKYGTDKDLRTAIKALHQSNMQVMADVVDNQVYNLSGQEVVSASRAGVYGNDVSTGFGTQLYAVNSVGGGKYQAQYGGEYLNELKQQYPDLFEAKTYDYWVKNYSNDGSDPYYTLSQNTRKDMPSSEVIKQWSAKYMNGTNVLGNGMGYVLKDWNTGEYFKIGEKNADFITN